(19) United States
(12) Patent Application Publication
(10) Pub. No.: US 2017/0137387 A1
(43) Pub. Date: May 18, 2017

(54) PHARMACOLOGICALLY ACTIVE QUINAZOLINEDIONE DERIVATIVES

(71) Applicant: ORION CORPORATION, Espoo (FI)

(72) Inventors: Peteris PRUSIS, Turku (FI); Iisa HÖGLUND, Turku (FI); Olli TÖRMAKANGAS, Turku (FI); Ari HIETANEN, Lieto (FI); Riina ARVELA, Turku (FI); Anniina VESALAINEN, Paimio (FI); Terhi HEIKKINEN, Lieto (FI)

(21) Appl. No.: 15/309,679

(22) PCT Filed: May 8, 2015
(86) PCT No.: PCT/FI2015/000020

§ 371 Date: Nov. 8, 2016

(30) Foreign Application Priority Data
May 9, 2014 (FI) 20140133

(51) Int. Cl. C07D 239/96; A61K 49/00; C07D 498/06; C07D 491/056; C07D 405/06

(52) U.S. Cl. CPC C07D 239/96; C07D 491/056; C07D 405/06; C07D 498/06; A61K 49/0052

(57) ABSTRACT

Compounds of formula (I),

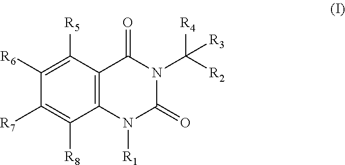

wherein R₁ through R₈ are as defined in the claims, exhibit a positive allosteric GABA_{B} modulator effect and are thus useful as positive allosteric modulators of the GABA_{B} receptor.

### FIELD OF THE INVENTION

The present disclosure relates to pharmacologically active quinazolinedione derivatives, or pharmaceutically acceptable salts or esters thereof, as well as to pharmaceutical compositions containing them and to their use as positive allosteric modulators of the γ-aminobutyric acid class B (GABA_{B}) receptor. They can be used in isotopically unlabeled or labeled form.

### BACKGROUND OF THE INVENTION

γ-Aminobutyric acid (GABA) is the main inhibitory neurotransmitter in the adult mammalian central nervous system (CNS) mediating its effect through ionotrophic GABA_{A} and GABA_{C} receptors and metabotrophic GABA_{B} receptors. GABA_{B} receptors are widely distributed in the CNS as well as in peripheral tissues. The receptor is present both on presynaptic terminals and postsynaptic neurons involved in fine-tuning of several neurotransmitter systems.

GABA_{B} receptors belong to class C of G protein-coupled receptors (GPCRs) in the same class as metabotrophic glutamate receptors, calcium sensing receptors, taste receptors, and a number of orphan receptors. A specific feature of GABA_{B} receptors, contrary to other GPCRs, is the function as obligatory heterodimers comprised of two subunits, GABA_{B2} and GABA_{B2}. The GABA_{B2} subunit is the site for binding of the orthosteric ligand and the GABA_{B2} subunit is responsible for signaling and coupling of the receptor to intracellular G proteins. Binding of an orthosteric ligand to the N-terminal Venus flytrap domain in the GABA_{B2} subunit induces a conformational change and activation of the GABA_{B2} subunit and further activation of intracellular signaling. GABA_{B} receptors couple via Gαᵢ proteins to inhibit adenylyl cyclase activity and via Gβ_{γ} subunits to regulate the activity of inwardly rectifying potassium channels and voltage-sensitive calcium channels. Mutational studies have shown that the allosteric site is distinct from the orthosteric site residing within the transmembrane domain of the GABA_{B2} subunit (Binet, V. et al. Journal of Biological Chemistry, 279 (2004) 29085).

Dysfunction in the GABAergic system or other neurotransmitter systems controlled by the activity of the GABAergic system has been implicated in a number of CNS disorders (Bowery, N. G. et al. Pharmacological Reviews, 54 (2002) 247). Therefore, GABA_{B} receptor activating compounds, such as positive allosteric modulators of the GABA_{B} receptor, could be useful in the treatment of several diseases, such as essential tremor, Parkinsonian tremor, levodopa-induced dyskinesia, Parkinsonian motor symptoms, Parkinsonian non-motor symptoms, spasticity related to multiple sclerosis, spasticity related to amyotrophic lateral sclerosis, spasticity related to spinal cord injury, spasticity related to cerebral injury, dystonia, chronic pain, addiction, anxiety, epilepsy, autism, fragile X syndrome, amyotrophic lateral sclerosis, post-traumatic stress disorder, depression, insomnia, narcolepsy, Alzheimer's disease, dementia, Charcot Marie Tooth 1A neuropathy, overactive bladder, gastro-esophageal reflux disease, inflammatory bowel disease, or chronic tinnitus.

The GABA_{B} receptor agonist baclofen is in clinical use for the treatment of spastic movement disorders and as a muscle relaxant. Despite the selectivity and potency as GABA_{B} agonist, the compound has limitations due to a poor pharmacokinetic profile for CNS indications and side effects, such as sedation and development of tolerance (Kumar, K. et al. Pharmacology, Biochemistry and Behavior, 110 (2013) 174).

In order to avoid sedation due to agonism, it is desirable that the positive allosteric modulator of the GABA_{B} receptor has weak intrinsic activity and efficacy as agonist. However, several of the positive allosteric modulators of the GABA_{B} receptor known in the art are associated with agonism.

A positive allosteric modulator offers the possibility of a more physiological way of modulating the receptor only in the presence of the endogenous agonist. A pure positive allosteric modulator without agonism exerts it effect by increasing the efficacy and/or potency of the endogenous agonist and being inactive in the absence of the endogenous neurotransmitter(s). Compared to traditional orthosteric agonists, allosteric modulation offers several advantages, such as spatial and temporal physiological activation of the targeted neurotransmitter system and corresponding receptor, and hence a safer profile with potential for less side-effects. The development of tachyphylaxis and tolerance can be avoided by an allosteric mode of action (De Amici, M. et al. Medicinal Research Reviews, 30 (2010) 463; Kenakin, T. Combinatorial Chemistry & High Throughput Screening, 11 (2008) 337).

Some compounds with a positive allosteric GABA_{B} modulator effect are known in the art. Pyrimidine derivatives as positive allosteric modulators of the GABA_{B} receptor have been disclosed in WO 2005/094828 and WO 2006/136442. Imidazole derivatives as positive allosteric modulators of the GABA_{B} receptor have been disclosed in WO 2006/001750, WO 2007/073298, WO 2007/073299, WO 2007/073300, and WO 2008/130313. Quinoline derivatives as positive allosteric modulators of the GABA_{B} receptor have been disclosed in WO 2006/048146, WO 2006/128802, and WO 2009/041904. Thieno[2,3-b]pyridine derivatives as positive allosteric modulators of the GABA_{B} receptor have been disclosed in WO 2006/063732. Phenylacetic acid derivatives, benzofuran-2(3H)-one derivatives, and indolin-2-one derivatives as positive allosteric modulators of the GABA_{B} receptor have been disclosed in WO 2007/014843. Thiazole derivatives and oxazole derivatives as positive allosteric modulators of the GABA_{B} receptor have been disclosed in WO 2007/073296. Pyrazole derivatives as positive allosteric modulators of the GABA_{B} receptor have been disclosed in WO 2007/073297. Triazinedione derivatives as positive allosteric modulators of the GABA_{B} receptor have been disclosed in WO 2008/056257. Xanthine derivatives as positive allosteric modulators of the GABA_{B} receptor have been disclosed in WO 2008/130314. Pteridinedione derivatives as positive allosteric modulators of the GABA_{B} receptor have been disclosed in WO 2009/041905.

As to known quinazolinedione derivatives, 3-(4-methoxybenzyl)-1-methylquinazoline-2,4(1H,3H)-dione has been disclosed in KR 2013074801. 1-Methyl-3-(3-methyl-4-nitrobenzyl)-6-(perfluoropropan-2-yl)quinazoline-2,4(1H,3H)-dione has been disclosed in WO 2013/065725. 1-Ethyl-3-(4-methoxyphenethyl)quinazoline-2,4(1H,3H)-dione, 1-ethyl-3-(3-methoxyphenethyl)quinazoline-2,4(1H,3H)-dione, and 1-ethyl-3-(2-methoxyphenethyl)quinazoline-2,4(1H,3H)-dione have been disclosed in Guerrero R., L. et al. Journal of the Mexican Chemical Society, 56 (2012) 201. 3-(4-Fluorophenethyl)-1-methyl-7-nitroquinazoline-2,4(1H,3H)-dione has been disclosed in WO 2004/112793. 3-(3-Methoxyphenethyl)-1-methylquinazoline-2,4(1H,3H)-dione, 3-(2-methoxyphenethyl)-1-methylquinazoline-2,4(1H,3H)-dione, and 3-(4-methoxyphenethyl)-1-methylquinazoline-2,4(1H,3H)-dione have been disclosed in Rivero, I. A. et al. Molecules, 9 (2004) 609. 3-(4-Chlorophenethyl)-1-(furan-2-ylmethyl)quinazoline-2,4(1H,3H)-dione and 3-(4-chlorophenethyl)-1-methylquinazoline-2,4(1H,3H)-dione have been disclosed in WO 2004/013068. 3-(4-Fluorobenzyl)-6-iodo-1-methylquinazoline-2,4(1H,3H)-dione, 3-(4-chlorobenzyl)-6-iodo-1-methylquinazoline-2,4(1H,3H)-dione, 3-(3-fluorobenzyl)-6-iodo-1-methylquinazoline-2,4(1H,3H)-dione, 3-(3-chlorobenzyl)-6-iodo-1-methylquinazoline-2,4(1H,3H)-dione, 3-(4-bromobenzyl)-6-iodo-1-methylquinazoline-2,4(1H,3H)-dione, and 3-(3,4-difluorobenzyl)-6-iodo-1-methylquinazoline-2,4(1H,3H)-dione have been disclosed in WO 2004/007469. 2-(3-(3,4-Dichlorobenzyl)-2,4-dioxo-3,4-dihydroquinazolin-1(2H)-yl)propanoic acid and ethyl 2-(3-(3,4-dichlorobenzyl)-2,4-dioxo-3,4-dihydroquinazolin-1(2H)-yl)propanoate have been disclosed in EP 0218999 A2. 4-((7-Nitro-2,4-dioxo-1-propyl-1,2-dihydroquinazolin-3(4H)-yl)methyl)benzonitrile has been disclosed in U.S. Pat. No. 6,200,976. 1-(2-Methylallyl)-3-(naphthalen-1-ylmethyl)quinazoline-2,4(1H,3H)-dione has been disclosed in Montginoul, C. et al. Annales pharmaceutiques françaises, 46 (1988) 223. Also 1-methyl-3-(4-methylbenzyl)quinazoline-2,4(1H,3H)-dione, 1-(2-(dimethylamino)ethyl)-6,7-dimethoxy-3-(4-methoxybenzyl)quinazoline-2,4(1H,3H)-dione, 3-(4-methoxybenzyl)-1-(2-(4-methylpiperazin-1-yl)ethyl)quinazoline-2,4(1H,3H)-dione, 1-(2-(diethylamino)ethyl)-6,7-dimethoxy-3-(4-methoxybenzyl)quinazoline-2,4(1H,3H)-dione, 1-(3-(dimethylamino)propyl)-6,7-dimethoxy-3-(4-methoxybenzyl)quinazoline-2,4(1H,3H)-dione, 3-(4-methoxybenzyl)-1-(3-(4-methylpiperazin-1-yl)propyl)quinazoline-2,4(1H,3H)-dione, 1-(2-(dimethylamino)ethyl)-3-(4-methoxybenzyl)quinazoline-2,4(1H,3H)-dione, 3-(benzo[d][1,3]dioxol-5-ylmethyl)-1-(2-oxo-2-phenylethyl)quinazoline-2,4(1H,3H)-dione, 1-(3,3-dimethyl-2-oxobutyl)-3-(3-methylphenethyl)quinazoline-2,4(1H,3H)-dione, and 1-(3-(dimethylamino)propyl)-3-(4-methoxybenzyl)quinazoline-2,4(1H,3H)-dione have been disclosed.

### SUMMARY OF THE INVENTION

An object of the present disclosure is to provide further positive allosteric modulators of the GABA_{B} receptor that can be used for the treatment of a disease where a positive allosteric modulator of the GABA_{B} receptor is indicated to be useful. Accordingly, an object of the present disclosure is to provide further compounds to be used as positive allosteric modulators of the GABA_{B} receptor in the treatment of mammals, such as humans. Furthermore, pharmaceuticals compositions containing said compounds are provided.

The positive allosteric modulators of the GABA_{B} receptor provided by the present disclosure possess enhanced primary pharmacological properties, that is positive allosteric GABA_{B} modulator effect. Additionally, the positive allosteric modulators of the GABA_{B} receptor provided by the present disclosure possess decreased agonism.

### DETAILED DESCRIPTION OF THE INVENTION

The present disclosure relates to compounds of formula I,

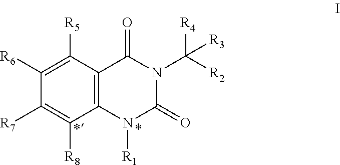

wherein
R₁ is (C₁-C₅)alkyl, (C₂-C₅)alkenyl, (C₂-C₅)alkynyl, (C₄-C₇)cycloalkyl, oxetan-2-yl, oxetan-3-yl, carboxy(C₂-C₅)alkyl, cyano(C₂-C₅)alkyl, aryl(C₂-C₅)alkyl, halohydroxy(C₁-C₅)alkyl, hydroxy(C₁-C₉)alkyl, (C₁-C₃)alkoxy(C₁-C₅)alkyl, methylthio(C₁-C₅)alkyl, methylsulfinyl(C₁-C₅)alkyl, methylsulfonyl(C₁-C₅)alkyl, amino(C₁-C₅)alkyl, ((C₁-C₃)alkylamino)(C₁-C₅)alkyl, (di(C₁-C₃)alkylamino)(C₁-C₅)alkyl, heterocyclyl(C₁-C₅)alkyl, heteroaryl(C₁-C₅)alkyl, (C₁-C₅)alkylcarbonyl(C₁-C₅)alkyl, (C₃-C₆)cycloalkylcarbonyl(C₁-C₅)alkyl, arylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, (di(C₁-C₃)alkylamino)carbonyl(C₂-C₅)alkyl, (N—(C₁-C₃)alkyl-N-methoxyamino)carbonyl(C₂-C₅)alkyl, heterocyclylcarbonyl(C₂-C₅)alkyl, halo(C₁-C₃)alkoxy(C₁-C₅)alkyl, hydroxy(C₁-C₃)alkoxy(C₁-C₅)alkyl, methoxy(C₁-C₃)alkoxy(C₁-C₅)alkyl, or (C₁-C₅)alkylcarbonylhydroxy(C₁-C₅)alkyl, wherein said (C₄-C₇)cycloalkyl, aryl, heterocyclyl, or heteroaryl as such or as part of another group is unsubstituted or substituted with 1 substituent being methyl or hydroxy;
R₂ is phenyl, phenylmethyl, or 2-phenylethyl, wherein said phenyl as such or as part of another group is substituted with 1, 2, or 3 substituent(s) R₉;
R₃ is H, (C₁-C₃)alkyl, phenyl, phenylmethyl, or methoxy(C₁-C₃)alkyl, wherein said phenyl as such or as part of another group is unsubstituted;
or R₂ and R₃ form, together with the carbon atom to which they are attached, cyclopentyl or cyclohexyl, wherein said cyclopentyl or cyclohexyl is substituted with 2 substituents R₁₀;

### R₁ is H;

or R₃ and R₄ form, together with the carbon atom to which they are attached, (C₃-C₆)cycloalkyl, wherein said (C₃-C₆)cycloalkyl is unsubstituted;
R₅ is H, halogen, or (C₁-C₅)alkoxy;
R₆ is H, methyl, halogen, hydroxy, (C₁-C₃)alkoxy, halo(C₁-C₃)alkyl, methoxy(C₁-C₃)alkyl, or halo(C₁-C₃)alkoxy;
R₇ is H, (C₁-C₅)alkyl, (C₄-C₇)cycloalkyl, halogen, (C₁-C₃)alkoxy, heterocyclyl, nitro, halo(C₁-C₃)alkyl, methoxy(C₁-C₃)alkyl, halo(C₁-C₃)alkoxy, or dimethylamino, wherein said (C₄-C₇)cycloalkyl or heterocyclyl is unsubstituted;
or R₆ and R₇ form, together with the carbon ring atoms to which they are attached, a 5- or 6-membered non-aromatic heterocyclic ring containing 2 ring heteroatoms being O, wherein said heterocyclic ring is unsubstituted;
R₃ is H, halogen, (C₁-C₃)alkoxy, or methoxy(C₁-C₃)alkoxy;
or R₁ and R₈ form together *—CHR₁₁—C(R₁₂)₂—O—*′, wherein * and *′ indicate respective point of attachment;
R₉ is, independently at each occurrence, methyl, cyano, halogen, methoxy, phenyloxy, nitro, phenylmethyl, halomethyl, halomethoxy, or dimethylamino, wherein said phenyl as part of another group is unsubstituted;
or R₉ and R₉ attached to adjacent carbon ring atoms form, together with the carbon ring atoms to which they are attached, a 5- or 6-membered non-aromatic heterocyclic ring containing 1 or 2 ring heteroatom(s) being O or a 6-membered aromatic carbocyclic ring, wherein said heterocyclic ring or carbocyclic ring is unsubstituted;
R₁₀ and R₁₀ attached to adjacent carbon ring atoms form, together with the carbon ring atoms to which they are attached, phenyl, wherein said phenyl is unsubstituted or substituted with 1, 2, 3, or 4 substituent(s) being, independently at each occurrence, halogen or methoxy;
R₁₁ is H, (C₁-C₅)alkyl, carboxy, hydroxy(C₁-C₅)alkyl, or (C₁-C₅)alkylcarbonyl;
R₁₂ is, independently at each occurrence, (C₁-C₅)alkyl, carboxy, hydroxy(C₁-C₅)alkyl, or (C₁-C₅)alkylcarbonyl;
or a pharmaceutically acceptable ester or salt thereof;
with the provisos that
a) when R₁ is (C₂-C₅)alkenyl, R₅, R₆, R₇, and R₈ are not simultaneously H;
b) when R₁ is (di(C₁-C₃)alkylamino)(C₁-C₅)alkyl, R₉ is not methoxy;
c) when R₂ is phenylmethyl, R₉ is not methoxy;
d) the compound is not 3-(4-methoxybenzyl)-1-methylquinazoline-2,4(1H,3H)-dione, 1-methyl-3-(3-methyl-4-nitrobenzyl)-6-(perfluoropropan-2-yl)quinazoline-2,4(1H,3H)-dione, 1-methyl-3-(4-methylbenzyl)quinazoline-2,4(1H,3H)-dione, 3-(4-fluorophenethyl)-1-methyl-7-nitroquinazoline-2,4(1H,3H)-dione, 3-(4-methoxybenzyl)-1-(2-(4-methylpiperazin-1-yl)ethyl)quinazoline-2,4(1H,3H)-dione, 3-(4-methoxybenzyl)-1-(3-(4-methylpiperazin-1-yl)propyl)quinazoline-2,4(1H,3H)-dione, 3-(benzo[d][1,3]dioxol-5-ylmethyl)-1-(2-oxo-2-phenylethyl)quinazoline-2,4(1H,3H)-dione, 1-(3,3-dimethyl-2-oxobutyl)-3-(3-methylphenethyl)quinazoline-2,4(1H,3H)-dione, 3-(4-chlorophenethyl)-1-(furan-2-ylmethyl)quinazoline-2,4(1H,3H)-dione, 3-(4-chlorophenethyl)-1-methylquinazoline-2,4(1H,3H)-dione, 3-(4-fluorobenzyl)-6-iodo-1-methylquinazoline-2,4(1H,3H)-dione, 3-(4-chlorobenzyl)-6-iodo-1-methylquinazoline-2,4(1H,3H)-dione, 3-(3-fluorobenzyl)-6-iodo-1-methylquinazoline-2,4(1H,3H)-dione, 3-(3-chlorobenzyl)-6-iodo-1-methylquinazoline-2,4(1H,3H)-dione, 3-(4-bromobenzyl)-6-iodo-1-methylquinazoline-2,4(1H,3H)-dione, 3-(3,4-difluorobenzyl)-6-iodo-1-methylquinazoline-2,4(1H,3H)-dione, 2-(3-(3,4-dichlorobenzyl)-2,4-dioxo-3,4-dihydroquinazolin-1(2H)-yl)propanoic acid, ethyl 2-(3-(3,4-dichlorobenzyl)-2,4-dioxo-3,4-dihydroquinazolin-1(2H)-yl)propanoate, or 4-((7-nitro-2,4-dioxo-1-propyl-1,2-dihydroquinazolin-3(4H)-yl)methyl)benzonitrile.

In one embodiment, the present disclosure relates to compounds of formula I, wherein R₁₁ is H or (C₁-C₅)alkyl.

In one embodiment, the present disclosure relates to compounds of formula I, wherein R₁₁ is H.

In one embodiment, the present disclosure relates to compounds of formula I, wherein R₂ is phenyl, wherein said phenyl is substituted with 1 or 2 substituent(s) R₉; or R₂ and R₃ form, together with the carbon atom to which they are attached, cyclopentyl or cyclohexyl, wherein said cyclopentyl or cyclohexyl is substituted with 2 substituents R₁₀.

In one embodiment, the present disclosure relates to compounds of formula I, wherein R₂ is phenyl, wherein said phenyl is substituted with 1 substituent R₉;

or R₂ and R₃ form, together with the carbon atom to which they are attached, cyclopentyl or cyclohexyl, wherein said cyclopentyl or cyclohexyl is substituted with 2 substituents R₁₀.

In one embodiment, the present disclosure relates to compounds of formula I, wherein R₄ is H.

In one embodiment, the present disclosure relates to compounds of formula I, wherein R₉ is, independently at each occurrence, cyano, halogen, methoxy, phenyloxy, nitro, halomethyl, halomethoxy, or dimethylamino, wherein said phenyl as part of another group is unsubstituted;

or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted.

In one embodiment, the present disclosure relates to compounds of formula I, wherein R₉ is, independently at each occurrence, cyano, halogen, methoxy, phenyloxy, nitro, halomethyl, or halomethoxy, wherein said phenyl as part of another group is unsubstituted; or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted.

In one embodiment, the present disclosure relates to compounds of formula I, wherein R₉ is, independently at each occurrence, cyano, halogen, phenyloxy, halomethyl, or halomethoxy, wherein said phenyl as part of another group is unsubstituted; or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted.

In one embodiment, the present disclosure relates to compounds of formula I, wherein R₉ is, independently at each occurrence, halogen, phenyloxy, halomethyl, or halomethoxy, wherein said phenyl as part of another group is unsubstituted;

or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted.

In one embodiment, the present disclosure relates to compounds of formula I, wherein R₉ is, independently at each occurrence, halogen, methoxy, or halomethoxy.

In one embodiment, the present disclosure relates to compounds of formula I, wherein R₉ is, independently at each occurrence, halogen or halomethoxy.

In one embodiment, the present disclosure relates to compounds of formula I, wherein R₇ is H, halogen, (C₁-C₃)alkoxy, or halo(C₁-C₃)alkyl;

or R₆ and R₇ form, together with the carbon ring atoms to which they are attached, a 5- or 6-membered non-aromatic heterocyclic ring containing 2 ring heteroatoms being O, wherein said heterocyclic ring is unsubstituted.

In one embodiment, the present disclosure relates to compounds of formula I, wherein R₇ is halogen or halo(C₁-C₃)alkyl;

or R₆ and R₇ form, together with the carbon ring atoms to which they are attached, a 5- or 6-membered non-aromatic heterocyclic ring containing 2 ring heteroatoms being O, wherein said heterocyclic ring is unsubstituted.

In one embodiment, the present disclosure relates to compounds of formula I, wherein R₇ is halogen or halo(C₁-C₃)alkyl.

In one embodiment, the present disclosure relates to compounds of formula I, wherein R₁ is (C₁-C₅)alkyl, (C₂-C₅)alkenyl, (C₂-C₅)alkynyl, oxetan-3-yl, carboxy(C₂-C₅)alkyl, cyano(C₂-C₅)alkyl, hydroxy(C₁-C₉)alkyl, (C₁-C₃)alkoxy(C₁-C₅)alkyl, heterocyclyl(C₁-C₅)alkyl, (C₁-C₅)alkylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl, wherein said heterocyclyl as part of another group is unsubstituted or substituted with 1 substituent being methyl or hydroxy;

or R₁ and R₈ form together *—CHR₁₁—C(R₁₂)₂—O—*′, wherein * and *′ indicate respective point of attachment.

In one embodiment, the present disclosure relates to compounds of formula I, wherein R₁ is (C₁-C₅)alkyl, carboxy(C₂-C₅)alkyl, cyano(C₂-C₅)alkyl, hydroxy(C₁-C₅)alkyl, (C₁-C₃)alkoxy(C₁-C₅)alkyl, heterocyclyl(C₁-C₅)alkyl, (C₁-C₅)alkylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl, wherein said heterocyclyl as part of another group is unsubstituted or substituted with 1 substituent being methyl or hydroxyl.

In one embodiment, the present disclosure relates to compounds of formula I, wherein R₁ is (C₁-C₅)alkyl, cyano(C₂-C₅)alkyl, hydroxy(C₁-C₅)alkyl, (C₁-C₃)alkoxy(C₁-C₅)alkyl, aminocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl.

In one embodiment, the present disclosure relates to compounds of formula I, wherein R₁ is (C₁-C₅)alkyl, oxetan-3-yl, carboxy(C₂-C₅)alkyl, cyano(C₂-C₅)alkyl, hydroxy(C₁-C₉)alkyl, heterocyclyl(C₁-C₅)alkyl, or (C₁-C₅)alkylcarbonyl(C₁-C₅)alkyl, wherein said heterocyclyl as part of another group is unsubstituted or substituted with 1 substituent being methyl or hydroxy;

or R₁ and R₈ form together *—CHR₁₁—C(R₁₂)₂—O—*′, wherein * and *′ indicate respective point of attachment.

In one embodiment, the present disclosure relates to compounds of formula I, wherein R₁ is (C₁-C₅)alkyl, carboxy(C₂-C₅)alkyl, cyano(C₂-C₅)alkyl, hydroxy(C₁-C₅)alkyl, heterocyclyl(C₁-C₅)alkyl, or (C₁-C₅)alkylcarbonyl(C₁-C₅)alkyl, wherein said heterocyclyl as part of another group is unsubstituted or substituted with 1 substituent being methyl or hydroxyl.

In one embodiment, the present disclosure relates to compounds of formula I, wherein R₁ is (C₁-C₅)alkyl, cyano(C₂-C₅)alkyl, or hydroxy(C₁-C₅)alkyl.

In one embodiment, the present disclosure relates to compounds of formula I, wherein R₃ is H or (C₁-C₃)alkyl;

or R₂ and R₃ form, together with the carbon atom to which they are attached, cyclopentyl or cyclohexyl, wherein said cyclopentyl or cyclohexyl is substituted with 2 substituents

In one embodiment, the present disclosure relates to compounds of formula I, wherein R₃ is H or (C₁-C₃)alkyl.

In one embodiment, the present disclosure relates to compounds of formula I, wherein R₆ is H, halogen, (C₁-C₃)alkoxy, halo(C₁-C₃)alkyl, or halo(C₁-C₃)alkoxy.

In one embodiment, the present disclosure relates to compounds of formula I, wherein R₆ is H or halo(C₁-C₃)alkoxy.

In one embodiment, the present disclosure relates to compounds of formula I, wherein R₆ is H, halogen, or (C₁-C₃)alkoxy.

In one embodiment, the present disclosure relates to compounds of formula I, wherein R₆ is H.

In one embodiment, the present disclosure relates to compounds of formula I, wherein R₅ is H or halogen.

In one embodiment, the present disclosure relates to compounds of formula I, wherein R₅ is H.

In one embodiment, the present disclosure relates to compounds of formula I, wherein R₈ is H, halogen, or (C₁-C₃)alkoxy;

or R₁ and R₈ form together *—CHR₁₁—C(R₁₂)₂—O—*′, wherein * and *′ indicate respective point of attachment.

In one embodiment, the present disclosure relates to compounds of formula I, wherein

R₁ is (C₁-C₅)alkyl, (C₄-C₇)cycloalkyl, carboxy(C₂-C₅)alkyl, cyano(C₂-C₅)alkyl, aryl(C₂-C₅)alkyl, hydroxy(C₁-C₅)alkyl, (C₁-C₃)alkoxy(C₁-C₅)alkyl, methylthio(C₁-C₅)alkyl, methylsulfinyl(C₁-C₅)alkyl, methylsulfonyl(C₁-C₅)alkyl, amino(C₁-C₅)alkyl, ((C₁-C₃)alkylamino)(C₁-C₅)alkyl, (di(C₁-C₃)alkylamino)(C₁-C₅)alkyl, heterocyclyl(C₁-C₅)alkyl, heteroaryl(C₁-C₅)alkyl, (C₁-C₅)alkylcarbonyl(C₁-C₅)alkyl, arylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, (di(C₁-C₃)alkylamino)carbonyl(C₂-C₅)alkyl, (N—(C₁-C₃)alkyl-N-methoxyamino)carbonyl(C₂-C₅)alkyl, heterocyclylcarbonyl(C₂-C₅)alkyl, halo(C₁-C₃)alkoxy(C₁-C₅)alkyl, hydroxy(C₁-C₃)alkoxy(C₁-C₅)alkyl, or methoxy(C₁-C₃)alkoxy(C₁-C₅)alkyl, wherein said (C₄-C₇)cycloalkyl, aryl, heterocyclyl, or heteroaryl as such or as part of another group is unsubstituted or substituted with 1 substituent being methyl or hydroxy;
R₂ is phenyl, phenylmethyl, or 2-phenylethyl, wherein said phenyl as such or as part of another group is substituted with 1 or 2 substituent(s) R₉;
R₃ is H, (C₁-C₃)alkyl, phenyl, phenylmethyl, or methoxy(C₁-C₃)alkyl, wherein said phenyl as such or as part of another group is unsubstituted;
or R₂ and R₃ form, together with the carbon atom to which they are attached, cyclopentyl or cyclohexyl, wherein said cyclopentyl or cyclohexyl is substituted with 2 substituents R₁₀;

### R₄ is H;

or R₃ and R₄ form, together with the carbon atom to which they are attached, (C₃-C₆)cycloalkyl, wherein said (C₃-C₆)cycloalkyl is unsubstituted;
R₅ is H or methoxy;
R₆ is H, methyl, halogen, hydroxy, (C₁-C₃)alkoxy, halo(C₁-C₃)alkyl, methoxy(C₁-C₃)alkyl, or halo(C₁-C₃)alkoxy;
R₇ is H, (C₁-C₃)alkyl, (C₄-C₇)cycloalkyl, halogen, (C₁-C₃)alkoxy, heterocyclyl, halo(C₁-C₃)alkyl, methoxy(C₁-C₃)alkyl, halo(C₁-C₃)alkoxy, or dimethylamino, wherein said (C₄-C₇)cycloalkyl or heterocyclyl is unsubstituted;
or R₆ and R₇ form, together with the carbon ring atoms to which they are attached, a 5- or 6-membered non-aromatic heterocyclic ring containing 2 ring heteroatoms being O, wherein said heterocyclic ring is unsubstituted;
R₈ is H, halogen, or (C₁-C₃)alkoxy;
R₉ is, independently at each occurrence, methyl, cyano, halogen, methoxy, phenyloxy, nitro, phenylmethyl, halomethyl, or halomethoxy, wherein said phenyl as part of another group is unsubstituted;
or R₉ and R₉ attached to adjacent carbon ring atoms form, together with the carbon ring atoms to which they are attached, a 5-membered non-aromatic heterocyclic ring containing 1 ring heteroatom being O or a 6-membered aromatic carbocyclic ring, wherein said heterocyclic ring or carbocyclic ring is unsubstituted.

In one embodiment, the present disclosure relates to compounds of formula I, wherein

R₁ is (C₁-C₅)alkyl, (C₂-C₅)alkenyl, (C₂-C₅)alkynyl, oxetan-3-yl, carboxy(C₂-C₅)alkyl, cyano(C₂-C₅)alkyl, hydroxy(C₁-C₉)alkyl, (C₁-C₃)alkoxy(C₁-C₅)alkyl, heterocyclyl(C₁-C₅)alkyl, (C₁-C₅)alkylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl, wherein said heterocyclyl as part of another group is unsubstituted or substituted with 1 substituent being methyl or hydroxy;
R₂ is phenyl, wherein said phenyl is substituted with 1 or 2 substituent(s) R₉;
R₃ is H or (C₁-C₃)alkyl;
or R₂ and R₃ form, together with the carbon atom to which they are attached, cyclopentyl or cyclohexyl, wherein said cyclopentyl or cyclohexyl is substituted with 2 substituents R₁₀;

### R₄ is H;

R₅ is H, halogen, or (C₁-C₅)alkoxy;
R₆ is H, halogen, (C₁-C₃)alkoxy, halo(C₁-C₃)alkyl, or halo(C₁-C₃)alkoxy;
R₇ is H, halogen, (C₁-C₃)alkoxy, or halo(C₁-C₃)alkyl;
or R₆ and R₇ form, together with the carbon ring atoms to which they are attached, a 5- or 6-membered non-aromatic heterocyclic ring containing 2 ring heteroatoms being O, wherein said heterocyclic ring is unsubstituted;
R₈ is H, halogen, or (C₁-C₃)alkoxy;
or R₁ and R₈ form together *—CHR₁₁—C(R₁₂)₂—O—*′, wherein * and *′ indicate respective point of attachment;
R₉ is, independently at each occurrence, cyano, halogen, methoxy, phenyloxy, nitro, halomethyl, halomethoxy, or dimethylamino, wherein said phenyl as part of another group is unsubstituted;
or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted;
R₁₁ is H or (C₁-C₅)alkyl.

In one embodiment, the present disclosure relates to compounds of formula I, wherein

R₁ is (C₁-C₅)alkyl, carboxy(C₂-C₅)alkyl, cyano(C₂-C₅)alkyl, hydroxy(C₁-C₅)alkyl, (C₁-C₃)alkoxy(C₁-C₅)alkyl, heterocyclyl(C₁-C₅)alkyl, (C₁-C₅)alkylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl, wherein said heterocyclyl as part of another group is unsubstituted or substituted with 1 substituent being methyl or hydroxy;
R₂ is phenyl, wherein said phenyl is substituted with 1 or 2 substituent(s) R₉;
R₃ is H or (C₁-C₃)alkyl;
or R₂ and R₃ form, together with the carbon atom to which they are attached, cyclopentyl or cyclohexyl, wherein said cyclopentyl or cyclohexyl is substituted with 2 substituents R₁₀;

### R₄ is H;

R₅ is H or methoxy;
R₆ is H, halogen, (C₁-C₃)alkoxy, halo(C₁-C₃)alkyl, or halo(C₁-C₃)alkoxy;
R₇ is H, halogen, (C₁-C₃)alkoxy, or halo(C₁-C₃)alkyl;
or R₆ and R₇ form, together with the carbon ring atoms to which they are attached, a 5- or 6-membered non-aromatic heterocyclic ring containing 2 ring heteroatoms being O, wherein said heterocyclic ring is unsubstituted;
R₈ is H, halogen, or (C₁-C₃)alkoxy;
R₉ is, independently at each occurrence, cyano, halogen, methoxy, phenyloxy, nitro, halomethyl, or halomethoxy, wherein said phenyl as part of another group is unsubstituted; or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted.

In one embodiment, the present disclosure relates to compounds of formula I, wherein

R₁ is (C₁-C₅)alkyl, cyano(C₂-C₅)alkyl, hydroxy(C₁-C₅)alkyl, (C₁-C₃)alkoxy(C₁-C₅)alkyl, aminocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl;
R₂ is phenyl, wherein said phenyl is substituted with 1 or 2 substituent(s) R₉;
R₃ is H or (C₁-C₃)alkyl;
or R₂ and R₃ form, together with the carbon atom to which they are attached, cyclopentyl or cyclohexyl, wherein said cyclopentyl or cyclohexyl is substituted with 2 substituents R₁₀;

### R₄ is H;

R₅ is H or methoxy;
R₆ is H or halo(C₁-C₃)alkoxy;
R₇ is halogen or halo(C₁-C₃)alkyl;
or R₆ and R₇ form, together with the carbon ring atoms to which they are attached, a 5- or 6-membered non-aromatic heterocyclic ring containing 2 ring heteroatoms being O, wherein said heterocyclic ring is unsubstituted;
R₈ is H, halogen, or (C₁-C₃)alkoxy;
R₉ is, independently at each occurrence, cyano, halogen, phenyloxy, halomethyl, or halomethoxy, wherein said phenyl as part of another group is unsubstituted;
or R₉ and R₉ attached to adjacent carbon ring atoms form, together with the carbon ring atoms to which they are attached, a 5-membered non-aromatic heterocyclic ring containing 1 ring heteroatom being O, wherein said heterocyclic ring is unsubstituted.

In one embodiment, the present disclosure relates to compounds of formula I, wherein

R₁ is (C₁-C₅)alkyl, oxetan-3-yl, carboxy(C₂-C₅)alkyl, cyano(C₂-C₅)alkyl, hydroxy(C₁-C₉)alkyl, heterocyclyl(C₁-C₅)alkyl, or (C₁-C₅)alkylcarbonyl(C₁-C₅)alkyl, wherein said heterocyclyl as part of another group is unsubstituted or substituted with 1 substituent being methyl or hydroxy;
R₂ is phenyl, wherein said phenyl is substituted with 1 or 2 substituent(s) R₉;
R₃ is H or (C₁-C₃)alkyl;

### R₄ is H;

R₅ is H or halogen;
R₆ is H, halogen, or (C₁-C₃)alkoxy;
R₇ is halogen or halo(C₁-C₃)alkyl;
R₈ is H, halogen, or (C₁-C₃)alkoxy;
or R₁ and R₈ form together *—CHR₁₁—C(R₁₂)₂—O—*′, wherein * and *′ indicate respective point of attachment;
R₉ is, independently at each occurrence, halogen, methoxy, or halomethoxy;

### R₁₁ is H.

In one embodiment, the present disclosure relates to compounds of formula I, wherein

R₁ is (C₁-C₅)alkyl, carboxy(C₂-C₅)alkyl, cyano(C₂-C₅)alkyl, hydroxy(C₁-C₅)alkyl, heterocyclyl(C₁-C₅)alkyl, or (C₁-C₅)alkylcarbonyl(C₁-C₅)alkyl, wherein said heterocyclyl as part of another group is unsubstituted or substituted with 1 substituent being methyl or hydroxy;
R₂ is phenyl, wherein said phenyl is substituted with 1 or 2 substituent(s) R₉;
R₃ is H or (C₁-C₃)alkyl;

### R₄ is H;

### R₅ is H;

R₆ is H, halogen, or (C₁-C₃)alkoxy;
R₇ is halogen or halo(C₁-C₃)alkyl;
R₈ is H, halogen, or (C₁-C₃)alkoxy;
R₉ is, independently at each occurrence, halogen, methoxy, or halomethoxy.

In one embodiment, the present disclosure relates to compounds of formula I, wherein

R₁ is (C₁-C₅)alkyl, cyano(C₂-C₅)alkyl, or hydroxy(C₁-C₅)alkyl;
R₂ is phenyl, wherein said phenyl is substituted with 1 substituent R₉;
R₃ is H or (C₁-C₃)alkyl;

### R₄ is H;

### R₅ is H;

### R₆ is H;

R₇ is halogen or halo(C₁-C₃)alkyl;
R₈ is H, halogen, or (C₁-C₃)alkoxy;
R₉ is, independently at each occurrence, halogen or halomethoxy.

In one embodiment, the present disclosure relates to compounds of formula I, wherein the compound is 3-(4-bromobenzyl)-5,7-dimethoxy-1-methylquinazoline-2,4(1H,3H)-dione, 3-(3,4-dichlorobenzyl)-1-(2-hydroxy-2-methylpropyl)-7-(trifluoromethyl)quinazoline-2,4(1H,3H)-dione, 6,7-difluoro-1-(2-hydroxy-2-methylpropyl)-3-(4-methoxybenzyl)quinazoline-2,4(1H,3H)-dione, 7-chloro-6-fluoro-1-(2-hydroxy-2-methylpropyl)-3-(4-methoxybenzyl)quinazoline-2,4(1H,3H)-dione, 3-(4-bromobenzyl)-6-(difluoromethoxy)-7-fluoro-1-methylquinazoline-2,4(1H,3H)-dione, 3-(4-bromobenzyl)-7-fluoro-6-methoxy-1-methylquinazoline-2,4(1H,3H)-dione, 3-(4-bromobenzyl)-7-fluoro-6-hydroxy-1-methylquinazoline-2,4(1H,3H)-dione, 7-(4-bromobenzyl)-5-methyl-[1,3]dioxolo[4,5-g]quinazoline-6,8(5H,7H)-dione, 3-(4-bromobenzyl)-1-isopropyl-6,7-dimethoxyquinazoline-2,4(1H,3H)-dione, 3-(4-bromobenzyl)-1-(2-hydroxy-2-methylpropyl)-6,7-dimethoxyquinazoline-2,4(1H,3H)-dione, 3-(4-bromobenzyl)-6,7-difluoro-1-methylquinazoline-2,4(1H,3H)-dione, 1-methyl-3-(1,2,3,4-tetrahydronaphthalen-1-yl)-7-(trifluoromethyDquinazoline-2,4(1H,3H)-dione, 3-(4-bromobenzyl)-1-methyl-6-(trifluoromethyl)quinazoline-2,4(1H,3H)-dione, 3-(3,4-dichlorobenzyl)-1-methyl-7-(trifluoromethyDquinazoline-2,4(1H,3H)-dione, 3-(4-bromobenzyl)-1-methyl-7-(trifluoromethyl)quinazoline-2,4(1H,3H)-dione, 7-fluoro-1-methyl-3-(4-(trifluoromethyl)benzyl)quinazoline-2,4(1H,3H)-dione, 7-fluoro-3-(3-fluoro-4-methoxybenzyl)-1-methylquinazoline-2,4(1H,3H)-dione, 3-(4-bromobenzyl)-7-chloro-1-methylquinazoline-2,4(1H,3H)-dione, 3-(1-(4-bromophenyl)ethyl)-7-fluoro-1-methylquinazoline-2,4(1H,3H)-dione, 3-((4-chlorophenyl)(phenyl)methyl)-1,7-dimethylquinazoline-2,4(1H,3H)-dione, 3-(4-bromobenzyl)-5,8-dimethoxy-1-methylquinazoline-2,4(1H,3H)-dione, 3-(3,4-dichlorobenzyl)-7-fluoro-1-methylquinazoline-2,4(1H,3H)-dione, 7-fluoro-3-(4-methoxybenzyl)-1-methylquinazoline-2,4(1H,3H)-dione, (S)-3-(1-(4-chlorophenyl)ethyl)-7-fluoro-1-methylquinazoline-2,4(1H,3H)-dione, (R)-3-(1-(4-chlorophenyl)ethyl)-6,7-dimethoxy-1-methylquinazoline-2,4(1H,3H)-dione, 3-(4-bromobenzyl)-1-(3,3-dimethyl-2-oxobutyl)-7-fluoroquinazoline-2,4(1H,3H)-dione, 3-(4-bromobenzyl)-7-fluoro-1-(3-oxobutan-2-yl)quinazoline-2,4(1H,3H)-dione, 3-(4-bromobenzyl)-7-fluoro-1-(2-methoxyethyl)quinazoline-2,4(1H,3H)-dione, 3-(4-bromobenzyl)-7-fluoro-1-(2-(2-methoxyethoxy)ethyDquinazoline-2,4(1H,3H)-dione, 2-(3-(4-bromobenzyl)-7-fluoro-2,4-dioxo-3,4-dihydroquinazolin-1(2H)-yl)-propanenitrile, 3-(4-bromobenzyl)-7-fluoro-1-(3-oxobutyl)quinazoline-2,4(1H,3H)-dione, 3-(4-bromobenzyl)-7-fluoro-1-(2-hydroxypropyl)quinazoline-2,4(1H,3H)-dione, 3-(4-bromobenzyl)-7-fluoro-1-methylquinazoline-2,4(1H,3H)-dione, 3-(3-(4-bromobenzyl)-7-fluoro-2,4-dioxo-3,4-dihydroquinazolin-1(2H)-yl)propanoic acid, 3-(3-(4-bromobenzyl)-7-fluoro-2,4-dioxo-3,4-dihydroquinazolin-1(2H)-yl)-propanamide, 3-(3-(4-bromobenzyl)-7-fluoro-2,4-dioxo-3,4-dihydroquinazolin-1(2H)-yl)-N,N-dimethylpropanamide, 2-(3-(4-bromobenzyl)-7-fluoro-2,4-dioxo-3,4-dihydroquinazolin-1(2H)-yl)propanamide, 3-(4-bromobenzyl)-7-fluoro-1-isopropylquinazoline-2,4(1H,3H)-dione, 3-(4-bromobenzyl)-7-fluoro-1-(2-hydroxy-2-methylpropyl)quinazoline-2,4(1H,3H)-dione, 7-fluoro-1-methyl-3-(4-nitrobenzyl)quinazoline-2,4(1H,3H)-dione, 3-(4-chloro-3-phenoxybenzyl)-7-fluoro-1-(2-hydroxy-2-methylpropyl)quinazoline-2,4(1H,3H)-dione, (R)-3-(1-(4-chlorophenyl)ethyl)-7-fluoro-1-methylquinazoline-2,4(1H,3H)-dione, 3-(1-(4-chlorophenyl)cyclopropyl)-7-fluoro-1-methylquinazoline-2,4(1H,3H)-dione, 3-(1-(4-chlorophenyl)-3-methoxypropyl)-7-fluoro-1-methylquinazoline-2,4(1H,3H)-dione, 3-(4-bromobenzyl)-7-chloro-1-(2-hydroxy-2-methylpropyl)quinazoline-2,4(1H,3H)-dione, 3-(4-bromobenzyl)-7-chloro-1-((3-methyloxetan-3-yl)methyl)quinazoline-2,4(1H,3H)-dione, 3-(4-bromobenzyl)-7-chloro-6-fluoro-1-methylquinazoline-2,4(1H,3H)-dione, 3-(4-bromobenzyl)-7-chloro-6-fluoro-1-(2-hydroxy-2-methylpropyl)quinazoline-2,4(1H,3H)-dione, 3-(4-bromobenzyl)-7-chloro-6-fluoro-1-((3-methyloxetan-3-yl)methyl)quinazoline-2,4(1H,3H)-dione, methyl 2-(3-(4-bromobenzyl)-7-chloro-6-fluoro-2,4-dioxo-3,4-dihydroquinazolin-1(2H)-yl)propanoate, 3-(4-bromobenzyl)-7-fluoro-1-neopentylquinazoline-2,4(1H,3H)-dione, 3-(4-bromobenzyl)-7-chloro-6-fluoro-1-(2-methoxy-2-methylpropyl)quinazoline-2,4(1H,3H)-dione, 7-chloro-6-fluoro-3-(4-methoxybenzyl)-1-((3-methyloxetan-3-yl)methyl)quinazoline-2,4(1H,3H)-dione, methyl 2-(7-chloro-6-fluoro-3-(4-methoxybenzyl)-2,4-dioxo-3,4-dihydroquinazolin-1(2H)-yl)propanoate, 7-chloro-6-fluoro-1-(3-hydroxy-3-methylbutan-2-yl)-3-(4-methoxybenzyl)quinazoline-2,4(1H,3H)-dione, (R)-7-chloro-3-(1-(4-chlorophenyl)ethyl)-6-fluoro-1-methylquinazoline-2,4(1H,3H)-dione, (R)-7-chloro-3-(1-(4-chlorophenyl)ethyl)-6-fluoro-1-((3-methyloxetan-3-yl)-methyl)quinazoline-2,4(1H,3H)-dione, 3-(4-bromobenzyl)-7,8-difluoro-1-methylquinazoline-2,4(1H,3H)-dione, 3-(4-bromobenzyl)-6,7,8-trifluoro-1-(2-hydroxy-2-methylpropyl)quinazoline-2,4(1H,3H)-dione, 3-(4-bromobenzyl)-6,7-difluoro-1-(2-hydroxy-2-methylpropyl)quinazoline-2,4(1H,3H)-dione, 3-(4-(difluoromethoxy)benzyl)-6,7-difluoro-1-(2-hydroxy-2-methylpropyl)quinazoline-2,4(1H,3H)-dione, 3-((2,3-dihydrobenzofuran-5-yl)methyl)-6,7-difluoro-1-methylquinazoline-2,4(1H,3H)-dione, 3-(4-bromobenzyl)-1-(but-3-yn-2-yl)-7-fluoroquinazoline-2,4(1H,3H)-dione, 6,7,8-trifluoro-1-(2-hydroxy-2-methylpropyl)-3-(4-methoxybenzyl)quinazoline-2,4(1H,3H)-dione, 6-(4-bromobenzyl)-9,10-difluoro-2,2-dimethyl-2H-[1,4]oxazino[2,3,4-ij]quinazoline-5,7(3H,6H)-dione, 10-chloro-6-(4-chlorobenzyl)-2,2-dimethyl-2H-[1,4]oxazino[2,3,4-ij]quinazoline-5,7(3H,6H)-dione, (R)-3-(4-bromobenzyl)-7-chloro-1-(2-hydroxypropyl)quinazoline-2,4(1H,3H)-dione, 7-chloro-1-((3-methyloxetan-3-yl)methyl)-3-(4-(trifluoromethoxy)benzyl)quinazoline-2,4(1H,3H)-dione, 2-(3-(4-bromobenzyl)-7-chloro-6-fluoro-2,4-dioxo-3,4-dihydroquinazolin-1(2H)-yl)-N-methylpropanamide, 6,8-difluoro-1-(2-hydroxy-2-methylpropyl)-3-(4-(trifluoromethoxy)benzyl)quinazoline-2,4(1H,3H)-dione, 3-(4-bromobenzyl)-6,7-difluoro-1-(3-oxobutyl)quinazoline-2,4(1H,3H)-dione, 7-chloro-3-(4-(dimethylamino)benzyl)-1-(2-hydroxy-2-methylpropyl)quinazoline-2,4(1H,3H)-dione, 6-chloro-8-fluoro-1-(2-hydroxy-2-methylpropyl)-3-(4-methoxybenzyl)quinazoline-2,4(1H,3H)-dione, 3-((2,3-dihydrobenzofuran-5-yl)methyl)-6,7-difluoro-1-(2-hydroxy-2-methylpropyl)quinazoline-2,4(1H,3H)-dione, (R)-3-(1-(4-chlorophenyl)ethyl)-1-methylquinazoline-2,4(1H,3H)-dione, 6,8-dichloro-1-(2-hydroxy-2-methylpropyl)-3-(4-methoxybenzyl)quinazoline-2,4(1H,3H)-dione, 6-(4-bromobenzyl)-10-fluoro-2,2-dimethyl-2H-[1,4]oxazino[2,3,4-ij]quinazoline-5,7(3H,6H)-dione, 7-chloro-3-(5-chloro-2,3-dihydro-1H-inden-1-yl)-1-(2-hydroxy-2-methylpropyl)quinazoline-2,4(1H,3H)-dione, 3-(4-bromobenzyl)-6-chloro-8-fluoro-1-(2-hydroxy-2-methylpropyl)quinazoline-2,4(1H,3H)-dione, 6-(4-chlorobenzyl)-10-fluoro-2,2-dimethyl-2H-[1,4]oxazino[2,3,4-ij]quinazoline-5,7(3H,6H)-dione, (R)-7-chloro-3-(4-chlorobenzyl)-8-fluoro-1-(2-hydroxypropyl)quinazoline-2,4(1H,3H)-dione, 10-chloro-6-(4-chlorobenzyl)-2-methyl-5,7-dioxo-3,5,6,7-tetrahydro-2H-[1,4]oxazino[2,3,4-ij]quinazoline-2-carboxylic acid, 6-(4-bromobenzyl)-9-fluoro-10-methoxy-2,2-dimethyl-2H-[1,4]oxazino[2,3,4-ij]quinazoline-5,7(3H,6H)-dione, 7-chloro-3-((2,3-dihydrobenzofuran-5-yl)methyl)-6-fluoro-1-(2-hydroxy-2-methylpropyl)quinazoline-2,4(1H,3H)-dione, 7-chloro-3-(4-chlorobenzyl)-8-fluoro-1-(2-hydroxy-2-methyl-3-oxobutyl)quinazoline-2,4(1H,3H)-dione, 3-(4-bromobenzyl)-1-ethyl-7-fluoroquinazoline-2,4(1H,3H)-dione, 7-chloro-3-(4-chloro-3-fluorobenzyl)-1-((3-methyloxetan-3-yl)methyl)quinazoline-2,4(1H,3H)-dione, 5,7-dichloro-3-(4-chlorobenzyl)-1-(2-hydroxy-2-methylpropyl)quinazoline-2,4(1H,3H)-dione, 7-chloro-3-(4-(difluoromethoxy)benzyl)-8-fluoro-1-(2-hydroxy-2-methylpropyl)quinazoline-2,4(1H,3H)-dione, (S)-3-(4-bromobenzyl)-7-chloro-8-fluoro-1-(2-hydroxypropyl)quinazoline-2,4(1H,3H)-dione, 3-(4-bromo-2-fluorobenzyl)-7-chloro-1-(2-hydroxy-2-methylpropyl)quinazoline-2,4(1H,3H)-dione, 3-(2,4-dichlorobenzyl)-6,7-difluoro-1-(2-hydroxy-2-methylpropyl)quinazoline-2,4(1H,3H)-dione, 9-chloro-6-(4-methoxybenzyl)-2,2-dimethyl-2H-[1,4]oxazino[2,3,4-ij]quinazoline-5,7(3H,6H)-dione, 3-(4-(difluoromethoxy)benzyl)-6,8-difluoro-1-(2-hydroxy-2-methylpropyl)quinazoline-2,4(1H,3H)-dione, 3-(4-bromobenzyl)-7-chloro-1-(2-hydroxy-2-methylpropyl)-8-(2-methoxyethoxy)quinazoline-2,4(1H,3H)-dione, 1-(3-bromo-2-(hydroxymethyl)-2-methylpropyl)-3-(4-bromobenzyl)-7-chloro-6-fluoroquinazoline-2,4(1H,3H)-dione, (S)-7-chloro-3-(1-(4-chlorophenyl)ethyl)-6-fluoro-1-methylquinazoline-2,4(1H,3H)-dione, 7-chloro-3-(4-chlorobenzyl)-8-fluoro-1-methylquinazoline-2,4(1H,3H)-dione, 3-(4-bromobenzyl)-7-chloro-6-fluoro-1-(oxetan-3-yl)quinazoline-2,4(1H,3H)-dione, 7-chloro-3-(4-chlorobenzyl)-8-fluoro-1-(2-hydroxyethyDquinazoline-2,4(1H,3H)-dione, 10-fluoro-6-(4-methoxybenzyl)-2,2-dimethyl-2H-[1,4]oxazino[2,3,4-ij]quinazoline-5,7(3H,6H)-dione, 3-(4-bromobenzyl)-7-chloro-1-(1-cyclopropyl-1-oxopropan-2-yl)-6-fluoroquinazoline-2,4(1H,3H)-dione, 3-(4-bromobenzyl)-7-chloro-8-fluoro-1-(2-hydroxy-2,3-dimethylbutyl)quinazoline-2,4(1H,3H)-dione, 7-chloro-8-fluoro-1-(2-hydroxy-2,3-dimethylbutyl)-3-(4-methoxybenzyl)quinazoline-2,4(1H,3H)-dione, 7-chloro-3-((2,3-dihydrobenzofuran-5-yl)methyl)-1-(2-hydroxy-2-methylpropyl)quinazoline-2,4(1H,3H)-dione, (S)-7-chloro-6-fluoro-1-(2-hydroxypropyl)-3-(4-methoxybenzyl)quinazoline-2,4(1H,3H)-dione, (S)-7-chloro-3-(1-(4-chlorophenyl)ethyl)-6-fluoro-1-((3-methyloxetan-3-yl)methyl)quinazoline-2,4(1H,3H)-dione, 10-chloro-6-(4-methoxybenzyl)-2,2-dimethyl-2H-[1,4]oxazino[2,3,4-ij]quinazoline-5,7(3H,6H)-dione, 6,8-difluoro-1-(2-hydroxy-2-methylpropyl)-3-(4-methoxybenzyl)quinazoline-2,4(1H,3H)-dione, 7-chloro-3-(4-(difluoromethoxy)benzyl)-1-(2-hydroxy-2-methylpropyl)quinazoline-2,4(1H,3H)-dione, (R)-7-chloro-3-(1-(4-chlorophenyl)ethyl)-1-methylquinazoline-2,4(1H,3H)-dione, 7-chloro-3-(3-chloro-4-methoxybenzyl)-1-(2-hydroxy-2-methylpropyl)quinazoline-2,4(1H,3H)-dione, 9,10-difluoro-6-(4-methoxybenzyl)-2,2-dimethyl-2H-[1,4]oxazino[2,3,4-ij]quinazoline-5,7(3H,6H)-dione, 7-chloro-3-(4-chloro-3-fluorobenzyl)-1-(2-hydroxy-2-methylpropyl)quinazoline-2,4(1H,3H)-dione, 7-chloro-3-(4-(difluoromethoxy)benzyl)-1-((3-methyloxetan-3-yl)methyl)quinazoline-2,4(1H,3H)-dione, (R)-7-chloro-3-(1-(4-chlorophenyl)ethyl)-1-(2-hydroxy-2-methylpropyl)quinazoline-2,4(1H,3H)-dione, (R)-7-chloro-6-fluoro-1-(2-hydroxypropyl)-3-(4-methoxybenzyl)quinazoline-2,4(1H,3H)-dione, 7-chloro-8-fluoro-1-(2-hydroxy-2-methylpropyl)-3-(4-(trifluoromethoxy)benzyl)quinazoline-2,4(1H,3H)-dione, (R)-7-chloro-6-fluoro-1-(2-hydroxy-2-methylpropyl)-3-(1-(4-methoxyphenyl)ethyl)quinazoline-2,4(1H,3H)-dione, 10-chloro-2-isopropyl-6-(4-methoxybenzyl)-2-methyl-2H-[1,4]oxazino[2,3,4-ij]quinazoline-5,7(3H,6H)-dione, (S)-3-(4-bromobenzyl)-7-chloro-1-(2-hydroxypropyl)quinazoline-2,4(1H,3H)-dione, 3-(4-bromobenzyl)-7-chloro-8-fluoro-1-(2-hydroxy-2-methylpropyl)quinazoline-2,4(1H,3H)-dione, (R)-7-chloro-3-(4-chlorobenzyl)-1-(2-hydroxypropyl)quinazoline-2,4(1H,3H)-dione, (S)-7-chloro-3-(1-(4-chlorophenyl)ethyl)-6-fluoro-1-(2-hydroxy-2-methylpropyl)quinazoline-2,4(1H,3H)-dione, (S)-7-chloro-3-(4-chlorobenzyl)-1-(2-hydroxypropyl)quinazoline-2,4(1H,3H)-dione, 6-(4-bromobenzyl)-10-chloro-2,2-dimethyl-2H-[1,4]oxazino[2,3,4-ij]quinazoline-5,7(3H,6H)-dione, 7-chloro-6-fluoro-1-(2-hydroxy-2,3-dimethylbutyl)-3-(4-methoxybenzyl)quinazoline-2,4(1H,3H)-dione, 9-fluoro-10-methoxy-6-(4-methoxybenzyl)-2,2-dimethyl-2H-[1,4]oxazino[2,3,4-ij]quinazoline-5,7(3H,6H)-dione, (Z)-7-chloro-3-(4-chlorobenzyl)-1-(prop-1-en-1-yl)quinazoline-2,4(1H,3H)-dione, 6-chloro-3-(4-chlorobenzyl)-8-fluoro-1-(2-hydroxy-2-methylpropyl)quinazoline-2,4(1H,3H)-dione, 7-chloro-3-(4-chlorobenzyl)-1-(2-hydroxy-2-methylpropyl)quinazoline-2,4(1H,3H)-dione, 3-(4-bromobenzyl)-6,8-difluoro-1-(2-hydroxy-2-methylpropyl)quinazoline-2,4(1H,3H)-dione, 7-chloro-8-fluoro-1-(2-hydroxy-2-methylpropyl)-3-(4-methoxybenzyl)quinazoline-2,4(1H,3H)-dione, 3-(benzo[d][1,3]dioxol-5-ylmethyl)-7-chloro-1-(2-hydroxy-2-methylpropyl)quinazoline-2,4(1H,3H)-dione, 3-(4-bromobenzyl)-6,7,8-trifluoro-1-methylquinazoline-2,4(1H,3H)-dione, (R)-7-chloro-3-(1-(4-chlorophenyl)ethyl)-1-((3-methyloxetan-3-yl)methyl)quinazoline-2,4(1H,3H)-dione, 3-(benzo[d][1,3]dioxol-5-ylmethyl)-7-chloro-1-((3-methyloxetan-3-yl)methyl)quinazoline-2,4(1H,3H)-dione, (R)-3-(4-bromobenzyl)-7-chloro-8-fluoro-1-(2-hydroxypropyl)quinazoline-2,4(1H,3H)-dione, 6-(4-bromobenzyl)-9-fluoro-2,2-dimethyl-2H-[1,4]oxazino[2,3,4-ij]quinazoline-5,7(3H,6H)-dione, 7-chloro-3-(3-chloro-4-methoxybenzyl)-1-((3-methyloxetan-3-yl)methyl)quinazoline-2,4(1H,3H)-dione, 6-(4-bromobenzyl)-9-chloro-2,2-dimethyl-2H-[1,4]oxazino[2,3,4-ij]quinazoline-5,7(3H,6H)-dione, (R)-3-(1-(4-chlorophenyl)ethyl)-1-methyl-7-nitroquinazoline-2,4(1H,3H)-dione, 7-chloro-3-(4-chlorobenzyl)-1-(2,3-dihydroxy-2-methylbutyl)-8-fluoroquinazoline-2,4(1H,3H)-dione, 9-fluoro-6-(4-methoxybenzyl)-2,2-dimethyl-2H-[1,4]oxazino[2,3,4-ij]quinazoline-5,7(3H,6H)-dione, 3-(4-bromo-2-fluorobenzyl)-7-chloro-1-((3-methyloxetan-3-yl)methyl)quinazoline-2,4(1H,3H)-dione, 2-acetyl-10-chloro-6-(4-chlorobenzyl)-2-methyl-2H-[1,4]oxazino[2,3,4-ij]quinazoline-5,7(3H,6H)-dione, diastereomer 1 of 10-chloro-6-(4-chlorobenzyl)-2-(1-hydroxyethyl)-2-methyl-2H-[1,4]oxazino[2,3,4-ij]quinazoline-5,7(3H,6H)-dione, or diastereomer 2 of 10-chloro-6-(4-chlorobenzyl)-2-(1-hydroxyethyl)-2-methyl-2H-[1,4]oxazino[2,3,4-ij]quinazoline-5,7(3H,6H)-dione.

In one embodiment, the present disclosure relates to compounds of formula I, wherein the compound is in isotopically unlabeled form.

In one embodiment, the present disclosure relates to compounds of formula I, wherein the compound is in isotopically labeled form.

In one embodiment, the present disclosure relates to compounds of formula I, wherein the compound is ³H labeled.

In one embodiment, the present disclosure relates to compounds of formula I, wherein the compound is ¹¹C labeled.

In one embodiment, the present disclosure relates to compounds of formula I, wherein the compound is ¹⁸F labeled.

The terms employed herein have the meanings indicated below. The term “at least one halogen” employed in the meanings below refers to one or several halogen(s), such as one halogen.

The term “(C₁-C₅)alkyl”, as employed herein as such or as part of another group, refers to a straight or branched chain saturated hydrocarbon group having 1, 2, 3, 4, or 5 carbon atom(s). Representative examples of (C₁-C₅)alkyl include, but are not limited to, methyl, ethyl, propyl, isopropyl, isobutyl, tert-butyl, 2-methylbutyl, and neopentyl.

The term “(C₂-C₅)alkenyl”, as employed herein, refers to a straight or branched chain hydrocarbon group having 2, 3, 4, or 5 carbon atoms and at least one carbon-carbon double bond. Representative examples of (C₂-C₅)alkenyl include, but are not limited to, vinyl and prop-1-en-1-yl.

The term “(C₂-C₅)alkynyl”, as employed herein, refers to a straight or branched chain hydrocarbon group having 2, 3, 4, or 5 carbon atoms and at least one carbon-carbon triple bond. Representative examples of (C₂-C₅)alkynyl include, but are not limited to, ethynyl and but-3-yn-2-yl.

The term “(C₄-C₇)cycloalkyl”, as employed herein, refers to a saturated cyclic hydrocarbon group having 4, 5, 6 or 7 carbon atoms. Representative examples of (C₄-C₇)cycloalkyl include, but are not limited to, cyclopentyl and cyclohexyl.

The term “(C₂-C₅)alkyl”, as employed herein as part of another group, refers to a straight or branched chain saturated hydrocarbon group having 2, 3, 4, or 5 carbon atoms. Representative examples of (C₂-C₅)alkyl include, but are not limited to, ethyl, propyl, and neopentyl.

The term “carboxy”, as employed herein as such or as part of another group, refers to a —COOH group.

The term “carboxy(C₂-C₅)alkyl”, as employed herein, refers to a carboxy group, as defined herein, appended to the parent molecular moiety through an (C₂-C₅)alkyl group, as defined herein. Representative examples of carboxy(C₂-C₅)alkyl include, but are not limited to, 2-carboxyethyl and 1-carboxy-2,2-dimethylpropyl.

The term “cyano”, as employed herein as such or as part of another group, refers to a —CN group.

The term “cyano(C₂-C₅)alkyl”, as employed herein, refers to one or two cyano group(s), as defined herein, appended to the parent molecular moiety through an (C₂-C₅)alkyl group, as defined herein. When there are two cyano groups, both cyano groups can be attached to the same carbon atom or the cyano groups can be attached to different carbon atoms. Representative examples of cyano(C₂-C₅)alkyl include, but are not limited to, 1-cyanoethyl and 1-cyano-2,2-dimethylpropyl

The term “aryl”, as employed herein as part of another group, refers to an aromatic monocyclic hydrocarbon group having 6 carbon atoms or to an aromatic bicyclic hydrocarbon group having 10 carbon atoms. Representative examples of aryl include, but are not limited to, phenyl and naphth-1-yl.

The term “aryl(C₂-C₅)alkyl”, as employed herein, refers to an aryl group, as defined herein, appended to the parent molecular moiety through an (C₂-C₅)alkyl group, as defined herein. Representative examples of aryl(C₂-C₅)alkyl include, but are not limited to, 1-phenylethyl and 1-phenylpropyl.

The term “halo” or “halogen”, as employed herein as such or as part of another group, refers to fluorine, chlorine, bromine or iodine.

The term “hydroxy”, as employed herein as such or as part of another group, refers to a —OH group.

The term “halohydroxy(C₁-C₅)alkyl”, as employed herein, refers to at least one halogen, as defined herein, and one or two hydroxy group(s), as defined herein, appended to the parent molecular moiety through an (C₁-C₅)alkyl group, as defined herein. When there are several halogens, the halogens can be identical or different. The halogen(s) and the hydroxy group(s) can be attached to different carbon atoms or several halogens and/or hydroxy groups can be attached to the same carbon atom. Representative examples of halohydroxy(C₁-C₅)alkyl include, but are not limited to, 4-chloro-2-hydroxybutyl and 3-bromo-2-(hydroxymethyl)-2-methylpropyl.

The term “(C₁-C₉)alkyl”, as employed herein as part of another group, refers to a straight or branched chain saturated hydrocarbon group having 1, 2, 3, 4, 5, 6, 7, 8, or 9 carbon atom(s). Representative examples of (C₁-C₉)alkyl include, but are not limited to, methyl, ethyl, propyl, isopropyl, isobutyl, tert-butyl, 2-methylbutyl, neopentyl, and 2,3-dimethylbutyl.

The term “hydroxy(C₁-C₉)alkyl”, as employed herein, refers to one or two hydroxy group(s), as defined herein, appended to the parent molecular moiety through an (C₁-C₉)alkyl group, as defined herein. When there are two hydroxy groups, both hydroxy groups can be attached to the same carbon atom or the hydroxy groups can be attached to different carbon atoms. Representative examples of hydroxy(C₁-C₉)alkyl include, but are not limited to, 1-hydroxyethyl, 2-hydroxyethyl, 2-hydroxypropyl, 2-hydroxy-2-methylpropyl, 3-hydroxy-3-methylbut-2-yl, 2,3-dihydroxy-2-methylbutyl, and 2-hydroxy-2,3-dimethylbutyl.

The term “(C₁-C₃)alkyl”, as employed herein as such or as part of another group, refers to a saturated hydrocarbon group having 1, 2, or 3 carbon atom(s). Representative examples of (C₁-C₃)alkyl include, but are not limited to, methyl, ethyl, and isopropyl.

The term “(C₁-C₃)alkoxy”, as employed herein as such or as part of another group, refers to an (C₁-C₃)alkyl group, as defined herein, appended to the parent molecular moiety through an oxygen atom. Representative examples of (C₁-C₃)alkoxy include, but are not limited to, methoxy and ethoxy.

The term “(C₁-C₃)alkoxy(C₁-C₅)alkyl”, as employed herein as such or as part of another group, refers to one or two (C₁-C₃)alkoxy group(s), as defined herein, appended to the parent molecular moiety through an (C₁-C₅)alkyl group, as defined herein. When there are two (C₁-C₃)alkoxy groups, the (C₁-C₃)alkoxy groups can be identical or different and both (C₁-C₃)alkoxy groups can be attached to the same carbon atom or the (C₁-C₃)alkoxy groups can be attached to different carbon atoms. Representative examples of (C₁-C₃)alkoxy(C₁-C₅)alkyl include, but are not limited to, 2-methoxyethyl and 2-methoxy-2-methylpropyl.

The term “methylthio(C₁-C₅)alkyl”, as employed herein, refers to one or two —SCH₃ group(s) appended to the parent molecular moiety through an (C₁-C₅)alkyl group, as defined herein. When there are two —SCH₃ groups, both —SCH₃ groups can be attached to the same carbon atom or the —SCH₃ groups can be attached to different carbon atoms. Representative examples of methylthio(C₁-C₅)alkyl include, but are not limited to, 2-methylthioethyl and 2-methyl-2-methylthiopropyl.

The term “methylsulfinyl(C₁-C₅)alkyl”, as employed herein, refers to a —(S═O)—CH₃ group appended to the parent molecular moiety through an (C₁-C₅)alkyl group, as defined herein. Representative examples of methylsulfinyl(C₁-C₅)alkyl include, but are not limited to, 2-(methylsulfinyl)ethyl and 2-methyl-2-(methylsulfinyl)propyl.

The term “methylsulfonyl(C₁-C₅)alkyl”, as employed herein, refers to a —(O═S═O)—CH₃ group appended to the parent molecular moiety through an (C₁-C₅)alkyl group, as defined herein. Representative examples of methylsulfonyl(C₁-C₅)alkyl include, but are not limited to, 2-(methylsulfonyl)ethyl and 2-methyl-2-(methylsulfonyl)propyl.

The term “amino(C₁-C₅)alkyl”, as employed herein, refers to a —NH₂ group appended to the parent molecular moiety through an (C₁-C₅)alkyl group, as defined herein. Representative examples of amino(C₁-C₅)alkyl include, but are not limited to, aminomethyl and 3-aminopropyl.

The term “(C₁-C₃)alkylamino”, as employed herein as part of another group, refers to an (C₁-C₃)alkyl group, as defined herein, appended to the parent molecular moiety through a —NH— group. Representative examples of (C₁-C₃)alkylamino include, but are not limited to, methylamino and isopropylamino.

The term “((C₁-C₃)alkylamino)(C₁-C₅)alkyl”, as employed herein, refers to an (C₁-C₃)alkylamino group, as defined herein, appended to the parent molecular moiety through an (C₁-C₅)alkyl group, as defined herein. Representative examples of ((C₁-C₃)alkylamino)(C₁-C₅)alkyl include, but are not limited to, methylaminomethyl and 3-isopropylaminopropyl.

The term “di(C₁-C₃)alkylamino”, as employed herein as part of another group, refers to two (C₁-C₃)alkyl groups, as defined herein, both appended to the parent molecular moiety through the same nitrogen atom. The (C₁-C₃)alkyl groups can be identical or different. Representative examples of di(C₁-C₃)alkylamino include, but are not limited to, dimethylamino and N-methyl-N-propylamino.

The term “(di(C₁-C₃)alkylamino)(C₁-C₅)alkyl”, as employed herein, refers to a di(C₁-C₃)alkylamino group, as defined herein, appended to the parent molecular moiety through an (C₁-C₅)alkyl group, as defined herein. Representative examples of (di(C₁-C₃)alkylamino)(C₁-C₅)alkyl include, but are not limited to, dimethylaminomethyl and 3-(N-methyl-N-propylamino)propyl.

The term “heterocyclyl”, as employed herein as such or as part of another group, refers to a 4-, 5-, 6-, or 7-membered non-aromatic monocyclic group containing 1 or 2 ring heteroatom(s) each independently selected from N, O, and S. Representative examples of heterocyclyl include, but are not limited to, oxetan-3-yl and piperidin-4-yl.

The term “heterocyclyl(C₁-C₅)alkyl”, as employed herein, refers to a heterocyclyl group, as defined herein, appended to the parent molecular moiety through an (C₁-C₅)alkyl group, as defined herein. Representative examples of heterocyclyl(C₁-C₅)alkyl include, but are not limited to, oxetan-3-ylmethyl and 1-(piperidin-4-yl)propyl.

The term “heteroaryl”, as employed herein as part of another group, refers to a 5-, 6-, or 7-membered aromatic monocyclic group containing 1 or 2 ring heteroatom(s) each independently selected from N, O, and S or to an 8-, 9-, or 10-membered aromatic bicyclic group containing 1 or 2 ring heteroatom(s) each independently selected from N, O, and S. Representative examples of heteroaryl include, but are not limited to, thiophen-3-yl and quinoxalin-5-yl.

The term “heteroaryl(C₁-C₅)alkyl”, as employed herein, refers to a heteroaryl group, as defined herein, appended to the parent molecular moiety through an (C₁-C₅)alkyl group, as defined herein. Representative examples of heteroaryl(C₁-C₅)alkyl include, but are not limited to, 1-(thiophen-3-yl)ethyl and 3-(quinoxalin-5-yl)propyl.

The term “(C₁-C₅)alkylcarbonyl”, as employed herein as such or as part of another group, refers to an (C₁-C₅)alkyl group, as defined herein, appended to the parent molecular moiety through a —(C═O)— group. Representative examples of (C₁-C₅)alkylcarbonyl include, but are not limited to, acetyl and pivaloyl.

The term “(C₁-C₅)alkylcarbonyl(C₁-C₅)alkyl”, as employed herein, refers to an (C₁-C₅)alkylcarbonyl group, as defined herein, appended to the parent molecular moiety through an (C₁-C₅)alkyl group, as defined herein. Representative examples of (C₁-C₅)alkylcarbonyl(C₁-C₅)alkyl include, but are not limited to, 3-oxobutyl, 3-oxobut-2-yl, and 3,3-dimethyl-2-oxobutyl.

The term “(C₃-C₆)cycloalkyl”, as employed herein as such or as part of another group, refers to a saturated cyclic hydrocarbon group having 3, 4, 5 or 6 carbon atoms. Representative examples of (C₃-C₆)cycloalkyl include, but are not limited to, cyclopropyl and cyclohexyl.

The term “(C₃-C₆)cycloalkylcarbonyl”, as employed herein as part of another group, refers to a (C₃-C₆)cycloalkyl group, as defined herein, appended to the parent molecular moiety through a —(C═O)— group. Representative examples of (C₃-C₆)cycloalkylcarbonyl include, but are not limited to, cyclopropylcarbonyl and cyclohexylcarbonyl.

The term “(C₃-C₆)cycloalkylcarbonyl(C₁-C₅)alkyl”, as employed herein, refers to an (C₃-C₆)cycloalkylcarbonyl group, as defined herein, appended to the parent molecular moiety through an (C₁-C₅)alkyl group, as defined herein. Representative examples of (C₃-C₆)cycloalkylcarbonyl(C₁-C₅)alkyl include, but are not limited to, 1-cyclopropyl-1-oxoprop-2-yl and 4-cyclohexyl-4-oxobutyl.

The term “arylcarbonyl”, as employed herein as part of another group, refers to an aryl group, as defined herein, appended to the parent molecular moiety through a —(C═O)— group. Representative examples of arylcarbonyl include, but are not limited to, benzoyl and 1-naphthoyl.

The term “arylcarbonyl(C₁-C₅)alkyl”, as employed herein, refers to an arylcarbonyl group, as defined herein, appended to the parent molecular moiety through an (C₁-C₅)alkyl group, as defined herein. Representative examples of arylcarbonyl(C₁-C₅)alkyl include, but are not limited to, 2-naphth-1-yl-2-oxoethyl and 4-oxo-4-phenylbutyl.

The term “(C₁-C₃)alkoxycarbonyl”, as employed herein as part of another group, refers to an (C₁-C₃)alkoxy group, as defined herein, appended to the parent molecular moiety through a —(C═O)— group. Representative examples of (C₁-C₃)alkoxycarbonyl include, but are not limited to, methoxycarbonyl and ethoxycarbonyl.

The term “(C₁-C₃)alkoxycarbonyl(C₂-C₅)alkyl”, as employed herein, refers to an (C₁-C₃)alkoxycarbonyl group, as defined herein, appended to the parent molecular moiety through an (C₂-C₅)alkyl group, as defined herein. Representative examples of (C₁-C₃)alkoxycarbonyl(C₂-C₅)alkyl include, but are not limited to, 2-ethoxy-2-oxoethyl and 1-methoxy-1-oxoprop-2-yl.

The term “aminocarbonyl(C₂-C₅)alkyl”, as employed herein, refers to a —(C═O)—NH₂ group appended to the parent molecular moiety through an (C₂-C₅)alkyl group, as defined herein.

Representative examples of aminocarbonyl(C₂-C₅)alkyl include, but are not limited to, 3-amino-3-oxopropyl and 1-amino-1-oxoprop-2-yl.

The term “((C₁-C₃)alkylamino)carbonyl”, as employed herein as part of another group, refers to an (C₁-C₃)alkylamino group, as defined herein, appended to the parent molecular moiety through a —(C═O)— group. Representative examples of ((C₁-C₃)alkylamino)carbonyl include, but are not limited to, methylaminocarbonyl and isopropylaminocarbonyl.

The term “((C₁-C₃)alkylamino)carbonyl(C₂-C₅)alkyl”, as employed herein, refers to an ((C₁-C₃)alkylamino)carbonyl group, as defined herein, appended to the parent molecular moiety through an (C₂-C₅)alkyl group, as defined herein. Representative examples of ((C₁-C₃)alkylamino)carbonyl(C₂-C₅)alkyl include, but are not limited to, 1-methylamino-1-oxoprop-2-yl and 4-isopropylamino-4-oxobutyl.

The term “(di(C₁-C₃)alkylamino)carbonyl”, as employed herein as part of another group, refers to a di(C₁-C₃)alkylamino group, as defined herein, appended to the parent molecular moiety through a —(C═O)— group. Representative examples of (di(C₁-C₃)alkylamino)carbonyl include, but are not limited to, dimethylaminocarbonyl and (N-methyl-N-propylamino)carbonyl.

The term “(di(C₁-C₃)alkylamino)carbonyl(C₂-C₅)alkyl”, as employed herein, refers to a (di(C₁-C₃)alkylamino)carbonyl group, as defined herein, appended to the parent molecular moiety through an (C₂-C₅)alkyl group, as defined herein. Representative examples of (di(C₁-C₃)alkylamino)carbonyl(C₂-C₅)alkyl include, but are not limited to, 3-dimethylamino-3-oxopropyl and 4-(N-methyl-N-propylamino)-4-oxobutyl.

The term “N—(C₁-C₃)alkyl-N-methoxyamino”, as employed herein as part of another group, refers to an (C₁-C₃)alkyl group, as defined herein, and a methoxy group, both appended to the parent molecular moiety through the same nitrogen atom. Representative examples of N—(C₁-C₃)alkyl-N-methoxyamino include, but are not limited to, N-methoxy-N-methylamino and N-isopropyl-N-methoxyamino.

The term “(N—(C₁-C₃)alkyl-N-methoxyamino)carbonyl”, as employed herein as part of another group, refers to an N—(C₁-C₃)alkyl-N-methoxyamino group, as defined herein, appended to the parent molecular moiety through a —(C═O)— group. Representative examples of (N—(C₁-C₃)alkyl-N-methoxyamino)carbonyl include, but are not limited to, N-methoxy-N-methylaminocarbonyl and N-isopropyl-N-methoxyaminocarbonyl.

The term “(N—(C₁-C₃)alkyl-N-methoxyamino)carbonyl(C₂-C₅)alkyl”, as employed herein, refers to an (N—(C₁-C₃)alkyl-N-methoxyamino)carbonyl group, as defined herein, appended to the parent molecular moiety through an (C₂-C₅)alkyl group, as defined herein. Representative examples of (N—(C₁-C₃)alkyl-N-methoxyamino)carbonyl(C₂-C₅)alkyl include, but are not limited to, 2-(N-isopropyl-N-methoxyamino)-2-oxoethyl and 3-(N-methoxy-N-methylamino)-3-oxoprop-2-yl.

The term “heterocyclylcarbonyl”, as employed herein as part of another group, refers to a heterocyclyl group, as defined herein, appended to the parent molecular moiety through a —(C═O)— group. Representative examples of heterocyclylcarbonyl include, but are not limited to, tetrahydrofuran-2-ylcarbonyl and morpholinocarbonyl.

The term “heterocyclylcarbonyl(C₂-C₅)alkyl”, as employed herein, refers to a heterocyclylcarbonyl group, as defined herein, appended to the parent molecular moiety through an (C₂-C₅)alkyl group, as defined herein. Representative examples of heterocyclylcarbonyl(C₂-C₅)alkyl include, but are not limited to, 2-morpholino-2-oxoethyl and 4-oxo-4-tetrahydrofuran-2-ylbutyl.

The term “halo(C₁-C₃)alkoxy”, as employed herein as such or as part of another group, refers to at least one halogen, as defined herein, appended to the parent molecular moiety through an (C₁-C₃)alkoxy group, as defined herein. When there are several halogens, the halogens can be identical or different and the halogens can be attached to different carbon atoms or several halogens can be attached to the same carbon atom. Representative examples of halo(C₁-C₃)alkoxy include, but are not limited to, difluoromethoxy and 2,2,2-trifluoroethoxy.

The term “halo(C₁-C₃)alkoxy(C₁-C₅)alkyl”, as employed herein, refers to one or two halo(C₁-C₃)alkoxy group(s), as defined herein, appended to the parent molecular moiety through an (C₁-C₅)alkyl group, as defined herein. When there are two halo(C₁-C₃)alkoxy groups, the halo(C₁-C₃)alkoxy groups can be identical or different and both halo(C₁-C₃)alkoxy groups can be attached to the same carbon atom or the halo(C₁-C₃)alkoxy groups can be attached to different carbon atoms. Representative examples of halo(C₁-C₃)alkoxy(C₁-C₅)alkyl include, but are not limited to, (2,2,2-trifluoroethoxy)methyl and 1-(difluoromethoxy)prop-2-yl.

The term “hydroxy(C₁-C₃)alkoxy”, as employed herein as part of another group, refers to one or two hydroxy group(s), as defined herein, appended to the parent molecular moiety through an (C₁-C₃)alkoxy group, as defined herein. When there are two hydroxy groups, both hydroxy groups can be attached to the same carbon atom or the hydroxy groups can be attached to different carbon atoms. Representative examples of hydroxy(C₁-C₃)alkoxy include, but are not limited to, hydroxymethoxy and 2-hydroxyethoxy.

The term “hydroxy(C₁-C₃)alkoxy(C₁-C₅)alkyl”, as employed herein, refers to one or two hydroxy(C₁-C₃)alkoxy group(s), as defined herein, appended to the parent molecular moiety through an (C₁-C₅)alkyl group, as defined herein. When there are two hydroxy(C₁-C₃)alkoxy groups, the hydroxy(C₁-C₃)alkoxy groups can be identical or different and both hydroxy(C₁-C₃)alkoxy groups can be attached to the same carbon atom or the hydroxy(C₁-C₃)alkoxy groups can be attached to different carbon atoms. Representative examples of hydroxy(C₁-C₃)alkoxy(C₁-C₅)alkyl include, but are not limited to, (2-hydroxyethoxy)methyl and 1-(hydroxymethoxy)prop-2-yl.

The term “methoxy(C₁-C₃)alkoxy”, as employed herein as such or as part of another group, refers to one or two methoxy group(s) appended to the parent molecular moiety through an (C₁-C₃)alkoxy group, as defined herein. When there are two methoxy groups, both methoxy groups can be attached to the same carbon atom or the methoxy groups can be attached to different carbon atoms. Representative examples of methoxy(C₁-C₃)alkoxy include, but are not limited to, methoxymethoxy and 2-methoxyethoxy.

The term “methoxy(C₁-C₃)alkoxy(C₁-C₅)alkyl”, as employed herein, refers to one or two methoxy(C₁-C₃)alkoxy group(s), as defined herein, appended to the parent molecular moiety through an (C₁-C₅)alkyl group, as defined herein. When there are two methoxy(C₁-C₃)alkoxy groups, the methoxy(C₁-C₃)alkoxy groups can be identical or different and both methoxy(C₁-C₃)alkoxy groups can be attached to the same carbon atom or the methoxy(C₁-C₃)alkoxy groups can be attached to different carbon atoms. Representative examples of methoxy(C₁-C₃)alkoxy(C₁-C₅)alkyl include, but are not limited to, 2-(2-methoxyethoxy)ethyl and 1-(methoxymethoxy)prop-2-yl.

The term “hydroxy(C₁-C₅)alkyl”, as employed herein as such or as part of another group, refers to one or two hydroxy group(s), as defined herein, appended to the parent molecular moiety through an (C₁-C₅)alkyl group, as defined herein. When there are two hydroxy groups, both hydroxy groups can be attached to the same carbon atom or the hydroxy groups can be attached to different carbon atoms. Representative examples of hydroxy(C₁-C₅)alkyl include, but are not limited to, 1-hydroxyethyl, 2-hydroxyethyl, 2-hydroxypropyl, 2-hydroxy-2-methylpropyl, 3-hydroxy-3-methylbut-2-yl, and 2,3-dihydroxy-2-methylbutyl.

The term “(C₁-C₅)alkylcarbonylhydroxy(C₁-C₅)alkyl”, as employed herein, refers to an (C₁-C₅)alkylcarbonyl group, as defined herein, appended to the parent molecular moiety through a hydroxy(C₁-C₅)alkyl group, as defined herein. Representative examples of (C₁-C₅)alkylcarbonylhydroxy(C₁-C₅)alkyl include, but are not limited to, 1-hydroxy-3-oxobut-2-yl and 2-hydroxy-2-methyl-3-oxobutyl.

The term “methoxy(C₁-C₃)alkyl”, as employed herein, refers to one or two methoxy group(s) appended to the parent molecular moiety through an (C₁-C₃)alkyl group, as defined herein. When there are two methoxy groups, both methoxy groups can be attached to the same carbon atom or the methoxy groups can be attached to different carbon atoms. Representative examples of methoxy(C₁-C₃)alkyl include, but are not limited to, 2-methoxyethyl and 1-methoxyprop-2-yl.

The term “(C₁-C₅)alkoxy”, as employed herein, refers to an (C₁-C₅)alkyl group, as defined herein, appended to the parent molecular moiety through an oxygen atom. Representative examples of (C₁-C₅)alkoxy include, but are not limited to, methoxy, ethoxy, and pentoxy.

The term “halo(C₁-C₃)alkyl”, as employed herein, refers to at least one halogen, as defined herein, appended to the parent molecular moiety through an (C₁-C₃)alkyl group, as defined herein. When there are several halogens, the halogens can be identical or different and the halogens can be attached to different carbon atoms or several halogens can be attached to the same carbon atom. Representative examples of halo(C₁-C₃)alkyl include, but are not limited to, trifluoromethyl and 2-chloroethyl.

The term “nitro”, as employed herein, refers to a —NO₂ group.

The term “halomethyl”, as employed herein, refers to at least one halogen, as defined herein, appended to the parent molecular moiety through a methyl group. When there are several halogens, the halogens can be identical or different. Representative examples of halomethyl include, but are not limited to, bromomethyl and trifluoromethyl.

The term “halomethoxy”, as employed herein, refers to at least one halogen, as defined herein, appended to the parent molecular moiety through a methoxy group. When there are several halogens, the halogens can be identical or different. Representative examples of halomethoxy include, but are not limited to, difluoromethoxy and trifluoromethoxy.

Pharmaceutically acceptable salts, such as metal salts and acid addition salts, with organic acids or inorganic acids are well known in the field of pharmaceuticals. Representative examples of pharmaceutically acceptable metal salts include, but are not limited to, lithium, sodium, potassium, calcium, magnesium, aluminum, and zinc salts. Representative examples of pharmaceutically acceptable acid addition salts include, but are not limited to, chlorides, bromides, sulfates, nitrates, phosphates, sulfonates, methane sulfonates, formates, tartrates, maleates, citrates, benzoates, salicylates, and ascorbates.

Pharmaceutically acceptable esters of carboxy groups may be prepared by known methods using pharmaceutically acceptable alcohols that are conventional in the field of pharmaceuticals. Representative examples of pharmaceutically acceptable esters of carboxy groups include, but are not limited to, esters formed with ethanol and propan-1-ol.

Pharmaceutically acceptable esters of hydroxy groups may be prepared by known methods using pharmaceutically acceptable carboxylic acids that are conventional in the field of pharmaceuticals. Representative examples of pharmaceutically acceptable esters of hydroxy groups include, but are not limited to, esters formed with acetic acid and propionic acid.

The present disclosure includes within its scope all the possible geometric isomers, e.g. Z and E isomers (cis and trans isomers), of the compounds. Furthermore, the present disclosure includes in its scope both the individual isomers and any mixtures thereof.

The present disclosure includes within its scope all the possible tautomers, or equilibrium mixtures thereof, of the compounds. In tautomers a hydrogen migrates from one atom of the compound to another atom of the compound. Representative examples of tautomers include, but are not limited to, keto/enol and nitroso/oxime.

The present disclosure includes within its scope all the possible isotopically labeled forms of the compounds.

An isotopically labeled (radio-labeled) form of a compound of formula I is a compound of formula I, wherein one or more atoms are replaced by an atom having a mass number different from the mass number typically found in nature. Representative examples of isotopes that can be incorporated in the compounds of formula I include, but are not limited to, isotopes of hydrogen, carbon, nitrogen, oxygen, fluorine, sulfur, chlorine, and iodine, such as ²H, ³H, ¹¹C, ¹³C, ¹⁴C, ¹³N, ¹⁵N, ¹⁵O, ¹⁷O, ¹⁸O, ¹⁸F, ³⁵S, ³⁶Cl, ¹²³I, and ¹²⁵I.

The radionuclide that is incorporated in an isotopically labeled compound will depend on the specific application of said compound. For example, ³H and ¹²⁵I are useful for autoradiography. Positron emitting isotopes, such as ¹¹C, ¹³N, ¹⁵O, and ¹⁸F, are useful for positron emission tomography (PET) studies.

Autoradiography can offer quantitative information on molecular recognition, such as receptor binding, ex vivo. The compounds of formula I in isotopically labeled form can be used as GABA_{B} receptor autoradiography ligands.

PET can offer quantitative information on molecular recognition, such as receptor binding, in vivo in a mammal, such as a human. The compounds of formula I in isotopically labeled form can be used as GABA_{B} receptor PET tracers in a mammal, such as a human.

The compounds of formula I can be prepared by a variety of synthetic routes analogously to or according to methods known in the literature using suitable starting materials. Some methods useful for the preparation of the compounds of formula I are described below.

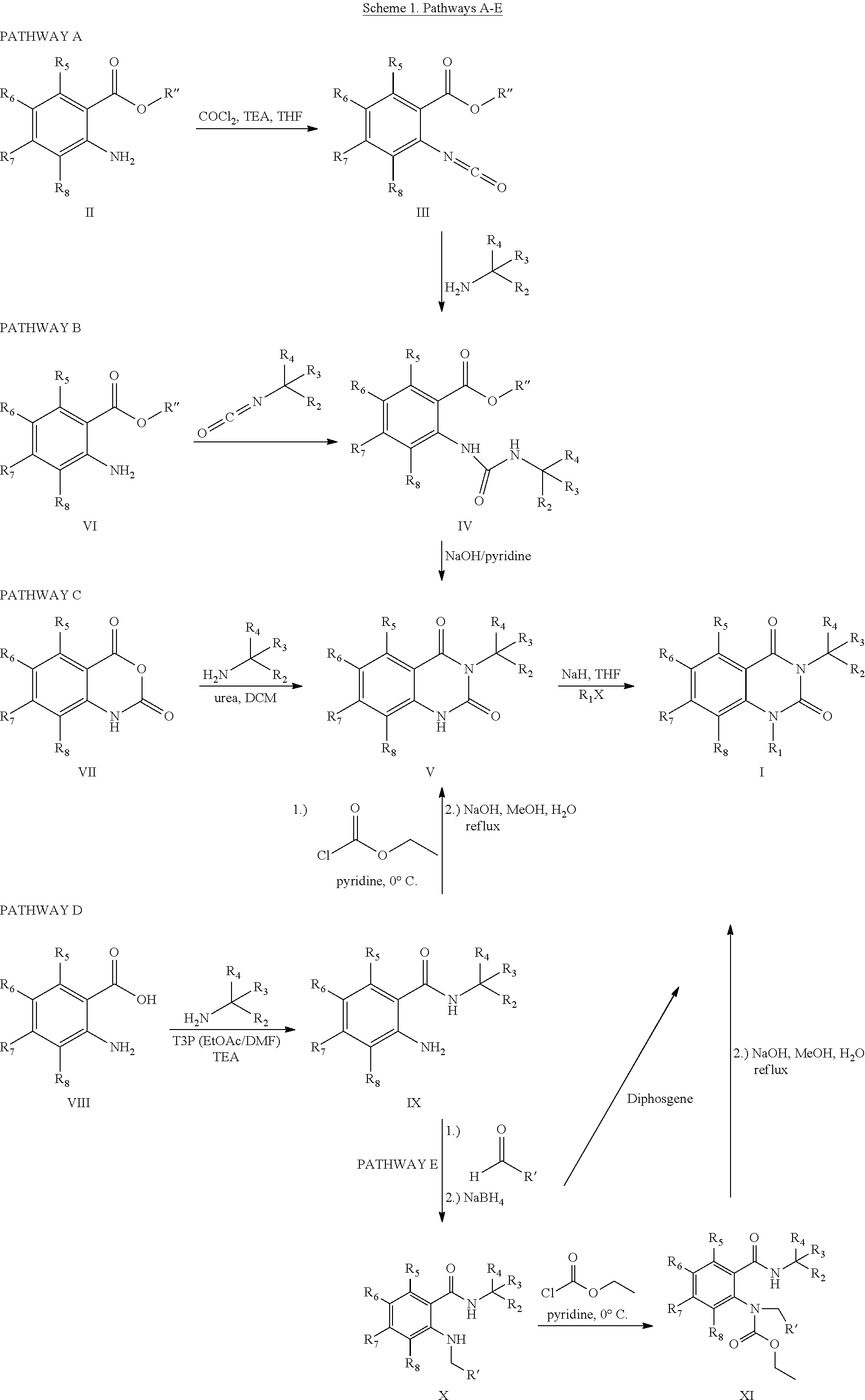

In Scheme 1, R₁, R₂, R₃, R₄, R₅, R₆, R₇, and R₈ are as defined above except that R₁ and R₈ do not form together —CHR₁₁—C(R₁₂)₂—O—, R′ is, for example, alkyl, and R″ is H or alkyl, such as ethyl.

The N-alkylation of V to yield I is carried out with a suitable base, such as NaH, NaOH, KOH, Cs₂CO₃, or K₂CO₃. The alkylation reagent is typically an electrophilic alkyl halide, R₁X, or an oxirane.

In pathway D, the amidation can be carried out by several methods and coupling reagents. Suitable coupling reagents are, for example, 1-propanephosphonic acid cyclic anhydride (T3P), 1-hydroxybenzotriazole (HOBt), or O-(benzotriazol-1-yl)-N,N,N′,N′— tetramethyluronium hexafluorophosphate (HBTU). The ring closure of IX to yield V can be carried out in one pot or the intermediate carbamate can be isolated. In addition to ethyl chloroformate, also other alkyl esters of chloroformic acid can be used. In addition to NaOH, also several other inorganic bases can be used in the ring closure. Also several organic bases can be used.

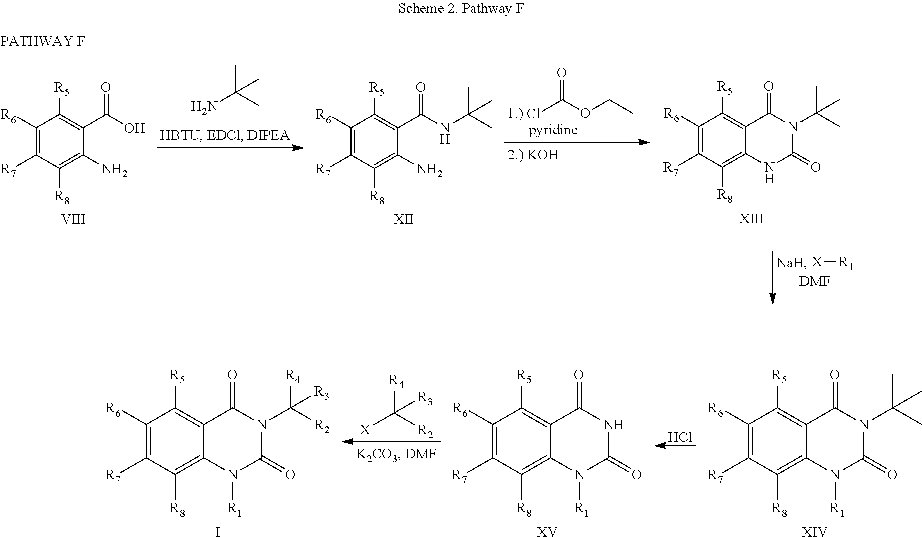

In Scheme 2, R₁, R₂, R₃, R₄, R₅, R₆, R₇, and R₈ are as defined above except that R₁ and R₈ do not form together —CHR₁₁—C(R₁₂)₂—O— and X is a suitable leaving group, e.g. chlorine or bromine. The amidation of VIII to yield XII can be carried out by several methods and coupling reagents. In addition to ethyl chloroformate, also other alkyl esters of chloroformic acid can be used in the ring closure.

Compounds of formula I, wherein R₁ and R₈ form together —CHR₁₁—C(R₁₂)₂—O—, can be prepared as depicted in Scheme 3.

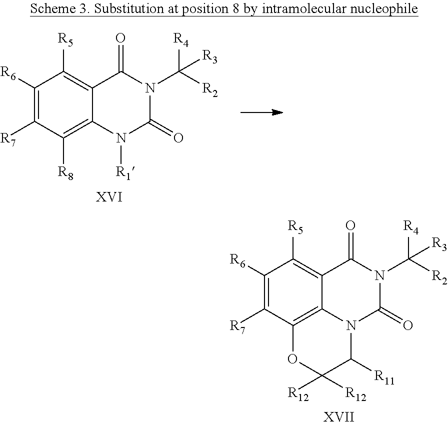

In Scheme 3, R₂, R₃, R₄, R₅, R₆, R₇, R₁₁, and R₁₂ are as defined above, R₁′ is a substituent having suitable nucleophilicity, such as alcohol functionality, R₈′ is a leaving group, such as fluorine, and m is 2, 3, or 4.

The ring closure is carried out with a suitable base, such as NaOH or KOH.

Compounds of formula I, wherein R₈ is (C₁-C₃)alkoxy or methoxy(C₁-C₃)alkoxy, can be prepared also as depicted in Scheme 4.

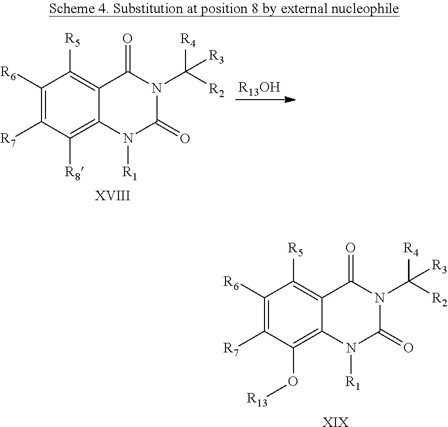

In Scheme 4, R₁, R₂, R₃, R₄, R₅, R₆, and R₇ are as defined above, R₈′ is a leaving group, such as fluorine, and R₁₃ is (C₁-C₃)alkyl or methoxy(C₁-C₃)alkyl.

The conversion of XVIII to XIX is carried out with a suitable base, such as NaOH or KOH.

Stepwise routes can be used. For instance, nucleophilic substitution at position 8 can be carried out prior to insertion of the substituent at position 1.

Any starting material or intermediate in the reactions to prepare compounds according to the present disclosure can be protected, if necessary, in a manner well known in the chemical field. Any protected functionality can subsequently be deprotected in a manner known in the art.

The synthetic routes described above are meant to illustrate the preparation of the compounds of formula I and the preparation is by no means limited thereto, that is there are also other possible synthetic methods which are within the general knowledge of a person skilled in the art.

The compounds of formula I can be purified with crystallization, column chromatography, preparative high-performance liquid chromatography (HPLC), or evaporation. Suitable crystallization solvents are, for example, ethyl acetate, diethyl ether, acetonitrile, ethanol, toluene, or mixtures thereof.

The compounds of formula I may be converted, if desired, into their pharmaceutically acceptable salt form using methods well known in the art.

The compounds of formula I in isotopically labeled form can be prepared by procedures analogous to those described above by using an isotopically labeled reagent instead of an isotopically unlabeled reagent.

The present disclosure will be explained in more detail by the following examples. The examples are meant for illustrating purposes only and do not limit the scope of the invention defined in the claims.

The abbreviations have the meanings indicated below.

ACN acetonitrile
AcOH acetic acid
DCE 1,2-dichloroethane
DCM dichloromethane

### DIPEA N,N-diisopropylethylamine

### DMA N,N-dimethylacetamide

### DMF N,N-dimethylformamide

DMSO dimethyl sulfoxide
EDCI 1-ethyl-3-(3-dimethylaminopropyl)carbodiimide
EtOAc ethyl acetate
EtOH ethanol
HBTU 2-(1H-benzotriazole-1-yl)-1,1,3,3-tetramethyluronium hexafluorophosphate
IPA isopropyl alcohol
MeOH methanol
rt room temperature
T3P 1-propanephosphonic acid cyclic anhydride
TBAB tetrabutylammonium bromide
TBAF tetrabutylammonium fluoride
TEA triethylamine
THF tetrahydrofuran

NMR spectrum multiplicities have the meanings indicated below.

br d broad doublet
br s broad singlet
d doublet
dd doublet of doublet
ddd doublet of doublet of doublet
dq doublet of quartet
m multiplet
q quartet
quint quintet
s singlet
t triplet
td triplet of doublet

### Example 1: 3-(4-Bromobenzyl)-5,7-dimethoxy-1-methylquinazoline-2,4(1H,3H)-dione

### 3-(4-Bromobenzyl)-5,7-dimethoxyquinazoline-2,4(1H,3H)-dione

2-Amino-4,6-dimethoxybenzoic acid (400 mg; 2.0 mmol), 10 ml of dry THF, and TEA (2 ml; 14.4 mmol) were placed in a reaction flask under nitrogen. 4-Bromobenzyl isocyanate (0.31 ml; 2.2 mmol) was added slowly with syringe and heated for 1 h at 80° C. to complete intermediate urea formation. The reaction mixture was evaporated to dryness. 3 ml of EtOH and sodium ethoxide solution (5 ml; 13.4 mmol; 21 m-% in EtOH) were added and the mixture was refluxed for 22 h to complete the reaction. The reaction mixture was cooled to rt, water was added, and pH was adjusted to ˜6 with 2 M HCl. The precipitation was filtered, washed with water, and dried to yield 832 mg of crude product, which was purified with EtOAc:heptane (1:1) trituration to give 696 mg of 3-(4-bromobenzyl)-5,7-dimethoxyquinazoline-2,4(1H,3H)-dione. LC-MS (ES+APCI, Pos) [M+1]: 391.1; (ES+APCI, Neg) [M−1]: 389.0.

### 3-(4-Bromobenzyl)-5,7-dimethoxy-1-methylquinazoline-2,4(1H,3H)-dione

Sodium hydride (89 mg; 2.2 mmol; 60% in oil) was placed in a reaction flask under nitrogen, 1 ml of dry THF and 3-(4-bromobenzyl)-5,7-dimethoxyquinazoline-2,4(1H,3H)-dione (350 mg; 0.44 mmol; 50% purity) in dry THF at 0° C. were added, and then 1 ml of dry DMF was added. The mixture was stirred for 30 min at rt. Iodomethane (0.14 ml; 2.24 mmol) was added dropwise and the mixture was stirred overnight at rt to complete the reaction. Water was added carefully. The mixture was extracted three times with DCM and combined organic phase was dried with a phase separator and evaporated to dryness. The crude product was purified with MS-Trigger to yield 84.6 mg of 3-(4-bromobenzyl)-5,7-dimethoxy-1-methylquinazoline-2,4(1H,3H)-dione. ¹H-NMR (400 MHz, CDCl₃): δ 3.54 (s, 3H), 3.91 (s, 3H), 3.97 (s, 3H), 5.15 (s, 2H), 6.21 (d, 1H), 6.28 (d, 1H), 7.37-7.44 (m, 4H).

### Example 2: 3-(3,4-Dichlorobenzyl)-1-(2-hydroxy-2-methylpropyl)-7-(trifluoromethyl)quinazoline-2,4(1H,3H)-dione

### 3-(3,4-Dichlorobenzyl)-7-(trifluoromethyl)quinazoline-2,4(1H,3H)-dione

2-Amino-4-(trifluoromethyl)benzoic acid (1.0 g; 4.9 mmol) and 8 ml of dry pyridine were placed in a microwave reaction vial and 3,4-dichlorobenzyl isocyanate (1.5 ml; 9.73 mmol) in dry pyridine (2 ml) was slowly added. The reaction mixture was heated at 100° C. for 3 h and at 200° C. for 15 min. After cooling to rt, aqueous HCl was added and the resulting precipitation was filtered and washed with water and DCM. The crude product was purified with normal and reverse phase column chromatography to give 310 mg of 3-(3,4-dichlorobenzyl)-7-(trifluoromethyl)quinazoline-2,4(1H,3H)-dione. ¹H-NMR (400 MHz, d₆-DMSO): δ 5.07 (s, 2H), 7.31-7.35 (m, 1H), 7.47-7.49 (m, 1H), 7.50-7.54 (m, 1H), 7.57 (d, 1H), 7.61-7.63 (m, 1H), 8.12-8.16 (m, 1H), 11.83 (br s, 1H).

### 3-(3,4-Dichlorobenzyl)-1-(2-hydroxy-2-methylpropyl)-7-(trifluoromethyl)quinazoline-2,4(1H,3H)-dione

3-(3,4-Dichlorobenzyl)-7-(trifluoromethyDquinazoline-2,4(1H,3H)-dione (200 mg; 0.51 mmol) and yttrium(III) nitrate hexahydrate (9.8 mg; 0.026 mmol) were placed in a microwave reaction vial. DMF (3 ml) and isobutylene oxide (9.13 ml; 103 mmol) were added and the reaction mixture was heated in a microwave reactor at 160° C. for 60 min. After cooling to rt, saturated sodium bicarbonate was added and the mixture was extracted with DCM. The combined organic layers were washed with water and brine, dried with a phase separator, and evaporated to dryness. The crude product was purified with column chromatography (EtOAc:heptane) to give 192 mg of 3-(3,4-dichlorobenzyl)-1-(2-hydroxy-2-methylpropyl)-7-(trifluoromethyDquinazoline-2,4(1H,3H)-dione. ¹H-NMR (400 MHz, CDCl₃): δ 1.35 (s, 6H), 2.17 (s, 1H), 4.26 (s, 2H), 5.22 (s, 2H), 7.33-7.40 (m, 2H), 7.47-7.52 (m, 1H), 7.59-7.62 (m, 1H), 7.82-7.86 (m, 1H), 8.33-8.37 (m, 1H).

### Example 3: 6,7-Difluoro-1-(2-hydroxy-2-methylpropyl)-3-(4-methoxybenzyl)quinazoline-2,4(1H,3H)-dione

### 6,7-Difluoro-3-(4-methoxybenzyl)quinazoline-2,4(1H,3H)-dione

6,7-Difluoro-3-(4-methoxybenzyl)quinazoline-2,4(1H,3H)-dione was prepared similarly to 3-(4-bromobenzyl)-5,7-dimethoxyquinazoline-2,4(1H,3H)-dione in Example 1. 2-Amino-4,5-difluorobenzoic acid (250 mg; 1.44 mmol) and dry pyridine (2.5 ml) were placed in a microwave reaction vial and 4-methoxybenzyl isocyanate (353 mg; 2.17 mmol) was slowly added. After heating in a microwave reactor at 200° C. for 30 min, aqueous sodium hydroxide (5 N; 0.43 ml; 2.17 mmol) was added and the reaction mixture was heated at 140° C. for 30 min. Addition of aqueous HCl resulted in a precipitation that was filtered, washed with water, and dried to give 290 mg of crude 6,7-difluoro-3-(4-methoxybenzyl)quinazoline-2,4(1H,3H)-dione. LC-MS (ES+APCI, Neg) [M−1]: 317.0.

### 6,7-Difluoro-1-(2-hydroxy-2-methylpropyl)-3-(4-methoxybenzyl)quinazoline-2,4(1H,3H)-dione

6,7-Difluoro-1-(2-hydroxy-2-methylpropyl)-3-(4-methoxybenzyl)quinazoline-2,4(1H,3H)-dione was prepared similarly to 3-(3,4-dichlorobenzyl)-1-(2-hydroxy-2-methylpropyl)-7-(trifluoromethyDquinazoline-2,4(1H,3H)-dione in Example 2. Crude 6,7-difluoro-3-(4-methoxybenzyl)quinazoline-2,4(1H,3H)-dione (285 mg; 0.448 mmol) and yttrium(III) nitrate hexahydrate (17.2 mg; 0.045 mmol) were placed in a microwave reaction vial. DMF (1 ml) and isobutylene oxide (2.98 ml; 33.6 mmol) were added and the reaction mixture was heated in a microwave reactor at 160° C. for 60 min. After work-up, the crude product was purified with column chromatography (EtOAc:heptane) to give 11 mg of 6,7-difluoro-1-(2-hydroxy-2-methylpropyl)-3-(4-methoxybenzyl)quinazoline-2,4-(1H,3H)-dione. ¹H-NMR (400 MHz, CDCl₃): δ 1.33 (s, 6H), 2.39 (br s, 1H), 3.76 (s, 3H), 4.13 (s, 2H), 5.19 (s, 2H), 6.80-6.85 (m, 2H), 7.40 (dd, 1H), 7.43-7.48 (m, 2H), 7.99 (dd, 1H).

### Example 4: 7-Chloro-6-fluoro-1-(2-hydroxy-2-methylpropyl)-3-(4-methoxybenzyl)quinazoline-2,4(1H,3H)-dione

### 7-Chloro-6-fluoro-3-(4-methoxybenzyl)quinazoline-2,4(1H,3H)-dione

7-Chloro-6-fluoro-3-(4-methoxybenzyl)quinazoline-2,4(1H,3H)-dione was prepared similarly to 3-(4-bromobenzyl)-5,7-dimethoxyquinazoline-2,4(1H,3H)-dione in Example 1. 2-Amino-4-chloro-5-fluorobenzoic acid (250 mg; 1.32 mmol) and dry pyridine (2.5 ml) were placed in a microwave reaction vial and 4-methoxybenzyl isocyanate (323 mg; 1.98 mmol) was slowly added. After heating in a microwave reactor at 200° C. for 30 min, aqueous sodium hydroxide (5 N; 0.40 ml; 1.98 mmol) was added and the reaction mixture was heated at 140° C. for 30 min. Addition of aqueous HCl resulted in a precipitation that was filtered, washed with water, and dried to give 500 mg of crude 7-chloro-6-fluoro-3-(4-methoxybenzyl)quinazoline-2,4(1H,3H)-dione. LC-MS (ES+APCI, Neg) [M−1]: 333.0.

### 7-Chloro-6-fluoro-1-(2-hydroxy-2-methylpropyl)-3-(4-methoxybenzyl)quinazoline-2,4(1H,3H)-dione

7-Chloro-6-fluoro-1-(2-hydroxy-2-methylpropyl)-3-(4-methoxybenzyl)quinazoline-2,4(1H,3H)-dione was prepared similarly to 3-(3,4-dichlorobenzyl)-1-(2-hydroxy-2-methylpropyl)-7-(trifluoromethyDquinazoline-2,4(1H,3H)-dione in Example 2. Crude 7-chloro-6-fluoro-3-(4-methoxybenzyl)quinazoline-2,4(1H,3H)-dione (500 mg; 0.64 mmol) and yttrium(III) nitrate hexahydrate (24.6 mg; 0.064 mmol) were placed in a microwave reaction vial. DMF (3 ml) and isobutylene oxide (8.56 ml; 96 mmol) were added and the reaction mixture was heated in a microwave reactor at 160° C. for 60 min. After work-up, the crude product was purified with column chromatography (EtOAc:heptane) and MS-Trigger to give 124 mg of 7-chloro-6-fluoro-1-(2-hydroxy-2-methylpropyl)-3-(4-methoxybenzyl)quinazoline-2,4(1H,3H)-dione. ¹H-NMR (400 MHz, CDCl₃): δ 1.33 (s, 6H), 2.34 (s, 1H), 3.77 (s, 3H), 4.16 (s, 2H), 5.19 (s, 2H), 6.79-6.85 (m, 2H), 7.42-7.48 (m, 2H), 7.62 (d, 1H), 7.95 (d, 1H).

### Example 5: 3-(4-Bromobenzyl)-6-(difluoromethoxy)-7-fluoro-1-methylquinazoline-2,4(1H,3H)-dione

3-(4-Bromobenzyl)-7-fluoro-6-hydroxy-1-methylquinazoline-2,4(1H,3H)-dione (150 mg; 0.40 mmol) and potassium hydroxide (0.44 g; 7.9 mmol) were placed in a reaction flask, which was then cooled to −20° C. ACN (3 ml) and water (3 ml) were added, followed by the addition of bromodifluoromethyl diethylphosphonate (275 mg; 0.99 mmol), and the reaction flask was allowed to warm to rt. After stirring overnight, the mixture was diluted with EtOAc and washed with water. The aqueous phase was extracted with EtOAc and the combined organic layers were washed with brine, dried with a phase separator, and evaporated to dryness. The crude product was purified with column chromatography (EtOAc:heptane) to give 96 mg of 3-(4-bromobenzyl)-6-(difluoro-methoxy)-7-fluoro-1-methylquinazoline-2,4(1H,3H)-dione. ¹H-NMR (400 MHz, CDCl₃): δ 3.56 (s, 3H), 5.19 (s, 2H), 6.57 (t, 1H), 7.00 (d, 1H), 7.37-7.46 (m, 4H), 8.10-8.14 (m, 1H).

### Example 6: 3-(4-Bromobenzyl)-7-fluoro-6-methoxy-1-methylquinazoline-2,4(1H,3H)-dione

### 3-(4-Bromobenzyl)-7-fluoro-6-methoxyquinazoline-2,4(1H,3H)-dione

Ethyl 2-amino-4-fluoro-5-methoxybenzoate (300 mg; 1.4 mmol), 2 ml of dry pyridine and 4-bromobenzyl isocyanate (0.22 ml; 1.55 mmol) were charged in a microwave tube and heated at 200° C. for 15 min. 4-Bromobenzyl isocyanate (0.06 ml; 0.42 mmol) was added and the reaction mixture was heated twice at 200° C. for 10 min and for 15 min. The reaction mixture was cooled to rt, water was added, and pH adjusted to neutral with 1 M HCl. The precipitation was filtered and the filtrate was extracted three times with EtOAc. Organic phases were combined, dried, and evaporated. The evaporation residue was purified with MS-Trigger and triturated with diethyl ether to yield 48 mg of 3-(4-bromobenzyl)-7-fluoro-6-methoxyquinazoline-2,4(1H,3H)-dione. ¹H-NMR (400 MHz, CDCl₃): δ 3.93 (s, 3H), 5.17 (s, 2H), 6.77 (d, 1H), 7.33-7.50 (m, 4H) 7.65 (d, 1H), 8.43 (br s, 1H).

### 3-(4-Bromobenzyl)-7-fluoro-6-methoxy-1-methylquinazoline-2,4(1H,3H)-dione

3-(4-Bromobenzyl)-7-fluoro-6-methoxyquinazoline-2,4(1H,3H)-dione (100 mg; 0.26 mmol), 2 ml of dry DMF and K₂CO₃ (72.9 mg; 0.53 mmol) were charged in a reaction flask under nitrogen. The mixture was stirred at rt for 15 min, iodomethane (0.033 ml; 0.53 mmol) was added, and the reaction mixture was stirred at rt over three nights. 0.1 M citric acid was added and the precipitation was filtered and washed with water. The crude product was purified by triturating with diethyl ether and CombiFlash (reverse phase silica) to yield 76 mg of 3-(4-bromobenzyl)-7-fluoro-6-methoxy-1-methylquinazoline-2,4(1H,3H)-dione. ¹H-NMR (400 MHz, CDCl₃): δ 3.54 (s, 3H), 3.95 (s, 3H), 5.21 (s, 2H), 6.96 (d, 1H), 7.35-7.48 (m, 4H), 7.76 (d, 1H).

### Example 7: 3-(4-Bromobenzyl)-7-fluoro-6-hydroxy-1-methylquinazoline-2,4(1H,3H)-dione

3-(4-Bromobenzyl)-7-fluoro-6-methoxy-1-methylquinazoline-2,4(1H,3H)-dione (40 mg; 0.10 mmol) prepared in Example 6 and 1 ml of dry DCM were charged in a reaction flask under nitrogen. The mixture was cooled to 0° C., 1 M boron tribromide solution in DCM (0.1 ml; 0.10 mmol) was added slowly, and the mixture was stirred at rt over three nights. DCM and 1 M boron tribromide solution in DCM (0.1 ml; 0.10 mmol) was added again and the mixture was stirred over two nights. 1 M boron tribromide solution in DCM (0.2 ml; 0.20 mmol) was added three times and the mixture was stirred overnight between the additions. The mixture was cooled, water and MeOH were added, and the mixture was extracted twice with DCM. Organic phases were combined, dried, and evaporated. The evaporation residue was purified with CombiFlash (normal phase silica) to yield 19 mg of 3-(4-bromobenzyl)-7-fluoro-6-hydroxy-1-methylquinazoline-2,4(1H,3H)-dione. ¹H-NMR (400 MHz, CDCl₃): δ 3.54 (s, 3H), 5.19 (s, 2H), 5.40-5.43 (m, 1H), 6.95 (d, 1H), 7.36-7.44 (m, 4H), 7.87 (d, 1H).

### Example 8: 7-(4-Bromobenzyl)-5-methyl-[1,3]dioxolo[4,5-g]quinazoline-6,8(5H,7H)-dione

### 7-(4-Bromobenzyl)-[1,3]dioxolo[4,5-g]quinazoline-6,8(5H,7H)-dione

6-Amino-1,3-benzodioxole-5-carboxylic acid (150 mg; 0.83 mmol) and 5 ml of dry THF were charged in a reaction flask under nitrogen. 4-Bromobenzyl isocyanate (0.128 ml; 0.91 mmol) and TEA (0.7 ml; 5.0 mmol) were added and the reaction mixture was heated to 80° C. for 1 h. The mixture was cooled and solvents were evaporated. 5 ml EtOH and 0.9 ml 2 M NaOH solution were added and the reaction mixture was refluxed for 2 h. The mixture was cooled, water was added, and pH was adjusted to neutral using 2 M HCl solution. The precipitation was filtered and the filtrate was evaporated to yield 217 mg of 7-(4-bromobenzyl)-[1,3]dioxolo[4,5-g]quinazoline-6,8-(5H,7H)-dione. LC-MS (ES+) [M+1]: 375.0.

### 7-(4-Bromobenzyl)-5-methyl-[1,3]dioxolo[4,5-g]quinazoline-6,8(5H,7H)-dione

Sodium hydride (45.8 mg; 1.15 mmol; 60%) and 1 ml of dry DMF were charged in a reaction flask under nitrogen and the mixture was cooled to 0° C. 7-(4-Bromobenzyl)-[1,3]dioxolo[4,5-g]quinazoline-6,8(5H,7H)-dione (215 mg; 0.573 mmol) was added, the mixture was stirred at rt for 30 min, and iodomethane (0.071 ml; 1.15 mmol) was added. The reaction mixture was stirred at rt for three days. MeOH was added and the mixture was evaporated. Water and DCM were added to the evaporation residue and the mixture was extracted twice with DCM. Organic phases were combined, dried, and evaporated. The crude product was purified with CombiFlash (normal phase silica) and triturated with MeOH to yield 19.5 mg of 7-(4-bromobenzyl)-5-methyl-[1,3]dioxolo[4,5-g]quinazoline-6,8(5H,7H)-dione. ¹H-NMR (400 MHz, CDCl₃): δ 3.54 (s, 3H), 5.19 (s, 2H), 6.08 (s, 2H), 6.67 (s, 1H), 7.34-7.47 (m, 4H), 7.57 (s, 1H).

### Example 9: 3-(4-Bromobenzyl)-1-isopropyl-6,7-dimethoxyquinazoline-2,4(1H,3H)-dione

### 3-(4-Bromobenzyl)-6,7-dimethoxyquinazoline-2,4(1H,3H)-dione

2-Amino-4,5-dimethoxybenzoic acid (250 mg; 1.27 mmol) and dry THF were charged in a reaction flask under nitrogen. 4-Bromobenzyl isocyanate (0.195 ml; 1.40 mmol) and TEA (1.1 ml; 7.89 mmol) were added and the reaction mixture was heated at 80° C. for 1 h. The mixture was cooled and solvents were evaporated. EtOH and 1.4 ml 2 M NaOH solution were added and the reaction mixture was refluxed for few hours. The mixture was stirred at rt overnight and the next day the mixture was refluxed several hours. 1.4 ml 2 M NaOH solution was added and the mixture was refluxed again for 9 h totally. The mixture was cooled, water was added, and pH was adjusted to neutral using 2 M HCl solution. The precipitation was filtered to yield 440 mg of 3-(4-bromobenzyl)-6,7-dimethoxyquinazoline-2,4(1H,3H)-dione. LC-MS (ES+) [M+1]: 391.0.

### 3-(4-Bromobenzyl)-1-isopropyl-6,7-dimethoxyquinazoline-2,4(1H,3H)-dione

Sodium hydride (20.9 mg; 0.52 mmol; 60%) and dry DMF were charged in a reaction flask under nitrogen and the mixture was cooled to 0° C. 3-(4-Bromobenzyl)-6,7-dimethoxyquinazoline-2,4(1H,3H)-dione (120 mg; 0.307 mmol) was dissolved in dry DMF and added to the reaction. The mixture was stirred at rt for 30 min and 2-iodopropane (0.031 ml; 0.307 mmol) was added. The reaction mixture was stirred at rt overnight. Sodium hydride and 2-iodopropane were added twice and the reaction mixture was stirred at rt for two more nights. MeOH was added and the mixture was evaporated. Water and DCM were added to the evaporation residue and the mixture was extracted twice with DCM. Organic phases were combined, dried, and evaporated. The crude product was purified with MS-Trigger to yield 43 mg of 3-(4-bromobenzyl)-1-isopropyl-6,7-dimethoxyquinazoline-2,4(1H,3H)-dione. ¹H-NMR (400 MHz, CDCl₃): δ 1.61 (d, 6H), 3.93 (s, 3H), 3.99 (s, 3H), 4.93-5.14 (m, 1H), 5.19 (s, 2H), 6.78 (s, 1H) 7.33-7.47 (m, 4H), 7.62 (s, 1H).

### Example 10: 3-(4-Bromobenzyl)-1-(2-hydroxy-2-methylpropyl)-6,7-dimethoxyquinazoline-2,4(1H,3H)-dione

3-(4-Bromobenzyl)-6,7-dimethoxyquinazoline-2,4(1H,3H)-dione (120 mg; 0,307 mmol) prepared in Example 9, dry THF, and K₂CO₃ (50.9 mg; 0.37 mmol) were charged in a reaction flask under nitrogen. Isobutylene oxide (0.055 ml; 0.61 mmol) was added slowly and the reaction mixture was refluxed several hours and stirred at rt over the weekend. Solvent was evaporated, DMF was added to the evaporation residue, and the mixture was charged in a microwave tube. K₂CO₃ (50.9 mg) and isobutylene oxide (0.055 ml) were added and the mixture was heated four times at 200° C. for 10-15 min. The reaction mixture was evaporated and EtOAc was added. Organic phase was washed once with 1 M Na₂CO₃ solution and twice with water. Organic phase was dried and evaporated. The crude product was purified with MS-Trigger to yield 16 mg of 3-(4-bromobenzyl)-1-(2-hydroxy-2-methylpropyl)-6,7-dimethoxyquinazoline-2,4(1H,3H)-dione. NMR (400 MHz, CDCl₃): 1.34 (s, 6H), 2.54 (s, 1H), 3.94 (s, 3H), 3.97 (s, 3H), 4.20 (s, 2H), 5.22 (s, 2H), 7.03 (s, 1H), 7.33-7.46 (m, 4H), 7.59 (s, 1H).

### Example 11: 3-(4-Bromobenzyl)-6,7-difluoro-1-methylquinazoline-2,4(1H,3H)-dione

### 3-(4-Bromobenzyl)-6,7-difluoroquinazoline-2,4(1H,3H)-dione

4,5-Difluoroanthranilic acid (300 mg; 1.73 mmol) and dry THF were charged in a reaction flask under nitrogen. 4-Bromobenzyl isocyanate (0.267 ml; 1.91 mmol) and TEA (1.5 ml; 10.76 mmol) were added and the reaction mixture was heated at 80° C. for 1 h and at rt overnight. Solvents were evaporated. EtOH and 2 ml 2 M NaOH solution were added and the reaction mixture was refluxed for 5 h. The mixture was cooled, water was added, and pH was adjusted to neutral using 2 M HCl solution. The precipitation was filtered to yield 648 mg of 3-(4-bromobenzyl)-6,7-difluoroquinazoline-2,4(1H,3H)-dione. LC-MS (ES−) [M−1]: 366.9.

### 3-(4-Bromobenzyl)-6,7-difluoro-1-methylquinazoline-2,4(1H,3H)-dione

Sodium hydride (37 mg; 0.93 mmol; 60%) and 1 ml of dry DMF were charged in a reaction flask under nitrogen and the mixture was cooled to 0° C. 3-(4-Bromobenzyl)-6,7-difluoroquinazoline-2,4(1H,3H)-dione (200 mg; 0.345 mmol) was dissolved in dry DMF and added to the reaction. The mixture was stirred at rt for 30 min and iodomethane (0.058 ml; 0.926 mmol) was added. The reaction mixture was stirred at rt over the weekend. MeOH was added and the mixture was evaporated. Water and DCM were added to the evaporation residue and the mixture was extracted twice with DCM. Organic phases were combined, dried, and evaporated. The crude product was purified with CombiFlash (normal phase silica) to yield 30 mg of 3-(4-bromobenzyl)-6,7-difluoro-1-methylquinazoline-2,4(1H,3H)-dione. ¹H-NMR (400 MHz, d₆-DMSO): δ 3.50 (s, 3H), 5.08 (s, 2H), 7.25-7.37 (m, 2H), 7.43-7.55 (m, 2H), 7.70 (dd, 1H), 8.02 (dd, 1H).

### Example 12: 1-Methyl-3-(1,2,3,4-tetrahydronaphthalen-1-yl)-7-(trifluoromethyl)quinazoline-2,4(1H,3H)-dione

2-Amino-4-(trifluoromethyl)benzoic acid (200 mg; 0.975 mmol) and dry THF were charged in a reaction flask under nitrogen. 1-Isocyanato-1,2,3,4-tetrahydronaphthalene (0.168 ml; 1.07 mmol) dissolved in a small amount of THF and TEA (0.9 ml; 6.46 mmol) were added and the reaction mixture was heated at 80° C. for 1 h. Solvents were evaporated and EtOH and 1.1 ml 2 M sodium hydroxide solution were added, and the reaction mixture was refluxed for 1½ h. The reaction mixture was stirred at rt overnight and the next day refluxed for 5 h. The previous procedure was repeated. Water was added and pH was adjusted to neutral using 2 M HCl solution. The precipitation was filtered to yield 227 mg of intermediate as a crude mixture.

Sodium hydride (50.4 mg; 1.26 mmol; 60%) and dry DMF were charged in a reaction flask under nitrogen and the mixture was cooled to 0° C. The intermediate (227 mg) dissolved in dry DMF was added, the mixture was stirred at rt for 30 min, and iodomethane (0.0578 ml; 1.26 mmol) was added. The reaction mixture was stirred at rt overnight. MeOH was added and the mixture was evaporated. Water and DCM were added to the evaporation residue and the mixture was extracted twice with DCM. Organic phases were combined, dried, and evaporated. The crude product was purified with CombiFlash (normal phase silica) and triturated with MeOH to yield 20 mg of 1-methyl-3-(1,2,3,4-tetrahydronaphthalen-1-yl)-7-(trifluoromethyDquinazoline-2,4(1H,3H)-dione. ¹H-NMR (400 MHz, CDCl₃): δ 1.79-1.95 (m, 1H), 2.05-2.18 (m, 2H), 2.41-2.56 (m, 1H), 2.74-2.87 (m, 1H), 2.96-3.11 (m, 1H), 3.60 (br s, 3H), 6.35 (br s, 1H), 6.89 (d, 1H), 7.00-7.08 (m, 1H), 7.08-7.18 (m, 2H), 7.43 (s, 1H), 7.50 (d, 1H), 8.34 (br s, 1H).

### Example 13: 3-(4-Bromobenzyl)-1-methyl-6-(trifluoromethyl)quinazoline-2,4(1H,3H)-dione

### 3-(4-Bromobenzyl)-6-(trifluoromethyl)quinazoline-2,4(1H,3H)-dione

2-Amino-5-(trifluoromethyl)benzoic acid (200 mg; 0.975 mmol) and dry THF were charged in a reaction flask under nitrogen. 4-Bromobenzyl isocyanate (0.150 ml; 1.07 mmol) dissolved in a small amount of THF and TEA (0.9 ml; 6.46 mmol) were added to the reaction and the reaction mixture was heated at 80° C. for 2 h. Solvents were evaporated and EtOH and 1.2 ml 2 M sodium hydroxide solution were added. The reaction mixture was refluxed for 14 h. The mixture was cooled, water was added, and pH was adjusted to neutral using 2 M HCl solution. The mixture was extracted three times with EtOAc. Organic phases were combined, evaporated, and dried in a vacuum oven to yield 381 mg of 3-(4-bromobenzyl)-6-(trifluoromethyDquinazoline-2,4(1H,3H)-dione. LC-MS (ES−) [M−1]: 398.9.

### 3-(4-Bromobenzyl)-1-methyl-6-(trifluoromethyl)quinazoline-2,4(1H,3H)-dione

Sodium hydride (65 mg; 1.62 mmol; 60%) and 1 ml of dry DMF were charged in a reaction flask under nitrogen and the mixture was cooled to 0° C. 3-(4-Bromobenzyl)-6-(trifluoromethyDquinazoline-2,4(1H,3H)-dione (380 mg; 0.952 mmol) in 2 ml of dry DMF was added and the mixture was stirred at rt for 30 min. Iodomethane (0.101 ml; 1.62 mmol) was added and the reaction mixture was stirred at rt overnight. MeOH was added and the mixture was evaporated. Water and DCM were added to the evaporation residue and the mixture was extracted twice with DCM. Organic phases were combined, dried, and evaporated. The crude product was purified with CombiFlash (normal phase silica) to yield 191 mg of 3-(4-bromobenzyl)-1-methyl-6-(trifluoromethyDquinazoline-2,4(1H,3H)-dione. ¹H-NMR (400 MHz, d₆-DMSO): δ 3.56 (s, 3H), 5.11 (s, 2H), 7.27-7.36 (m, 2H), 7.45-7.54 (m, 2H), 7.68 (d, 1H), 8.12 (dd, 1H), 8.28 (d, 1H).

### Example 14: 3-(3,4-Dichlorobenzyl)-1-methyl-7-(trifluoromethyl)quinazoline-2,4(1H,3H)-dione

### 3-(3,4-Dichlorobenzyl)-7-(trifluoromethyl)quinazoline-2,4(1H,3H)-dione

2-Amino-4-(trifluoromethyl)benzoic acid (150 mg; 0.731 mmol) and 5 ml of dry THF were charged in a reaction flask under nitrogen. 3,4-Dichlorobenzyl isocyanate (0.118 ml; 0.80 mmol) dissolved in a small amount of THF and TEA (0.7 ml; 5.02 mmol) were added to the reaction mixture and the reaction mixture was heated at 80° C. for 1 h. Solvents were evaporated and EtOH and 1 ml 2 M sodium hydroxide solution were added. The reaction mixture was refluxed for 6 h and stirred at rt overnight. Water was added and pH was adjusted to neutral using 2 M HCl solution. The mixture was extracted three times with EtOAc. Organic phases were combined, dried, and evaporated to yield 308 mg of 3-(3,4-dichlorobenzyl)-7-(trifluoromethyl)quinazoline-2,4(1H,3H)-dione. LC-MS (ES−) [M−1]: 387.0.

### 3-(3,4-Dichlorobenzyl)-1-methyl-7-(trifluoromethyl)quinazoline-2,4(1H,3H)-dione

Sodium hydride (47 mg; 1.19 mmol; 60%) and 1 ml of dry DMF were charged in a reaction flask under nitrogen and the mixture was cooled to 0° C. 3-(3,4-Dichlorobenzyl)-7-(trifluoromethyl)quinazoline-2,4(1H,3H)-dione (308 mg; 0.79 mmol) in 2 ml of dry DMF was added and the mixture was stirred at rt for 30 min. Iodomethane (0.074 ml; 1.19 mmol) was added and the reaction mixture was stirred at rt overnight. MeOH was added and the mixture was evaporated to dryness. Water and DCM were added to the evaporation residue and the mixture was extracted twice with DCM. Organic phases were combined, dried, and evaporated. The crude product was purified by triturating with MeOH to yield 127 mg of 3-(3,4-dichlorobenzyl)-1-methyl-7-(trifluoromethyl)quinazoline-2,4(1H,3H)-dione. ¹H-NMR (400 MHz, d₆-DMSO): δ 3.59 (s, 3H), 5.13 (s, 2H), 7.35 (dd, 1H), 7.50-7.69 (m, 3H), 7.76 (s, 1H), 8.26 (d, 1H).

### Example 15: 3-(4-Bromobenzyl)-1-methyl-7-(trifluoromethyl)quinazoline-2,4(1H,3H)-dione

### 3-(4-Bromobenzyl)-7-(trifluoromethyl)quinazoline-2,4(1H,3H)-dione

2-Amino-4-(trifluoromethyl)benzoic acid (400 mg; 1.95 mmol) and 10 ml of dry THF were charged in a reaction flask under nitrogen. 4-Bromobenzyl isocyanate (0.300 ml; 2.15 mmol) dissolved in a small amount of THF and TEA (1.7 ml; 12.2 mmol) were added to the reaction mixture and the reaction mixture was heated at 80° C. for 1 h. Solvents were evaporated. EtOH and 2 ml 2 M sodium hydroxide solution were added and the reaction mixture was refluxed for 3 h. Water was added and pH was adjusted to neutral using 2 M HCl solution. The mixture was extracted three times with EtOAc. Organic phases were combined, dried, and evaporated to yield 630 mg of 3-(4-bromobenzyl)-7-(trifluoromethyl)quinazoline-2,4(1H,3H)-dione. LC-MS (ES−) [M−1]: 398.99.

### 3-(4-Bromobenzyl)-1-methyl-7-(trifluoromethyl)quinazoline-2,4(1H,3H)-dione

Sodium hydride (107 mg; 2.68 mmol; 60%) and dry DMF were charged in a reaction flask under nitrogen and the mixture was cooled to 0° C. 3-(4-Bromobenzyl)-7-(trifluoromethyl)quinazoline-2,4(1H,3H)-dione (630 mg; 1.58 mmol) in 3 ml of dry DMF was added and the mixture was stirred at rt for 1 h. Iodomethane (0.167 ml; 2.68 mmol) was added and the reaction mixture was stirred at rt overnight. MeOH was added and the mixture was evaporated. Water and DCM were added to the evaporation residue and the mixture was extracted twice with DCM. Organic phases were combined, dried, and evaporated. The crude product was purified with CombiFlash (normal phase silica) to yield 390 mg of 3-(4-bromobenzyl)-1-methyl-7-(trifluoromethyl)quinazoline-2,4(1H,3H)-dione. ¹H-NMR (400 MHz, d₆-DMSO): δ 3.59 (s, 3H), 5.11 (s, 2H), 7.28-7.34 (m, 2H), 7.46-7.53 (m, 2H), 7.62-7.66 (m, 1H), 7.76 (br s, 1H), 8.26 (d, 1H).

### Example 16: 7-Fluoro-1-methyl-3-(4-(trifluoromethyl)benzyl)quinazoline-2,4(1H,3H)-dione

### 7-Fluoro-3-(4-(trifluoromethyl)benzyl)quinazoline-2,4(1H,3H)-dione

7-Fluoro-1H-benzo[d][1,3]oxazine-2,4-dione (109 mg; 0.6 mmol), (4-(trifluoromethyl)phenyl)methanamine (127 mg; 0.7 mmol), urea (54 mg; 0.9 mmol), and 0.5 ml of DMA were charged in a microwave tube and heated for 20 min at 250° C. The reaction mixture was cooled to rt, 5 ml of water was added, and the precipitation formed was filtered and dried to give 202 mg of crude product. LC-MS (ES−) [M−1]: 337.0.

### 7-Fluoro-1-methyl-3-(4-(trifluoromethyl)benzyl)quinazoline-2,4(1H,3H)-dione

Sodium hydride (40.6 mg; 1.0 mmol; 60% in oil) was charged in a reaction flask under nitrogen, the mixture was cooled to 0° C., and 1 nil of dry DMF was added. 7-Fluoro-3-(4-(trifluoromethyl)benzyl)quinazoline-2,4(1H,3H)-dione (202 mg; 0.60 mmol) in 2 ml of dry DMF was added dropwise and the mixture was stirred for 30 min at rt. Iodomethane (0.063 ml; 1.0 mmol) was added slowly at 0° C. and the reaction mixture was stirred overnight at rt. MeOH (0.5 ml) was added and the mixture was evaporated to dryness. DCM and water were added and phases were extracted. Organic phase was additionally washed twice with water, dried with a phase separator filtration, and evaporated to dryness. The crude product was purified with MeOH trituration and MS-Trigger to give 29 mg of 7-fluoro-1-methyl-3-(4-(trifluoromethyl)benzyl)quinazoline-2,4(1H,3H)-dione. ¹H-NMR (400 MHz, CDCl₃): δ 3.57 (s, 3H), 5.30 (s, 2H), 6.89 (dd, 1H), 6.98 (ddd, 1H), 7.54-7.64 (m, 4H), 8.25 (ddd, 1H).

### Example 17: 7-Fluoro-3-(3-fluoro-4-methoxybenzyl)-1-methylquinazoline-2,4(1H,3H)-dione

### 7-Fluoro-3-(3-fluoro-4-methoxybenzyl)quinazoline-2,4(1H,3H)-dione

7-Fluoro-3-(3-fluoro-4-methoxybenzyl)quinazoline-2,4(1H,3H)-dione was prepared similarly to 7-fluoro-3-(4-(trifluoromethyl)benzyl)quinazoline-2,4(1H,3H)-dione in Example 16. 7-Fluoro-1H-benzo[d][1,3]oxazine-2,4-dione (109 mg; 0.6 mmol), 3-fluoro-4-methoxybenzylamine (112 mg; 0.72 mmol), urea (54 mg; 0.90 mmol), and 0.5 ml of DMF were charged in a microwave tube and heated for 20 min at 250° C. The reaction mixture was cooled to rt, 5 ml of water was added, and the precipitation formed was filtered and dried to give 219 mg of crude product. LC-MS (ES−) [M−1]: 317.0.

### 7-Fluoro-3-(3-fluoro-4-methoxybenzyl)-1-methylquinazoline-2,4(1H,3H)-dione

7-Fluoro-3-(3-fluoro-4-methoxybenzyl)-1-methylquinazoline-2,4(1H,3H)-dione was prepared similarly to 7-fluoro-1-methyl-3-(4-(trifluoromethyl)benzyl)quinazoline-2,4(1H,3H)-dione in Example 16. Sodium hydride (40.8 mg; 1.02 mmol; 60% in oil) and 1 ml of DMF were charged and 7-fluoro-3-(3-fluoro-4-methoxybenzyl)quinazoline-2,4(1H,3H)-dione (191 mg; 0.60 mmol) in 2 ml of dry DMF was added. Iodomethane (0.064 ml; 1.02 mmol) was added and the reaction mixture was stirred overnight at rt. MeOH (0.5 ml), DCM, and water were added, water phase was washed twice with 10 ml of DCM and combined organic phases were dried with a phase separator, evaporated to dryness, and triturated with MeOH to give 134 mg of the product. Further CombiFlash (normal phase silica) purification gave 20 mg of 7-fluoro-3-(3-fluoro-4-methoxybenzyl)-1-methylquinazoline-2,4(1H,3H)-dione. ¹H-NMR (400 MHz, CDCl₃): δ 3.56 (s, 3H), 3.85 (s, 3H), 5.17 (s, 2H), 6.85-6.91 (m, 2H), 6.96 (ddd, 1H) 7.23-7.31 (m, 2H), 8.24 (dd, 1H).

### Example 18: 3-(4-Bromobenzyl)-7-chloro-1-methylquinazoline-2,4(1H,3H)-dione

### 3-(4-Bromobenzyl)-7-chloroquinazoline-2,4(1H,3H)-dione

3-(4-Bromobenzyl)-7-chloroquinazoline-2,4(1H,3H)-dione was prepared similarly to 7-fluoro-3-(4-(trifluoromethyl)benzyl)quinazoline-2,4(1H,3H)-dione in Example 16. 7-Chloro-1H-benzo[d][1,3]oxazine-2,4-dione (0.200 g; 1.01 mmol), (4-bromophenyl)methanamine (0.153 ml; 1.22 mmol), and urea (91 mg; 1.52 mmol) were used to obtain 0.370 g of crude product. ¹H-NMR (400 MHz, d₆-DMSO): δ 5.03 (s, 2H), 7.19-7.26 (m, 2H), 7.26-7.30 (m, 2H), 7.47-7.52 (m, 2H), 7.94 (d, 1H), 11.63 (br s, 1H).

### 3-(4-Bromobenzyl)-7-chloro-1-methylquinazoline-2,4(1H,3H)-dione

3-(4-Bromobenzyl)-7-chloro-1-methylquinazoline-2,4(1H,3H)-dione was prepared similarly to 7-fluoro-1-methyl-3-(4-(trifluoromethyl)benzyl)quinazoline-2,4(1H,3H)-dione in Example 16 using 3-(4-bromobenzyl)-7-chloroquinazoline-2,4(1H,3H)-dione (0.389 g; 1.07 mmol) as starting material. The crude product was triturated with MeOH to yield 0.305 g of 3-(4-bromobenzyl)-7-chloro-1-methylquinazoline-2,4(1H,3H)-dione. ¹H-NMR (400 MHz, d₆-DMSO): δ 3.52 (s, 3H), 5.08 (s, 2H), 7.27-7.32 (m, 2H), 7.36 (dd, 1H), 7.46-7.52 (m, 2H), 7.59 (d, 1H), 8.05 (d, 1H).

### Example 19: 3-(1-(4-Bromophenyl)ethyl)-7-fluoro-1-methylquinazoline-2,4(1H,3H)-dione

### 3-(1-(4-Bromophenyl)ethyl)-7-fluoroquinazoline-2,4(1H,3H)-dione

7-Fluoro-1H-benzo[d][1,3]oxazine-2,4-dione (0.500 g; 2.76 mmol), 1-(4-bromophenyl)ethanamine (0.474 ml; 3.31 mmol), urea (0.249 g; 4.14 mmol), and 3 ml of DMA were charged in a microwave tube and heated for 10 min at 250° C. The reaction mixture was cooled to rt, mixed with water, and extracted twice with EtOAc. Combined organic phases were dried over Na₂SO₄ and evaporated to dryness. The crude product was purified with CombiFlash (normal phase silica) to yield 389 mg of 3-(1-(4-bromophenyl)ethyl)-7-fluoroquinazoline-2,4(1H,3H)-dione. ¹H-NMR (400 MHz, d₆-DMSO): δ 1.77 (d, 3H), 6.11 (q, 1H), 6.22 (d, 1H), 6.60 (dd, 1H), 7.19-7.26 (m, 2H), 7.45-7.50 (m, 2H), 7.66 (d, 1H), 10.96 (br s, 1H).

### 3-(1-(4-Bromophenyl)ethyl)-7-fluoro-1-methylquinazoline-2,4(1H,3H)-dione

3-(1-(4-Bromophenyl)ethyl)-7-fluoro-1-methylquinazoline-2,4(1H,3H)-dione was prepared similarly to 7-fluoro-1-methyl-3-(4-(trifluoromethyl)benzyl)quinazoline-2,4(1H,3H)-dione in Example 16 using 3-(1-(4-bromophenyl)ethyl)-7-fluoroquinazoline-2,4(1H,3H)-dione (0.389 g; 1.07 mmol) as starting material. The crude product was purified with CombiFlash (normal phase silica) to yield 0.226 g of 3-(1-(4-bromophenyl)ethyl)-7-fluoro-1-methylquinazoline-2,4(1H,3H)-dione. ¹H-NMR (400 MHz, CDCl₃): δ 1.89 (d, 3H), 3.50 (s, 3H), 6.37 (q, 1H), 6.85 (dd, 1H), 6.95 (ddd, 1H), 7.30-7.35 (m, 2H), 7.40-7.45 (m, 2H), 8.21 (dd, 1H).

### Example 20: 3-((4-Chlorophenyl)(phenyl)methyl)-1,7-dimethylquinazoline-2,4(1H,3H)-dione

### 3-((4-Chlorophenyl)(phenyl)methyl)-7-methylquinazoline-2,4(1H,3H)-dione

4-Methylisatoic anhydride (80 mg; 0.45 mmol), (4-chlorophenyl)(phenyl)methanamine hydrochloride (138 mg; 0.54 mmol), urea (40.7 mg; 0.68 mmol), TEA (0.076 ml; 0.54 mmol), and 0.5 ml of DMA were charged in a microwave tube and heated for 15 min at 250° C. The microwave reaction was run again for the same time at the same temperature and then for 60 min at 250° C. The reaction mixture was cooled to rt, mixed with 5 ml of water, and extracted twice with 5 ml of EtOAc. Combined organic phases were dried over Na₂SO₄ and evaporated to dryness. The crude product was purified with CombiFlash (EtOAc:heptane; normal phase silica) to yield 30 mg of 3-((4-chlorophenyl)(phenyl)methyl)-7-methylquinazoline-2,4(1H,3H)-dione. LC-MS (ES−) [M−1]: 375.03.

### 3-((4-Chlorophenyl)(phenyl)methyl)-1,7-dimethylquinazoline-2,4(1H,3H)-dione

3-((4-Chlorophenyl)(phenyl)methyl)-1,7-dimethylquinazoline-2,4(1H,3H)-dione was prepared similarly to 7-fluoro-1-methyl-3-(4-(trifluoromethyl)benzyl)quinazoline-2,4(1H,3H)-dione in Example 16. 3-((4-Chlorophenyl)(phenyl)methyl)-7-methylquinazoline-2,4(1H,3H)-dione (30 mg; 0.080 mmol), sodium hydride (9.55 mg; 0.24 mmol; 60% in oil), and iodomethane (0.24 ml; 0.24 mmol; 1.0 M in DMF) were dissolved in 1 ml of DMF and stirred overnight at it to complete the reaction. The reaction was quenched with 0.5 ml of MeOH and the mixture was evaporated. Water (5 ml) was added and the mixture was washed three times with 5 ml of DCM, dried, and evaporated. CombiFlash purification (normal phase silica) gave 4 mg of 3-((4-chlorophenyl)(phenyl)methyl)-1,7-dimethylquinazoline-2,4(1H,3H)-dione. ¹H-NMR (400 MHz, CDCl₃): δ 2.49 (s, 3H), 3.55 (s, 3H), 6.97-7.00 (m, 1H), 7.05-7.09 (m, 1H), 7.26-7.41 (m, 9H), 7.50 (br s, 1H), 8.08 (d, 1H).

### Example 21: 3-(4-Bromobenzyl)-5,8-dimethoxy-1-methylquinazoline-2,4(1H,3H)-dione

### 3-(4-Bromobenzyl)-5,8-dimethoxyquinazoline-2,4(1H,3H)-dione

3-(4-Bromobenzyl)-5,8-dimethoxyquinazoline-2,4(1H,3H)-dione was prepared similarly to 7-fluoro-3-(4-(trifluoromethyl)benzyl)quinazoline-2,4(1H,3H)-dione in Example 16 using 5,8-dimethoxy-1H-benzo[d][1,3]oxazine-2,4-dione (0.200 g; 0.90 mmol), (4-bromophenyl)methanamine (0.136 ml; 1.08 mmol), and urea (81 mg; 1.34 mmol). 344 mg of crude product was obtained. ¹H-NMR (400 MHz, d₆-DMSO): δ 3.76 (s, 3H), 3.82 (s, 3H), 4.99 (s, 2H), 6.69 (d, 1H), 7.23-7.28 (m, 3H), 7.46-7.52 (m, 2H).

### 3-(4-Bromobenzyl)-5,8-dimethoxy-1-methylquinazoline-2,4(1H,3H)-dione

3-(4-Bromobenzyl)-5,8-dimethoxy-1-methylquinazoline-2,4(1H,3H)-dione was prepared similarly to 7-fluoro-1-methyl-3-(4-(trifluoromethyl)benzyl)quinazoline-2,4(1H,3H)-dione in Example 16 using 3-(4-bromobenzyl)-5,8-dimethoxyquinazoline-2,4(1H,3H)-dione (0.200 g; 0.51 mmol) as starting material. The crude product was triturated with MeOH to yield 126 mg of 3-(4-bromobenzyl)-5,8-dimethoxy-1-methylquinazoline-2,4(1H,3H)-dione. ¹H-NMR (400 MHz, d₆-DMSO): δ 3.58 (s, 3H), 3.79 (s, 3H), 3.81 (s, 3H), 5.01 (s, 2H), 6.87 (d, 1H), 7.23-7.28 (m, 2H), 7.42 (d, 1H), 7.46-7.51 (m, 2H).

### Example 22: 3-(3,4-Dichlorobenzyl)-7-fluoro-1-methylquinazoline-2,4(1H,3H)-dione

### 3-(3,4-Dichlorobenzyl)-7-fluoroquinazoline-2,4(1H,3H)-dione

3-(3,4-Dichlorobenzyl)-7-fluoroquinazoline-2,4(1H,3H)-dione was prepared similarly to 7-fluoro-3-(4-(trifluoromethyl)benzyl)quinazoline-2,4(1H,3H)-dione in Example 16 using 7-fluoro-1H-benzo[d][1,3]oxazine-2,4-dione (0.109 g; 0.60 mmol), (3,4-dichlorophenyl)methanamine (0.096 ml; 0.72 mmol), and urea (54 mg; 0.90 mmol). 219 mg of crude product was obtained. LC-MS (ES−) [M−1]: 338.9.

### 3-(3,4-Dichlorobenzyl)-7-fluoro-1-methylquinazoline-2,4(1H,3H)-dione

3-(3,4-Dichlorobenzyl)-7-fluoro-1-methylquinazoline-2,4(1H,3H)-dione was prepared similarly to 7-fluoro-1-methyl-3-(4-(trifluoromethyl)benzyl)quinazoline-2,4(1H,3H)-dione in Example 16 using 3-(3,4-dichlorobenzyl)-7-fluoroquinazoline-2,4(1H,3H)-dione (0.203 g; 0.60 mmol) as starting material. The crude product was triturated with MeOH and purified with CombiFlash (normal phase silica) to yield 88 mg of 3-(3,4-dichlorobenzyl)-7-fluoro-1-methylquinazoline-2,4(1H,3H)-dione. ¹H-NMR (400 MHz, CDCl₃): δ 3.57 (s, 3H), 5.19 (s, 2H), 6.89 (dd, 1H), 6.98 (ddd, 1H), 7.34-7.39 (m, 2H), 7.60-7.62 (m, 1H), 8.25 (ddd, 1H).

### Example 23: 7-Fluoro-3-(4-methoxybenzyl)-1-methylquinazoline-2,4(1H,3H)-dione

### 7-Fluoro-3-(4-methoxybenzyl)quinazoline-2,4(1H,3H)-dione

7-Fluoro-3-(4-methoxybenzyl)quinazoline-2,4(1H,3H)-dione was prepared similarly to 7-fluoro-3-(4-(trifluoromethyl)benzyl)quinazoline-2,4(1H,3H)-dione in Example 16 using 7-fluoro-1H-benzo[d][1,3]oxazine-2,4-dione (0.109 g; 0.60 mmol), (4-methoxyphenyl)methanamine (99 mg; 0.72 mmol), and urea (54 mg; 0.90 mmol). 172 mg of crude product was obtained. LC-MS (ES−) [M−1]: 299.0.

### 7-Fluoro-3-(4-methoxybenzyl)-1-methylquinazoline-2,4(1H,3H)-dione

7-Fluoro-3-(4-methoxybenzyl)-1-methylquinazoline-2,4(1H,3H)-dione was prepared similarly to 7-fluoro-1-methyl-3-(4-(trifluoromethyl)benzyl)quinazoline-2,4(1H,3H)-dione in Example 16 using 7-fluoro-3-(4-methoxybenzyl)quinazoline-2,4(1H,3H)-dione (0.172 g; 0.57 mmol) as starting material. The crude product was triturated with MeOH and purified with CombiFlash to yield 77 mg of 7-fluoro-3-(4-methoxybenzyl)-1-methylquinazoline-2,4(1H,3H)-dione. ¹H-NMR (400 MHz, d₆-DMSO): δ 3.50 (s, 3H), 3.71 (s, 3H), 5.05 (s, 2H), 6.83-6.88 (m, 2H), 7.12-7.19 (m, 1H), 7.26-7.32 (m, 2H), 7.35-7.41 (m, 1H), 8.09-8.15 (m, 1H).

### Example 24: (S)-3-(1-(4-Chlorophenyl)ethyl)-7-fluoro-1-methylquinazoline-2,4(1H,3H)-dione

### (S)-3-(1-(4-Chlorophenyl)ethyl)-7-fluoroquinazoline-2,4(1H,3H)-dione

4-Fluoroisatoic anhydride (300 mg; 1.66 mmol), 2 ml of dry DMA, (S)-1-(4-chlorophenyl)ethanamine (0.279 ml; 1.99 mmol), and urea (149 mg; 2.49 mmol) were charged in a microwave tube and heated at 250° C. for 15 min. Water was added to the mixture and the mixture was extracted twice with EtOAc. Organic phases were combined, dried, and evaporated. EtOAc was added to the evaporation residue and the precipitation was filtered. The filtrate was evaporated to yield 480 mg of (S)-3-(1-(4-chlorophenyl)ethyl)-7-fluoroquinazoline-2,4(1H,3H)-dione. LC-MS (ES−) [M−1]: 316.9.

### (S)-3-(1-(4-Chlorophenyl)ethyl)-7-fluoro-1-methylquinazoline-2,4(1H,3H)-dione

Sodium hydride (90 mg; 2.26 mmol; 60%) and 1 ml of dry DMF were charged in a reaction flask under nitrogen and the mixture was cooled to 0° C. (S)-3-(1-(4-chlorophenyl)ethyl)-7-fluoroquinazoline-2,4(1H,3H)-dione (480 mg; 1.51 mmol) in a small amount of DMF was added to the mixture. The reaction mixture was stirred at rt for 30 min and iodomethane (0.141 ml; 2.26 mmol) was added. The reaction mixture was stirred at rt overnight. MeOH was added and the mixture was evaporated. Water and DCM were added to the evaporation residue and the mixture was extracted three times with DCM. Organic phases were combined, dried, and evaporated. The crude product was purified with MS-Trigger and with CombiFlash (normal phase silica) to yield 99 mg of (S)-3-(1-(4-chlorophenyl)ethyl)-7-fluoro-1-methylquinazoline-2,4(1H,3H)-dione. ¹H-NMR (400 MHz, d₆-DMSO): δ 1.79 (d, 3H), 3.45 (s, 3H), 6.21 (q, 1H), 7.14 (td, 1H), 7.33-7.39 (m, 5H), 8.08 (dd, 1H).

### Example 25: (R)-3-(1-(4-Chlorophenyl)ethyl)-6,7-dimethoxy-1-methylquinazoline-2,4(1H,3H)-dione

### (R)-3-(1-(4-chlorophenyl)ethyl)-6,7-dimethoxyquinazoline-2,4(1H,3H)-dione

4,5-Dimethoxyisatoic anhydride (300 mg; 1.34 mmol), 2 ml of dry DMA, (R)-1-(4-chlorophenyl)ethanamine (0.266 ml; 1.61 mmol), and urea (121 mg; 2.02 mmol) were charged in a microwave tube and heated at 250° C. for 20 min. (R)-1-(4-chlorophenyl)ethanamine (95 μl) and urea (40 mg) were added and the reaction mixture was heated again at 250° C. for 20 min. Water was added to the mixture and the precipitation was filtered and dried to yield 179 mg of (R)-3-(1-(4-chlorophenyl)ethyl)-6,7-dimethoxyquinazoline-2,4(1H,3H)-dione. LC-MS (ES−) [M−1]: 359.0.

### (R)-3-(1-(4-chlorophenyl)ethyl)-6,7-dimethoxy-1-methylquinazoline-2,4(1H,3H)-dione

Sodium hydride (59.5 mg; 1.49 mmol; 60%) and 1 ml of dry DMF were charged in a reaction flask under nitrogen and the mixture was cooled to 0° C. (R)-3-(1-(4-Chlorophenyl)ethyl)-6,7-dimethoxyquinazoline-2,4(1H,3H)-dione (179 mg; 0.50 mmol) was added in a small amount of DMF to the reaction mixture and the mixture was stirred at rt for 30 min. Iodomethane (0.093 ml; 1.49 mmol) was added and the reaction mixture was stirred at rt for three days. MeOH was added and the mixture was evaporated. Water and DCM were added to the evaporation residue and the mixture was extracted twice with DCM. Organic phases were combined, dried, and evaporated. The crude product was purified with MS-Trigger to yield 27 mg of (R)-3-(1-(4-chlorophenyl)ethyl)-6,7-dimethoxy-1-methylquinazoline-2,4(1H,3H)-dione. ¹H-NMR (400 MHz, d₆-DMSO): δ 1.90 (d, 3H), 3.54 (s, 3H), 3.93 (s, 3H), 4.00 (s, 3H), 6.41 (m, 1H), 6.57 (s, 1H), 7.24-7.30 (m, 2H), 7.35-7.45 (m, 2H), 7.59 (s, 1H).

### Example 26: 3-(4-Bromobenzyl)-1-(3,3-dimethyl-2-oxobutyl)-7-fluoroquinazoline-2,4(1H,3H)-dione

### 2-Amino-N-(4-bromobenzyl)-4-fluorobenzamide

2-Amino-4-fluorobenzoic acid (1.0 g; 7.1 mmol), 5 ml of EtOAc and TEA (2.7 ml; 7.7 mmol) were placed in a reaction flask under nitrogen. (4-Bromophenyl)methanamine (0.90 ml; 7.1 mmol) was added slowly and then T3P was added keeping the temperature below 30° C. The reaction was close to complete at 5 h, but the mixture was stirred at rt overnight. EtOAc (5 ml) was added and the mixture was washed three times with 5 ml of water. Few drop of brine was used in the last separation. Organic phases were combined and evaporated to dryness to yield 1.68 g of crude material, which was crystallized from 7.5 ml of toluene by heating and then cooling slowly to rt and finally to 0° C. The precipitation was filtered, washed with 4 ml of cold toluene, and dried in a vacuum oven at 40° C. to give 1.226 g of 2-amino-N-(4-bromobenzyl)-4-fluorobenzamide. ¹H-NMR (400 MHz, CDCl₃): δ 4.54 (d, 2H), 5.77 (br s, 2H), 6.22 (br s, 1H), 6.30-6.39 (m, 2H), 7.20-7.25 (m, 2H), 7.29 (dd, 1H), 7.45-7.50 (m, 2H).

### 3-(4-Bromobenzyl)-7-fluoroquinazoline-2,4(1H,3H)-dione

2-Amino-N-(4-bromobenzyl)-4-fluorobenzamide (1.22 g; 3.8 mmol), 8+2 ml of dry THF, and 1.2 ml of pyridine were placed in a reaction flask under nitrogen. Ethyl chloroformate (1.1 ml; 11.3 mmol) was added dropwise at 0° C. The reaction mixture was stirred at rt for 2 h to complete carbamate formation. 5 M NaOH (3 ml; 15.1 mmol) was added slowly at 0° C. and the mixture was heated at 50° C. for 1 h to complete ring closure. Water (5 ml) was added and pH adjusted to 2.2 with concentrated HCl. The precipitation was stirred at rt overnight, filtered, washed twice with 5 ml of water, and dried in a vacuum oven at 50° C. to give 0.98 g of 3-(4-bromobenzyl)-7-fluoroquinazoline-2,4(1H,3H)-dione. Additional 0.18 g of the precipitation was filtered from the filtrate of the crystallization. ¹H-NMR (400 MHz, d₆-DMSO): δ 5.03 (s, 2H), 6.93 (dd, 1H), 7.04-7.11 (m, 1H), 7.25-7.30 (m, 2H), 7.48-7.53 (m, 2H), 8.00 (dd, 1H), 11.68 (br s, 1H).

### 3-(4-Bromobenzyl)-1-(3,3-dimethyl-2-oxobutyl)-7-fluoroquinazoline-2,4(1H,3H)-dione

3-(4-Bromobenzyl)-7-fluoroquinazoline-2,4(1H,3H)-dione (0.2 g; 0.57 mmol) and 1 ml of DMF were placed under nitrogen. Solid NaOH (34 mg; 0.86 mmol) was added and the mixture was stirred at rt for 15 min. 1-Bromo-3,3-dimethyl-2-butanone (0.077 ml; 0.57 mmol) was added and the mixture was stirred for 3 h at rt. Additional 1-bromo-3,3-dimethyl-2-butanone (0.039 ml; 0.28 mmol) was added. The mixture was stirred for 1 h at rt and then 3 h at 50° C. to complete the reaction. The reaction mixture was cooled to rt, 1.5 ml of water was added, and the mixture was stirred overnight. The precipitation was filtered off, washed twice with 1 ml of water, and dried at 50° C. under vacuum for 2 h to give 0.217 g of crude product. The product was crystallized from 1.9 ml of ACN:EtOH (95:5) by heating and cooling to 0° C. The precipitation was filtered, washed with a small amount of ACN, and dried under vacuum at 50° C. to yield 0.15 g of 3-(4-bromobenzyl)-1-(3,3-dimethyl-2-oxobutyl)-7-fluoroquinazoline-2,4(1H,3H)-dione. ¹H-NMR (400 MHz, d₆-DMSO): δ 1.24 (s, 9H), 5.08 (s, 2H), 5.24 (s, 2H), 7.14-7.21 (m, 1H), 7.22-7.29 (m, 3H), 7.48-7.54 (m, 2H), 8.15 (dd, 1H).

### Example 27: 3-(4-Bromobenzyl)-7-fluoro-1-(3-oxobutan-2-yl)quinazoline-2,4(1H,3H)-dione

3-(4-Bromobenzyl)-7-fluoroquinazoline-2,4(1H,3H)-dione (200 mg; 0.57 mmol) prepared in Example 26, 6 ml of dry THF, and 3-chloro-2-butanone (183 mg; 1.72 mmol) were charged in a reaction flask under nitrogen, and TBAF (1 M in THF; 225 mg; 0.86 mmol) was added. The reaction mixture was stirred at rt overnight. Additional 1 M TBAF (1 M in THF; 225 mg; 0.86 mmol) was added and the mixture was heated refluxed. After 4 h, TBAF (1 M in THF; 225 mg; 0.86 mmol) was added again and the mixture was refluxed overnight. The reaction mixture was evaporated to dryness. Ice-cold water, DCM, and MeOH were added to the evaporation residue, layers were separated, and organic phase was dried and evaporated. The crude product was purified with CombiFlash (normal phase silica) to yield 47 mg of 3-(4-bromobenzyl)-7-fluoro-1-(3-oxobutan-2-yl)quinazoline-2,4(1H,3H)-dione. ¹H-NMR (400 MHz, CDCl₃): δ 1.62 (d, 3H), 2.11 (s, 3H), 5.03-5.13 (m, 1H), 5.19 (dd, 2H), 6.73 (dd, 1H), 6.96-7.04 (m, 1H), 7.36-7.46 (m, 4H), 8.29 (dd, 1H).

### Example 28: 3-(4-Bromobenzyl)-7-fluoro-1-(2-methoxyethyl)quinazoline-2,4(1H,3H)-dione

Sodium hydride (34.3 mg; 0.86 mmol; 60%) and 2 ml of dry DMF were charged in a flask under nitrogen. The mixture was cooled to 0° C., 3-(4-bromobenzyl)-7-fluoroquinazoline-2,4(1H,3H)-dione (100 mg; 0.286 mmol) prepared in Example 26 was added, and the mixture was stirred at rt for 30 min. 2-Chloroethyl methyl ether (0.078 ml; 0.86 mmol) was added at 0° C. The reaction mixture was stirred at rt. After 3 h, the mixture was heated at 50° C. for 4 h and finally the mixture was heated at 100° C. for 14 h. The mixture was cooled to 0° C. and water was added. The precipitation was filtered and washed with water. The crude product was crystallized from EtOH to yield 25 mg of 3-(4-bromobenzyl)-7-fluoro-1-(2-methoxyethyDquinazoline-2,4(1H,3H)-dione. ¹H-NMR (400 MHz, CDCl₃): δ 3.33 (s, 3H), 3.70 (t, 2H), 4.25 (t, 2H), 5.19 (s, 2H), 6.94 (td, 1H), 7.11 (dd, 1H), 7.34-7.49 (m, 4H), 8.22 (dd, 1H).

### Example 29: 3-(4-Bromobenzyl)-7-fluoro-1-(2-(2-methoxyethoxy)ethyl)quinazoline-2,4(1H,3H)-dione

The preparation of 3-(4-bromobenzyl)-7-fluoroquinazoline-2,4(1H,3H)-dione is described in Example 26. K₂CO₃ (95 mg; 0.69 mmol), 3-(4-bromobenzyl)-7-fluoroquinazoline-2,4(1H,3H)-dione (200 mg; 0.57 mmol), 1-chloro-2-(2-methoxyethoxy)ethane (0.093 ml; 0.69 mmol), and 3 ml of dry THF were charged in a microwave tube and heated at 125° C. for 10 min (absorption high). Cs₂CO₃ (280 mg; 0.86 mmol) was added and the reaction continued 10 min at 125° C. and then 10+20+20 min at 175° C. The mixture was cooled, water was added, and the mixture was washed twice with EtOAc. Organic layers were combined, dried, and evaporated. The crude product was purified with CombiFlash (first EtOAc:heptane; normal phase silica and then DCM:MeOH; normal phase silica) to yield 100 mg of impure product. Purification with MS-Trigger gave 58 mg of 3-(4-bromobenzyl)-7-fluoro-1-(2-(2-methoxyethoxy)ethyl)quinazoline-2,4(1H,3H)-dione ¹H-NMR (400 MHz, CDCl₃): δ 3.31 (s, 3H), 3.42-3.48 (m, 2H), 3.57-3.62 (m, 2H), 3.81 (t, 2H), 4.27 (t, 2H), 5.18 (s, 2H), 6.91-6.97 (m, 1H), 7.18 (dd, 1H), 7.37-7.45 (m, 4H), 8.21 (dd, 1H).

### Example 30: 2-(3-(4-Bromobenzyl)-7-fluoro-2,4-dioxo-3,4-dihydroquinazolin-1(2H)-yl)-propanenitrile

Sodium hydride (68.7 mg; 1.72 mmol; 60%) and 2 ml of dry DMF were charged in a reaction flask under nitrogen. 3-(4-Bromobenzyl)-7-fluoroquinazoline-2,4(1H,3H)-dione (200 mg; 0.573 mmol) prepared in Example 26 was added at 0° C. and the mixture was stirred at rt for 30 min. 2-Bromopropanenitrile (0.149 ml; 1.72 mmol) was added and the reaction mixture was stirred at rt overnight. The mixture was heated at 50° C. for 2 h and then at 70° C. for 2.5 h. The mixture was cooled to 0° C., water and EtOH were added, and the precipitation was filtered and washed with water. The crude product was crystallized from EtOH and purified with CombiFlash (normal phase silica) to yield 40 mg of 2-(3-(4-bromobenzyl)-7-fluoro-2,4-dioxo-3,4-dihydroquinazolin-1(2H)-yl)propanenitrile. ¹H-NMR (400 MHz, CDCl₃): δ 1.83 (d, 3H), 5.18 (dd, 2H), 6.33 (q, 1H), 7.01-7.10 (m, 1H), 7.22 (dd, 1H), 7.35-7.48 (m, 4H), 8.31 (dd, 1H).

### Example 31: 3-(4-Bromobenzyl)-7-fluoro-1-(3-oxobutyl)quinazoline-2,4(1H,3H)-dione

### 4-Chlorobutan-2-one

4-Hydroxy-2-butanone (0.881 g; 10 mmol), 2 ml of DCM, and thionyl chloride (1.46 ml; 20 mmol) were charged in a reaction flask and stirred at rt overnight. The mixture was evaporated and dried by nitrogen flow to yield 1.06 g of 2-chlorobutan-2-one. ¹H-NMR (400 MHz, d₆-DMSO): δ 2.12 (s, 3H), 2.94 (t, 2H), 3.74 (t, 2H).

### 3-(4-Bromobenzyl)-7-fluoro-1-(3-oxobutyl)quinazoline-2,4(1H,3H)-dione

Sodium hydride (68.7 mg; 1.72 mmol; 60% in oil) and 2 ml of dry DMF were charged in a reaction flask under nitrogen and stirred for 30 min at 0° C. 3-(4-Bromobenzyl)-7-fluoroquinazoline-2,4(1H,3H)-dione (200 mg; 0.573 mmol) prepared in Example 26 was added and the mixture was stirred at rt for 30 min. 4-Chlorobutan-2-one (183 mg; 1.72 mmol) was added. The reaction mixture was stirred at rt for three days and then heated at 50° C. for 4 h. The mixture was cooled to 0° C. and water was added. The precipitation was filtered and washed with water. The crude product was crystallized from EtOH to yield 40 mg of 3-(4-bromobenzyl)-7-fluoro-1-(3-oxobutyl)quinazoline-2,4(1H,3H)-dione. ¹H-NMR (400 MHz, d₆-DMSO): δ 2.13 (s, 3H) 2.80-2.87 (m, 2H), 4.20-4.28 (m, 2H), 5.07 (s, 2H), 7.16 (td, 1H), 7.26-7.32 (m, 2H), 7.44-7.53 (m, 3H), 8.12 (dd, 1H).

### Example 32: 3-(4-Bromobenzyl)-7-fluoro-1-(2-hydroxypropyl)quinazoline-2,4(1H,3H)-dione

### 3-(4-Bromobenzyl)-7-fluoro-1-(oxiran-2-ylmethyl)quinazoline-2,4(1H,3H)-dione

3-(4-Bromobenzyl)-7-fluoroquinazoline-2,4(1H,3H)-dione (100 mg; 0.27 mmol) prepared in Example 26, K₂CO₃ (237 mg; 1.72 mmol), epibromohydrin (235 mg; 1.72 mmol), and 4 ml of dry DMF were charged in a microwave tube and heated at 110° C. for 1 h. The reaction mixture was poured into water and extracted three times with EtOAc. Organic phase was washed once with brine, dried, and evaporated. The evaporation residue was purified by CombiFlash (normal phase silica) to yield 57 mg of 3-(4-bromobenzyl)-7-fluoro-1-(oxiran-2-ylmethyl)quinazoline-2,4(1H,3H)-dione. ¹H-NMR (400 MHz, CDCl₃): δ 2.66-2.75 (m, 1H), 2.89 (t, 1H), 3.23-3.31 (m, 1H), 3.79 (dd, 1H), 4.76 (dd, 1H), 5.20 (s, 2H) 6.93-7.01 (m, 1H), 7.11 (dd, 1H), 7.36-7.47 (m, 4H), 8.23 (dd, 1H).

### 3-(4-Bromobenzyl)-7-fluoro-1-(2-hydroxypropyl)quinazoline-2,4(1H,3H)-dione

3-(4-Bromobenzyl)-7-fluoro-1-(oxiran-2-ylmethyl)quinazoline-2,4(1H,3H)-dione (57 mg; 0.14 mmol) and 2 ml of dry THF were charged in a reaction flask under nitrogen and sodium borohydride (6.6 mg; 0.18 mmol) and 2 ml of dry IPA were added. The reaction mixture was stirred at rt overnight. Additional NaBH₄ (6.5 mg; 0.18 mmol) was added and the mixture was stirred for 1 h. A third portion of NaBH₄ (6.5 mg; 0.18 mmol) was added and the mixture was heated at 60° C. for 1 h. The reaction mixture was cooled, poured slowly into cold water, and extracted three times with EtOAc. Organic phases were combined, washed with brine, dried, and evaporated. The crude product was purified with CombiFlash (normal phase silica) to yield 19 mg of 3-(4-bromobenzyl)-7-fluoro-1-(2-hydroxypropyl)quinazoline-2,4(1H,3H)-dione. ¹H-NMR (400 MHz, CDCl₃): δ 1.35 (d, 3H), 2.28 (d, 1H), 3.98-4.17 (m, 2H), 4.23 (br s, 1H), 5.17 (s, 2H), 6.87-7.11 (m, 2H), 7.32-7.48 (m, 4H), 8.22 (dd, 1H).

### Example 33: 3-(4-Bromobenzyl)-7-fluoro-1-methylquinazoline-2,4(1H,3H)-dione

3-(4-Bromobenzyl)-7-fluoro-1-methylquinazoline-2,4(1H,3H)-dione was prepared similarly to 3-(4-bromobenzyl)-1-(3,3-dimethyl-2-oxobutyl)-7-fluoroquinazoline-2,4(1H,3H)-dione in Example 26. 3-(4-Bromobenzyl)-7-fluoroquinazoline-2,4(1H,3H)-dione (0.2 g; 0.57 mmol) and 1 ml of DMF were placed in a reaction flask under nitrogen. Solid NaOH (34 mg; 0.86 mmol) was added and the mixture was stirred at rt for 15 min. Iodomethane (0.053 ml; 0.86 mmol) was added slowly and the reaction was complete in 1 h at rt. Water (1.5 ml) was added and the mixture was stirred overnight at rt. The precipitation was filtered off, washed twice with 1 ml of water, and dried under vacuum at 50° C. to obtain 0.19 g of crude product. The product was crystallized from 1 ml of ACN:EtOH (95:5), stirred overnight at rt and then 30 min at 0° C., and filtered. The precipitation was dried under vacuum at 50° C. to yield 0.164 g of 3-(4-bromobenzyl)-7-fluoro-1-methylquinazoline-2,4(1H,3H)-dione. ¹H-NMR (400 MHz, d₆-DMSO): δ 3.50 (s, 3H), 5.08 (s, 2H), 7.13-7.20 (m, 1H), 7.26-7.32 (m, 2H), 7.40 (dd, 1H), 7.47-7.52 (m, 2H), 8.12 (dd, 1H).

### Example 34: 3-(3-(4-Bromobenzyl)-7-fluoro-2,4-dioxo-3,4-dihydroquinazolin-1(2H)-yl)propanoic acid

### Ethyl 3-(3-(4-bromobenzyl)-7-fluoro-2,4-dioxo-3,4-dihydroquinazolin-1(2H)-yl)propanoate

The preparation of 3-(4-bromobenzyl)-7-fluoroquinazoline-2,4(1H,3H)-dione is described in Example 26. 3-(4-Bromobenzyl)-7-fluoroquinazoline-2,4(1H,3H)-dione (0.50 g; 1.43 mmol), K₂CO₃ (594 mg; 4.30 mmol), 4 ml of dry DMF, and ethyl 3-bromopropionate (0.55 ml; 4.30 mmol) were placed in a microwave reaction vial and heated at 100° C. for 2 h (absorption high). K₂CO₃ (198 mg; 1.43 mmol) and ethyl 3-bromopropionate (0.18 ml; 1.43 mmol) were added again and the mixture was heated at 100° C. for 2 h. K₂CO₃ (198 mg; 1.43 mmol) and ethyl 3-bromopropionate (0.18 ml; 1.43 mmol) were added once more and the mixture was heated for 4 h at 100° C. 50 ml of water was added to give precipitation (513 mg). CombiFlash (normal phase silica) purification and ACN trituration of the product fractions gave 390 mg of ethyl 3-(3-(4-bromobenzyl)-7-fluoro-2,4-dioxo-3,4-dihydroquinazolin-1(2H)-yl)propanoate. ¹H-NMR (400 MHz, CDCl₃): δ 1.25 (t, 3H), 2.70-2.76 (m, 2H), 4.15 (q, 2H), 4.33-4.39 (m, 2H), 5.18 (s, 2H), 6.93-7.00 (m, 2H), 7.37-7.45 (m, 4H), 8.22-8.28 (m, 1H).

### 3-(3-(4-Bromobenzyl)-7-fluoro-2,4-dioxo-3,4-dihydroquinazolin-1(2H)-yl)propanoic acid

Ethyl 3-(3-(4-bromobenzyl)-7-fluoro-2,4-dioxo-3,4-dihydroquinazolin-1(2H)-yl)propanoate (109 mg; 0.24 mmol), THF (1 ml), MeOH (1 ml), and 1 M LiOH (1 ml; lmmol) were placed in a reaction flask and stirred for 2 h at rt. The reaction mixture was diluted with water and brine and pH was adjusted to acidic with 1 M HCl. The mixture was washed three times with EtOAc. Organic phases were combined, dried, and evaporated to dryness to give 105 mg of 3-(3-(4-bromobenzyl)-7-fluoro-2,4-dioxo-3,4-dihydroquinazolin-1(2H)-yl)propanoic acid. ¹H-NMR (400 MHz, CDCl₃): δ 2.78-2.85 (m, 2H), 4.33-4.42 (m, 2H), 5.18 (s, 2H), 6.92-7.02 (m, 2H), 7.36-7.46 (m, 4H), 8.26 (dd, 1H).

### Example 35: 3-(3-(4-Bromobenzyl)-7-fluoro-2,4-dioxo-3,4-dihydroquinazolin-1(2H)-yl)-propanamide

The preparation of 3-(3-(4-bromobenzyl)-7-fluoro-2,4-dioxo-3,4-dihydroquinazolin-1(2H)-yl)propanoic acid is described in Example 34. 3-(3-(4-Bromobenzyl)-7-fluoro-2,4-dioxo-3,4-dihydroquinazolin-1(2H)-yl)propanoic acid (100 mg; 0.24 mmol), 3 ml of dry chloroform, and thionyl chloride (50 μl; 0.685 mmol) were charged in a reaction flask and refluxed for 1 h. The reaction mixture was evaporated and the acyl chloride intermediate was formed. The evaporation residue was dissolved in 1 ml of chloroform. The mixture was cooled using ice bath and 1 ml of ammonia hydroxide solution was added. The mixture was stirred at rt for seven days followed by addition of water. The reaction mixture was extracted three times with DCM. Organic phases were combined, washed with saturated NaHCO₃, dried, and evaporated. The crude product was purified with MS-Trigger to yield 6 mg of 3-(3-(4-bromobenzyl)-7-fluoro-2,4-dioxo-3,4-dihydroquinazolin-1(2H)-yl)propanamide. ¹H-NMR (400 MHz, d₄-MeOD/CDCl₃): δ 2.59-2.67 (m, 2H), 4.32-4.40 (m, 2H), 5.18 (s, 2H), 6.99 (dd, 1H), 7.21 (dd, 1H), 7.34-7.40 (m, 2H), 7.40-7.46 (m, 2H), 8.24 (dd, 1H).

### Example 36: 3-(3-(4-Bromobenzyl)-7-fluoro-2,4-dioxo-3,4-dihydroquinazolin-1(2H)-yl)-N,N-dimethylpropanamide

The preparation of 3-(3-(4-bromobenzyl)-7-fluoro-2,4-dioxo-3,4-dihydroquinazolin-1(2H)-yl)propanoic acid is described in Example 34. 3-(3-(4-Bromobenzyl)-7-fluoro-2,4-dioxo-3,4-dihydroquinazolin-1(2H)-yl)propanoic acid (55 mg; 0.131 mmol) was dissolved in 1 ml of DCM in a reaction flask under nitrogen. TEA (0.055 ml; 0.39 mmol), dimethylamine (0.1 ml; 0.2 mmol; 2 M in THF), and T3P (0.074 ml; 0.20 mmol; 50% in EtOAc) were added slowly and the reaction mixture was stirred at rt overnight. DCM was added and the mixture was washed three times with water and dried with a phase separator. Organic layer was evaporated to dryness (60 mg). MS-Trigger purification gave 32 mg of the product, which was further purified with CombiFlash (normal phase silica) to give 18 mg of 3-(3-(4-bromobenzyl)-7-fluoro-2,4-dioxo-3,4-dihydroquinazolin-1(2H)-yl)-N,N-dimethylpropanamide. ¹H-NMR (400 MHz, CDCl₃): δ 2.69-2.76 (m, 2H), 2.97 (s, 3H), 2.99 (s, 3H), 4.36-4.42 (m, 2H), 5.18 (s, 2H), 6.96 (ddd, 1H), 7.08 (dd, 1H), 7.37-7.45 (m, 4H), 8.24 (dd, 1H).

### Example 37: 2-(3-(4-Bromobenzyl)-7-fluoro-2,4-dioxo-3,4-dihydroquinazolin-1(2H)-yl)propanamide

### Methyl 2-(3-(4-bromobenzyl)-7-fluoro-2,4-dioxo-3,4-dihydroquinazolin-1(2H)-yl)propanoate

The preparation of 3-(4-bromobenzyl)-7-fluoroquinazoline-2,4(1H,3H)-dione is described in Example 26. 3-(4-Bromobenzyl)-7-fluoroquinazoline-2,4(1H,3H)-dione (1.0 g; 2.86 mmol), K₂CO₃ (792 mg; 5.73 mmol), 10 ml of THF, TBAB (0.15 g; 0.47 mmol), and methyl 2-bromopropionate (0.64 ml; 5.73 mmol) were placed in a microwave reaction vial and heated at 120° C. for 10 min (absorption high). Water and EtOAc were added. Phases were separated and the aqueous phase was washed twice with EtOAc. Organic phases were combined, dried, and evaporated to dryness. CombiFlash (normal phase silica) purification gave 923 mg of methyl 2-(3-(4-bromobenzyl)-7-fluoro-2,4-dioxo-3,4-dihydroquinazolin-1(2H)-yl)propanoate. ¹H-NMR (400 MHz, CDCl₃): δ 1.62 (d, 3H), 3.75 (s, 3H), 5.27 (dd, 2H), 5.51 (q, 1H), 6.99-7.07 (m, 2H), 7.33-7.38 (m, 2H), 7.41-7.47 (m, 2H), 8.15-8.22 (m, 1H).

### 2-(3-(4-Bromobenzyl)-7-fluoro-2,4-dioxo-3,4-dihydroquinazolin-1(2H)-yl)propanoic acid

Methyl 2-(3-(4-bromobenzyl)-7-fluoro-2,4-dioxo-3,4-dihydroquinazolin-1(2H)-yl)propanoate (300 mg; 0.69 mmol), THF (2 ml), LiOH (33 mg; 1.38 mmol), and water (1.5 ml) were placed in a reaction flask and stirred overnight at rt. Water and brine were added, pH was adjusted to acidic with 1 M HCl, and the mixture was washed three times with EtOAc. Organic phases were combined, dried, and evaporated to dryness to give 263 mg of 2-(3-(4-bromobenzyl)-7-fluoro-2,4-dioxo-3,4-dihydroquinazolin-1(2H)-yl)propanoic acid. ¹H-NMR (400 MHz, CDCl₃): δ 1.72 (d, 3H), 5.17 (dd, 2H), 5.28 (br s, 2H), 6.79 (dd, 1H), 6.99 (dd, 1H), 7.33-7.44 (m, 4H), 8.27 (dd, 1H).

### 2-(3-(4-Bromobenzyl)-7-fluoro-2,4-dioxo-3,4-dihydroquinazolin-1(2H)-yl)propanamide

2-(3-(4-Bromobenzyl)-7-fluoro-2,4-dioxo-3,4-dihydroquinazolin-1(2H)-yl)propanoic acid (130 mg; 0.31 mmol), chloroform (3 ml), and thionyl chloride (0.051 ml; 0.69 mmol) were charged in a reaction flask and refluxed for 1.5 h. The reaction mixture was evaporated to dryness. The residue was dissolved in 2 ml of chloroform, ammonia (2 ml; 1.0 mmol; 0.5 M in dioxane) was added dropwise, and the mixture was stirred at rt over the weekend. Water (20 ml) and DCM (20 ml) were added. Organic phase was washed with 1 M NaHCO₃, dried, and evaporated to dryness. CombiFlash (normal phase silica) purification gave 13.5 mg of 2-(3-(4-bromobenzyl)-7-fluoro-2,4-dioxo-3,4-dihydroquinazolin-1(2H)-yl)propanamide. ¹H-NMR (400 MHz, CDCl₃): δ 1.67 (d, 3H), 5.19 (q, 2H), 5.68 (br s, 1H), 5.47 (br s, 1H), 5.78 (br s, 1H), 6.94-7.02 (m, 2H), 7.37-7.47 (m, 4H), 8.24-8.29 (m, 1H).

### Example 38: 3-(4-Bromobenzyl)-7-fluoro-1-isopropylquinazoline-2,4(1H,3H)-dione

3-(4-Bromobenzyl)-7-fluoroquinazoline-2,4(1H,3H)-dione (200 mg; 0.57 mmol) prepared in Example 26, 2 ml of dry THF, TBAB (50 mg; 0.155 mmol), K₂CO₃ (237 mg; 1.72 mmol), and 2-iodopropane (0.172 ml; 1.72 mmol) were charged in a microwave tube and heated at 150° C. for 15 min. Water was added and the precipitation was filtered. The crude product was purified twice with CombiFlash (normal phase silica and then reverse phase silica) and then with MS-Trigger to yield 44 mg of 3-(4-bromobenzyl)-7-fluoro-1-isopropylquinazoline-2,4(1H,3H)-dione. ¹H-NMR (400 MHz, CDCl₃): δ 1.59 (d, 6H), 4.97 (m, 1H), 5.17 (s, 2H), 6.94 (m, 1H), 7.04 (dd, 1H), 7.32-7.48 (m, 4H), 8.25 (dd, 1H).

### Example 39: 3-(4-Bromobenzyl)-7-fluoro-1-(2-hydroxy-2-methylpropyl)quinazoline-2,4(1H,3H)-dione

The preparation of 3-(4-bromobenzyl)-7-fluoroquinazoline-2,4(1H,3H)-dione is described in Example 26. 3-(4-Bromobenzyl)-7-fluoroquinazoline-2,4(1H,3H)-dione (500 mg; 1.43 mmol) and yttrium(III) nitrate hexahydrate (54.8 mg; 0.14 mmol) were placed in a microwave reaction vial. DMF (4 ml) and isobutylene oxide (6.36 ml; 71.6 mmol) were added and the reaction mixture was heated in a microwave reactor at 160° C. for 60 min. After cooling to rt, saturated NaHCO₃ was added and the mixture was extracted twice with DCM. The combined organic layers were washed twice with water and once with brine, dried with a phase separator, and evaporated to dryness. The crude product was purified with column chromatography (EtOAc:heptane; normal phase silica). The fractions were evaporated and triturated first with MeOH and then with diethyl ether. Combined precipitations and the first trituration filtrate were combined, evaporated, and crystallized from MeOH/heptane (5 ml:10 ml) to give 474 mg of the product. The product was once more purified with column chromatography (CombiFlash, reverse phase silica) and the product was precipitated in evaporation, filtered, and dried to give 371 mg of 3-(4-bromobenzyl)-7-fluoro-1-(2-hydroxy-2-methylpropyl)quinazoline-2,4(1H,3H)-dione. ¹H-NMR (400 MHz, CDCl₃): δ 1.33 (s, 6H), 2.51 (s, 1H), 4.16 (s, 2H), 5.20 (s, 2H), 6.96 (ddd, 1H), 7.20 (dd, 1H), 7.34-7.46 (m, 4H), 8.23 (dd, 1H).

### Example 40: 7-Fluoro-1-methyl-3-(4-nitrobenzybquinazoline-2,4(1H,3H)-dione

### 2-Amino-N-(tert-butyl)-4-fluorobenzamide

2-Amino-N-(tert-butyl)-4-fluorobenzamide was prepared similarly to 3-(4-bromobenzyl)-5,7-dimethoxyquinazoline-2,4(1H,3H)-dione in Example 26. 2-Amino-4-fluorobenzoic acid (2.0 g; 12.9 mmol), 20 ml of DCM, TEA (5.4 ml; 38.7 mmol), and tert-butylamine (1.49 ml; 14.2 mmol) were placed in a reaction flask under nitrogen. The solution was cooled to 0° C. and T3P (9.12 ml; 15.5 mmol, 50% solution) was added slowly. The reaction mixture was stirred for 30 min at 0° C. and overnight at rt. Water and DCM were added and water phase was washed twice with water and once with brine. Organic phase was dried and evaporated to dryness to give 1.33 g of crude 2-amino-N-(cert-butyl)-4-fluorobenzamide. ¹H-NMR (400 MHz, d₆-DMSO): δ 1.35 (s, 9H), 3.1-4.1 (br s, 2H), 6.22-6.29 (m, 1H), 6.42 (dd, 1H), 7.47 (m, 1H), 7.49 (br s, 1H).

### 3-(tert-ButyI)-7-fluoroquinazoline-2,4(1H,3H)-dione

Crude 2-amino-N-(tert-butyl)-4-fluorobenzamide (1.23 g; 5.85 mmol) and 12 ml of pyridine were placed in a reaction flask under nitrogen. The reaction mixture was cooled to 0° C. and ethyl chloroformate (1.68 ml; 17.6 mmol) was added dropwise. The reaction mixture was stirred overnight at rt to complete carbamate formation. EtOAc and 1 M HCl were added, layers were separated, and the aqueous phase was extracted with EtOAc. The combined organic layers were washed with 1 M HCl, water, and brine, dried with a phase separator, and evaporated to dryness. To the residue, EtOH (20 ml) and KOH (1.83 g; 32.6 mmol) were added and the mixture was refluxed for approximately 24 h. The mixture was cooled to 0° C. and 1 M HCl (40 ml) was slowly added. The resulting precipitation was filtered, washed with water, and dried in a vacuum oven at 40° C. to give 0.47 g of crude 3-(tert-butyl)-7-fluoroquinazoline-2,4(1H,3H)-dione. ¹H-NMR (400 MHz, d₆-DMSO): δ 1.66 (s, 9H), 6.81 (dd, 1H), 6.97 (ddd, 1H), 7.89 (dd, 1H), 11.14 (s, 1H).

### 3-(tert-Butyl)-7-fluoro-1-methylquinazoline-2,4(1H,3H)-dione

3-(tert-Butyl)-7-fluoroquinazoline-2,4(1H,3H)-dione (460 mg; 1.95 mmol), sodium hydride (165 mg, 3.89 mmol, 60% in oil), and DMF (6 ml) were placed in a reaction flask under nitrogen and the mixture was stirred at rt for 15 min. Iodomethane (0.97 ml; 15.6 mmol) was carefully added and the reaction mixture was stirred for 3 h, followed by the addition of MeOH. The mixture was concentrated and the residue was diluted with EtOAc. The mixture was washed with 1 M HCl, water, and brine, dried with a phase separator, and evaporated to dryness to give 506 mg of crude 3-(tert-butyl)-7-fluoro-1-methylquinazoline-2,4(1H,3H)-dione. ¹H-NMR (400 MHz, d₆-DMSO): δ 1.65 (s, 9H), 3.41 (s, 3H), 7.06 (ddd, 1H), 7.27 (dd, 1H), 7.96 (dd, 1H).

### 7-Fluoro-1-methylquinazoline-2,4(1H,3H)-dione

3-(tert-Butyl)-7-fluoro-1-methylquinazoline-2,4(1H,3H)-dione (0.50 g; 2.0 mmol) in EtOH (15 ml) and concentrated HCl (1.97 ml; 24 mmol) was stirred under reflux for approximately 36 h. The reaction mixture was cooled to rt and concentrated. The residue was diluted with EtOAc, washed with saturated NaHCO₃, water, and brine, dried with a phase separator, and evaporated to dryness. The crude product was purified with MS-Trigger to give 94 mg of 7-fluoro-1-methylquinazoline-2,4(1H,3H)-dione. ¹H-NMR (400 MHz, CDCl₃): δ 3.54 (s, 3H), 6.91 (dd, 1H), 6.9d (ddd, 1H), 8.19 (br s, 1H), 8.22 (dd, 1H).

### 7-Fluoro-1-methyl-3-(4-nitrobenzyl)quinazoline-2,4(1H,3H)-dione

7-Fluoro-1-methylquinazoline-2,4(1H,3H)-dione (20 mg; 0.10 mmol), K₂CO₃ (29 mg; 0.21 mmol), and DMF (2 ml) were placed in a reaction flask under nitrogen and the mixture was stirred at rt for 15 min. 4-Nitrobenzyl bromide (24.5 mg; 0.11 mmol) dissolved in 0.5 ml of DMF was added and the reaction mixture was stirred at rt for 3 h. Water was added and the resulting precipitation was filtered, washed with water, and dried in a vacuum oven at 40° C. to give 20 mg of 7-fluoro-1-methyl-3-(4-nitrobenzyl)quinazoline-2,4(1H,3H)-dione. ¹H-NMR (400 MHz, CDCl₃): δ 3.58 (s, 3H), 5.33 (s, 2H), 6.90 (dd, 1H), 7.00 (ddd, 1H), 7.62-7.68 (m, 2H), 8.14-8.19 (m, 2H), 8.26 (dd, 1H).

### Example 41: 3-(4-Chloro-3-phenoxybenzyl)-7-fluoro-1-(2-hydroxy-2-methylpropyl)quinazoline-2,4(1H,3H)-dione

### 2-Amino-N-(4-chloro-3-phenoxybenzyl)-4-fluorobenzamide

2-Amino-4-fluorobenzoic acid (92 mg; 0.59 mmol), 5 ml of DCM, and TEA (0.25 ml; 1.77 mmol) were placed in reaction flask under nitrogen. (4-Chloro-3-phenoxyphenyl)methanamine (0.90 ml; 7.1 mmol) was added, the solution was cooled to 0° C., and T3P (0.42 ml; 0.71 mmol; 50% in DMF) was added slowly. The reaction mixture was stirred overnight at rt. DCM was added. The mixture was washed twice with water, dried with a phase separator, and evaporated to dryness. ¹H-NMR (400 MHz, CDCl₃): δ 4.49 (d, 2H), 5.71 (br s, 2H), 6.22-6.29 (m, 1H), 6.28-6.39 (m, 2H), 6.94-6.99 (m, 3H), 7.05-7.09 (m, 1H), 7.08-7.14 (m, 1H), 7.25 (dd, 1H), 7.30-7.37 (m, 2H), 7.43 (d, 1H).

### Ethyl-(2-((4-chloro-3-phenoxybenzyl)carbamoyl)-5-fluorophenyl)carbamate

2-Amino-N-(4-chloro-3-phenoxybenzyl)-4-fluorobenzamide (219 mg; 0.59 mmol) was dissolved in dry pyridine (5 ml) under nitrogen and cooled to 0° C. Ethyl chloroformate (0.17 ml; 1.77 mmol) was added slowly and the reaction mixture was stirred at rt overnight. EtOAc (10 ml) and 1 M HCl (10 ml) were added carefully, phases were separated, and water phase was washed twice with EtOAc. Organic layers were combined, washed twice with 1 M HCl and twice with water, dried with a phase separator, and evaporated to dryness. The product was dissolved in EtOAc and evaporated in order to remove pyridine residues. ¹H-NMR (400 MHz, CDCl₃): δ 1.31 (t, 3H), 4.21 (q, 2H), 4.51 (d, 2H), 6.52-6.59 (m, 1H), 6.66 (ddd, 1H), 6.91-6.98 (m, 3H), 7.04-7.08 (m, 1H), 7.09-7.14 (m, 1H), 7.30-7.36 (m, 2H), 7.36-7.41 (m, 1H), 7.44 (d, 1H), 7.38 (d, 1H), 8.20 (dd, 1H), 10.62 (br s, 1H).

### 3-(4-Chloro-3-phenoxybenzyl)-7-fluoroquinazoline-2,4(1H,3H)-dione

Ethyl (2-((4-chloro-3-phenoxybenzyl)carbamoyl)-5-fluorophenyl)carbamate (205 mg; 0.46 mmol), 10 ml of EtOH, and 0.46 ml of 2 M NaOH were charged and refluxed for 2 h. Water was added and the reaction mixture was neutralized with HCl at rt. The precipitation was filtered and dried under vacuum overnight at 40° C. The product was triturated with EtOH, filtered, and dried to give 161 mg of 3-(4-chloro-3-phenoxybenzyl)-7-fluoroquinazoline-2,4(1H,3H)-dione. ¹H-NMR (400 MHz, d₆-DMSO): δ 5.01 (s, 2H), 6.88-6.95 (m, 3H), 7.02-7.18 (m, 4H), 7.33-7.40 (m, 2H), 7.53 (d, 1H), 7.98 (d, 1H), 11.65 (br s, 1H).

### 3-(4-Chloro-3-phenoxybenzyl)-7-fluoro-1-(2-hydroxy-2-methylpropyl)quinazoline-2,4(1H,3H)-dione

3-(4-Chloro-3-phenoxybenzyl)-7-fluoroquinazoline-2,4(1H,3H)-dione (50 mg; 0.13 mmol), K₂CO₃ (26.1 mg, 0.19 mmol), isobutylene oxide (0.022 ml; 0.25 mmol), and 1 ml of DMF were placed in a microwave reaction vial and heated for 1 h at 130° C. and then 1 h at 140° C. Isobutylene oxide (0.05 ml; 0.50 mmol) was added and the mixture was heated for 2 h at 140° C. The reaction mixture was evaporated to dryness. The residue was dissolved in EtOAc and washed with 1 M NaHCO₃ and twice with water. Organic phases were combined, dried, and evaporated. The crude product was purified first with CombiFlash (EtOAc:heptane, normal phase silica) and then by LC-MS Trigger to yield 5.6 mg of 3-(4-chloro-3-phenoxybenzyl)-7-fluoro-1-(2-hydroxy-2-methylpropyl)quinazoline-2,4(1H,3H)-dione. ¹H-NMR (400 MHz, CDCl₃): δ 1.30 (s, 6H), 2.39 (br s, 1H), 4.14 (s, 2H), 5.18 (s, 2H), 6.88-6.93 (m, 2H), 6.96 (ddd, 1H), 7.05-7.10 (m, 1H), 7.16 (d, 1H), 7.18-7.21 (m, 1H), 7.22 (d, 1H), 7.27-7.33 (m, 2H), 7.38 (d, 1H), 8.21 (dd, 1H).

### Example 42: (R)-3-(1-(4-Chlorophenyl)ethyl)-7-fluoro-1-methylquinazoline-2,4(1H,3H)-dione

### (R)-2-Amino-N-(1-(4-chlorophenyl)ethyl)-4-fluorobenzamide

2-Amino-4-fluorobenzoic acid (4.98 g; 32.1 mmol), 30 ml of DCM, TEA (13.4 ml; 96.0 mmol), and (R)-1-(4-chlorophenyl)ethanamine (4.50 ml; 32.1 mmol) were placed in a reaction flask under nitrogen. The solution was cooled to 0° C. and T3P (22.7 ml; 38.6 mmol) was added slowly. The reaction was stirred at rt overnight. 70 ml of EtOAc was added and washed twice with 100 ml of water. Organic phase was dried over Na₂SO₄, filtered, and evaporated to dryness. The crude product was dissolved in toluene and some EtOAc and evaporated to dryness. The evaporation residue was dried at 50° C. under vacuum to give 7.92 g of (R)-2-amino-N-(1-(4-chlorophenyl)ethyl)-4-fluorobenzamide. ¹H-NMR (400 MHz, CDCl₃): δ 1.56 (d, 3H), 5.16-5.26 (m, 1H), 5.72 (br s, 2H), 6.09 (d, 1H) 6.30-6.37 (m, 2H), 7.27-7.35 (m, 5H), 7.45-7.50 (m, 2H).

### (R)-3-(1-(4-Chlorophenyl)ethyl)-7-fluoroquinazoline-2,4(1H,3H)-dione

(R)-2-Amino-N-(1-(4-chlorophenyl)ethyl)-4-fluorobenzamide (7.9 g; 27.0 mmol) and 35 ml of dry pyridine were placed in a reaction flask under nitrogen and cooled to 0° C. Ethyl chloroformate (7.7 ml; 81.0 mmol) was added carefully and the mixture was stirred at rt overnight. 2 M NaOH (68 ml; 135 mmol) was added slowly at 0° C. The mixture was heated at 50° C. for 2 h and then to 75° C., allowed to cool to rt, and stirred over the weekend. The reaction mixture was evaporated to dryness, 150 ml of DCM was added and pH was adjusted to 4 with 2 M HCl. Organic phase was washed twice with 50 ml of water, dried, and evaporated to dryness to give 8.57 g of (R)-3-(1-(4-chlorophenyl)ethyl)-7-fluoroquinazoline-2,4(1H,3H)-dione. ¹H-NMR (400 MHz, d₆-DMSO): δ 1.79 (d, 3H), 6.14 (q, 1H), 6.91 (dd, 1H), 7.05 (td, 1H), 7.29-7.41 (m, 4H), 7.96 (dd, 1H), 11.48 (br s, 1H).

### (R)-3-(1-(4-Chlorophenyl)ethyl)-7-fluoro-1-methylquinazoline-2,4(1H,3H)-dione

(R)-3-(1-(4-Chlorophenyl)ethyl)-7-fluoro-1-methylquinazoline-2,4(1H,3H)-dione was prepared similarly to 3-(4-bromobenzyl)-1-(3,3-dimethyl-2-oxobutyl)-7-fluoroquinazoline-2,4(1H,3H)-dione in Example 26. (R)-3-(1-(4-Chlorophenyl)ethyl)-7-fluoroquinazoline-2,4(1H,3H)-dione (8.5 g; 26.7 mmol), 50 ml of DMF, and NaOH (1.6 g; 40 mmol) were placed in a reaction flask under nitrogen and stirred at rt for 15 min. Iodomethane (2.49 ml; 40 mmol) was added slowly and the reaction mixture was stirred for 2.5 h at rt. Water (1.5 ml) was added and the mixture was stirred overnight at rt. 50 ml of water and 150 ml of EtOAc were added, phases were separated, and organic phase was washed five times with 150 ml of water. Organic phase was dried over Na₂SO₄, filtered, and evaporated to dryness to yield 8.08 g of (R)-3-(1-(4-chlorophenyl)ethyl)-7-fluoro-1-methylquinazoline-2,4(1H,3H)-dione. ¹H-NMR (400 MHz, d₆-DMSO): δ 1.79 (d, 3H), 3.45 (s, 3H), 6.21 (q, 1H), 7.14 (td, 1H), 7.33-7.39 (m, 5H), 8.08 (dd, 1H).

### Example 43: 3-(1-(4-Chlorophenyl)cyclopropyl)-7-fluoro-1-methylquinazoline-2,4(1H,3H)-dione

### 2-Amino-N-(1-(4-chlorophenyl)cyclopropyl)-4-fluorobenzamide

2-Amino-4-fluorobenzoic acid (0.4 g; 2.58 mmol), 8 ml of DCM, TEA (1.08 ml; 7.74 mmol), and 1-(4-chlorophenyl)cyclopropanamine (0.43 mg; 2.58 mmol) were placed in a reaction flask under nitrogen. T3P (1.82 ml; 3.09 mmol; 50% in DMF) was added slowly at 0° C. The reaction mixture was stirred overnight at rt, for 3 h at 50° C., and at rt over the weekend. DCM was added and the mixture was washed twice with water. Organic phase was dried and evaporated to dryness to give 706 mg of 2-amino-N-(1-(4-chlorophenyl)cyclopropyl)-4-fluorobenzamide. ¹H-NMR (400 MHz, d₆-DMSO): δ 1.20-1.30 (m, 4H), 6.26-6.36 (m, 1H), 6.45 (dd, 1H), 6.73 (br s, 2H) 7.16-7.24 (m, 2H), 7.25-7.39 (m, 2H), 7.68 (dd, 1H), 8.95 (s, 1H).

### Ethyl (2-((1-(4-chlorophenyl)cyclopropyl)carbamoyl)-5-fluorophenybcarbamate

2-Amino-N-(1-(4-chlorophenyl)cyclopropyl)-4-fluorobenzamide (0.932 g; 3.06 mmol) was dissolved in 5 ml of dry pyridine and ethyl chloroformate (0.57 ml; 6.00 mmol) was added slowly at 0° C. The reaction mixture was stirred at rt overnight. 10 ml of EtOAc was added and pH was adjusted to acidic with 10 ml of 1 M HCl. The layers were separated and water phase was washed twice with EtOAc. Organic phases were combined, washed twice with 1 M HO and twice with water, dried, and evaporated to dryness to give 692 mg of ethyl (2-((1-(4-chlorophenyl)cyclopropyl)carbamoyl)-5-fluorophenyl)carbamate. ¹H-NMR (400 MHz, d₆-DMSO): δ 1.22 (t, 3H), 1.25-1.33 (m, 4H), 4.12 (q, 2H), 6.98 (ddd, 1H), 7.19-7.24 (m, 2H), 7.31-7.36 (m, 2H), 7.98 (dd, 1H), 8.03 (dd, 1H), 9.49 (s, 1H), 11.22 (s, 1H).

### 3-(1-(4-Chlorophenyl)cyclopropyl)-7-fluoroquinazoline-2,4(1H,3H)-dione

Ethyl (2-((1-(4-chlorophenyl)cyclopropyl)carbamoyl)-5-fluorophenyl)carbamate (0.692 g; 1.84 mmol), 7 ml of EtOH, and 2 M NaOH (1.84 ml; 3.67 mmol) were placed in a reaction flask and refluxed for 1 h. Water (7 ml) was added and the mixture was neutralized with 2 M HCl. Water phase was extracted three times with EtOAc, dried, and evaporated to dryness to give 545 mg of 3-(1-(4-chlorophenyl)cyclopropyl)-7-fluoroquinazoline-2,4(1H,3H)-dione. ¹H-NMR (400 MHz, d₆-DMSO): δ 1.37-1.57 (m, 3H), 6.91 (dd, 1H), 7.05 (td, 1H), 7.18-7.24 (m, 2H), 7.28-7.33 (m, 2H), 7.98 (dd, 1H), 11.51 (br s, 1H).

### 3-(1-(4-Chlorophenyl)cyclopropyl)-7-fluoro-1-methylquinazoline-2,4(1H,3H)-dione

3-(1-(4-Chlorophenyl)cyclopropyl)-7-fluoroquinazoline-2,4(1H,3H)-dione (75 mg; 0.23 mmol), K₂CO₃ (64 mg; 0.45 mmol), and dry DMF (2 ml) were placed in a reaction flask and stirred for 15 min. Iodomethane (0.028 ml; 0.45 mmol) was added and the reaction mixture was stirred for 2 h at rt 0.1 M Citric acid (2.5 ml) was added to neutralize the reaction mixture. EtOAc (10 ml) was added, the mixture was washed twice with 10 ml of water, and organic phase was dried and evaporated. CombiFlash (normal phase silica) purification gave 85 mg of 3-(1-(4-chlorophenyl)cyclopropyl)-7-fluoro-1-methylquinazoline-2,4(1H,3H)-dione. ¹H-NMR (400 MHz, d₆-DMSO): δ 1.38-1.46 (m, 2H), 1.50-1.57 (m, 2H), 3.47 (s, 3H) 7.10-7.18 (m, 1H), 7.21-7.26 (m, 2H) 7.27-7.32 (m, 2H), 7.36 (dd, 1H), 8.08 (dd, 1H).

### Example 44: 3-(1-(4-Chlorophenyl)-3-methoxypropyl)-7-fluoro-1-methylquinazoline-2,4(1H,3H)-dione

### 2-Amino-N-(1-(4-chlorophenyl)-3-methoxypropyl)-4-fluorobenzamide

2-Amino-4-fluorobenzoic acid (250 mg; 1.61 mmol), 3 ml of DCM, TEA (0.67 ml; 4.83 mmol), and 1-(4-chlorophenyl)-3-methoxypropan-1-amine (354 mg; 1.77 mmol) were charged in a reaction flask under nitrogen. The reaction mixture was cooled to 0° C. and T3P was added slowly. The reaction mixture was stirred at rt overnight, diluted with DCM, and washed four times with water. Organic phase was dried and evaporated to yield 438 mg of 2-amino-N-(1-(4-chlorophenyl)-3-methoxypropyl)-4-fluorobenzamide. LC-MS (ES+) [M+1]: 337.1.

### Ethyl 2-(1-(4-chlorophenyl)-3-methoxypropylcarbamoyl)-5-fluorophenylcarbamate

2-Amino-N-(1-(4-chlorophenyl)-3-methoxypropyl)-4-fluorobenzamide (438 mg; 1.30 mmol) and 5 ml of pyridine were charged in a reaction flask under nitrogen. The reaction mixture was cooled to 0° C., ethyl chloroformate (0.373 ml; 3.90 mmol) was added slowly, and the mixture was stirred at rt overnight. DCM and 1 M HCl solution were added to the reaction mixture, phases were separated, and water phase was extracted twice with DCM. Organic phases were combined and washed twice with 1 M HCl and twice with water. Organic phase was dried and evaporated to yield 371 mg of ethyl 2-(1-(4-chlorophenyl)-3-methoxypropylcarbamoyl)-5-fluorophenylcarbamate. LC-MS (ES+) [M+1]: 409.2.

### 3-(1-(4-Chlorophenyl)-3-methoxypropyl)-7-fluoroquinazoline-2,4(1H,3H)-dione

Ethyl 2-(1-(4-chlorophenyl)-3-methoxypropylcarbamoyl)-5-fluorophenylcarbamate (370 mg; 0.905 mmol), 1 ml of EtOH, and 0.9 ml of 2 M NaOH solution were charged in a reaction flask and refluxed for 2 h. 0.1 ml of 2 M NaOH solution was added and the mixture was refluxed for 1 h and stirred at rt overnight. Water was added to the reaction mixture and pH was adjusted to neutral with 1 M HCl. The precipitation was filtered, washed with water, and dried in a vacuum oven to yield 262 mg of 3-(1-(4-chlorophenyl)-3-methoxypropyl)-7-fluoroquinazoline-2,4(1H,3H)-dione. LC-MS (ES−) [M−1]: 361.1.

### 3-(1-(4-Chlorophenyl)-3-methoxypropyl)-7-fluoro-1-methylquinazoline-2,4(1H,3H)-dione

3-(1-(4-Chlorophenyl)-3-methoxypropyl)-7-fluoroquinazoline-2,4(1H,3H)-dione (100 mg; 0.276 mmol), K₂CO₃ (78 mg; 0.55 mmol), and 1 ml of DMF were charged in a reaction flask under nitrogen. The reaction mixture was stirred at rt for 15 min, iodomethane (0.034 ml; 0.55 mmol) was added, and the mixture was stirred at rt for 1½ h. 0.1 M Citric acid was added and the mixture was extracted three times with DCM. Organic phases were combined, dried, and evaporated. The crude product was purified with CombiFlash (normal phase silica) to yield 56 mg of 3-(1-(4-chlorophenyl)-3-methoxypropyl)-7-fluoro-1-methylquinazoline-2,4(1H,3H)-dione. ¹H-NMR (400 MHz, CDCl₃): δ 2.54-2.65 (m, 1H), 2.76-2.90 (m, 1H), 3.24 (s, 3H), 3.43 (t, 2H), 3.50 (s, 3H), 6.33-6.45 (m, 1H), 6.84 (dd, 1H), 6.90-6.98 (m, 1H), 7.23-7.31 (m, 2H), 7.44-7.51 (m, 2H), 8.16-8.27 (m, 1H).

### Example 45: 3-(4-Bromobenzyl)-7-chloro-1-methylquinazoline-2,4(1H,3H)-dione

### 2-Amino-N-(4-bromobenzyl)-4-chlorobenzamide

2-Amino-4-chlorobenzoic acid (1.0 g; 5.8 mmol), 20 ml of EtOAc, TEA (2.4 ml; 17.5 mmol), and (4-bromophenyl)methanamine (0.81 ml; 6.4 mmol) were placed in a reaction flask under nitrogen. The solution was cooled to 0° C. and T3P (4.2 ml; 7.0 mmol) was added slowly. The reaction mixture was stirred for 30 min at 0° C. and overnight at rt. Water and DCM were added and water phase was washed twice with water and once with brine. Organic phase was dried and evaporated to dryness to give 2.10 g of crude 2-amino-N-(4-bromobenzyl)-4-chlorobenzamide. ¹H-NMR (400 MHz, CDCl₃): δ 4.54 (d, 2H), 5.69 (br s, 2H), 6.26 (br s, 1H), 6.59 (dd, 1H), 6.67-6.70 (m, 1H), 7.19-7.24 (m, 3H), 7.45-7.50 (m, 2H).

### 3-(4-Bromobenzyl)-7-chloroquinazoline-2,4(1H,3H)-dione

2-Amino-N-(4-bromobenzyl)-4-chlorobenzamide (2 g; 5.9 mmol) and pyridine were loaded into a reaction flask under nitrogen and cooled to 0° C. Ethyl chloroformate (1.7 ml; 17.7 mmol) was added carefully and the mixture was stirred at rt overnight. 2 M NaOH (11.8 ml; 23.6 mmol) was added slowly at 0° C. and the mixture was heated at 50° C. for 2 h to complete ring closure. The reaction mixture was evaporated to dryness, ˜25 ml of DCM was added, and pH was adjusted acidic with 1 M HCl. The precipitation was filtered, washed with water, and dried to give 1.11 g of 3-(4-bromobenzyl)-7-chloroquinazoline-2,4(1H,3H)-dione. ¹H-NMR (400 MHz, d₆-DMSO): δ 5.03 (s, 2H), 7.20-7.32 (m, 4H), 7.47-7.53 (m, 2H), 7.94 (d, 1H), 11.65 (br s,

### 3-(4-Bromobenzyl)-7-chloro-1-methylquinazoline-2,4(1H,3H)-dione

Sodium hydride (81 mg; 2.02 mmol; 60% in oil) was charged in a dry reaction flask under nitrogen and cooled to 0° C. 1 ml of DMF was added. 3-(4-Bromobenzyl)-7-chloroquinazoline-2,4(1H,3H)-dione (370 mg; 1.01 mmol) in 3 ml of DMF/THF was added dropwise and the mixture was stirred for 1 h at rt. Iodomethane (0.13 ml; 2.02 mmol) was added dropwise at 0° C. and the reaction mixture was stirred at rt overnight. 2 ml of MeOH was added carefully and the reaction mixture was evaporated to dryness. DCM and water were added and water phase was washed three times with 10 ml of DCM. Organic layers were combined, dried with a phase separator, and evaporated to dryness to give 390 mg of crude product. The residue was purified by trituration in MeOH, filtered, and dried to give 309 mg of 3-(4-bromobenzyl)-7-chloro-1-methylquinazoline-2,4(1H,3H)-dione. ¹H-NMR (400 MHz, d₆-DMSO): δ 3.51 (s, 3H), 5.08 (s, 2H), 7.26-7.32 (m, 2H), 7.37 (dd, 1H), 7.46-7.52 (m, 2H), 7.59 (d, 1H), 8.05 (d, 1H).

### Example 46: 3-(4-Bromobenzyl)-7-chloro-1-(2-hydroxy-2-methylpropyl)quinazoline-2,4(1H,3H)-dione

3-(4-Bromobenzyl)-7-chloroquinazoline-2,4(1H,3H)-dione (400 mg; 1.09 mmol) prepared in Example 45 and yttrium(III) nitrate hexahydrate (42 mg; 0.11 mmol) were placed in a microwave reaction vial. DMF (1 ml) and isobutylene oxide (2.91 ml; 32.8 mmol) were added and the reaction mixture was heated in a microwave reactor at 200° C. for 60 min. After cooling to rt, saturated NaHCO₃ was added and the mixture was extracted with DCM. The combined organic layers were washed with water and brine, dried with a phase separator, and evaporated to dryness. The crude product was purified with column chromatography (EtOAc:heptane) to give 310 mg of 3-(4-bromobenzyl)-7-chloro-1-(2-hydroxy-2-methylpropyl)quinazoline-2,4(1H,3H)-dione. ¹H-NMR (400 MHz, CDCl₃): δ 1.32 (s, 6H), 2.51 (s, 1H), 4.17 (s, 2H), 5.19 (s, 2H), 7.21 (dd, 1H), 7.34-7.44 (m, 4H), 7.52 (d, 1H), 8.14 (d, 1H).

### Example 47: 3-(4-Bromobenzyl)-7-chloro-1-((3-methyloxetan-3-yl)methyl)quinazoline-2,4(1H,3H)-dione

3-(4-Bromobenzyl)-7-chloroquinazoline-2,4(1H,3H)-dione (200 mg; 0.55 mmol) prepared in Example 45, sodium hydride (44 mg; 1.09 mmol, 60% in oil), and DMF (1 ml) were placed in a microwave reaction vial under nitrogen and the mixture was stirred at rt for 15 min. 3-(Chloromethyl)-3-methyloxetane (0.24 ml; 2.19 mmol) in 0.5 ml of DMF was added and the reaction mixture was heated in a microwave reactor at 160° C. for 3 h. After cooling to rt, MeOH was added and the mixture was concentrated. The residue was diluted with DCM and the mixture was washed with NaHCO₃, water, and brine, dried with a phase separator, and evaporated to dryness. The crude product was purified with column chromatography to give 100 mg of 3-(4-bromobenzyl)-7-chloro-1-((3-methyloxetan-3-yl)methyl)quinazoline-2,4(1H,3H)-dione. ¹H-NMR (400 MHz, d₆-DMSO): δ 1.32 (s, 3H), 4.07 (d, 2H), 4.24 (s, 2H), 4.54 (d, 2H), 5.09 (s, 2H), 7.26-7.31 (m, 2H), 7.37 (dd, 1H), 7.48-7.52 (m, 2H), 7.78 (d, 1H), 8.07 (d, 1H).

### Example 48: 3-(4-Bromobenzyl)-7-chloro-6-fluoro-1-methylquinazoline-2,4(1H,3H)-dione

### 2-Amino-N-(4-bromobenzyl)-4-chloro-5-fluorobenzamide

2-Amino-4-chloro-5-fluorobenzoic acid (2.5 g; 13.2 mmol), 20 ml of DCM, TEA (5.5 ml; 39.6 mmol), and (4-bromophenyl)methanamine (1.83 ml; 14.5 mmol) were placed in a reaction flask under nitrogen and cooled to 0° C. T3P (9.4 ml; 15.8 mmol) was added slowly. The reaction mixture was allowed to warm to rt after 30 min and stirred overnight. Water (15 ml) was added and the precipitation formed was filtered, washed three times with 10 ml of water, and dried under vacuum at 50° C. to give 3.01 g of the product. Additional product was precipitated from the filtrate, filtered, washed, and dried to give 0.44 g of the product. ¹H-NMR (400 MHz, d₆-DMSO): δ 4.37 (d, 2H), 6.54 (br s, 2H), 6.88 (d, 1H), 7.24-7.29 (m, 2H), 7.49-7.54 (m, 2H), 7.61 (d, 1H), 8.92 (t, 1H).

### 3-(4-Bromobenzyl)-7-chloro-6-fluoroquinazoline-2,4(1H,3H)-dione

2-Amino-N-(4-bromobenzyl)-4-chloro-5-fluorobenzamide (3.5 g; 9.8 mmol) and 15 ml of pyridine were charged in a reaction flask under nitrogen and cooled to 0° C. Ethyl chloroformate (2.8 ml; 29.4 mmol) was added carefully and the mixture was stirred at rt overnight. 2 M NaOH (19.6 ml; 40.0 mmol) was added slowly at 0° C. and the mixture was heated at 50° C. for 2 h to complete ring closure. The reaction mixture was evaporated to dryness, ˜25 ml of DCM was added, and pH was adjusted to <4 with 1 M HCl. The precipitation was filtered, washed with water, and dried. The crude product was triturated in heptane, filtered, and dried to give 3.30 g of 3-(4-bromobenzyl)-7-chloro-6-fluoroquinazoline-2,4(1H,3H)-dione. ¹H-NMR (400 MHz, d₆-DMSO): δ 5.03 (s, 2H), 7.26-7.31 (m, 2H), 7.35 (d, 1H), 7.47-7.53 (m, 2H), 7.84 (d, 1H), 11.68 (br s, 1H).

### 3-(4-Bromobenzyl)-7-chloro-6-fluoro-1-methylquinazoline-2,4(1H,3H)-dione

Sodium hydride (60 mg; 1.5 mmol; 60% in oil), 3-(4-bromobenzyl)-7-chloro-6-fluoroquinazoline-2,4(1H,3H)-dione (564 mg; 0.75 mmol; 51% purity), and 4 ml of dry DMF were charged in a reaction flask under nitrogen, cooled to 0° C., and stirred for 30 min. Iodomethane (0.093 ml; 1.5 mmol) was added dropwise at 0° C. and the reaction mixture was stirred at rt overnight. 1 ml of MeOH was added carefully. The mixture was stirred for 30 min and evaporated to dryness. Water was added and the mixture was washed three times with DCM. Combined organic phases were washed with brine, dried, and evaporated to dryness to give 334 mg of crude product. DCM and water were added and water phase was washed three times with 10 ml of DCM. Organic layers were combined, dried with a phase separator, and evaporated to dryness to give 390 mg of crude product. CombiFlash/MS-Trigger purification provided 48 mg of 3-(4-bromobenzyl)-7-chloro-6-fluoro-1-methylquinazoline-2,4(1H,3H)-dione. ¹H-NMR (400 MHz, CDCl₃): δ 3.57 (s, 3H), 5.19 (s, 2H), 7.24-7.27 (m, 1H), 7.36-7.45 (m, 4H), 7.97 (d, 1H).

### Example 49: 3-(4-Bromobenzyl)-7-chloro-6-fluoro-1-(2-hydroxy-2-methylpropyl)quinazoline-2,4(1H,3H)-dione

3-(4-Bromobenzyl)-7-chloro-6-fluoro-1-(2-hydroxy-2-methylpropyl)quinazoline-2,4(1H,3H)-dione was prepared similarly to 3-(3,4-dichlorobenzyl)-1-(2-hydroxy-2-methylpropyl)-7-(trifluoromethyDquinazoline-2,4(1H,3H)-dione in Example 2. 3-(4-Bromobenzyl)-7-chloro-6-fluoroquinazoline-2,4(1H,3H)-dione (194 mg; 0.51 mmol) prepared in Example 48 and yttrium(III) nitrate hexahydrate (9.7 mg; 0.025 mmol) were placed in a microwave reaction vial. DMF (3 ml) and isobutylene oxide (8.98 ml; 101 mmol) were added and the reaction mixture was heated in a microwave reactor at 160° C. for 60 min. After work-up, the crude product was purified with column chromatography (EtOAc:heptane) to give 178 mg of 3-(4-bromobenzyl)-7-chloro-6-fluoro-1-(2-hydroxy-2-methylpropyl)quinazoline-2,4(1H,3H)-dione. ¹H-NMR (400 MHz, CDCl₃): δ 1.33 (s, 6H), 2.24 (s, 1H), 4.16 (s, 2H), 5.20 (s, 2H), 7.35-7.39 (m, 2H), 7.41-7.45 (m, 2H), 7.66 (d, 1H), 7.95 (d, 1H).

### Example 50: 3-(4-Bromobenzyl)-7-chloro-6-fluoro-1-((3-methyloxetan-3-yl)methyl)quinazoline-2,4(1H,3H)-dione

3-(4-Bromobenzyl)-7-chloro-6-fluoroquinazoline-2,4(1H,3H)-dione (200 mg; 0.52 mmol) prepared in Example 48, sodium hydride (31 mg; 0.78 mmol, 60% in oil), and DMF (2 ml) were placed in a microwave reaction vial under nitrogen and the mixture was stirred at rt for 15 min. 3-(Chloromethyl)-3-methyloxetane (0.11 ml; 1.04 mmol) in 0.5 ml of DMF was added and the reaction mixture was heated in a microwave reactor at 160° C. for 1 h. After cooling to rt, MeOH was added and the mixture was concentrated. The residue was diluted with DCM and the mixture was washed with saturated NaHCO₃, water, and brine, dried with a phase separator, and evaporated to dryness. The crude product was purified with column chromatography (EtOAc:heptane) to give 112 mg of 3-(4-bromobenzyl)-7-chloro-6-fluoro-1-((3-methyloxetan-3-yl)methyl)quinazoline-2,4(1H,3H)-dione. ¹H-NMR (400 MHz, CDCl₃): δ 1.47 (s, 3H), 4.14 (s, 2H), 4.26 (d, 2H), 4.66 (d, 2H), 5.19 (s, 2H), 7.07 (d, 1H), 7.35-7.40 (m, 2H), 7.41-7.46 (m, 2H), 8.00 (d, 1H).

### Example 51: Methyl 2-(3-(4-bromobenzyl)-7-chloro-6-fluoro-2,4-dioxo-3,4-dihydroquinazolin-1(2H)-yl)propanoate

3-(4-Bromobenzyl)-7-chloro-6-fluoroquinazoline-2,4(1H,3H)-dione (1.50 g; 3.9 mmol) prepared in Example 48, K₂CO₃ (1.62 g; 11.7 mmol), and DMF (10 ml) were placed in a microwave reaction vial under nitrogen and the mixture was stirred at rt for 30 min. Methyl 2-bromopropionate (2.61 g; 15.6 mmol) was added and the reaction mixture was heated in a microwave reactor at 80-120° C. for 75 min. The reaction mixture was cooled to rt and water was added. After extracting with DCM, the combined organic layers were washed with water and brine, dried with a phase separator, and evaporated to dryness. The crude product was purified with column chromatography (EtOAc:heptane) to give 1.22 g of methyl 2-(3-(4-bromobenzyl)-7-chloro-6-fluoro-2,4-dioxo-3,4-dihydroquinazolin-1(2H)-yl)propanoate. ¹H-NMR (400 MHz, CDCl₃): δ 1.70 (d, 3H), 3.69 (s, 3H), 5.17 (dd, 2H), 5.19-5.31 (m, 1H), 7.11 (d, 1H), 7.33-7.40 (m, 2H), 7.40-7.46 (m, 2H), 8.00 (d, 1H).

### Example 52: 3-(4-Bromobenzyl)-7-fluoro-1-neopentylquinazoline-2,4(1H,3H)-dione

### N-(4-Bromobenzyl)-4-fluoro-2-(neopentylamino)benzamide

2-Amino-N-(4-bromobenzyl)-4-fluorobenzamide (2.5 g; 7.7 mmol), 20 ml of DCE, trimethylacetaldehyde (0.84 ml; 7.7 mmol), and glacial AcOH (1.1 ml; 19.3 mmol) were placed in a reaction flask. The mixture was cooled to 0° C. and sodium triacetoxyborohydride (3.28 g; 15.5 mmol) was added carefully. The reaction was stirred at rt for 3 h. Water (10 ml) was added carefully at 0° C. and layers were allowed to separate. Organic phase was washed with 1 M Na₂CO₃ and brine, dried over Na₂SO₄, filtered, and evaporated to dryness to give 2.84 g of crude product. CombiFlash purification (normal phase silica) provided 1.87 g of N-(4-bromobenzyl)-4-fluoro-2-(neopentylamino)benzamide. ¹H-NMR (400 MHz, CDCl₃): δ 1.04 (s, 9H), 2.89 (d, 2H), 4.55 (d, 2H), 6.15-6.24 (m, 2H), 6.35 (dd, 1H), 7.19-7.24 (m, 2H), 7.29 (dd, 1H), 7.44-7.50 (m, 2H), 8.11 (br s, 1H).

### Ethyl (2-((4-bromobenzyl)carbamoyl)-5-fluorophenyl)(neopentyl)carbamate

N-(4-Bromobenzyl)-4-fluoro-2-(neopentylamino)benzamide (800 mg; 2.03 mmol) and 9 ml of dry pyridine were placed in a reaction flask under nitrogen and cooled to 0° C. Ethyl chloroformate (0.97 ml; 10.2 mmol) was added slowly and the reaction mixture was allowed to warm to rt and stirred for 2 h, then 2 h at 130° C., and additionally 2 h at rt. The reaction was not complete, but 10 ml of EtOAc and 10 ml of 1 M HCl were added and phases were separated. Water layer was washed twice with EtOAc. Organic phases were combined, washed twice with 1 M HCl and twice with water, dried, and evaporated to dryness. CombiFlash purification (normal phase silica) gave 293 mg of ethyl (2-((4-bromobenzyl)carbamoyl)-5-fluorophenyl)(neopentyl)carbamate. ¹H-NMR (400 MHz, CDCl₃): δ 0.81 (s, 9H), 1.10-1.21 (m, 3H), 3.30-3.60 (m, 2H), 3.85-4.08 (m, 2H), 4.34-4.64 (m, 2H), 6.96 (dd, 1H), 7.05 (ddd, 1H), 7.19-7.24 (m, 2H), 7.44-7.49 (m, 2H), 7.61 (dd, 1H).

### 3-(4-Bromobenzyl)-7-fluoro-1-neopentylquinazoline-2,4(1H,3H)-dione

Ethyl (2-((4-bromobenzyl)carbamoyl)-5-fluorophenyl)(neopentyl)carbamate (290 mg; 0.62 mmol) and 6 ml of EtOH were placed in a reaction flask. 2 M NaOH (0.62 ml; 1.25 mmol) was added and the reaction mixture was stirred at rt for 1 h to complete the reaction. Water (10 ml) was added and the reaction mixture was neutralized with HCl to give precipitation, which was filtered. The filtrate was extracted twice with EtOAc. Organic phases were combined with the precipitation and evaporated to dryness. CombiFlash purification (normal phase silica) provided 136 mg of 3-(4-bromobenzyl)-7-fluoro-1-neopentylquinazoline-2,4(1H,3H)-dione. ¹H-NMR (400 MHz, CDCl₃): δ 0.99 (s, 9H), 3.99 (br s, 2H), 5.20 (s, 2H), 6.93 (ddd, 1H), 6.99 (dd, 1H), 7.36-7.44 (m, 4H), 8.23 (dd, 1H).

### Example 53: 3-(4-Bromobenzyl)-7-chloro-6-fluoro-1-(2-methoxy-2-methylpropyl)quinazoline-2,4(1H,3H)-dione

3-(4-Bromobenzyl)-7-chloro-6-fluoro-1-(2-hydroxy-2-methylpropyl)quinazoline-2,4(1H,3H)-dione (100 mg; 0.22 mmol) prepared in Example 49, sodium hydride (18 mg; 0.44 mmol, 60% in oil), and DMF (2 ml) were placed in a reaction flask under nitrogen and the mixture was stirred at rt for 15 min. Iodomethane (0.055 ml; 0.88 mmol) in 0.5 ml of DMF was added and the reaction mixture was stirred at rt for 2 h. MeOH was added and the mixture was concentrated. The residue was diluted with DCM and the mixture was washed with water and brine, dried with a phase separator, and evaporated to dryness. The crude product was purified with column chromatography (EtOAc:heptane) to give 13 mg of 3-(4-bromobenzyl)-7-chloro-6-fluoro-1-(2-methoxy-2-methylpropyl)quinazoline-2,4(1H,3H)-dione. ¹H-NMR (400 MHz, CDCl₃): δ 1.23 (s, 6H), 3.14 (s, 3H), 3.6-4.9 (m, 2H), 5.19 (s, 2H), 7.34-7.38 (m, 2H), 7.39-7.44 (m, 2H), 7.87-7.92 (m, 2H).

### Example 54: 7-Chloro-6-fluoro-3-(4-methoxybenzyl)-1-((3-methyloxetan-3-yl)methyl)quinazoline-2,4(1H,3H)-dione

### 2-Amino-4-chloro-5-fluoro-N-(4-methoxybenzyl)benzamide

2-Amino-4-chloro-5-fluorobenzoic acid (1.5 g; 7.91 mmol), DCM (25 ml), TEA (3.3 ml; 24 mmol), and (4-methoxyphenyl)methanamine (1.19 g; 8.70 mmol) were placed in a reaction flask under nitrogen. The solution was cooled to 0° C. and T3P (5.65 ml; 9.50 mmol, 50% solution) was added slowly. The reaction mixture was stirred for 30 min at 0° C. and 3 h at rt. The mixture was diluted with DCM, water was added, and the resulting precipitation was filtered, washed with water, and dried in a vacuum oven at 40° C. The phases of the filtrate were separated and the organic phase was washed with water and brine, dried with a phase separator, and evaporated to dryness. The evaporation residue and the precipitation were combined to give 1.94 of 2-amino-4-chloro-5-fluoro-N-(4-methoxybenzyl)benzamide. ¹H-NMR (400 MHz, d₆-DMSO): δ 3.73 (s, 3H), 4.34 (d, 2H), 6.54 (br s, 2H), 6.84-6.92 (m, 3H), 7.20-7.26 (m, 2H), 7.59 (d, 1H), 8.83 (t, 1H).

### 7-Chloro-6-fluoro-3-(4-methoxybenzyl)quinazoline-2,4(1H,3H)-dione

2-Amino-4-chloro-5-fluoro-N-(4-methoxybenzyl)benzamide (0.97 g; 3.15 mmol) and 5 ml of pyridine were placed in a reaction flask under nitrogen. The reaction mixture was cooled to 0° C. and ethyl chloroformate (0.90 ml; 9.44 mmol) was added dropwise. The reaction mixture was stirred overnight at rt and then cooled to 0° C. 5 M NaOH (7.86 ml; 15.7 mmol) was carefully added and the mixture was heated at 50° C. for 3 h. The mixture was concentrated and the residue was diluted with DCM. Addition of 1 M HCl gave a precipitation, which was filtered, washed with water, and dried in a vacuum oven at 40° C. to give 1.07 g of crude 7-chloro-6-fluoro-3-(4-methoxybenzyl)quinazoline-2,4(1H,3H)-dione. ¹H-NMR (400 MHz, d₆-DMSO): δ 3.71 (s, 3H), 4.99 (s, 2H), 6.82-6.88 (m, 2H), 7.25-7.32 (m, 3H), 7.81 (d, 1H).

### 7-Chloro-6-fluoro-3-(4-methoxybenzyl)-1-((3-methyloxetan-3-yl)methyl)quinazoline-2,4(1H,3H)-dione

7-Chloro-6-fluoro-3-(4-methoxybenzyl)quinazoline-2,4(1H,3H)-dione (150 mg; 0.49 mmol), sodium hydride (36 mg; 0.90 mmol, 60% in oil), and 2 ml of DMF were placed in a microwave reaction vial under nitrogen and the mixture was stirred at rt for 15 min. 3-(Chloromethyl)-3-methyloxetane (0.15 ml; 1.34 mmol) in 0.5 ml of DMF was added and the reaction mixture was heated in a microwave reactor at 120° C. for 3 h. After cooling to rt, MeOH was added and the mixture was concentrated. The residue was diluted with DCM and the mixture was washed with saturated NaHCO₃, water and brine, dried with a phase separator, and evaporated to dryness. The crude product was purified with column chromatography (EtOAc:heptane) and MS-Trigger to give 8 mg of 7-chloro-6-fluoro-3-(4-methoxybenzyl)-1-((3-methyloxetan-3-yl)methyl)quinazoline-2,4(1H,3H)-dione. ¹H-NMR (400 MHz, CDCl₃): δ 1.47 (s, 3H), 3.77 (s, 3H), 4.13 (s, 2H), 4.26 (d, 2H), 4.67 (d, 2H), 5.19 (s, 2H), 6.81-6.86 (m, 2H), 7.05 (d, 1H), 7.43-7.48 (m, 2H), 8.00 (d, 1H).

### Example 55: Methyl 2-(7-chloro-6-fluoro-3-(4-methoxybenzyl)-2,4-dioxo-3,4-dihydroquinazolin-1(2H)-yl)propanoate

The preparation of 7-chloro-6-fluoro-3-(4-methoxybenzyl)quinazoline-2,4(1H,3H)-dione is described in Examples 4 and 54. 7-Chloro-6-fluoro-3-(4-methoxybenzyl)quinazoline-2,4(1H,3H)-dione (2.00 g; 5.97 mmol), K₂CO₃ (2.48 g; 17.9 mmol), and ACN (15 ml) were placed in a microwave reaction vial under nitrogen and the mixture was stirred at rt for 30 min. Methyl 2-bromopropionate (2.67 ml; 23.9 mmol) was added and the reaction mixture was heated in a microwave reactor at 120° C. for 1 h. The reaction mixture was cooled to rt and water was added. After extracting with DCM, the combined organic layers were washed with water and brine, dried with a phase separator, and evaporated to dryness. The crude product was purified with column chromatography (ACN:water) to give 1.69 g of methyl 2-(7-chloro-6-fluoro-3-(4-methoxybenzyl)-2,4-dioxo-3,4-dihydroquinazolin-1(2H)-yl)propanoate. ¹H-NMR (400 MHz, CDCl₃): δ 1.70 (d, 3H), 3.69 (s, 3H), 3.77 (s, 3H), 5.16 (d, 2H), 5.20-5.33 (br s, 1H), 6.80-6.86 (m, 2H), 7.09 (d, 1H), 7.43-7.49 (m, 2H), 7.99 (d, 1H).

### Example 56: 7-Chloro-6-fluoro-1-(3-hydroxy-3-methylbutan-2-yl)-3-(4-methoxybenzyl)quinazoline-2,4(1H,3H)-dione

The preparation of methyl 2-(7-chloro-6-fluoro-3-(4-methoxybenzyl)-2,4-dioxo-3,4-dihydroquinazolin-1(2H)-yl)propanoate is described in Example 55. Methyl 2-(7-chloro-6-fluoro-3-(4-methoxybenzyl)-2,4-dioxo-3,4-dihydroquinazolin-1(2H)-yl)propanoate (175 mg; 0.42 mmol) and THF (3 ml) were placed in a reaction flask under nitrogen and the mixture was cooled to 0° C. Methylmagnesium bromide (0.416 ml; 1.25 mmol; 3 M in THF) was added dropwise and the mixture was stirred at 0° C. for 30 min and then at rt overnight. The mixture was cooled to 0° C. and saturated NR₄Cl was carefully added while allowing the mixture to warm to rt. EtOAc was added and the mixture was washed with water and brine. The organic phase was dried with a phase separator, evaporated to dryness, and purified with MS-Trigger to give 0.4 mg of 7-chloro-6-fluoro-1-(3-hydroxy-3-methylbutan-2-yl)-3-(4-methoxybenzyl)quinazoline-2,4(1H,3H)-dione. ¹H-NMR (400 MHz, CDCl₃): δ 1.10 (s, 3H), 1.39 (s, 3H), 1.55 (s, 3H), 3.77 (s, 3H), 4.23 (q, 1H), 5.18 (dd, 2H), 5.30 (br s, 1H), 6.81-6.86 (m, 2H), 7.28-7.32 (m, 1H), 7.43-7.48 (m, 2H), 8.00 (d, 1H).

### Example 57: (R)-7-Chloro-3-(1-(4-chlorophenyl)ethyl)-6-fluoro-1-methylquinazoline-2,4(1H,3H)-dione

### (R)-2-Amino-4-chloro-N-(1-(4-chlorophenyl)ethyl)-5-fluorobenzamide

2-Amino-4-chloro-5-fluorobenzoic acid (1.0 g; 5.28 mmol), DCM (10 ml), and TEA (2.21 ml; 15.83 mmol) were placed in a reaction flask under nitrogen. (R)-1-(4-Chlorophenyl)ethanamine (0.74 ml; 5.28 mmol) was added slowly and then T3P (3.73 ml; 6.33 mmol; 50% solution) was added slowly. The reaction mixture was stirred overnight at rt. DCM (30 ml) was added and the reaction mixture was washed four times with 50 ml of water. Organic phase was dried, evaporated to dryness, and dried at 40° C. under vacuum to give 1.63 g of (R)-2-amino-4-chloro-N-(1-(4-chlorophenyl)ethyl)-5-fluorobenzamide. ¹H-NMR (400 MHz, CDCl₃): δ 1.59 (d, 3H), 5.24 (quint, 1H), 5.80 (br s, 2H), 6.23 (d, 1H), 7.29-7.37 (m, 4H), 7.42-7.44 (m, 1H), 7.50 (dd, 1H).

### (R)-7-Chloro-3-(1-(4-chlorophenyl)ethyl)-6-fluoroquinazoline-2,4(1H,3H)-dione

(R)-2-Amino-4-chloro-N-(1-(4-chlorophenyl)ethyl)-5-fluorobenzamide (1.63 g; 4.98 mmol) and 7.5 ml of dry pyridine were placed in a reaction flask under nitrogen. Ethyl chloroformate (1.43 ml; 14.95 mmol) was added dropwise at 0° C. and the reaction mixture was stirred overnight at rt. 2 M NaOH (12 ml) was added slowly at 0° C. to give precipitation. The reaction mixture was stirred for 2 h at 50° C. to complete ring closure. The reaction mixture was evaporated to dryness, 25 ml of DCM was added, and pH was adjusted to ˜5 with 2 M HCl. Organic phase was washed twice with 15 ml of water. 15 ml of DCM was added during both separations to dissolve precipitation formed. Organic phase was dried with a phase separator and evaporated to dryness to give 1.75 g of crude product. The evaporation residue was dissolved in 20 ml of DCM and washed with 10 ml of 1 M HCl and then twice with 10 ml of water. Organic phase was dried by filtration through a phase separator and evaporated to dryness to give 1.56 g of (R)-7-chloro-3-(1-(4-chlorophenyl)ethyl)-6-fluoroquinazoline-2,4(1H,3H)-dione. ¹H-NMR (400 MHz, d₆-DMSO): δ 1.79 (d, 3H), 6.13 (q, 1H), 7.31 (d, 1H), 7.33-7.36 (m, 4H), 7.81 (d, 1H), 11.55 (br s, 1H).

### (R)-7-Chloro-3-(1-(4-chlorophenyl)ethyl)-6-fluoro-1-methylquinazoline-2,4(1H,3H)-dione

(R)-7-chloro-3-(1-(4-chlorophenyl)ethyl)-6-fluoroquinazoline-2,4(1H,3H)-dione (200 mg; 0.57 mmol), K₂CO₃ (157 mg; 1.13 mmol), and 3 ml of DMF were placed in a reaction flask under nitrogen and the mixture was stirred at rt for 15 min. Iodomethane (0.14 ml; 2.27 mmol) was added and the mixture was stirred for two days at rt. Water was added and the mixture was extracted with DCM. The combined organic layers were washed with water and brine, dried with a phase separator, and evaporated to dryness. The crude product was purified with column chromatography (EtOAc:heptane) to give 145 mg of (R)-7-chloro-3-(1-(4-chlorophenyl)ethyl)-6-fluoro-1-methylquinazoline-2,4(1H,3H)-dione. ¹H-NMR (400 MHz, CDCl₃): δ 1.89 (d, 3H), 3.51 (s, 3H), 6.36 (q, 1H), 7.23 (d, 1H), 7.25-7.30 (m, 2H), 7.35-7.41 (m, 2H), 7.94 (d, 1H).

### Example 58: (R)-7-Chloro-3-(1-(4-chlorophenyl)ethyl)-6-fluoro-1-((3-methyloxetan-3-yl)-methyl)quinazoline-2,4(1H,3H)-dione

(R)-7-chloro-3-(1-(4-chlorophenyl)ethyl)-6-fluoroquinazoline-2,4(1H,3H)-dione (200 mg; 0.57 mmol) prepared in Example 57, sodium hydride (45 mg; 1.13 mmol, 60% in oil), and 3 ml of DMF were placed in a microwave reaction vial under nitrogen and the mixture was stirred at rt for 15 min. 3-(Chloromethyl)-3-methyloxetane (0.25 ml; 2.27 mmol) dissolved in 0.5 ml of DMF was added and the reaction mixture was heated in a microwave reactor at 160° C. for 3 h. After cooling to rt, MeOH was added and the mixture was concentrated. The residue was diluted with DCM and the mixture was washed with saturated NaHCO₃, water, and brine, dried with a phase separator, and evaporated to dryness. The crude product was purified with column chromatography (EtOAc:heptane) to give 205 mg of (R)-7-chloro-3-(1-(4-chlorophenyl)ethyl)-6-fluoro-1-((3-methyloxetan-3-yl)methyl)quinazoline-2,4(1H,3H)-dione. ¹H-NMR (400 MHz, CDCl₃): δ 1.43 (s, 3H), 1.89 (d, 3H), 4.07 (q, 2H), 4.18-4.24 (m, 2H), 4.54 (d, 1H), 4.62 (d, 1H), 6.35 (q, 1H), 7.06 (d, 1H), 7.24-7.30 (m, 2H), 7.33-7.38 (m, 2H), 7.97 (d, 1H).

### Example 59: 3-(4-Bromobenzyl)-7,8-difluoro-1-methylquinazoline-2,4(1H,3H)-dione

### 2-Amino-N-(4-bromobenzyl)-3,4-difluorobenzamide

2-Amino-3,4-difluorobenzoic acid (500 mg; 2.89 mmol), 15 ml of DCM, TEA (1.21 ml; 8.66 mmol), and (4-bromophenyl)methanamine (0.40 ml; 3.18 mmol) were charged in a reaction flask under nitrogen and T3P (2.04 ml; 3.47 mmol; 50% in DMF) was added slowly. The reaction mixture was stirred at rt overnight, diluted with DCM and washed four times with water. Organic phase was dried and evaporated to yield 940 mg of 2-amino-N-(4-bromobenzyl)-3,4-difluorobenzamide. ¹H-NMR (400 MHz, CDCl₃): δ 4.54 (d, 2H), 5.84 (br s, 2H), 6.24 (br s, 1H), 6.42 (ddd, 1H), 7.06 (ddd, 1H), 7.19-7.24 (m, 2H), 7.45-7.51 (m, 2H).

### Ethyl (6-((4-bromobenzyl)carbamoyl)-2,3-difluorophenyl)carbamate

2-Amino-N-(4-bromobenzyl)-3,4-difluorobenzamide (940 mg; 2.76 mmol) and 10 ml of pyridine were charged in a reaction flask under nitrogen. The reaction mixture was cooled to 0° C., ethyl chloroformate (0.790 ml; 8.27 mmol) was added slowly, and the mixture was stirred at rt overnight. EtOAc and 1 M HCl solution were added to the reaction mixture. Phases were separated and water phase was extracted twice with EtOAc. Organic phases were combined and washed twice with 1 M HCl and twice with water. Organic phase was dried and evaporated to yield 1.18 g of ethyl (6-((4-bromobenzyl)carbamoyl)-2,3-difluoro-phenyl)carbamate. LC-MS (ES+) [M+1]: 415.1.

### 3-(4-Bromobenzyl)-7,8-difluoroquinazoline-2,4(1H,3H)-dione

Ethyl (6-((4-bromobenzyl)carbamoyl)-2,3-difluorophenyl)carbamate (1.18 g; 2.86 mmol), 25 ml of EtOH, and 2.86 ml of 2 M NaOH were charged in a reaction flask, refluxed for 1½ h, and cooled to rt. Water was added and pH was adjusted to neutral. The precipitation was filtered, washed with water, and dried in a vacuum oven to yield 632 mg of 3-(4-bromobenzyl)-7,8-difluoroquinazoline-2,4(1H,3H)-dione. ¹H-NMR (400 MHz, CDCl₃): 5.17 (s, 2H), 7.04 (ddd, 1H), 7.38-7.46 (m, 4H), 7.92 (ddd, 1H), 8.93 (br s, 1H).

### 3-(4-Bromobenzyl)-7,8-difluoro-1-methylquinazoline-2,4(1H,3H)-dione

3-(4-Bromobenzyl)-7,8-difluoroquinazoline-2,4(1H,3H)-dione (100 mg; 0.27 mmol), K₂CO₃ (75 mg; 0.55 mmol), and 3 ml of DMF were charged in a reaction flask under nitrogen. The reaction mixture was stirred at rt for 1 h. Iodomethane (0.034 ml; 0.55 mmol) was added and the reaction mixture was stirred at rt for 1 h. 0.1 M Citric acid was added. The precipitation was filtered, washed with water, and dried in a vacuum oven to yield 80 mg of 3-(4-bromobenzyl)-7,8-difluoro-1-methylquinazoline-2,4(1H,3H)-dione. ¹H-NMR (400 MHz, CDCl₃): δ 3.80 (d, 3H), 5.19 (s, 2H), 7.06 (ddd, 1H), 7.36-7.46 (m, 4H), 8.04 (ddd, 1H).

### Example 60: 3-(4-Bromobenzyl)-6,7,8-trifluoro-1-(2-hydroxy-2-methylpropyl)quinazoline-2,4(1H,3H)-dione

### 2-Amino-N-(4-bromobenzyl)-3,4,5-trifluorobenzamide

2-Amino-3,4,5-trifluorobenzoic acid (0.50 g; 2.62 mmol), DCM (10 ml), TEA (1.09 ml; 7.85 mmol), and (4-bromophenyl)methanamine (0.54 g; 2.88 mmol) were placed in a reaction flask under nitrogen. The mixture was cooled to 0° C. and T3P (1.87 ml; 3.14 mmol, 50% solution) was added slowly. The reaction mixture was stirred for 30 min at 0° C. and overnight at rt. The mixture was diluted with DCM, washed with water and brine, dried with a phase separator, and evaporated to dryness to give 0.80 g of 2-amino-N-(4-bromobenzyl)-3,4,5-trifluorobenzamide. ¹H-NMR (400 MHz, d₆-DMSO): δ 4.39 (d, 2H), 6.61 (s, 2H), 7.24-7.30 (m, 2H), 7.49-7.56 (m, 2H), 7.56-7.64 (m, 1H), 8.98 (t, 1H).

### 3-(4-Bromobenzyl)-6,7,8-trifluoroquinazoline-2,4(1H,3H)-dione

2-Amino-N-(4-bromobenzyl)-3,4,5-trifluorobenzamide (0.80 g; 2.23 mmol) and pyridine (5 ml) were placed in a reaction flask under nitrogen. The reaction mixture was cooled to 0° C. and ethyl chloroformate (0.64 ml; 6.68 mmol) was added dropwise. The reaction mixture was stirred at rt for 2 h and then cooled to 0° C. 2 M NaOH (4.46 ml; 8.91 mmol) was carefully added and stirring was continued overnight at P. The mixture was concentrated and the residue was diluted with DCM. Addition of 1 M HCl gave a precipitation, which was filtered, washed with water, and dried in a vacuum oven 40° C. to give 0.41 g of crude 3-(4-bromobenzyl)-6,7,8-trifluoroquinazoline-2,4(1H,3H)-dione. ¹H-NMR (400 MHz, d₆-DMSO): δ 5.04 (s, 2H), 7.26-7.32 (m, 2H), 7.48-7.54 (m, 2H), 7.78-7.85 (m, 1H), 12.07 (br s, 1H).

### 3-(4-Bromobenzyl)-6,7,8-trifluoro-1-(2-hydroxy-2-methylpropyl)quinazoline-2,4(1H,3H)-dione

3-(4-Bromobenzyl)-6,7,8-trifluoroquinazoline-2,4(1H,3H)-dione (0.41 g; 1.07 mmol) and yttrium(III) nitrate hexahydrate (41 mg; 0.11 mmol) were placed in a microwave reaction vial. DMF (1 ml) and isobutylene oxide (2.84 ml; 31.9 mmol) were added and the reaction mixture was heated in a microwave reactor at 120° C. for 60 min. After work-up, the crude product was purified with MS-Trigger to give 145 mg of 3-(4-bromobenzyl)-6,7,8-trifluoro-1-(2-hydroxy-2-methylpropyl)quinazoline-2,4(1H,3H)-dione. ¹H-NMR (400 MHz, CDCl₃): δ 1.25 (s, 6H), 2.43 (s, 1H), 4.46 (s, 2H), 5.20 (s, 2H), 7.33-7.39 (m, 2H), 7.40-7.45 (m, 2H), 7.91 (ddd, 1H).

### Example 61: 3-(4-Bromobenzyl)-6,7-difluoro-1-(2-hydroxy-2-methylpropyl)quinazoline-2,4(1H,3H)-dione

### 2-Amino-N-(4-bromobenzyl)-4,5-difluorobenzamide

2-Amino-4,5-difluorobenzoic acid (5.0 g; 28.9 mmol), 50 ml of DCM, TEA (12.1 ml; 87 mmol), and (4-bromophenyl)methanamine (4.0 ml; 31.8 mmol) were placed in a reaction flask under nitrogen and cooled to 0° C. T3P (34 ml; 57.8 mmol; 50% in EtOAc) was added slowly. The reaction mixture was stirred at rt for 3 h. DCM was added and the mixture was washed twice with water, dried with a phase separator, and evaporated to dryness to give 12.48 g of crude 2-amino-N-(4-bromobenzyl)-4,5-difluorobenzamide. The product was used in the next step without purification. ¹H-NMR (400 MHz, d₆-DMSO): δ 4.37 (d, 2H), 3.87 (br s, 2H), 6.66 (dd, 2H), 6.88 (d, 1H), 7.23-7.31 (m, 2H), 7.47-7.55 (m, 2H), 7.67 (dd, 1H), 8.85 (t, 1H).

### Ethyl (2((4-bromobenzyl)carbamoyl)-4,5-difluorophenyl)carbamate

2-Amino-N-(4-bromobenzyl)-4,5-difluorobenzamide (9.85 g; 28.9 mmol) was dissolved in 75 ml of pyridine under nitrogen. Ethyl chloroformate (8.28 ml; 87 mmol) was added slowly at 0° C. and the mixture was stirred for 2 h at rt. EtOAc (75 ml) and 1 M HCl (75 ml) were added and water phase was washed twice with EtOAc. Organic phases were combined, washed three times with 1 M HCl and three times with water, and dried with a phase separator funnel. Organic phase was evaporated to dryness, triturated in DCM, filtered, and dried to give 8.898 g of ethyl (2-((4-bromobenzyl)carbamoyl)-4,5-difluorophenyl)carbamate. ¹H-NMR (400 MHz, d₆-DMSO): δ 1.23 (t, 3H), 4.14 (q, 2H), 4.43 (d, 2H), 7.27-7.33 (m, 2H), 7.50-7.56 (m, 2H), 7.97 (dd, 1H), 8.21 (dd, 1H), 9.38 (t, 1H), 11.09 (br s, 1H).

### 3-(4-Bromobenzyl)-6,7-difluoroquinazoline-2,4(1H,3H)-dione

Ethyl (2-((4-bromobenzyl)carbamoyl)-4,5-difluorophenyl)carbamate (8.989 g; 21.5 mmol), EtOH (100 ml), and 2 M NaOH (21.5 ml; 43.1 mmol) were placed in a reaction flask and refluxed for 2 h. Water (10 ml) was added and pH was adjusted to 5.5 with 6 M HCl. The precipitation was filtered, washed twice with water, and dried to give 7.73 g of 3-(4-bromobenzyl)-6,7-difluoroquinazoline-2,4(1H,3H)-dione. ¹H-NMR (400 MHz, d₆-DMSO): δ 5.03 (s, 2H), 7.15 (dd, 1H), 7.25-7.31 (m, 2H), 7.47-7.53 (m, 2H), 7.91 (dd, 1H), 11.71 (br s, 1H).

### 3-(4-Bromobenzyl)-6,7-difluoro-1-(2-hydroxy-2-methylpropyl)quinazoline-2,4(1H,3H)-dione

3-(4-Bromobenzyl)-6,7-difluoroquinazoline-2,4(1H,3H)-dione (150 mg; 0.41 mmol), 1 ml of DMF, isobutylene oxide (0.073 ml; 0.82 mmol), and K₂CO₃ (85 mg; 0.61 mmol) were charged in a microwave reaction vial and heated at 120° C. for 1 h (absorption high). The reaction mixture was reheated at 130° C. for 1 h without completion of the reaction. Isobutylene oxide (0.07 ml; 0.81 mmol) was added and the reaction mixture was reheated at 140° C. for 1 h. The reaction mixture was evaporated. EtOAc was added and the mixture was washed with 1 M Na₂CO₃ and then twice with water. Organic phase was dried with a phase separator funnel, evaporated to dryness, and purified with CombiFlash (normal phase silica; EtOAc:heptane) to give 96 mg of 3-(4-bromobenzyl)-6,7-difluoro-1-(2-hydroxy-2-methylpropyl)quinazoline-2,4(1H,3H)-dione. ¹H-NMR (400 MHz, CDCl₃): δ 1.33 (s, 6H), 2.18 (s, 1H), 4.14 (br s, 2H), 5.20 (s, 2H), 7.34-7.47 (m, 5H), 8.00 (dd, 1H).

### Example 62: 3-(4-(Difluoromethoxy)benzyl)-6,7-difluoro-1-(2-hydroxy-2-methylpropyl)quinazoline-2,4(1H,3H)-dione

### 2-Amino-N-(4-(difluoromethoxy)benzyl)-4,5-difluorobenzamide

2-Amino-4,5-difluorobenzoic acid (0.85 g; 4.91 mmol), 10 ml of DCM, TEA (2.05 nil; 14.7 mmol), and (4-(difluoromethoxy)phenyl)methanamine (0.85 g; 4.91 mmol) were placed in a reaction flask under nitrogen and cooled to 0° C. T3P (3.74 ml; 5.89 mmol) was added slowly. The reaction mixture was stirred overnight at rt, then 4 h at 40° C., and finally heated by microwave at 100° C. for 5 min without completion of the reaction. The reaction mixture was diluted by EtOAc, washed twice with water, dried, and evaporated to dryness. Toluene was added and the mixture was evaporated again. The residue was purified twice with CombiFlash (first reverse phase silica and then normal phase silica). The product remained impure and was used in the next step without further purification.

### 3-(4-(Difluoromethoxy)benzyl)-6,7-difluoroquinazoline-2,4(1H,3H)-dione

2-Amino-N-(4-(difluoromethoxy)benzyl)-4,5-difluorobenzamide (354 mg; 1.08 mmol), 3 ml of dry pyridine, and ethyl chloroformate (0.31 nil; 3.24 mmol) were stirred at rt overnight. 2 M NaOH (2.5 ml; 5 mmol) was added and the mixture was stirred at 50° C. for 3 h. Work-up was done like for ethyl (2-((1-(4-chlorophenyl)cyclopropyl)carbamoyl)-5-fluorophenyl)carbamate in Example 43, but 46 mg of the product that precipitated in the phase separator was filtered. The precipitation was dried to give 3-(4-(difluoromethoxy)benzyl)-6,7-difluoroquinazoline-2,4(1H,3H)-dione. ¹H-NMR (400 MHz, d₆-DMSO): δ 5.05 (s, 2H), 7.09-7.18 (m, 3H), 7.18 (t, 1H), 7.35-7.41 (m, 2H), 7.91 (dd, 1H), 11.69 (br s, 1H).

### 3-(4-(Difluoromethoxy)benzyl)-6,7-difluoro-1-(2-hydroxy-2-methylpropyl)quinazoline-2,4(1H,3H)-dione

3-(4-(Difluoromethoxy)benzyl)-6,7-difluoroquinazoline-2,4(1H,3H)-dione (64 mg; 0.18 mmol), K₂CO₃ (37 mg; 0.27 mmol), yttrium(III) nitrate hexahydrate (6.9 mg; 0.018 mmol), isobutylene oxide (0.16 ml; 1.81 mmol), and 1 ml of dry DMF were placed in a microwave reaction vial and heated for 15 min at 150° C. (absorption high). Isobutylene oxide (1.6 ml; 18.1 mmol) was added and the same microwave program was repeated. The reaction mixture was cooled to rt and neutralized with 1 M HCl. 10 ml of water was added, the mixture was extracted twice with 10 ml of DCM, and combined organic phases were evaporated to dryness. The residue was dissolved in DCM, washed four times with water to remove DMF, and evaporated to dryness. CombiFlash (normal phase silica) purification gave 45.7 mg of 3-(4-(difluoromethoxy)benzyl)-6,7-difluoro-1-(2-hydroxy-2-methylpropyl)quinazoline-2,4(1H,3H)-dione. ¹H-NMR (400 MHz, CDCl₃): δ 1.33 (s, 6H), 2.18 (s, 1H), 4.14 (br s, 2H), 5.23 (s, 2H), 6.46 (t, 1H), 7.02-7.08 (m, 2H), 7.43 (dd, 1H), 7.48-7.54 (m, 2H), 8.00 (dd, 1H).

### Example 63: 3-((2,3-Dihydrobenzofuran-5-yl)methyl)-6,7-difluoro-1-methylquinazoline-2,4(1H,3H)-dione

### 2-Amino-N-((2,3-dihydrobenzofuran-5-yl)methyl)-4,5-dilluorobenzamide

2-Amino-4,5-difluorobenzoic acid (0.52 g; 3.02 mmol), 5 ml of DCM, TEA (1.26 ml; 9.05 mmol), and (2,3-dihydrobenzofuran-5-yl)methanamine (0.45 g; 3.02 mmol) were placed in a reaction flask under nitrogen and cooled to 0° C. T3P (2.13 ml; 3.62 mmol) was added slowly. The reaction mixture was stirred at rt for 3 days and then refluxed for 3 h without completion of the reaction. The reaction mixture was diluted with EtOAc, washed twice with water, dried, and evaporated to dryness. Toluene was added and the mixture was evaporated again to give 608 mg of 2-amino-N-((2,3-dihydrobenzofuran-5-yl)methyl)-4,5-difluorobenzamide. ¹H-NMR (400 MHz, CDCl₃): δ 3.20 (t, 2H), 4.48 (d, 2H), 4.58 (t, 2H), 5.56 (br s, 2H), 6.08 (br s, 1H), 6.45 (dd, 1H), 6.76 (d, 1H), 7.05-7.09 (m, 1H), 7.12 (dd, 1H), 7-16-7.21 (m, 1H).

### Ethyl (2-(((2,3-dihydrobenzofuran-5-yl)methyl)carbamoyl)-4,5-difluorophenyl)carbamate

2-Amino-N-((2,3-dihydrobenzofuran-5-yl)methyl)-4,5-difluorobenzamide (0.608 g; 2.00 mmol) was dissolved in 3 ml of dry pyridine and cooled to 0° C. Ethyl chloroformate (0.57 ml; 6.00 mmol) was added slowly. The reaction mixture was stirred at rt overnight, 10 ml of EtOAc was added, and pH was adjusted to acidic with 10 ml of 1 M HCl. The layers were separated and water phase was washed twice with EtOAc. The organic phases were combined, washed three times with 1 M HCl and three times with water, and dried with a phase separator. Evaporation to dryness gave 657 mg of ethyl (2-(((2,3-dihydrobenzofuran-5-yl)methyl)carbamoyl)-4,5-difluorophenyl)carbamate. ¹H-NMR (400 MHz, CDCl₃): δ 1.24 (t, 3H), 3.15 (t, 2H), 4.14 (q, 2H), 4.37 (d, 2H), 4.50 (t, 2H), 6.71 (d, 1H), 7.03-7.08 (m, 1H), 7.18-7.22 (m, 1H), 7.96 (dd, 1H), 8.21 (dd, 1H), 9.27 (t, 1H), 11.19 (br s, 1H).

### 3-((2,3-Dihydrobenzofuran-5-yl)methyl)-6,7-difluoroquinazoline-2,4(1H,3H)-dione

Ethyl (2-(((2,3-dihydrobenzofuran-5-yl)methyl)carbamoyl)-4,5-difluorophenyl)carbamate (657 mg; 1.75 mmol), 5 ml of THF, and 2 M NaOH (1.75 ml; 3.49 mmol) were placed in a reaction flask and stirred for 90 min at 50° C. to complete the reaction. 5 ml of water was added and pH was adjusted to neutral with 1 M HCl to give precipitation. Trituration with DCM, filtration, and drying gave 266 mg of 3-((2,3-dihydrobenzofuran-5-yl)methyl)-6,7-difluoroquinazoline-2,4(1H,3H)-dione. ¹H-NMR (400 MHz, d₆-DMSO): δ 3.12 (t, 2H), 4.47 (t, 2H), 4.97 (s, 2H), 6.67 (d, 1H), 7.06-7.10 (m, 1H), 7.13 (dd, 1H), 7.20-7.23 (m, 1H), 7.91 (dd, 1H), 11.63 (br s, 1H).

### 3-((2,3-Dihydrobenzofuran-5-yl)methyl)-6,7-difluoro-1-methylquinazoline-2,4(1H,3H)-dione

3-((2,3-Dihydrobenzofuran-5-yl)methyl)-6,7-difluoroquinazoline-2,4(1H,3H)-dione (75 mg; 0.23 mmol), K₂CO₃ (47 mg; 0.34 mmol), and 2 ml of dry DMF were placed in a reaction flask. Iodomethane (0.021 ml; 0.34 mmol) was added and the mixture was stirred for 2 at rt to complete the reaction. The reaction mixture was neutralized with 1 M HCl and 20 ml of water and EtOAc were added. Organic phase was washed four times with 5 ml of water, dried with a phase separator, and evaporated to dryness to give 74 mg of 3-((2,3-dihydrobenzofuran-5-yemethyl)-6,7-difluoro-1-methylquinazoline-2,4(1H,3H)-dione. ¹H-NMR (400 MHz, CDCl₃): δ 3.17 (t, 2H), 3.55 (s, 3H), 4.53 (t, 2H), 5.16 (s, 2H), 6.71 (d, 1H), 6.99 (dd, 1H), 7.29-7.33 (m, 1H), 7.37-7.41 (m, 1H), 8.03 (dd, 1H).

### Example 64: 3-(4-Bromobenzyl)-1-(but-3-yn-2-yl)-7-fluoroquinazoline-2,4(1H,3H)-dione

3-(4-Bromobenzyl)-7-fluoroquinazoline-2,4(1H,3H)-dione (100 mg; 0.29 mmol) prepared in Example 26, 1 ml of THF, TBAB (30 mg; 0.093 mmol), K₂CO₃ (119 mg; 0.86 mmol), and 3-bromobut-1-yne (0.08 ml; 0.86 mmol) were charged in a microwave reaction vial and heated at 150° C. for 90 min. TBAB (30 mg; 0.093 mmol), K₂CO₃ (119 mg; 0.86 mmol), and 3-bromobut-1-yne (0.08 ml; 0.86 mmol) were added again and the microwave reaction continued for 60 min at 150° C. K₂CO₃ (119 mg; 0.86 mmol) and 3-bromobut-1-yne (0.08 ml; 0.86 mmol) were added once more and the microwave reaction continued for 2 h at 150° C. Water and EtOAc were added and water phase was washed twice with EtOAc. Organic phases were combined, dried, and evaporated. The crude product was purified twice by CombiFlash (first EtOAc:heptane gradient; normal phase silica and then ACN:water gradient; reverse phase silica) to give 34 mg of 3-(4-bromobenzyl)-1-(but-3-yn-2-yl)-7-fluoroquinazoline-2,4(1H,3H)-dione. ¹H-NMR (400 MHz, CDCl₃): δ 1.71 (d, 3H), 2.54 (d, 1H), 5.18 (dd, 2H), 6.36 (dq, 1H), 6.98 (ddd, 1H), 7.36-7.48 (m, 4H), 7.61 (dd, 1H), 8.26 (dd, 1H).

### Example 65: 6,7,8-Trifluoro-1-(2-hydroxy-2-methylpropyl)-3-(4-methoxybenzyl)quinazoline-2,4(1H,3H)-dione

### 2-Amino-3,4,5-trifluoro-N-(4-methoxybenzyl)benzamide

2-Amino-3,4,5-trifluorobenzoic acid (0.5 g; 2.6 mmol), 7 ml of dry DCM, and TEA (1.1 ml; 7.9 mmol) were placed in a reaction flask under nitrogen. 4-Methoxybenzylamine (0.38 ml; 2.9 mmol) was added slowly and then T3P (1.9 ml; 3.1 mmol; 50% in EtOAc) was added keeping the temperature below 30° C. The mixture was stirred at rt for 3 h. DCM was added and the mixture was washed twice with water and once with saturated aqueous NaCl. The organic phase was dried with a phase separator and concentrated under reduced pressure to yield 0.79 g of 2-amino-3,4,5-trifluoro-N-(4-methoxybenzyl)benzamide. ¹H-NMR (400 MHz, d₆-DMSO): δ 3.73 (s, 3H), 4.35 (d, 2H), 6.55-6.65 (br s, 2H), 6.86-6.91 (m, 2H), 7.21-7.26 (m, 2H), 7.58 (ddd, 1H), 8.89 (t, 1H).

### 6,7,8-Trifluoro-3-(4-methoxybenzyl)quinazoline-2,4(1H,3H)-dione

2-Amino-3,4,5-trifluoro-N-(4-methoxybenzyl)benzamide (0.79 g; 2.5 mmol) and 5 ml of dry pyridine were placed in a reaction flask under nitrogen. Ethyl chloroformate (0.73 ml; 7.6 mmol) was added dropwise at 0° C. The mixture was stirred at rt overnight to complete carbamate formation. 2 M NaOH (5.1 ml; 10.2 mmol) was added dropwise and the mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 40° C. to yield 0.33 g of 6,7,8-trifluoro-3-(4-methoxybenzyl)quinazoline-2,4(1H,3H)-dione. ¹H-NMR (400 MHz, d₆-DMSO): δ 3.71 (s, 3H), 5.01 (s, 2H), 6.84-6.89 (m, 2H), 7.26-7.31 (m, 2H), 7.80 (ddd, 1H), 11.98-12.06 (br s, 1H).

### 6,7,8-Trifluoro-1-(2-hydroxy-2-methylpropyl)-3-(4-methoxybenzyl)quinazoline-2,4(1H,3H)-dione

6,7,8-Trifluoro-3-(4-methoxybenzyl)quinazoline-2,4(1H,3H)-dione (330 mg; 0.98 mmol), yttrium(III) nitrate hexahydrate (37 mg; 0.10 mmol), 2 ml of dry DMF, and isobutylene oxide (1.3 ml; 15 mmol) were charged in a microwave tube and heated at 120° C. for 1 h and at 160° C. for 0.5 h. An additional batch of isobutylene oxide (0.87 ml; 9.8 mmol) was added and the mixture was heated at 160° C. for 0.5 h. After cooling to rt, the mixture was diluted with DCM and washed with saturated aqueous NaHCO₃, water, and saturated aqueous NaCl. The organic phase was dried with a phase separator and concentrated under reduced pressure. The residue was purified with MS-Trigger to yield 140 mg of 6,7,8-trifluoro-1-(2-hydroxy-2-methylpropyl)-3-(4-methoxybenzyl)quinazoline-2,4(1H,3H)-dione. ¹H-NMR (400 MHz, CDCl₃): δ 1.25 (s, 6H), 2.40-3.00 (br s, 1H), 3.77 (s, 3H), 4.45 (s, 2H), 5.19 (s, 2H), 6.79-6.85 (m, 2H), 7.41-7.47 (m, 2H), 7.87-7.95 (m, 1H).

### Example 66: 6-(4-Bromobenzyl)-9,10-difluoro-2,2-dimethyl-2H-[1,4]oxazino[2,3,4-ij]quinazoline-5,7(3H,6H)-dione

The preparation of 3-(4-bromobenzyl)-6,7,8-trifluoro-1-(2-hydroxy-2-methylpropyl)quinazoline-2,4(1H,3H)-dione is described in Example 60. 3-(4-Bromobenzyl)-6,7,8-trifluoro-1-(2-hydroxy-2-methylpropyl)quinazoline-2,4(1H,3H)-dione (230 mg; 0.49 mmol), sodium hydride (39 mg; 0.98 mmol; 60% in oil), and 8 ml of dry THF were stirred under nitrogen at rt for 1 h after which the reaction was quenched with dropwise addition of MeOH. The mixture was diluted with water and extracted twice with DCM. The organic phase was dried with a phase separator and concentrated under reduced pressure. The residue was purified with CombiFlash (normal phase silica) to yield 130 mg of 6-(4-bromobenzyl)-9,10-difluoro-2,2-dimethyl-2H-[1,4]oxazino[2,3,4-ij]quinazoline-5,7(3H,6H)-dione. ¹H-NMR (400 MHz, CDCl₃): δ 1.46 (s, 6H), 3.88 (s, 2H), 5.18 (s, 2H), 7.38-7.46 (m, 4H), 7.55 (dd, 1H).

### Example 67: 10-Chloro-6-(4-chlorobenzyl)-2,2-dimethyl-2H-[1,4]oxazino[2,3,4-ij]quinazoline-5,7(3H,6H)-dione

### 2-Amino-4-chloro-N-(4-chlorobenzyl)-3-fluorobenzamide

2-Amino-4-chloro-3-fluorobenzoic acid (0.50 g; 2.6 mmol), 10 ml of dry DCM, and TEA (1.5 ml; 11 mmol) were placed in a reaction flask under nitrogen. 4-Chlorobenzylamine (0.39 ml; 12 mmol) was added slowly and then T3P (3.1 ml; 5.3 mmol; 50% in EtOAc) was added keeping the temperature below 30° C. The mixture was stirred at rt overnight. DCM was added and the mixture was washed twice with water. The organic phase was dried with a phase separator and concentrated under reduced pressure to yield 0.79 g of 2-amino-4-chloro-N-(4-chlorobenzyl)-3-fluorobenzamide. ¹H-NMR (400 MHz, d₆-DMSO): δ 4.41 (d, 2H), 6.69 (br s, 1H), 6.67-6.73 (m, 1H), 7.31-7.41 (m, 4H), 7.45 (dd, 1H), 9.02 (t, 1H).

### 7-Chloro-3-(4-chlorobenzyl)-8-fluoroquinazoline-2,4(1H,3H)-dione

2-Amino-4-chloro-N-(4-chlorobenzyl)-3-fluorobenzamide (0.79 g; 2.5 mmol) and 4 ml of dry pyridine were placed in a reaction flask under nitrogen. Ethyl chloroformate (0.72 ml; 7.6 mmol) was added dropwise at 0° C. The mixture was stirred at rt overnight to complete carbamate formation. The mixture was cooled to 0° C. and 2 M NaOH (3.8 ml; 7.6 mmol) was added dropwise. The mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 50° C. to yield 0.43 g of 7-chloro-3-(4-chlorobenzyl)-8-fluoroquinazoline-2,4(1H,3H)-dione. ¹H-NMR (400 MHz, d₆-DMSO): δ 5.06 (s, 2H), 7.34-7.40 (m, 5H), 7.77 (dd, 1H), 11.94 (s, 1H).

### 7-Chloro-3-(4-chlorobenzyl)-8-fluoro-1-(2-hydroxy-2-methylpropyl)quinazoline-2,4(1H,3H)-dione

7-Chloro-3-(4-chlorobenzyl)-8-fluoroquinazoline-2,4(1H,3H)-dione (200 mg; 0.59 mmol), yttrium(III) nitrate hexahydrate (23 mg; 0.059 mmol), 2 ml of dry DMF, and isobutylene oxide (1.57 ml; 17.7 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with CombiFlash (normal phase silica) to yield 130 mg of 7-chloro-3-(4-chlorobenzyl)-8-fluoro-1-(2-hydroxy-2-methylpropyl)quinazoline-2,4(1H,3H)-dione. ¹H-NMR (400 MHz, CDCl₃): δ 1.25 (s, 6H), 2.70 (s, 1H), 4.50 (d, 2H), 5.22 (s, 2H), 7.25-7.31 (m, 3H), 7.41-7.45 (m, 2H), 8.01 (dd, 1H).

### 10-Chloro-6-(4-chlorobenzyl)-2,2-dimethyl-2H-[1,4]oxazino[2,3,4-ij]quinazoline-5,7(3H,6H)-dione

7-Chloro-3-(4-chlorobenzyl)-8-fluoro-1-(2-hydroxy-2-methylpropyl)quinazoline-2,4(1H,3H)-dione (120 mg; 0.29 mmol), sodium hydride (23 mg; 0.58 mmol; 60% in oil), and 2 ml of dry THF were stirred under nitrogen at rt for 2 h after which the reaction was quenched with dropwise addition of water. The mixture was diluted with DCM and washed twice with water. The organic phase was dried with a phase separator and concentrated under reduced pressure. The residue was purified with CombiFlash (normal phase silica) to yield 85 mg of 10-chloro-6-(4-chlorobenzyl)-2,2-dimethyl-2H-[1,4]oxazino[2,3,4-ij]quinazoline-5,7(3H,6H)-dione. ¹H-NMR (400 MHz, CDCl₃): δ 1.44 (s, 6H), 3.89 (s, 2H), 5.19 (s, 2H), 7.21 (d, 1H), 7.24-7.29 (m, 2H), 7.44-7.49 (m, 2H), 7.67 (d, 1H).

### Example 68: (R)-3-(4-Bromobenzyl)-7-chloro-1-(2-hydroxypropyl)quinazoline-2,4(1H,3H)-dione

The preparation of 3-(4-bromobenzyl)-7-chloroquinazoline-2,4(1H,3H)-dione is described in Example 18. 3-(4-Bromobenzyl)-7-chloroquinazoline-2,4(1H,3H)-dione (300 mg; 0.74 mmol), yttrium(III) nitrate hexahydrate (28 mg; 0.072 mmol), 3 ml of dry DMF, and (R)-2-methyloxirane (0.52 ml; 7.4 mmol) were charged in a microwave tube and heated at 160° C. for 1 h. After cooling to rt, the mixture was diluted with DCM and washed with saturated NaHCO₃, water, and saturated aqueous NaCl. The organic phase was dried with a phase separator and concentrated under reduced pressure to yield 220 mg of (R)-3-(4-bromobenzyl)-7-chloro-1-(2-hydroxypropyl)quinazoline-2,4(1H,3H)-dione. ¹H-NMR (400 MHz, CDCl₃): δ 1.35 (d, 3H), 2.15-2.45 (br s, 1H), 4.02-4.02 (dd, 1H), 4.10-4.18 (dd, 1H), 4.19-4.29 (m, 1H), 5.17 (s, 2H), 7.21 (dd, 1H), 7.32-7.44 (m, 5H), 8.13 (d, 1H).

### Example 69: 7-Chloro-1-((3-methyloxetan-3-yl)methyl)-3-(4-(trifluoromethoxy)benzyl)quinazoline-2,4(1H,3H)-dione

### 2-Amino-4-chloro-N-(4-(trifluoromethoxy)benzyl)benzamide

2-Amino-4-chlorobenzoic acid (0.50 g; 2.9 mmol), 15 ml of dry DCM, and TEA (1.6 ml; 12 mmol) were placed in a reaction flask under nitrogen. 4-(Trifluoromethoxy)benzylamine (0.67 ml; 4.4 mmol) was added slowly and then T3P (3.4 ml; 5.8 mmol; 50% in EtOAc) was added keeping the temperature below 30° C. The mixture was stirred at rt overnight. DCM was added and the mixture was washed twice with water. The organic phase was dried with a phase separator and concentrated under reduced pressure to yield 1.2 g of crude product. ¹H-NMR (400 MHz, d₆-DMSO): δ 4.44 (d, 2H), 6.54 (dd, 1H), 6.60-6.90 (br s, 2H), 6.77 (d, 1H), 7.30-7.34 (m, 2H), 7.41-7.44 (m, 2H), 7.58 (d, 1H), 8.91 (t, 1H).

### 7-Chloro-3-(4-(trifluoromethoxy)benzyl)quinazoline-2,4(1H,3H)-dione

2-Amino-4-chloro-N-(4-(trifluoromethoxy)benzyl)benzamide (1.0 g; 2.9 mmol) and 5 ml of dry pyridine were placed in a reaction flask under nitrogen. Ethyl chloroformate (0.83 ml; 8.8 mmol) was added dropwise at 0° C. The mixture was stirred at rt overnight to complete carbamate formation. 2 M NaOH (4.4 ml; 8.8 mmol) was added dropwise and the mixture was stirred at rt for 2 h. An additional batch of 2 M NaOH (1.4 ml; 2.8 mmol) was added and the mixture was heated at 50° C. for 1 h. The mixture was partially concentrated and the residue was diluted with DCM. Water and 1 M HCl were added till pH<4 and the precipitation formed was filtered, washed with water, and dried under reduced pressure at 50° C. to yield 0.78 g of 7-chloro-3-(4-(trifluoromethoxy)benzyl)quinazoline-2,4(1H,3H)-dione. ¹H-NMR (400 MHz, d₆-DMSO): δ 5.09 (s, 2H), 7.21-7.23 (m, 1H), 7.27 (dd, 1H), 7.29-7.33 (m, 2H), 7.43-7.48 (m, 2H), 7.94 (d, 1H), 11.50-11.80 (br s, 1H).

### 7-Chloro-1-((3-methyloxetan-3-yl)methyl)-3-(4-(trifluoromethoxy)benzyl)quinazoline-2,4(1H,3H)-dione

7-Chloro-3-(4-(trifluoromethoxy)benzyl)quinazoline-2,4(1H,3H)-dione (150 mg; 0.41 mmol), sodium hydride (32 mg; 0.81 mmol; 60% in oil), and 2 ml of dry ACN were charged in a microwave tube and stirred under nitrogen at rt for 30 min. 3-(Chloromethyl)-3-methyloxetane (0.18 ml; 1.6 mmol) was added and the mixture was heated at 160° C. for 1 h. An additional batch of 3-(chloromethyl)-3-methyloxetane (0.09 ml; 0.8 mmol) was added and the mixture was heated again at 160° C. for 1 h to complete the reaction. The reaction was quenched with addition of MeOH. The mixture was concentrated under reduced pressure. The residue was diluted with DCM and washed twice with water and then with saturated aqueous NaCl. The organic phase was dried with a phase separator, concentrated under reduced pressure, and purified with MS-Trigger to yield 52 mg of 7-chloro-1-((3-methyloxetan-3-yl)methyl)-3-(4-(trifluoromethoxy)benzyl)quinazol ine-2,4(1H,3H)-dione. ¹H-NMR (400 MHz, CDCl₃): δ 1.48 (s, 3H), 4.15 (s, 2H), 4.26 (d, 2H), 4.67 (d, 2H), 5.25 (s, 2H), 7.00 (d, 1H), 7.12-7.17 (m, 2H), 7.22-7.26 (dd, 1H), 7.51-7.57 (m, 2H), 8.20 (d, 1H).

### Example 70: 2-(3-(4-Bromobenzyl)-7-chloro-6-fluoro-2,4-dioxo-3,4-dihydroquinazolin-1(2H)-yl)-N-methylpropanamide

### Methyl 2-(3-(4-bromobenzyl)-7-chloro-6-fluoro-2,4-dioxo-3,4-dihydroquinazolin-1(2H)-yl)propanoate

3-(4-Bromobenzyl)-7-chloro-6-fluoroquinazoline-2,4(1H,3H)-dione (1.5 g; 3.9 mmol) prepared in Example 48, K₂CO₃ (1.6 g; 12 mmol), and 10 ml of dry DMF were charged in a microwave tube and stirred at rt for 30 min. Methyl 2-bromopropionate (2.6 g; 16 mmol) was added and the mixture was heated at 120° C. for 30 min. The mixture was concentrated under reduced pressure and the residue was diluted with water. The mixture was extracted twice with DCM and the combined organic phase was washed with water and with saturated aqueous NaCl. The organic phase was dried with a phase separator, concentrated under reduced pressure, and purified with CombiFlash (normal phase silica) to yield 1.2 g of methyl 2-(3-(4-bromobenzyl)-7-chloro-6-fluoro-2,4-dioxo-3,4-dihydroquinazolin-1(2H)-yl)propanoate. ¹H-NMR (400 MHz, CDCl₃): δ 1.71 (d, 3H), 3.69 (s, 3H), 5.17 (d, 2H), 5.19-5.32 (br s, 1H), 7.12 (d, 1H), 7.35-7.39 (m, 2H), 7.41-7.45 (m, 2H), 8.00 (d, 2H).

### 2-(3-(4-Bromobenzyl)-7-chloro-6-fluoro-2,4-dioxo-3,4-dihydroquinazolin-1(2H)-yl)propanoic acid

Methyl 2-(3-(4-bromobenzyl)-7-chloro-6-fluoro-2,4-dioxo-3,4-dihydroquinazolin-1(2H)-yl)propanoate (250 mg; 0.53 mmol), 1.5 ml of ACN, 0.5 ml of MeOH, and 2 M NaOH (0.53 ml; 1.06 mmol) were placed in a reaction flask and stirred at rt for 1 h. The mixture was partially concentrated under reduced pressure to form a precipitation. The mixture was filtered and the precipitation was washed with water, n-heptane, and diethyl ether. The precipitation was purified with CombiFlash (reverse phase silica) to yield 70 mg of 2-(3-(4-bromobenzyl)-7-chloro-6-fluoro-2,4-dioxo-3,4-dihydroquinazolin-1(2H)-yl)propanoic acid. LC-MS (ES) [M−H]⁻: 452.9.

### 2-(3-(4-Bromobenzyl)-7-chloro-6-fluoro-2,4-dioxo-3,4-dihydroquinazolin-1(2H)-yl)-N-methylpropanamide

2-(3-(4-Bromobenzyl)-7-chloro-6-fluoro-2,4-dioxo-3,4-dihydroquinazolin-1(2H)-yl)propanoic acid (65 mg; 0.14 mmol), 3 ml of dry DCM, and TEA (0.060 ml; 0.43 mmol) were placed in a reaction flask under nitrogen. Methylamine (0.078 ml; 0.16 mmol, 2 M solution in THF) was added slowly and then T3P (0.10 ml; 0.17 mmol; 50% in EtOAc) was added keeping the temperature below 30° C. The mixture was stirred at rt overnight. DCM was added and the mixture was washed twice with water and once with saturated aqueous NaCl. The organic phase was dried with a phase separator, concentrated under reduced pressure, and purified with MS-Trigger to yield 4 mg of 2-(3-(4-bromobenzyl)-7-chloro-6-fluoro-2,4-dioxo-3,4-dihydroquinazolin-1(2H)-yl)-N-methylpropanamide. ¹H-NMR (400 MHz, CDCl₃): δ 1.65 (d, 3H), 2.83 (d, 3H), 5.18 (m, 2H), 5.57-5.74 (br s, 1H), 5.83-5.87 (br s, 1H), 7.37-7.42 (m, 3H), 7.42-7.47 (m, 2H), 7.97 (d, 1H).

### Example 71: 6,8-Difluoro-1-(2-hydroxy-2-methylpropyl)-3-(4-(trifluoromethoxy)benzyl)quinazoline-2,4(1H,3H)-dione

### 2-Amino-3,5-difluoro-N-(4-(trifluoromethoxy)benzyl)benzamide

2-Amino-3,5-difluorobenzoic acid (500 mg; 2.89 mmol), 10 ml of dry DCM, and TEA (1.61 ml; 11.55 mmol) were placed in a reaction flask under nitrogen. 4-(Trifluoromethoxy)benzylamine (0.529 ml; 3.47 mmol) was added slowly and then T3P (3.4 ml; 5.78 mmol; 50% in EtOAc) was added keeping the temperature at rt. The mixture was stirred at rt overnight. The reaction mixture was diluted with DCM and washed three times with water. The organic phase was dried with a phase separator and evaporated to dryness to yield 1.09 g of 2-amino-3,5-difluoro-N-(4-(trifluoromethoxy)benzyl)benzamide. ¹H-NMR (400 MHz, d₆-DMSO): δ 3.33 (s, 6H), 4.45 (d, 2H), 6.24 (s, 2H), 7.22-7.40 (m, 4H), 7.41-7.51 (m, 2H), 9.02 (t, 1H).

### 6,8-Difluoro-3-(4-(trifluoromethoxy)benzyl)quinazoline-2,4(1H,3H)-dione

2-Amino-3,5-difluoro-N-(4-(trifluoromethoxy)benzyl)benzamide (1.0 g; 2.89 mmol), 15 ml of dry THF, and 5 ml of dry pyridine were placed in a reaction flask under nitrogen. Ethyl chloroformate (0.825 ml; 8.66 mmol) was added dropwise at 0° C. The mixture was stirred at rt for 2 h to complete carbamate formation. 2 M NaOH (4.33 ml; 8.66 mmol) was added dropwise and the mixture was stirred at rt overnight. The mixture was evaporated almost to dryness and the residue was diluted with DCM. Water and 1 M HCl were added till pH was acidic and the precipitation formed was filtered, washed with water, and dried in a vacuum oven to yield 0.6 g of 6,8-difluoro-3-(4-(trifluoromethoxy)benzyl)quinazoline-2,4(1H,3H)-dione. ¹H-NMR (400 MHz, d₆-DMSO): δ 5.11 (s, 2H), 7.28-7.35 (m, 2H), 7.43-7.50 (m, 2H), 7.53-7.58 (m, 1H), 7.73-7.83 (m, 1H), 11.80 (br s, 1H).

### 6,8-Difluoro-1-(2-hydroxy-2-methylpropyl)-3-(4-(trifluoromethoxy)benzyl)quinazoline-2,4(1H,3H)-dione

6,8-Difluoro-3-(4-(trifluoromethoxy)benzyl)quinazoline-2,4(1H,3H)-dione (200 mg; 0.537 mmol), yttrium(III) nitrate hexahydrate (20.58 mg; 0.054 mmol), 1 ml of dry DMF, and isobutylene oxide (1.431 ml; 16.12 mmol) were charged in a microwave tube and heated at 160° C. for 1 h. After cooling to rt, the mixture was diluted with DCM and washed four times with water. The organic phase was dried with a phase separator and concentrated under reduced pressure. The residue was purified with flash chromatography to yield 64 mg of 6,8-difluoro-1-(2-hydroxy-2-methylpropyl)-3-(4-(trifluoromethoxy)benzyl)quinazoline-2,4(1H,3H)-dione. ¹H-NMR (400 MHz, CDCl₃): δ 1.24 (s, 6H), 2.72 (s, 1H), 4.48 (s, 2H), 5.26 (s, 2H), 7.11-7.23 (m, 3H), 7.50-7.57 (m, 2H), 7.77-7.82 (m, 1H).

### Example 72: 3-(4-Bromobenzyl)-6,7-difluoro-1-(3-oxobutyl)quinazoline-2,4(1H,3H)-dione

### Ethyl 3-(3-(4-bromobenzyl)-6,7-difluoro-2,4-dioxo-3,4-dihydroquinazolin-1(2H)-yl)propanoate

The preparation of 3-(4-bromobenzyl)-6,7-difluoroquinazoline-2,4(1H,3H)-dione is described in Examples 11 and 61. 3-(4-Bromobenzyl)-6,7-difluoroquinazoline-2,4(1H,3H)-dione (2.0 g; 5.45 mmol), K₂CO₃ (2.26 g; 16.34 mmol), and 25 ml of dry DMF were charged in a microwave reaction vessel and ethyl 3-bromopropionate (1.40 ml; 10.89 mmol) was added. The microwave reaction (absorption high) was not complete in 15 min at 150° C. Ethyl 3-bromopropionate (0.70 ml; 5.44 mmol) and TBAB (0.35 g; 1.09 mmol) were added and the microwave reaction was continued at 150° C. for 30 min. EtOAc (100 ml) was added and the mixture was washed four times with 150 ml of water. Organic phase was dried by filtration through a phase separator funnel and evaporated to dryness. DCM was added, the precipitation was filtered off, and the mother liquor was purified with column chromatography (normal phase silica; EtOAc:heptane gradient) to yield 0.66 mg of ethyl 3-(3-(4-bromobenzyl)-6,7-difluoro-2,4-dioxo-3,4-dihydroquinazolin-1(2H)-yl)propanoate. ¹H-NMR (400 MHz, d₆-DMSO): δ 1.13 (t, 3H), 2.63-2.70 (m, 2H), 4.01 (q, 2H), 4.28-4.36 (m, 1H), 5.07 (s, 2H), 7.26-7.32 (m, 2H), 7.47-7.53 (m, 2H), 7.85 (dd, 1H), 8.02 (dd, 1H).

### 3-(3-(4-Bromobenzyl)-6,7-difluoro-2,4-dioxo-3,4-dihydroquinazolin-1(2H)-yl)propanoic acid

Ethyl 3-(3-(4-bromobenzyl)-6,7-difluoro-2,4-dioxo-3,4-dihydroquinazolin-1(2H)-yl)propanoate (0.66 g; 1.41 mmol) was dissolved in 20 ml of THF, 1 M LiOH (2.82 ml; 2.82 mmol) was added, and the mixture was stirred at rt for 1 h. The reaction mixture was neutralized with 1 M HCl and evaporated to dryness. 15 ml of EtOAc was added and the mixture was washed twice with 5 ml of water. Organic phase was dried by filtration through a phase separator funnel and evaporated to dryness to obtain 548 mg of 3-(3-(4-bromobenzyl)-6,7-difluoro-2,4-dioxo-3,4-dihydroquinazolin-1(2H)-yl)propanoic acid. ¹H-NMR (400 MHz, d₆-DMSO): δ 2.57-2.64 (m, 2H), 4.24-4.32 (m, 2H), 5.07 (s, 2H), 7.25-7.32 (m, 2H), 7.47-7.53 (m, 2H), 7.84 (dd, 1H), 8.01 (dd, 1H), 12.43 (br s, 1H).

### 3-(3-(4-Bromobenzyl)-6,7-difluoro-2,4-dioxo-3,4-dihydroquinazolin-1(2H)-yl)-N-methoxy-N-methylpropanamide

3-(3-(4-Bromobenzyl)-6,7-difluoro-2,4-dioxo-3,4-dihydroquinazolin-1(2H)-yl)propanoic acid (0.49 g; 1.12 mmol) was dissolved in 5 ml of dry DCM under nitrogen. DIPEA (0.58 ml; 3.35 mmol), EDCI (0.32 g; 191.70 mmol), HBTU (0.64 g; 1.67 mmol), and N,O-dimethylhydroxylamine hydrochloride (0.11 g; 1.12 mmol) were added in this order and the mixture was stirred at rt overnight. The reaction mixture was washed with 10 ml of 1 M NaHCO₃ and 10 ml of water. Organic phase was dried by filtration through a phase separation funnel and evaporated to dryness. Column chromatography (normal phase silica; EtOAc:heptane gradient) purification gave 367 mg of 3-(3-(4-bromobenzyl)-6,7-difluoro-2,4-dioxo-3,4-dihydroquinazolin-1(2H)-yl)-N-methoxy-N-methylpropanamide. ¹H-NMR (400 MHz, d₆-DMSO): δ 2.71-2.81 (m, 2H), 3.03 (s, 3H), 3.59 (s, 3H), 4.25-4.34 (m, 2H), 5.07 (s, 2H), 7.26-7.33 (m, 2H), 7.47-7.53 (m, 2H), 7.80 (dd, 1H), 8.19 (dd, 1H).

### 3-(4-Bromobenzyl)-6,7-difluoro-1-(3-oxobutyl)quinazoline-2,4(1H,3H)-dione

3-(3-(4-Bromobenzyl)-6,7-difluoro-2,4-dioxo-3,4-dihydroquinazolin-1(2H)-yl)-N-methoxy-N-methylpropanamide (100 mg; 0.207 mmol) and 5 ml of dry THF were charged in a reaction flask under nitrogen and cooled to −78° C. Cyclopropylmagnesium bromide (0.5 M; 0.85 ml; 0.425 mmol) was added and the mixture was stirred overnight at rt. Methylmagnesium bromide (3 M; 0.14 ml; 0.415 mmol) was added and the mixture was stirred at 0° C. for 2 h. 1 ml of water and 1 ml of 1 M HCl were added dropwise and the mixture was evaporated to dryness. EtOAc was added and the mixture was washed with water, dried by filtration through a phase separator funnel, and evaporated to dryness. DCM was added, the precipitation was filtered off, and the mother liquor was purified with column chromatography (normal phase silica; EtOAc:heptane gradient). Final purification by crystallization from EtOAc gave 44 mg of 3-(4-bromobenzyl)-6,7-difluoro-1-(3-oxobutyl)quinazoline-2,4(1H,3H)-dione. ¹H-NMR (400 MHz, d₆-DMSO): δ 2.13 (s, 3H), 2.79-2.87 (m, 2H), 4.19-4.27 (m, 2H), 5.07 (s, 2H), 7.24-7.32 (m, 2H), 7.47-7.53 (m, 2H), 7.79 (dd, 1H), 8.02 (dd, 1H).

### Example 73: 7-Chloro-3-(4-(dimethylamino)benzyl)-1-(2-hydroxy-2-methylpropyl)quinazoline-2,4(1H,3H)-dione

### 2-Amino-4-chloro-N-(4-(dimethylamino)benzyl)benzamide

2-Amino-4-chlorobenzoic acid (1.0 g; 5.83 mmol), 15 ml of dry DCM, and TEA (4.87 ml; 35.0 mmol) were placed in a reaction flask under nitrogen. 4-(Dimethylamino)benzylamine dihydrochloride (1.561 g; 6.99 mmol) was added slowly and then T3P (6.87 ml; 11.66 mmol; 50% in EtOAc) was added keeping the temperature at rt. The mixture was stirred at rt for 2 h. The reaction mixture was diluted with DCM and washed three times with water. The organic phase was dried with a phase separator and evaporated to dryness to yield 1.49 g of 2-amino-4-chloro-N-(4-(dimethylamino)benzyl)benzamide. ¹H-NMR (400 MHz, d₆-DMSO): δ 2.85 (s, 6H), 4.28 (d, 2H), 6.51 (dd, 1H), 6.62-6.70 (m, 4H), 6.75 (d, 1H), 7.07-7.17 (m, 2H), 7.52 (d, 1H), 8.70 (t, 1H).

### 7-Chloro-3-(4-(dimethylamino)benzyl)quinazoline-2,4(1H,3H)-dione

2-Amino-4-chloro-N-(4-(dimethylamino)benzyl)benzamide (1.49 g; 4.90 mmol) and 4 ml of dry pyridine were placed in a reaction flask under nitrogen. Ethyl chloroformate (1.401 ml; 14.71 mmol) was added dropwise at 0° C. The mixture was stirred at rt overnight to complete carbamate formation. 2 M NaOH (7.36 ml; 14.71 mmol) was added dropwise and the mixture was heated and stirred at 50° C. for 2.5 h. After cooling, the mixture was evaporated to dryness and the residue was diluted with DCM. Water and 1 M HCl were added till pH was acidic and the precipitation formed was filtered, washed with water, and dried in a vacuum oven to yield 1.339 g of 7-chloro-3-(4-(dimethylamino)benzyl)quinazoline-2,4(1H,3H)-dione. ¹H-NMR (400 MHz, d₆-DMSO): δ 2.85 (s, 6H), 4.95 (s, 2H), 6.68 (br d, 2H), 7.18-7.23 (m, 3H), 7.25 (dd, 1H), 7.93 (d, 1H), 11.60 (s, 1H).

### 7-Chloro-3-(4-(dimethylamino)benzyl)-1-(2-hydroxy-2-methylpropyl)quinazoline-2,4(1H,3H)-dione

7-Chloro-3-(4-(dimethylamino)benzyl)quinazoline-2,4(1H,3H)-dione (150 mg; 0.455 mmol), yttrium(III) nitrate hexahydrate (17.42 mg; 0.045 mmol), 1 ml of dry DMF, and isobutylene oxide (1.212 ml; 13.65 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography and MS-Trigger to yield 115 mg of 7-chloro-3-(4-(dimethylamino)benzyl)-1-(2-hydroxy-2-methylpropyl)quinazoline-2,4(1H,3H)-dione. ¹H-NMR (400 MHz, CDCl₃): δ 1.33 (s, 6H), 2.90 (s, 6H), 4.17 (s, 2H), 5.17 (s, 2H), 6.59-6.69 (m, 2H), 7.19 (dd, 1H), 7.39-7.47 (m, 3H), 8.15 (d, 1H).

### Example 74: 6-Chloro-8-fluoro-1-(2-hydroxy-2-methylpropyl)-3-(4-methoxybenzyl)quinazoline-2,4(1H,3H)-dione

### 2-Amino-5-chloro-3-fluorobenzoic acid

2-Amino-3-fluorobenzoic acid (5.0 g, 32.2 mmol) and 25 ml of dry DMF were placed in a reaction flask. N-Chlorosuccinimide (5.60 g, 41.9 mmol) was added and the reaction mixture was stirred overnight at rt under nitrogen. DCM (30 ml) was added and the mixture was washed twice with 50 ml of water. During the second wash, a precipitation was formed. The precipitation was filtered, washed twice with 20 ml of water, and dried to give 2.64 g of 2-amino-5-chloro-3-fluorobenzoic acid. A second precipitation was formed in the mother liquor. 50 ml of DCM was added to the mother liquor to dissolve the precipitation and phases were separated. Organic phase was washed with 50 ml of water, dried by filtration through a phase separation funnel, and evaporated to dryness to give additionally 3.78 g of 2-amino-5-chloro-3-fluoro-benzoic acid. ¹H-NMR (400 MHz, d₆-DMSO): δ 3.31 (br s, 3H), 7.45 (dd, 1H), 7.51 (dd, 1H).

### 2-Amino-5-chloro-3-fluoro-N-(4-methoxybenzyl)benzamide

2-Amino-5-chloro-3-fluorobenzoic acid (1.0 g; 5.28 mmol), 10 ml of DCM, TEA (2.94 ml; 21.10 mmol), and 4-methoxybenzylamine (0.83 ml; 6.33 mmol) were placed in a reaction flask under nitrogen. T3P (6.22 ml; 10.55 mmol; 50% in EtOAc) was added slowly and the reaction mixture was stirred at rt for 3 days. 15 ml of DCM was added and the mixture was washed three times with 20 ml of water. The organic phase was dried and evaporated to dryness to obtain 1.45 g of 2-amino-5-chloro-3-fluoro-N-(4-methoxybenzyl)benzamide. 1H-NMR (400 MHz, d₆-DMSO): δ 3.73 (s, 3H), 4.34 (d, 2H), 6.50 (br s, 2H), 6.86-6.92 (m, 2H), 7.21-7.26 (m, 2H), 7.35 (dd, 1H), 7.51 (dd, 1H), 8.96 (t, 1H).

### 6-Chloro-8-fluoro-3-(4-methoxybenzyl)quinazoline-2,4(1H,3H)-dione

2-Amino-5-chloro-3-fluoro-N-(4-methoxybenzyl)benzamide (1.45 g; 4.70 mmol) and 7 ml of dry pyridine were placed in a reaction flask under nitrogen and cooled to 0° C. Ethyl chloroformate (1.34 ml, 14.09 mmol) was slowly added and the mixture was stirred for 2 h at rt. The mixture was cooled to 0° C., 2 M NaOH (7.04 ml; 14.09 mmol) was carefully added, and the mixture was stirred at rt overnight. The reaction mixture was evaporated close to dryness, 15 ml of DCM and 20 ml of water were added, and pH was adjusted to acidic with 1 M HCl. The precipitation formed was filtered, washed with water, and dried to obtain 1.32 g of 6-chloro-8-fluoro-3-(4-methoxybenzyl)quinazoline-2,4(1H,3H)-dione. ¹H-NMR (400 MHz, d₆-DMSO): δ 3.71 (s, 3H), 5.01 (s, 2H), 6.83-6.89 (m, 2H), 7.26-7.32 (m, 2H), 7.2-7.75 (m, 1H), 7.84 (dd, 1H), 11.83 (s, 1H).

### 6-Chloro-8-fluoro-1-(2-hydroxy-2-methylpropyl)-3-(4-methoxybenzyl)quinazoline-2,4(1H,3H)-dione

6-Chloro-8-fluoro-3-(4-methoxybenzyl)quinazoline-2,4(1H,3H)-dione (200 mg, 0.597 mmol), yttrium(III) nitrate hexahydrate (22.88 mg, 0.060 mmol), and 2 ml of dry DMF were placed in a microwave reaction vial. Isobutylene oxide (1.59 ml, 17.92 mmol) was added and the mixture was stirred (absorption high) at 160° C. for 30 min. 15 ml of DCM was added and the mixture was washed three times with 25 ml of water. Organic phase was dried and evaporated to dryness. The crude material was purified by column chromatography (normal phase silica; EtOAc:heptane gradient) to obtain 78 mg of 6-chloro-8-fluoro-1-(2-hydroxy-2-methylpropyl)-3-(4-methoxybenzyl)quinazoline-2,4(1H,3H)-dione. ¹H-NMR (400 MHz, CDCl₃): δ 1.23 (s, 6H), 2.82 (br s, 1H), 3.77 (s, 3H), 4.46 (s, 2H), 5.20 (s, 2H), 6.80-6.85 (m, 2H), 7.36 (dd, 1H), 7.42-7.47 (m, 2H), 8.06 (dd, 1H).

### Example 75: 34(2,3-Dihydrobenzofuran-5-yl)methyl)-6,7-difluoro-1-(2-hydroxy-2-methylpropyl)quinazoline-2,4(1H,3H)-dione

The preparation of 3-((2,3-dihydrobenzofuran-5-yl)methyl)-6,7-difluoroquinazoline-2,4(1H,3H)-dione is described in Example 63. 3-((2,3-Dihydrobenzofuran-5-yl)methyl)-6,7-difluoroquinazoline-2,4(1H,3H)-dione (100 mg; 0.30 mmol), yttrium(III) nitrate hexahydrate (12 mg; 0.03 mmol), 3 ml of dry DMF, K₂CO₃ (63 mg; 0.45 mmol), and isobutylene oxide (0.27 ml; 3.03 mmol) were placed in a microwave reaction vial and the mixture was heated (absorption high) at 125° C. for 15 min. More isobutylene oxide was added and the reaction was continued at 150° C. for 40 min. The reaction mixture was neutralized with 1 M HCl. 20 ml of water was added and the mixture was washed twice with 20 ml of EtOAc. Organic phases were combined, washed four times with water, dried by filtration through a phase separator funnel, and evaporated to dryness. The crude product was crystallized from toluene to obtain 111 mg of 3-((2,3-dihydrobenzofuran-5-yl)methyl)-6,7-difluoro-1-(2-hydroxy-2-methylpropyl)quinazoline-2,4(1H,3H)-dione. ¹H-NMR (400 MHz, CDCl₃): δ 1.16 (s, 6H), 3.11 (t, 2H), 4.10 (br s, 2H), 4.47 (t, 2H), 4.68 (s, 1H), 5.05 (s, 2H), 6.66 (d, 1H), 7.09 (dd, 1H), 7.23 (d, 1H), 7.89 (dd, 1H), 7.96 (dd, 1H).

### Example 76: (R)-3-(1-(4-Chlorophenyl)ethyl)-1-methylquinazoline-2,4(1H,3H)-dione

### (R)-2-Amino-N-(1-(4-chlorophenyl)ethyl)benzamide

Anthranilic acid (2.5 g; 18.23 mmol), DCM (15 ml), and TEA (10.16 ml; 72.9 mmol) were placed in a reaction vessel under nitrogen. (R)-1-(4-Chlorophenyl)ethylamine (3.07 ml; 21.88 mmol) and T3P (21.48 ml; 36.5 mmol; 50% in EtOAc) were added in this order slowly keeping the temperature stable with ice bath. The mixture was stirred overnight at rt. DCM was added and the mixture was washed three times with 40 ml of water. The organic layer was dried and evaporated to dryness to obtain 2.739 g of (R)-2-amino-N-(1-(4-chlorophenyl)ethyl)benzamide. ¹H-NMR (400 MHz, CDCl₃): δ 1.56 (d, 3H), 5.23 (quint, 1H), 5.51 (br s, 2H), 6.21 (br d, 1H), 6.61-6.70 (m, 2H), 7.17-7.24 (m, 1H), 7.27-7.35 (m, 5H).

### (R)-3-(1-(4-Chlorophenyl)ethyl)quinazoline-2,4(1H,3H)-dione

(R)-2-Amino-N-(1-(4-chlorophenyl)ethyl)benzamide (2.7 g; 9.83 mmol) was dissolved in 15 ml of dry pyridine under nitrogen and cooled to 0° C. Ethyl chloroformate (2.81 ml; 29.50 mmol) was added slowly and the mixture was stirred overnight at rt. The solution was cooled to 0° C. and 2 M NaOH (14.7 ml; 29.50 mmol) was added slowly. The solution was stirred for 3 h at 50° C. and then overnight at rt. The solution was evaporated close to dryness. 35 ml of DCM was added and pH was adjusted to acidic with 1 M HCl. The phases were separated. Organic phase was washed twice with water and dried by filtration through a phase separation funnel. Evaporated crude product was purified with column chromatography (normal phase silica; EtOAc:heptane gradient) to obtain 239 mg of (R)-3-(1-(4-chlorophenyl)ethyl)quinazoline-2,4(1H,3H)-dione ¹H-NMR (400 MHz, d₆-DMSO): 1.47 (d, 3H), 5.08-5.18 (m, 1H), 7.12 (ddd, 1H), 7.37-7.45 (m, 4H), 7.48-7.54 (m, 1H), 7.85 (dd, 1H), 8.17 (dd, 1H), 9.10 (d, 1H), 10.70 (br s, 1H).

### (R)-3-(1-(4-Chlorophenyl)ethyl)-1-methylquinazoline-2,4(1H,3H)-dione

(R)-3-(1-(4-Chlorophenyl)ethyl)quinazoline-2,4(1H,3H)-dione (239 mg; 0.80 mmol), K₂CO₃ (220 mg; 1.59 mmol), and 5.5 ml of dry ACN were mixed under nitrogen and stirred for 30 min at rt. Iodomethane (0.20 ml; 3.18 mmol) was added slowly and the reaction mixture was stirred overnight at it 25 ml of DCM was added and the mixture was washed three times with 25 ml of water. Organic phase was dried and evaporated to dryness to obtain 246 mg of crude product. The product was purified with column chromatography (normal phase; EtOAc:heptane gradient) to give 210 mg of (R)-3-(1-(4-chlorophenyl)ethyl)-1-methylquinazoline-2,4(1H,3H)-dione. ¹H-NMR (400 MHz, CDCl₃): δ 1.90 (d, 3H), 3.54 (s, 3H), 6.42 (q, 1H), 7.17 (d, 1H), 7.22-7.30 (m, 3H), 7.36-7.43 (m, 2H), 7.67 (ddd, 1H), 8.21 (dd, 1H).

### Example 77: 6,8-Dichloro-1-(2-hydroxy-2-methylpropyl)-3-(4-methoxybenzyl)quinazoline-2,4(1H,3H)-dione

### 2-Amino-3,5-dichloro-N-(4-methoxybenzyl)benzamide

2-Amino-3,5-dichlorobenzoic acid (1.0 g; 4.85 mmol), 10 ml of dry DCM, and TEA (2.03 ml; 14.56 mmol) were placed in a reaction flask under nitrogen. The reaction mixture was cooled down. 4-Methoxybenzylamine (0.634 ml; 4.85 mmol) was added slowly and then T3P (3.46 ml; 5.82 mmol; 50% in EtOAc) was added keeping the temperature at rt. The mixture was stirred at rt over three nights. Water was added and the precipitation was filtered and dried in a vacuum oven to yield 0.86 g of 2-amino-3,5-dichloro-N-(4-methoxybenzyl)benzamide. ¹H-NMR (400 MHz, d₆-DMSO): δ 3.73 (s, 3H), 4.35 (d, 2H), 6.67 (br s, 1H), 6.85-6.93 (m, 2H), 7.20-7.27 (m, 2H), 7.52 (d, 1H), 7.62 (d, 1H), 9.03 (t, 1H).

### 6,8-Dichloro-3-(4-methoxybenzyl)quinazoline-2,4(1H,3H)-dione

2-Amino-3,5-dichloro-N-(4-methoxybenzyl)benzamide (1.2 g; 3.71 mmol), 7 ml of dry THF, and 2 ml of dry pyridine were placed in a reaction flask under nitrogen. Ethyl chloroformate (1.06 ml; 11.13 mmol) was added dropwise at 0° C. The mixture was stirred at rt for 2 h, ethyl chloroformate (0.35 ml; 3.71 mmol) was added, and the reaction mixture was stirred at rt overnight. On the next day the reaction mixture was stirred at 50° C. for 3 h to complete carbamate formation. 2 M NaOH (9.27 ml; 18.54 mmol) was added dropwise and the mixture was stirred at rt over three nights. 2 M NaOH (9.27 ml; 18.54 mmol) was added dropwise again and the mixture was stirred at 50° C. for 1 h. On the next day the mixture was evaporated to dryness and the residue was diluted with DCM. Water and 1 M HCl were added till pH was acidic and the precipitation formed was filtered, washed with water, and dried in a vacuum oven to yield 0.376 g of 6,8-dichloro-3-(4-methoxybenzyl)quinazoline-2,4(1H,3H)-dione. ¹H-NMR (400 MHz, d₆-DMSO): δ 3.71 (s, 3H), 5.02 (s, 2H), 6.81-6.90 (m, 2H), 7.25-7.33 (m, 2H), 7.89 (d, 1H), 8.00 (d, 1H), 11.25 (br s, 1H).

### 6,8-Dichloro-1-(2-hydroxy-2-methylpropyl)-3-(4-methoxybenzyl)quinazoline-2,4(1H,3H)-dione

6,8-Dichloro-3-(4-methoxybenzyl)quinazoline-2,4(1H,3H)-dione (367 mg; 1.045 mmol), yttrium(III) nitrate hexahydrate (40.0 mg; 0.105 mmol), 2 ml of dry DMF, and isobutylene oxide (1.392 ml; 15.68 mmol) were charged in a microwave tube and heated at 120° C. for 1 h and at 160° C. for 30 min. Isobutylene oxide (0.464 ml; 5.23 mmol) was added and the mixture was heated at 160° C. for 1 h. After cooling to rt, the mixture was diluted with DCM and washed with saturated NaHCO₃ solution, water, and brine. The organic phase was dried with a phase separator and concentrated under reduced pressure. The residue was purified with flash chromatography to yield 16 mg of 6,8-dichloro-1-(2-hydroxy-2-methylpropyl)-3-(4-methoxybenzyl)quinazoline-2,4(1H,3H)-dione. ¹H-NMR (400 MHz, CDCl₃): δ 1.13 (s, 6H), 2.21 (s, 1H), 3.77 (s, 3H), 4.86 (s, 2H), 5.18 (s, 2H), 6.79-6.86 (m, 2H), 7.39-7.47 (m, 2H), 7.61 (d, 1H), 8.17 (d, 1H).

### Example 78: 6-(4-Bromobenzyl)-10-fluoro-2,2-dimethyl-2H-[1,4]oxazino[2,3,4-ij]quinazoline-5,7(3H,6H)-dione

### 2-Amino-N-(4-bromobenzyl)-3,4-difluorobenzamide

2-Amino-3,4-difluorobenzoic acid (1.0 g; 5.8 mmol), 10 ml of dry DCM, and TEA (3.2 ml; 23 mmol) were placed in a reaction flask under nitrogen. 4-Bromobenzylamine (0.88 ml; 6.9 mmol) was added slowly and then T3P (6.8 ml; 12 mmol; 50% in EtOAc) was added keeping the temperature below 30° C. The mixture was stirred at rt overnight. DCM was added and the mixture was washed twice with water. The organic phase was dried with a phase separator and concentrated under reduced pressure to yield 1.8 g of 2-amino-N-(4-bromobenzyl)-3,4-difluorobenzamide. ¹H-NMR (400 MHz, d₆-DMSO): δ 4.39 (d, 2H), 6.58 (ddd, 1H), 6.73 (s, 2H), 7.25-7.30 (m, 2H), 7.47 (ddd, 1H), 7.50-7.55 (m, 2H), 8.96 (t, 1H).

### 3-(4-Bromobenzyl)-7,8-difluoroquinazoline-2,4(1H,3H)-dione

2-Amino-N-(4-bromobenzyl)-3,4-difluorobenzamide (1.8 g; 5.2 mmol) and 8 ml of dry pyridine were placed in a reaction flask under nitrogen. Ethyl chloroformate (1.5 ml; 16 mmol) was added dropwise at 0° C. The mixture was stirred at rt overnight to complete carbamate formation. 2 M NaOH (7.8 ml; 16 mmol) was added dropwise and the mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 50° C. to yield 1.1 g of 3-(4-bromobenzyl)-7,8-difluoroquinazoline-2,4(1H,3H)-dione. ¹H-NMR (400 MHz, d₆-DMSO): δ 5.05 (s, 2H), 7.18-7.32 (m, 3H), 7.48-7.53 (m, 2H), 7.81 (ddd, 1H), 11.85-12.15 (br s, 1H).

### 3-(4-Bromobenzyl)-7,8-difluoro-1-(2-hydroxy-2-methylpropyl)quinazoline-2,4(1H,3H)-dione

3-(4-Bromobenzyl)-7,8-difluoroquinazoline-2,4(1H,3H)-dione (200 mg; 0.55 mmol), yttrium(III) nitrate hexahydrate (21 mg; 0.054 mmol), 2 ml of dry DMF, and isobutylene oxide (1.45 ml; 16.3 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with CombiFlash (normal phase silica) to yield 140 mg of 3-(4-bromobenzyl)-7,8-difluoro-1-(2-hydroxy-2-methylpropyl)quinazoline-2,4(1H,3H)-dione. ¹H-NMR (400 MHz, CDCl₃): δ 1.26 (s, 6H), 2.68 (s, 1H), 4.49 (s, 2H), 5.20 (s, 2H), 7.08 (ddd, 1H), 7.34-7.39 (m, 2H), 7.40-7.45 (m, 2H), 8.07 (ddd, 1H).

### 6-(4-Bromobenzyl)-10-fluoro-2,2-dimethyl-2H-[1,4]oxazino[2,3,4-ij]quinazoline-5,7(3H,6H)-dione

3-(4-Bromobenzyl)-7,8-difluoro-1-(2-hydroxy-2-methylpropyl)quinazoline-2,4(1H,3H)-dione (120 mg; 0.27 mmol), sodium hydride (22 mg; 0.54 mmol; 60% in oil), and 2 ml of dry THF were stirred under nitrogen at rt for 2 h after which the reaction was quenched with dropwise addition of water. The mixture was diluted with DCM and washed twice with water. The organic phase was dried with a phase separator and concentrated under reduced pressure. The residue was purified with CombiFlash (normal phase silica) to yield 61 mg of 6-(4-bromobenzyl)-10-fluoro-2,2-dimethyl-2H-[1,4]oxazino[2,3,4-ij]quinazoline-5,7(3H,6H)-dione. ¹H-NMR (400 MHz, CDCl₃): δ 1.45 (s, 6H), 3.89 (s, 2H), 5.18 (s, 2H), 6.99 (dd, 1H), 7.38-7.45 (m, 4H), 7.73 (dd, 1H).

### Example 79: 7-Chloro-3-(5-chloro-2,3-dihydro-1H-inden-1-yl)-1-(2-hydroxy-2-methylpropyl)quinazoline-2,4(1H,3H)-dione

### 2-Amino-4-chloro-N-(5-chloro-2,3-dihydro-1H-inden-1-yl)benzamide

2-Amino-4-chlorobenzoic acid (300 mg; 1.75 mmol), 5 ml of DCM, TEA (0.73 ml; 5.25 mmol), and 5-chloro-2,3-dihydro-1H-inden-1-amine (0.322 g; 1.92 mmol) were charged in a reaction flask under nitrogen and cooled to 0° C. T3P (1.25 ml; 2.10 mmol; 50% in EtOAc) was added slowly and the reaction mixture was stirred at rt for 2 h. Water was added and the precipitation formed was filtered, washed with water, and dried under vacuum to yield 398 mg of 2-amino-4-chloro-N-(5-chloro-2,3-dihydro-1H-inden-1-yl)benzamide. ¹H-NMR (400 MHz, d₆-DMSO): δ 2.00 (ddd, 1H), 2.37-2.55 (m, 1H), 2.78-2.90 (m, 1H), 2.93-3.04 (m, 1H), 5.46 (q, 1H), 6.51 (dd, 1H), 6.70 (br s, 2H), 6.77 (d, 1H), 7.23 (d, 2H), 7.31-7.35 (m, 1H), 7.53 (d, 1H), 8.57 (d, 1H).

### 7-Chloro-3-(5-chloro-2,3-dihydro-1H-inden-1-yl)quinazoline-2,4(1H,3H)-dione

2-Amino-4-chloro-N-(5-chloro-2,3-dihydro-1H-inden-1-yl)benzamide (0.398 g; 1.24 mmol) was dissolved in 2 ml of dry pyridine under nitrogen and cooled to 0° C. Ethyl chloroformate (0.36 ml, 3.72 mmol) was added slowly and the reaction mixture was stirred 2 h at rt. 2 M NaOH (4.20 ml; 8.40 mmol) was added slowly at 0° C. and the mixture was stirred overnight at rt and then for 3 h at 50° C. to complete ring closure. The reaction mixture was evaporated to dryness, 20 ml of DCM was added, and pH was adjusted to acidic with 1 M HCl. EtOH and water were added. Organic phase was washed with water and brine, dried by filtration through a phase separator funnel, and evaporated to dryness to obtain 293 mg of 7-chloro-3-(5-chloro-2,3-dihydro-1H-inden-1-yl)quinazoline-2,4(1H,3H)-dione. ¹H-NMR (400 MHz, d₆-DMSO): δ 2.34-2.49 (m, 2H), 2.88-3.00 (m, 1H), 3.11-3.23 (m, 1H), 6.34-6.48 (m, 1H), 7.74-7.15 (m, 2H), 7.20 (d, 1H), 7.23 (dd, 1H), 7.28-7.33 (m, 1H), 8.88 (br s, 1H), 11.50 (br s, 1H).

### 7-Chloro-3-(5-chloro-2,3-dihydro-1H-inden-1-yl)-1-(2-hydroxy-2-methylpropyl)quinazoline-2,4(1H,3H)-dione

7-Chloro-3-(5-chloro-2,3-dihydro-1H-inden-1-yl)quinazoline-2,4(1H,3H)-dione (290 mg; 0.84 mmol), 3 ml of dry DMF, yttrium(Ill) nitrate hexahydrate (32 mg; 0.084 mmol), and isobutylene oxide (0.74 ml; 8.35 mmol) were placed in a microwave reaction vial and the mixture was heated (absorption high) for 1 h at 160° C. More isobutylene oxide (0.37 ml; 4.18 mmol) was added and the same microwave program was repeated. Still more isobutylene oxide (0.37 mmol; 4.18 mmol) was required and also the third microwave heating was performed for 1 h at 160° C. Saturated NaHCO₃ was added and the mixture was washed twice with 12 ml of DCM. Organic phases were combined, washed twice with 30 ml of water and once with 25 ml of brine, dried, and evaporated to dryness. Column chromatography purification (normal phase silica; EtOAc:heptane gradient) gave 284 mg of 7-chloro-3-(5-chloro-2,3-dihydro-1H-inden-1-yl)-1-(2-hydroxy-2-methylpropyl)quinazoline-2,4(1H,3H)-dione. ¹H-NMR (400 MHz, CDCl₃): δ 1.27 (s, 6H), 2.33 (br s, 1H), 2.38-2.50 (m, 1H), 2.51-2.64 (m, 1H), 2.94-3.06 (m, 1H), 3.26-3.40 (m, 1H), 4.04-4.22 (m, 2H), 6.64 (dd, 1H), 6.94 (d, 1H), 7.05-7.12 (m, 1H), 7.22 (dd, 1H), 7.23-7.29 (m, 1H), 7.50 (d, 1H), 8.14 (d, 1H).

### Example 80: 3-(4-Bromobenzyl)-6-chloro-8-fluoro-1-(2-hydroxy-2-methylpropyl)quinazoline-2,4(1H,3H)-dione

### 2-Amino-N-(4-bromobenzyl)-5-chloro-3-fluorobenzamide

2-Amino-5-chloro-3-fluorobenzoic acid (1.0 g; 5.28 mmol), DCM (10 ml), and TEA (2.94 ml; 21.10 mmol) were placed in a reaction flask under nitrogen. 4-Bromobenzylamine (0.80 ml; 6.33 mmol) was added. T3P (6.22 ml; 10.55 mmol; 50% in EtOAc) was added and the reaction mixture was stirred at rt for 3 days. 15 ml of DCM was added and the mixture was washed three times with 20 ml of water. The mixture was dried and evaporated to give 2.23 g of 2-amino-N-(4-bromobenzyl)-5-chloro-3-fluorobenzamide. ¹H-NMR (400 MHz, d₆-DMSO): δ 4.38 (d, 2H), 6.51 (br s, 2H), 7.25-7.30 (m, 2H), 7.37 (dd, 1H), 7.50-7.56 (m, 3H), 9.03 (t, 1H).

### 3-(4-Bromobenzyl)-6-chloro-8-fluoroquinazoline-2,4(1H,3H)-dione

2-Amino-N-(4-bromobenzyl)-5-chloro-3-fluorobenzamide (1.8 g, 5.03 mmol) was dissolved in 7 ml of dry pyridine in a reaction flask under nitrogen. Ethyl chloroformate (1.44 ml; 15.10 mmol) was added at 0° C. slowly and the reaction mixture was stirred at rt for 2 h. 2 M NaOH (7.55 ml; 15.10 mmol) was carefully added at 0° C. and stirring was continued at rt overnight to complete ring closure. The mixture was evaporated close to dryness. The residue was dissolved in 15 ml of DCM and 20 ml of water was added. 1 M HCl was added to adjust pH to <4. The precipitation formed was filtered, washed with water, and dried to obtain 1.698 g of 3-(4-bromobenzyl)-6-chloro-8-fluoroquinazoline-2,4(1H,3H)-dione. ¹H-NMR (400 MHz, d₆-DMSO): δ 5.05 (s, 2H), 7.25-7.35 (m, 2H), 7.47-7.54 (m, 2H), 7.74 (dd, 1H), 7.86 (dd, 1H), 11.83 (br s, 1H).

### 3-(4-Bromobenzyl)-6-chloro-8-fluoro-1-(2-hydroxy-2-methylpropyl)quinazoline-2,4(1H,3H)-dione

3-(4-Bromobenzyl)-6-chloro-8-fluoroquinazoline-2,4(1H,3H)-dione (400 mg; 1.043 mmol), yttrium(III) nitrate hexahydrate (39.9 mg; 0.104 mmol), and 2 ml of dry DMF were placed in a microwave reaction vial. Isobutylene oxide (2.78 ml; 31.3 mmol) was added and the mixture was stirred (absorption high) for 30 min at 160° C. 15 ml of DCM was added and the mixture was washed three times with 25 ml of water. The organic phase was dried and evaporated to dryness. Purification with column chromatography (normal phase silica; EtOAc:heptane gradient) yielded 200 mg of the product. The product was further purified with trituration in DCM to obtain 15 mg of the product. The mother liquor was evaporated to dryness and further triturated in diethyl ether using ultrasound sonication. Filtered precipitations were combined and dried to obtain 115 mg of 3-(4-bromobenzyl)-6-chloro-8-fluoro-1-(2-hydroxy-2-methylpropyl)quinazoline-2,4(1H,3H)-dione. ¹H-NMR (400 MHz, CDCl₃): δ 1.23 (s, 6H), 2.62 (br s, 1H), 4.47 (s, 2H), 5.20, (s, 2H), 7.34-7.45 (m, 5H), 8.06 (dd, 1H).

### Example 81: 6-(4-Chlorobenzyl)-10-fluoro-2,2-dimethyl-2H-[1,4]oxazino[2,3,4-ij]quinazoline-5,7(3H,6H)-dione

### 2-Amino-N-(4-chlorobenzyl)-3,4-difluorobenzamide

2-Amino-3,4-difluorobenzoic acid (1.0 g; 5.8 mmol), 10 ml of dry DCM, and TEA (3.2 ml; 23 mmol) were placed in a reaction flask under nitrogen. 4-Chlorobenzylamine (0.84 ml; 6.9 mmol) was added slowly and then T3P (6.8 ml; 12 mmol; 50% in EtOAc) was added keeping the temperature below 30° C. The mixture was stirred at rt overnight. DCM was added and the mixture was washed twice with water. The organic phase was dried with a phase separator and concentrated under reduced pressure to yield 1.5 g of 2-amino-N-(4-chlorobenzyl)-3,4-difluorobenzamide. ¹H-NMR (400 MHz, d₆-DMSO): δ 4.41 (d, 2H), 6.58 (ddd, 1H), 6.73 (s, 2H), 7.31-7.33 (m, 2H), 7.37-7.41 (m, 2H), 7.47 (ddd, 1H), 8.96 (t, 1H).

### 3-(4-Chlorobenzyl)-7,8-difluoroquinazoline-2,4(1H,3H)-dione

2-Amino-N-(4-chlorobenzyl)-3,4-difluorobenzamide (1.5 g; 4.9 mmol) and 7 ml of dry pyridine were placed in a reaction flask under nitrogen. Ethyl chloroformate (1.4 ml; 15 mmol) was added dropwise at 0° C. The mixture was stirred at rt overnight to complete carbamate formation. 2 M NaOH (7.3 ml; 15 mmol) was added dropwise and the mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 50° C. to yield 0.93 g of 3-(4-chlorobenzyl)-7,8-difluoroquinazoline-2,4(1H,3H)-dione. ¹H-NMR (400 MHz, d₆-DMSO): δ 5.06 (s, 2H), 7.23-7.32 (m, 1H), 7.33-7.40 (m, 4H), 7.82 (ddd, 1H), 11.90-12.10 (br s, 1H).

### 3-(4-Chlorobenzyl)-7,8-difluoro-1-(2-hydroxy-2-methylpropyl)quinazoline-2,4(1H,3H)-dione

3-(4-Chlorobenzyl)-7,8-difluoroquinazoline-2,4(1H,3H)-dione (200 mg; 0.62 mmol), yttrium(III) nitrate hexahydrate (24 mg; 0.062 mmol), 2 ml of dry DMF, and isobutylene oxide (1.65 ml; 18.6 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with CombiFlash (normal phase silica) to yield 140 mg of 3-(4-chlorobenzyl)-7,8-difluoro-1-(2-hydroxy-2-methylpropyl)quinazoline-2,4(1H,3H)-dione. ¹H-NMR (400 MHz, CDCl₃): δ 1.26 (s, 6H), 2.69 (s, 1H), 4.49 (s, 2H), 5.22 (s, 2H), 7.08 (td, 1H), 7.25-7.29 (m, 2H), 7.40-7.46 (m, 2H), 8.07 (ddd, 1H).

### 6-(4-Chlorobenzyl)-10-fluoro-2,2-dimethyl-2H-[1,4]oxazino[2,3,4-ij]quinazoline-5,7(3H,6H)-dione

3-(4-Chlorobenzyl)-7,8-difluoro-1-(2-hydroxy-2-methylpropyl)quinazoline-2,4(1H,3H)-dione (120 mg; 0.30 mmol), sodium hydride (24 mg; 0.60 mmol; 60% in oil), and 2 ml of dry THF were stirred under nitrogen at rt for 2 h after which the reaction was quenched with dropwise addition of water. The mixture was diluted with DCM and washed twice with water. The organic phase was dried with a phase separator and concentrated under reduced pressure. The residue was purified with CombiFlash (normal phase silica) to yield 79 mg of 6-(4-chlorobenzyl)-10-fluoro-2,2-dimethyl-2H-[1,4]oxazino[2,3,4-ij]quinazoline-5,7(3H,6H)-dione. ¹H-NMR (400 MHz, CDCl₃): δ 1.45 (s, 6H), 3.89 (s, 2H), 5.19 (s, 2H), 6.98 (dd, 1H), 7.24-7.29 (m, 2H), 7.44-7.49 (m, 2H), 7.73 (dd, 1H).

### Example 82: (R)-7-Chloro-3-(4-chlorobenzyl)-8-fluoro-1-(2-hydroxypropyl)quinazoline-2,4(1H,3H)-dione

### (R)-7-Chloro-3-(4-chlorobenzyl)-8-fluoro-1-(2-hydroxypropyl)quinazoline-2,4(1H,3H)-dione

The preparation of 7-chloro-3-(4-chlorobenzyl)-8-fluoroquinazoline-2,4(1H,3H)-dione is described in Example 67. 7-Chloro-3-(4-chlorobenzyl)-8-fluoroquinazoline-2,4(1H,3H)-dione (200 mg; 0.59 mmol), yttrium(III) nitrate hexahydrate (23 mg; 0.059 mmol), 1.5 ml of dry DMF, and (R)-2-methyloxirane (0.41 ml; 5.9 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with CombiFlash (normal phase silica) to yield 75 mg of (R)-7-chloro-3-(4-chlorobenzyl)-8-fluoro-1-(2-hydroxypropyl)quinazoline-2,4(1H,3H)-dione. ¹H-NMR (400 MHz, CDCl₃): δ 1.33 (d, 3H), 2.37 (d, 1H), 4.11-4.23 (m, 2H), 4.42-4.53 (m, 1H), 5.20 (m, 2H), 7.25-7.32 (m, 3H), 7.41-7.46 (m, 2H), 8.02 (dd, 1H).

### Example 83: 10-Chloro-6-(4-chlorobenzyl)-2-methyl-5,7-dioxo-3,5,6,7-tetrahydro-2H-[1,4]oxazino[2,3,4-ij]quinazoline-2-carboxylic acid

### Methyl 3-(7-chloro-3-(4-chlorobenzyl)-8-fluoro-2,4-dioxo-3,4-dihydroquinazolin-1(2H)-yl)-2-hydroxy-2-methylpropanoate

The preparation of 7-chloro-3-(4-chlorobenzyl)-8-fluoroquinazoline-2,4(1H,3H)-dione is described in Example 67. 7-Chloro-3-(4-chlorobenzyl)-8-fluoroquinazoline-2,4(1H,3H)-dione (250 mg; 0.737 mmol), yttrium(III) nitrate hexahydrate (28.2 mg; 0.074 mmol), 1 ml of dry DMF, and methyl 2-methylglycidate (0.078 ml; 0.737 mmol) were charged in a microwave tube and heated at 160° C. for 1 h. Methyl 2-methylglycidate (0.390 ml; 3.69 mmol) was added and the mixture was heated at 160° C. for 1 h. After cooling to rt, the mixture was diluted with DCM and washed four times with water. The organic phase was dried with a phase separator and concentrated under reduced pressure. The residue was purified with flash chromatography to yield 120 mg of methyl 3-(7-chloro-3-(4-chlorobenzyl)-8-fluoro-2,4-dioxo-3,4-dihydroquinazolin-1(2H)-yl)-2-hydroxy-2-methylpropanoate. LC-MS (ES) [M+1]: 456.8.

### 10-Chloro-6-(4-chlorobenzyl)-2-methyl-5,7-dioxo-3,5,6,7-tetrahydro-2H-[1,4]oxazino[2,3,4-ij]quinazoline-2-carboxylic acid

Methyl 3-(7-chloro-3-(4-chlorobenzyl)-8-fluoro-2,4-dioxo-3,4-dihydroquinazolin-1(2H)-yl)-2-hydroxy-2-methylpropanoate (120 mg; 0.264 mmol), sodium hydride (21.08 mg; 0.527 mmol; 60% in oil), and 5 ml of dry THF were placed in a reaction flask under nitrogen. The reaction mixture was stirred at rt for 1 h. DCM was added and the mixture was extracted three times with water. The water phase was acidified with 1 M HCl and extracted twice with DCM. The organic phase was dried with a phase separator and evaporated to dryness. The residue was purified with chromatography to yield 12 mg of 10-chloro-6-(4-chlorobenzyl)-2-methyl-5,7-dioxo-3,5,6,7-tetrahydro-2H-[1,4]oxazino[2,3,4-ij]quinazoline-2-carboxylic acid. ¹H-NMR (400 MHz, CDCl₃): δ 1.83 (s, 3H), 3.57 (d, 1H), 4.95 (d, 1H), 5.07-5.26 (q, 2H), 7.21-7.30 (m, 3H), 7.41-7.48 (m, 2H), 7.71 (d, 1H).

### Example 84: 6-(4-Bromobenzyl)-9-fluoro-10-methoxy-2,2-dimethyl-2H-[1,4]oxazino[2,3,4-ij]quinazoline-5,7(3H,6H)-dione

3-(4-Bromobenzyl)-6,7,8-trifluoro-1-(2-hydroxy-2-methylpropyl)quinazoline-2,4(1H,3H)-dione (50 mg; 0.11 mmol) prepared in Example 60, sodium hydride (9 mg; 0.22 mmol; 60% in oil), and 2 ml of dry DMF were stirred under nitrogen at rt for 1 h. MeOH (2 ml) was added and the mixture was concentrated under reduced pressure. The residue was diluted with DCM and washed twice with water and once with saturated aqueous NaCl. The organic phase was dried with a phase separator, concentrated under reduced pressure, and purified with CombiFlash (normal phase silica) to yield 17 mg of 6-(4-bromobenzyl)-9-fluoro-10-methoxy-2,2-dimethyl-2H-[1,4]oxazino[2,3,4-ij]quinazoline-5,7(3H,6H)-dione. ¹H-NMR (400 MHz, CDCl₃): δ 1.45 (s, 6H), 3.87 (s, 2H), 4.04 (d, 3H), 5.18 (s, 2H), 7.36-7.46 (m, 4H), 7.49 (d, 1H).

### Example 85: 7-Chloro-3-((2,3-dihydrobenzofuran-5-yl)methyl)-6-fluoro-1-(2-hydroxy-2-methylpropyl)quinazoline-2,4(1H,3H)-dione

### 2-Amino-4-chloro-N-((2,3-dihydrobenzofuran-5-yl)methyl)-5-fluorobenzamide

2-Amino-4-chloro-5-fluorobenzoic acid (500 mg; 2.64 mmol), 15 ml of DCM, TEA (1.47 ml; 10.55 mmol), and (2,3-dihydrobenzofuran-5-yl)methanamine (0.51 mg; 3.96 mmol) were charged in a reaction flask under nitrogen and cooled to 0° C. T3P (3.11 ml; 5.28 mmol; 50% in EtOAc) was added slowly and the reaction mixture was stirred at rt overnight. DCM was added. Organic phase was washed three times with water, dried by filtration through a phase separator funnel, and evaporated to obtain 788 mg of 2-amino-4-chloro-N-((2,3-dihydrobenzofuran-5-yl)methyl)-5-fluorobenzamide. ¹H-NMR (400 MHz, d₆-DMSO): δ 3.09-3.13 (m, 2H), 4.31 (d, 2H), 4.49 (t, 2H), 6.55 (br s, 2H), 6.69 (d, 1H), 6.86 (d, 1H), 7.00-7.05 (m, 1H), 7.15-7.19 (m, 1H), 7.59 (d, 1H), 8.80 (t, 1H).

### 7-Chloro-3-((2,3-dihydrobenzofuran-5-yl)methyl)-6-fluoroquinazoline-2,4(1H,3H)-dione

2-Amino-4-chloro-N-((2,3-dihydrobenzofuran-5-yl)methyl)-5-fluorobenzamide (0.788 g; 2.46 mmol) was dissolved in 5 ml of dry pyridine under nitrogen and cooled to 0° C. Ethyl chloroformate (0.70 ml, 7.37 mmol) was added slowly and the reaction mixture was stirred at rt overnight. 2 M NaOH (3.69 ml; 7.37 mmol) was added slowly at 0° C. and the mixture was stirred 90 min at 50° C. to complete ring closure. The reaction mixture was evaporated to dryness. 20 ml of DCM and 20 ml of water were added and pH was adjusted to acidic with 1 M HCl. The precipitation formed was filtered, washed with water, and dried to obtain 539 mg of 7-chloro-3-((2,3-dihydrobenzofuran-5-yl)methyl)-6-fluoroquinazoline-2,4(1H,3H)-dione. ¹H-NMR (400 MHz, d₆-DMSO): δ 3.11 (t, 2H), 4.47 (t, 2H), 4.97 (s, 2H), 6.66 (d, 1H), 7.06-7.12 (m, 1H), 7.19-7.24 (m, 1H), 7.32 (d, 1H), 7.85 (d, 1H), 11.63 (br s, 1H).

### 7-Chloro-3-((2,3-dihydrobenzofuran-5-yl)methyl)-6-fluoro-1-(2-hydroxy-2-methylpropyl)quinazoline-2,4(1H,3H)-dione

7-Chloro-3-((2,3-dihydrobenzofuran-5-yl)methyl)-6-fluoroquinazoline-2,4(1H,3H)-dione (150 mg; 0.43 mmol), 1 ml of dry DMF, yttrium(III) nitrate hexahydrate (16.6 mg; 0.043 mmol), and isobutylene oxide (1.15 ml; 12.98 mmol) were placed in a microwave vial under nitrogen and the mixture was heated (absorption high) for 1 h at 160° C. 15 ml of DCM was added. The mixture was washed three times with 25 ml of water. Organic phase was dried and evaporated to dryness to obtain 220 mg of crude product. Column chromatography purification (normal phase silica; EtOAc:heptane gradient) gave 110 mg of 7-chloro-3-((2,3-dihydrobenzofuran-5-yl)methyl)-6-fluoro-1-(2-hydroxy-2-methylpropyl)quinazoline-2,4(1H,3H)-dione. ¹H-NMR (400 MHz, CDCl₃): δ 1.34 (s, 6H), 2.33 (s, 1H), 3.16 (t, 2H), 4.16 (br s, 2H), 4.53 (t, 2H), 5.17 (s, 2H), 6.70 (d, 1H), 7.25-7.35 (m, 1H), 7.35-7.39 (m, 1H), 7.62 (d, 1H), 7.95 (d, 1H).

### Example 86: 7-Chloro-3-(4-chlorobenzyl)-8-fluoro-1-(2-hydroxy-2-methyl-3-oxobutyl)quinazoline-2,4(1H,3H)-dione

The preparation of 7-chloro-3-(4-chlorobenzyl)-8-fluoroquinazoline-2,4(1H,3H)-dione is described in Example 67. 7-Chloro-3-(4-chlorobenzyl)-8-fluoroquinazoline-2,4(1H,3H)-dione (50 mg; 0.15 mmol), yttrium(III) nitrate hexahydrate (6 mg; 0.015 mmol), 1 ml of dry DMF, and 1-(2-methyloxiran-2-yl)ethanone (0.14 ml; 1.5 mmol) were charged in a microwave tube and heated at 160° C. for 1.5 h. After cooling to rt, the mixture was diluted with DCM and washed three times with water. The organic phase was dried with a phase separator and concentrated under reduced pressure. The residue was purified with MS-Trigger to yield 2 mg of 7-chloro-3-(4-chlorobenzyl)-8-fluoro-1-(2-hydroxy-2-methyl-3-oxobutyl)quinazoline-2,4(1H,3H)-dione. ¹H-NMR (400 MHz, CDCl₃): δ 1.48 (s, 3H), 2.40 (s, 3H), 4.07 (d, 1H), 4.64 (dd, 1H), 4.78 (dd, 1H), 5.06 (d, 1H), 5.19 (d, 1H), 7.24-7.30 (m, 3H), 7.33-7.38 (m, 2H), 7.96 (dd, 1H).

### Example 87: 3-(4-Bromobenzyl)-1-ethyl-7-fluoroquinazoline-2,4(1H,3H)-dione

The preparation of 3-(4-bromobenzyl)-7-fluoroquinazoline-2,4(1H,3H)-dione is described in Example 26. 3-(4-Bromobenzyl)-7-fluoroquinazoline-2,4(1H,3H)-dione (0.2 g; 0.57 mmol), bromoethane (0.107 ml; 1.43 mmol), and 1 ml of ACN were heated for 20 min at 75° C. under microwaves. More bromoethane (0.1 ml; 1.43 mmol) was added and the same microwave program was repeated. Water was added to the reaction mixture and the precipitation was washed with water and dried at 50° C. under vacuum. The product mixture (175 mg) was dissolved in 1 ml of ACN/EtOH (0.95 ml:0.05 ml) by heating to reflux. The solution was allowed to cool to rt and then to 0° C. The precipitation was filtered and dried at 50° C. under vacuum to give 84 mg of 3-(4-bromobenzyl)-1-ethyl-7-fluoroquinazoline-2,4(1H,3H)-dione. ¹H-NMR (400 MHz, d₆-DMSO): δ 1.20 (t, 3H), 4.13 (q, 2H), 5.09 (s, 2H), 7.16 (td, 1H), 7.25-7.32 (m, 2H), 7.44-7.53 (m, 3H), 8.13 (dd, 1H).

### Example 88: 7-Chloro-3-(4-chloro-3-fluorobenzyl)-1-((3-methyloxetan-3-yl)methyl)quinazoline-2,4(1H,3H)-dione

### 2-Amino-4-chloro-N-(4-chloro-3-fluorobenzyl)benzamide

2-Amino-4-chlorobenzoic acid (0.50 g; 2.9 mmol), 15 ml of dry DCM, and TEA (1.63 ml; 11.6 mmol) were placed in a reaction flask under nitrogen. 4-Chloro-3-fluorobenzylamine (0.55 ml; 4.4 mmol) was added slowly and then T3P (3.4 ml; 5.8 mmol; 50% in EtOAc) was added keeping the temperature below 30° C. The mixture was stirred at rt overnight. DCM was added and the mixture was washed twice with water. The organic phase was dried with a phase separator and concentrated under reduced pressure to yield 1.0 g of crude product. LC-MS (ES) [M+H]⁺: 313.0.

### 7-Chloro-3-(4-chloro-3-fluorobenzyl)quinazoline-2,4(1H,3H)-dione

2-Amino-4-chloro-N-(4-chloro-3-fluorobenzyl)benzamide (0.91 g; 2.9 mmol) and 5 ml of dry pyridine were placed in a reaction flask under nitrogen. Ethyl chloroformate (0.83 ml; 8.7 mmol) was added dropwise at 0° C. The mixture was stirred at rt overnight to complete carbamate formation. 2 M NaOH (4.4 ml; 8.7 mmol) was added dropwise and the mixture was heated at 50° C. for 2 h. Additional 2 M NaOH (1.5 ml; 3.0 mmol) was added and the mixture was stirred at 50° C. for 1 h. The mixture was partially concentrated and the residue was diluted with DCM. Water and 1 M HCl were added till pH<4 and the precipitation formed was filtered, washed with water, and dried under reduced pressure at 50° C. to yield 0.47 g of 7-chloro-3-(4-chloro-3-fluorobenzyl)quinazoline-2,4(1H,3H)-dione. ¹H-NMR (400 MHz, d₆-DMSO): δ 5.06 (s, 2H), 7.15-7.20 (m, 1H), 7.22 (d, 1H), 7.27 (dd, 1H), 7.37 (dd, 1H), 7.52 (t, 1H), 7.94 (d, 1H), 11.66 (br s, 1H).

### 7-Chloro-3-(4-chloro-3-fluorobenzyl)-1-((3-methyloxetan-3-yl)methyl)quinazoline-2,4(1H,3H)-dione

7-Chloro-3-(4-chloro-3-fluorobenzyl)quinazoline-2,4(1H,3H)-dione (150 mg; 0.44 mmol), sodium hydride (35 mg; 0.88 mmol; 60% in oil), 2 ml of dry ACN, and 3-(chloromethyl)-3-methyloxetane (210 mg; 1.8 mmol) were charged in a microwave tube. The mixture was flushed with nitrogen and heated at 160° C. for 1 h. After cooling to rt, an additional batch of 3-(chloromethyl)-3-methyloxetane (110 mg; 0.89 mmol) was added and the mixture was heated at 160° C. for 1 h. The mixture was allowed to cool to rt and MeOH (2 ml) was added. The mixture was concentrated under reduced pressure and the residue was diluted with DCM. The solution was washed twice with water and once with saturated aqueous NaCl. The organic phase was dried with a phase separator and concentrated under reduced pressure. The residue was purified with MS-Trigger to yield 13 mg of 7-chloro-3-(4-chloro-3-fluorobenzyl)-1-((3-methyloxetan-3-yl)methyl)quinazoline-2,4(1H,3H)-dione. ¹H-NMR (400 MHz, CDCl₃): δ 1.48 (s, 3H), 4.15 (s, 2H), 4.26 (d, 2H), 4.67 (d, 2H), 5.20 (s, 2H), 7.01 (d, 1H), 7.21-7.26 (m, 2H), 7.28-7.35 (m, 2H), 8.19 (d, 1H).

### Example 89: 5,7-Dichloro-3-(4-chlorobenzyl)-1-(2-hydroxy-2-methylpropyl)quinazoline-2,4(1H,3H)-dione

### 2-Amino-4,6-dichloro-N-(4-chlorobenzyl)benzamide

2-Amino-4,6-dichlorobenzoic acid (0.5 g; 2.427 mmol), 5 ml of dry DCM, and TEA (1.353 nil; 9.71 mmol) were placed in a reaction flask under nitrogen. 4-Chlorobenzylamine (0.384 ml; 3.15 mmol) was added slowly and then T3P (2.86 ml; 4.85 mmol; 50% in EtOAc) was added keeping the temperature at rt. The mixture was stirred at rt for 2 h. The reaction mixture was diluted with DCM and washed three times with water. The organic phase was dried with a phase separator and evaporated to dryness to yield 0.814 g of 2-amino-4,6-dichloro-N-(4-chlorobenzyl)benzamide. ¹H-NMR (400 MHz, d₆-DMSO): δ 4.42 (d, 2H), 5.56 (br s, 2H), 6.69 (dd, 2H), 7.31-7.45 (m, 4H), 8.99 (t, 1H).

### 5,7-Dichloro-3-(4-chlorobenzyl)quinazoline-2,4(1H,3H)-dione

2-Amino-4,6-dichloro-N-(4-chlorobenzyl)benzamide (0.8 g; 2.427 mmol), 5 ml of dry THF, and 2 ml of dry pyridine were placed in a reaction flask under nitrogen. Ethyl chloroformate (0.693 ml; 7.28 mmol) was added dropwise at 0° C. The mixture was stirred at rt for 2 h to complete carbamate formation. 2 M NaOH (3.64 ml; 7.28 mmol) was added dropwise and the mixture was stirred at rt overnight. 2 M NaOH (3.64 ml; 7.28 mmol) was added and the mixture was stirred at 50° C. for 3 h to complete the reaction. The mixture was evaporated to dryness and the residue was diluted with DCM. Water and 1 M HCl were added till pH was acidic and the precipitation formed was filtered, washed with water, and dried in a vacuum oven to yield 0.489 g of 5,7-dichloro-3-(4-chlorobenzyl)quinazoline-2,4(1H,3H)-dione. 1H-NMR (400 MHz, d₆-DMSO): δ 5.01 (s, 2H), 7.18 (d, 1H), 7.28-7.45 (m, 5H), 11.77 (br s, 1H).

### 5,7-Dichloro-3-(4-chlorobenzyl)-1-(2-hydroxy-2-methylpropyl)quinazoline-2,4(1H,3H)-dione

5,7-Dichloro-3-(4-chlorobenzyl)quinazoline-2,4(1H,3H)-dione (150 mg; 0.422 mmol), yttrium(III) nitrate hexahydrate (16.16 mg; 0.042 mmol), 1 ml of dry DMF, and isobutylene oxide (1.124 ml; 12.65 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 115 mg of 5,7-dichloro-3-(4-chlorobenzyl)-1-(2-hydroxy-2-methylpropyl)quinazoline-2,4(1H,3H)-dione. ¹H-NMR (400 MHz, CDCl₃): δ 1.32 (s, 6H), 2.22 (s, 1H), 4.20 (s, 2H), 5.20 (s, 2H), 7.25-7.30 (m, 3H), 7.43-7.48 (m, 2H), 7.54 (d, 1H).

### Example 90: 7-Chloro-3-(4-(difluoromethoxy)benzyl)-8-fluoro-1-(2-hydroxy-2-methylpropyl)quinazoline-2,4(1H,3H)-dione

### 2-Amino-4-chloro-N-(4-(difluoromethoxy)benzyl)-3-fluorobenzamide

2-Amino-4-chloro-3-fluorobenzoic acid (0.75 g; 4.0 mmol), 10 ml of dry DCM, and TEA (2.2 ml; 16 mmol) were placed in a reaction flask under nitrogen. 4-(Difluoromethoxy)benzylamine (0.63 ml; 4.4 mmol) was added slowly and then T3P (4.7 ml; 7.9 mmol; 50% in EtOAc) was added keeping the temperature below 30° C. The mixture was stirred at rt overnight. DCM was added and the mixture was washed three times with water. The organic phase was dried with a phase separator and concentrated under reduced pressure to yield 1.6 g of crude product. ¹H-NMR (400 MHz, d₆-DMSO): δ 4.42 (d, 2H), 6.64-6.74 (m, 3H), 7.10-7.17 (m, 2H), 7.19 (t, 1H), 7.34-7.40 (m, 2H), 7.45 (dd, 1H), 9.02 (t, 1H).

### 7-Chloro-3-(4-(difluoromethoxy)benzyl)-8-fluoroquinazoline-2,4(1H,3H)-dione

2-Amino-4-chloro-N-(4-(difluoromethoxy)benzyl)-3-fluorobenzamide (1.4 g; 4.0 mmol) and 7 ml of dry pyridine were placed in a reaction flask under nitrogen. Ethyl chloroformate (1.1 ml; 12 mmol) was added dropwise at 0° C. The mixture was stirred at rt overnight to complete carbamate formation. The mixture was diluted with 5 ml of DCM. 2 M NaOH (5.9 ml; 12 mmol) was added dropwise and the mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 50° C. to yield 0.93 g of 7-chloro-3-(4-(difluoromethoxy)benzyl)-8-fluoroquinazoline-2,4(1H,3H)-dione. ¹H-NMR (400 MHz, d₆-DMSO): δ 5.07 (s, 2H), 7.09-7.15 (m, 2H), 7.19 (t, 1H), 7.34-7.42 (m, 3H), 7.77 (dd, 1H), 11.8-12.1 (br s, 1H).

### 7-Chloro-3-(4-(difluoromethoxy)benzyl)-8-fluoro-1-(2-hydroxy-2-methylpropyl)quinazoline-2,4(1H,3H)-dione

7-Chloro-3-(4-(difluoromethoxy)benzyl)-8-fluoroquinazoline-2,4(1H,3H)-dione (250 mg; 0.67 mmol), yttrium(III) nitrate hexahydrate (26 mg; 0.067 mmol), 1 ml of dry DMF, and isobutylene oxide (1.2 ml; 14 mmol) were charged in a microwave tube and heated at 160° C. for 1 h. After cooling to rt, the mixture was diluted with DCM and washed four times with water. The organic phase was dried with a phase separator and concentrated under reduced pressure. The residue was purified with CombiFlash (normal phase silica) to yield 130 mg of 7-chloro-3-(4-(difluoromethoxy)benzyl)-8-fluoro-1-(2-hydroxy-2-methylpropyl)quinazoline-2,4(1H,3H)-dione. ¹H-NMR (400 MHz, CDCl₃): δ 1.25 (s, 6H), 2.72 (s, 1H), 4.50 (s, 2H), 5.24 (s, 2H), 6.46 (t, 1H), 7.02-7.08 (m, 2H), 7.29 (dd, 1H), 7.48-7.53 (m, 2H), 8.02 (dd, 1H).

### Example 91: (S)-3-(4-Bromobenzyl)-7-chloro-8-fluoro-1-(2-hydroxypropyl)quinazoline-2,4(1H,3H)-dione

### 2-Amino-N-(4-bromobenzyl)-4-chloro-3-fluorobenzamide

2-Amino-4-chloro-3-fluorobenzoic acid (0.75 g; 4.0 mmol), 20 ml of dry DCM, and TEA (2.2 ml; 16 mmol) were placed in a reaction flask under nitrogen. 4-Bromobenzylamine (0.65 ml; 5.1 mmol) was added slowly and then T3P (4.7 ml; 7.9 mmol; 50% in EtOAc) was added keeping the temperature below 30° C. The mixture was stirred at rt overnight. DCM was added and the mixture was washed twice with water and once with saturated aqueous NaCl. The organic phase was dried with a phase separator and concentrated under reduced pressure to yield 1.4 g of 2-amino-N-(4-bromobenzyl)-4-chloro-3-fluorobenzamide. ¹H-NMR (400 MHz, d₆-DMSO): δ 4.39 (d, 2H), 6.65-6.74 (m, 3H), 7.25-7.29 (m, 2H), 7.45 (dd, 1H), 7.50-7.54 (m, 2H), 9.02 (t, 1H).

### 3-(4-Bromobenzyl)-7-chloro-8-fluoroquinazoline-2,4(1H,3H)-dione

2-Amino-N-(4-bromobenzyl)-4-chloro-3-fluorobenzamide (1.4 g; 4.0 mmol) and 7 ml of dry pyridine were placed in a reaction flask under nitrogen. Ethyl chloroformate (1.13 ml; 12 mmol) was added dropwise at 0° C. The mixture was stirred at rt overnight to complete carbamate formation. 2 M NaOH (7.9 ml; 16 mmol) was added dropwise and the mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 40° C. to yield 0.86 g of 3-(4-bromobenzyl)-7-chloro-8-fluoroquinazoline-2,4(1H,3H)-dione. ¹H-NMR (400 MHz, d₆-DMSO): δ 5.05 (s, 2H), 7.27-7.32 (m, 2H), 7.37 (dd, 1H), 7.48-7.53 (m, 2H), 7.77 (dd, 1H), 11.85-12.05 (br s, 1H).

### (S)-3-(4-Bromobenzyl)-7-chloro-8-fluoro-1-(2-hydroxypropyl)quinazoline-2,4(1H,3H)-dione

3-(4-Bromobenzyl)-7-chloro-8-fluoroquinazoline-2,4(1H,3H)-dione (150 mg; 0.39 mmol), yttrium(III) nitrate hexahydrate (15 mg; 0.039 mmol), 1.5 ml of dry DMF, and (S)-2-methyloxirane (0.27 ml; 3.9 mmol) were charged in a microwave tube and heated at 160° C. for 1 h. After cooling to rt, the mixture was diluted with DCM and washed with saturated NaHCO₃, water, and saturated aqueous NaCl. The organic phase was dried with a phase separator and concentrated under reduced pressure. The residue was purified with MS-Trigger to yield 100 mg of (S)-3-(4-bromobenzyl)-7-chloro-8-fluoro-1-(2-hydroxypropyl)quinazoline-2,4(1H,3H)-dione. ¹H-NMR (400 MHz, CDCl₃): δ 1.33 (d, 3H), 2.36 (d, 1H), 4.12-4.23 (m, 2H), 4.47 (ddd, 1H), 5.13-5.24 (m, 2H), 7.29 (dd, 1H), 7.35-7.40 (m, 2H), 7.41-7.46 (m, 2H), 8.01 (dd, 1H).

### Example 92: 3-(4-Bromo-2-fluorobenzyl)-7-chloro-1-(2-hydroxy-2-methylpropyl)quinazoline-2,4(1H,3H)-dione

### 2-Amino-N-(4-bromo-2-fluorobenzyl)-4-chlorobenzamide

2-Amino-4-chlorobenzoic acid (2.5 g; 14.57 mmol), DCM (25 ml), TEA (8.12 ml; 58.3 mmol), and 4-bromo-2-fluorobenzylamine hydrochloride (4.21 g; 17.48 mmol) were charged in a reaction vessel. T3P (17.17 ml; 29.1 mmol; 50% in EtOAc) was added slowly keeping the temperature at rt with ice bath. The reaction was complete in 2 h, but the mixture was stirred at rt overnight. DCM was added and the mixture was washed three times with water. Organic layer was dried by filtration through a phase separator funnel and evaporated to dryness to obtain 6.61 g of crude 2-amino-N-(4-bromo-2-fluorobenzyl)-4-chlorobenzamide, which was not further purified. ¹H-NMR (400 MHz, d₆-DMSO): δ 4.40 (d, 2H), 6.54 (dd, 1H), 6.77 (d, 1H), 7.27-7.35 (m, 1H), 7.39 (dd, 1H), 7.51 (dd, 1H), 7.58 (d, 1H), 8.87 (t, 1H).

### 3-(4-Bromo-2-fluorobenzyl)-7-chloroquinazoline-2,4(1H,3H)-dione

2-Amino-N-(4-bromo-2-fluorobenzyl)-4-chlorobenzamide (5.21 g; 14.57 mmol) and 30 ml of pyridine were cooled to 0° C. under nitrogen. Ethyl chloroformate (4.16 ml; 43.7 mmol) was added slowly and the mixture stirred at rt for 90 min to complete carbamate formation. The reaction was cooled to 0° C. and 2 M NaOH (21.85 ml; 43.7 mmol) was added slowly. The reaction mixture was heated at 50° C. for 150 min, allowed to cool, and stirred at rt over the weekend. 2 M NaOH (21.84 ml; 43.7 mmol) was added again and the mixture was heated at 50° C. for 4 h to complete ring closure. The reaction mixture was concentrated. 50 ml of DCM was added and pH was adjusted to very acidic with 1 M HCl. The precipitation was filtered, washed with water, and dried under vacuum at 50° C. overnight to obtain 4.03 g of 3-(4-bromo-2-fluorobenzyl)-7-chloroquinazoline-2,4(1H,3H)-dione. ¹H-NMR (400 MHz, d₆-DMSO): δ 5.07 (s, 2H), 7.15-7.22 (m, 1H), 7.24 (dd, 1H), 7.28 (dd, 1H), 7.33 (dd, 1H), 7.54 (dd, 1H), 7.94 (d, 1H), 11.72 (br s, 1H).

### 3-(4-Bromo-2-fluorobenzyl)-7-chloro-1-(2-hydroxy-2-methylpropyl)quinazoline-2,4(1H,3H)-dione

3-(4-Bromo-2-fluorobenzyl)-7-chloroquinazoline-2,4(1H,3H)-dione (120 mg; 0.31 mmol), yttrium(III) nitrate hexahydrate (12 mg; 0.03 mmol), 1 ml of DCM, and isobutylene oxide (0.83 ml; 9.38 mmol) were placed in a microwave vial and the mixture was heated (absorption high) for 1 h at 160° C. DCM (15 ml) was added to the cooled mixture and the mixture was washed three times with 25 ml of water. Organic phase was dried and evaporated to dryness to obtain 125 mg of crude material, which was purified with preparative LC-MS to obtain 41 mg of 3-(4-bromo-2-fluorobenzyl)-7-chloro-1-(2-hydroxy-2-methylpropyl)quinazoline-2,4(1H,3H)-dione. ¹H-NMR (400 MHz, CDCl₃): δ 1.33 (s, 6H), 2.35 (s, 1H), 4.19 (s, 2H), 5.29 (s, 2H), 7.14-7.26 (m, 4H), 7.55 (d, 1H), 8.16 (d, 1H).

### Example 93: 3-(2,4-Dichlorobenzyl)-6,7-difluoro-1-(2-hydroxy-2-methylpropyl)quinazoline-2,4(1H,3H)-dione

### 2-Amino-N-(2,4-dichlorobenzyl)-4,5-difluorobenzamide

2-Amino-4,5-difluorobenzoic acid (2.0 g; 11.55 mmol), DCM (15 ml), TEA (4.83 ml; 34.7 mmol), and 2,4-dichlorobenzylamine (2.034 g; 11.55 mmol) were charged in a reaction vessel and cooled to 0° C. T3P (8.17 ml; 13.86 mmol; 50% in EtOAc) was added slowly and the reaction mixture was stirred at rt overnight. The reaction mixture was diluted with DCM and washed twice with water. Organic layer was dried and evaporated to dryness. Toluene was added and the mixture was evaporated again to obtain 3.31 g of crude 2-amino-N-(2,4-dichlorobenzyl)-4,5-difluorobenzamide. ¹H-NMR (400 MHz, CDCl₃+d₄-MeOH): δ 4.59 (d, 2H), 6.49 (dd, 1H), 7.19-7.30 (m, 3H), 7.36 (d, 1H), 7.41 (d, 1H).

### Ethyl (2-((2,4-dichlorobenzyl)carbamoyl)-4,5-difluorophenyl)carbamate

2-Aniino-N-(2,4-dichlorobenzyl)-4,5-difluorobenzamide (2.0 g; 6.04 mmol) was dissolved in 10 ml of dry pyridine and cooled to 0° C. Ethyl chloroformate (1.73 ml, 18.12 mmol) was added slowly and the reaction mixture was stirred overnight at rt. 25 ml of EtOAc was added and then 25 ml of 1 M HCl was added slowly. Water phase was separated and washed twice with 25 ml of EtOAc. Organic phases were combined and washed twice with 1 M HCl and twice with water. Organic phase was dried, evaporated to dryness, and dried under vacuum at 50° C. to obtain 2.13 g of ethyl (2-((2,4-dichlorobenzyl)carbamoyl)-4,5-difluorophenyl)carbamate. ¹H-NMR (400 MHz, d₆-DMSO): δ 1.23 (t, 3H), 4.14 (t, 2H), 4.50 (d, 2H), 7.42 (dd, 1H), 7.45 (d, 1H), 7.64 (d, 1H), 8.00 (dd, 1H), 8.21 (dd, 1H), 9.36 (t, 1H), 10.98 (br s, 1H).

### 3-(2,4-Dichlorobenzyl)-6,7-difluoroquinazoline-2,4(1H,3H)-dione

Ethyl (2-((2,4-dichlorobenzyl)carbamoyl)-4,5-difluorophenyl)carbamate (2.13 g; 5.28 mmol) was dissolved in 20 ml of dry THF. 2 M NaOH (5.28 ml; 10.57 mmol) was added and the mixture was stirred for 2 h at rt. 20 ml of water was added and the reaction mixture was neutralized with HCl. The precipitation was filtered, washed with water, and dried under vacuum at 50° C. to obtain 1.7 g of 3-(2,4-dichlorobenzyl)-6,7-difluoroquinazoline-2,4(1H,3H)-dione. ¹H-NMR (400 MHz, d₆-DMSO): δ 5.08 (s, 2H), 7.14 (d, 1H), 7.18 (dd, 1H), 7.32 (dd, 1H), 7.66 (d, 1H), 7.93 (dd, 1H), 11.78 (br s, 1H).

### 3-(2,4-Dichlorobenzyl)-6,7-difluoro-1-(2-hydroxy-2-methylpropyl)quinazoline-2,4(1H,3H)-dione

3-(2,4-Dichlorobenzyl)-6,7-difluoroquinazoline-2,4(1H,3H)-dione (200 mg; 0.56 mmol), 2 ml of dry DMF, K₂CO₃ (116 mg; 0.84 mmol), and isobutylene oxide (0.50 ml; 5.60 mmol) were placed in a microwave vial and the mixture was heated (absorption high) first for 15 min at 125° C. and then for 1 h at 150° C. The reaction mixture was neutralized with 1 M HCl. 20 ml of water was added and the mixture was washed twice with 25 ml of EtOAc. Organic phase was dried with Na₂SO₄, filtered, and evaporated to dryness. The crude product was crystallized from ACN:water and the filtered product was dried under vacuum at 50° C. to obtain 33 mg of 3-(2,4-dichlorobenzyl)-6,7-difluoro-1-(2-hydroxy-2-methylpropyl)quinazoline-2,4(1H,3H)-dione. ¹H-NMR (400 MHz, d₆-DMSO): δ 1.16 (s, 6H), 4.11 (br s, 2H), 4.70 (s, 1H), 5.15 (s, 2H), 7.14 (d, 1H), 7.32 (dd, 1H), 7.66 (d, 1H), 7.92-8.02 (m, 2H).

### Example 94: 9-Chloro-6-(4-methoxybenzyl)-2,2-dimethyl-2H-[1,4]oxazino[2,3,4-ij]quinazoline-5,7(3H,6H)-dione

The preparation of 6-chloro-8-fluoro-1-(2-hydroxy-2-methylpropyl)-3-(4-methoxybenzyl)quinazoline-2,4(1H,3H)-dione is described in Example 74. 6-Chloro-8-fluoro-1-(2-hydroxy-2-methylpropyl)-3-(4-methoxybenzyl)quinazoline-2,4(1H,3H)-dione (50 mg; 0.123 mmol), sodium hydride (9.83 mg; 0.246 mmol; 60% in oil), and 3 ml of dry THF were placed in a reaction flask under nitrogen. The reaction mixture was stirred at rt for 1.5 h. The reaction mixture was diluted with DCM and washed twice with water. The organic phase was dried with a phase separator and evaporated to dryness. The residue was purified with chromatography to yield 42 mg of 9-chloro-6-(4-methoxybenzyl)-2,2-dimethyl-2H-[1,4]oxazino[2,3,4-ij]quinazoline-5,7(3H,6H)-dione. ¹H-NMR (400 MHz, CDCl₃): δ 1.39 (s, 6H), 3.77 (s, 3H), 3.85 (s, 2H), 5.17 (s, 2H), 6.78-6.88 (m, 2H), 7.12 (d, 1H), 7.43-7.53 (m, 2H), 7.71 (d, 1H).

### Example 95: 3-(4-(Difluoromethoxy)benzyl)-6,8-difluoro-1-(2-hydroxy-2-methylpropyl)quinazoline-2,4(1H,3H)-dione

### 2-Amino-N-(4-(difluoromethoxy)benzyl)-3,5-difluorobenzamide

2-Amino-3,5-difluorobenzoic acid (500 mg; 2.89 mmol), 10 ml of DCM, TEA (1.61 ml; 11.55 mmol), and 4-(difluoromethoxy)benzylamine (0.50 ml; 3.47 mmol) were charged in a reaction flask. T3P (3.40 ml; 5.78 mmol; 50% in EtOAc) was added slowly and the reaction mixture was stirred at rt overnight. The reaction mixture was diluted with DCM and washed three times with water. Organic layer was dried by filtration through a phase separator funnel and evaporated to dryness to obtain 919 mg of 2-amino-N-(4-(difluoromethoxy)benzyl)-3,5-difluorobenzamide. ¹H-NMR (400 MHz, d₆-DMSO): δ 4.42 (d, 2H), 6.23 (br s, 2H), 7.10-7.17 (m, 2H), 7.24-7.33 (m, 1H), 7.33-7.40 (m, 4H), 8.99 (t, 1H).

### 3-(4-(Difluoromethoxy)benzyl)-6,8-difluoroquinazoline-2,4(1H,3H)-dione

2-Amino-N-(4-(difluoromethoxy)benzyl)-3,5-difluorobenzamide (0.919 g; 2.80 mmol) was dissolved in 5 ml of dry pyridine and cooled to 0° C. Ethyl chloroformate (0.80 ml, 8.40 mmol) was added slowly and the reaction mixture was stirred for 2 h at rt. 2 M NaOH (4.20 ml; 8.40 mmol) was added and the mixture was stirred overnight at rt. The reaction mixture was evaporated close to dryness. 15 ml of DCM and 20 ml of water were added and pH was adjusted to acidic with 1 M HCl. The precipitation was filtered, washed with water, and dried under vacuum at 50° C. to obtain 0.44 g of 3-(4-(difluoromethoxy)benzyl)-6,8-difluoroquinazoline-2,4(1H,3H)-dione. ¹H-NMR (400 MHz, d₆-DMSO): δ 5.07 (s, 2H), 7.08-7.15 (m, 2H), 7.34-7.42 (m, 2H), 7.55 (ddd, 1H), 7.77 (ddd, 1H), 11.78 (br s, 1H).

### 3-(4-(Difluoromethoxy)benzyl)-6,8-difluoro-1-(2-hydroxy-2-methylpropyl)quinazoline-2,4(1H,3H)-dione

3-(4-(Difluoromethoxy)benzyl)-6,8-difluoroquinazoline-2,4(1H,3H)-dione (200 mg; 0.57 mmol), 1 ml of dry DMF, yttrium(III) nitrate hexahydrate (21.6 mg; 0.056 mmol), and isobutylene oxide (1.50 ml; 16.94 mmol) were placed in a microwave vial and the mixture was heated (absorption high) for 1 h at 150° C. 15 ml of DCM was added and the mixture was washed three times with 25 ml of water. Brine was added to the first wash. The crude product was dried and evaporated to dryness. Column chromatography purification (normal phase silica; EtOAc:heptane gradient) gave 87 mg of 3-(4-(difluoromethoxy)benzyl)-6,8-difluoro-1-(2-hydroxy-2-methylpropyl)quinazoline-2,4(1H,3H)-dione. ¹H-NMR (400 MHz, CDCl₃): δ 1.24 (s, 6H), 2.75 (s, 1H), 4.47 (s, 2H), 5.24 (s, 2H), 6.46 (t, 1H), 7.02-7.08 (m, 2H), 7.18 (ddd, 1H), 7.48-7.54 (m, 2H), 7.80 (ddd, 1H).

### Example 96: 3-(4-Bromobenzyl)-7-chloro-1-(2-hydroxy-2-methylpropyl)-8-(2-methoxyethoxy)quinazoline-2,4(1H,3H)-dione

### 3-(4-Bromobenzyl)-7-chloro-8-(2-methoxyethoxy)quinazoline-2,4(1H,3H)-dione

3-(4-Bromobenzyl)-7-chloro-8-fluoroquinazoline-2,4(1H,3H)-dione (0.25 g; 0.65 mmol) prepared in Example 91, sodium hydride (130 mg; 3.3 mmol, 60% in oil), and 2.5 ml of dry THF were charged in a microwave tube. 2-Methoxyethanol (0.51 ml; 6.5 mmol) was added and the mixture was heated at 120° C. for 1 h. After cooling to it, the reaction was quenched with dropwise addition of MeOH. The mixture was diluted with water and extracted twice with DCM. The combined organic phase was washed twice with water and once with saturated aqueous NaCl. The organic phase was dried with a phase separator, concentrated under reduced pressure, and purified with CombiFlash (normal phase silica) to yield 0.23 g of 3-(4-bromobenzyl)-7-chloro-8-(2-methoxyethoxy)quinazoline-2,4(1H,3H)-dione. ¹H-NMR (400 MHz, CDCl₃): δ 3.62 (s, 3H), 3.73-3.78 (m, 2H), 4.19-4.23 (m, 2H), 5.15 (s, 2H), 7.15 (d, 1H), 7.38-7.45 (m, 4H), 7.81 (dd, 1H), 10.16 (s, 1H).

### 3-(4-Bromobenzyl)-7-chloro-1-(2-hydroxy-2-methylpropyl)-8-(2-methoxyethoxy)quinazoline-2,4(1H,3H)-dione

3-(4-Bromobenzyl)-7-chloro-8-(2-methoxyethoxy)quinazoline-2,4(1H,3H)-dione (120 mg; 0.28 mmol), yttrium(III) nitrate hexahydrate (11 mg; 0.03 mmol), 0.5 ml of dry DMF, and isobutylene oxide (0.25 ml; 2.8 mmol) were charged in a microwave tube and heated at 160° C. for 1 h. After cooling to rt, the mixture was diluted with DCM and washed with saturated NaHCO₃, water, and saturated aqueous NaCl. The organic phase was dried with a phase separator and concentrated under reduced pressure. The residue was purified with CombiFlash (normal phase silica) to yield 6 mg of 3-(4-bromobenzyl)-7-chloro-1-(2-hydroxy-2-methylpropyl)-8-(2-methoxyethoxy)quinazoline-2,4(1H,3H)-dione. ¹H-NMR (400 MHz, CDCl₃): δ 1.11 (s, 6H), 2.22 (s, 1H), 3.42 (s, 3H), 3.73-3.77 (m, 2H), 4.02-4.07 (m, 2H), 4.86 (s, 2H), 5.20 (s, 2H), 7.28 (d, 1H), 7.34-7.38 (m, 2H), 7.40-7.44 (m, 2H), 7.98 (d, 1H).

### Example 97: 1-(3-Bromo-2-(hydroxymethyl)-2-methylpropyl)-3-(4-bromobenzyl)-7-chloro-6-fluoroquinazoline-2,4(1H,3H)-dione

The preparation of 3-(4-bromobenzyl)-7-chloro-6-fluoro-1-((3-methyloxetan-3-yl)methyl)quinazoline-2,4(1H,3H)-dione is described in Example 50. 3-(4-Bromobenzyl)-7-chloro-6-fluoro-1-((3-methyloxetan-3-yl)methyl)quinazoline-2,4(1H,3H)-dione (50 mg; 0.11 mmol) and 1 ml of dry THF were placed in a reaction flask. Concentrated hydrobromic acid (0.025 ml, 0.21 mmol) was added at 0° C. and the mixture was stirred at rt overnight. The mixture was diluted with saturated NaHCO₃, extracted twice with DCM, and washed twice with water and once with saturated aqueous NaCl. The organic phase was dried with a phase separator and concentrated under reduced pressure. The residue was purified with CombiFlash (normal phase silica) to yield 37 mg of 1-(3-bromo-2-(hydroxymethyl)-2-methylpropyl)-3-(4-bromobenzyl)-7-chloro-6-fluoroquinazoline-2,4(1H,3H)-dione. ¹H-NMR (400 MHz, CDCl₃): δ 0.90 (s, 3H), 3.25-3.35 (m, 1H), 3.39 (dd, 1H), 3.45 (d, 1H), 3.60-3.80 (m, 2H), 4.00-4.20 (br s, 1H), 4.25-4.50 (br s, 1H), 5.19 (q, 2H), 7.34-7.39 (m, 3H), 7.40-7.45 (m, 2H), 8.28 (d, 1H).

### Example 98: (S)-7-Chloro-3-(1-(4-chlorophenyl)ethyl)-6-fluoro-1-methylquinazoline-2,4(1H,3H)-dione

### (S)-2-Amino-4-chloro-N-(1-(4-chlorophenyl)ethyl)-5-fluorobenzamide

2-Amino-4-chloro-5-fluorobenzoic acid (2.5 g; 13.19 mmol), DCM (25 ml), and TEA (7.35 ml; 52.8 mmol) were placed in a reaction vessel under nitrogen. (S)-1-(4-Chlorophenyl)-ethylamine (2.22 ml; 15.83 mmol) and T3P (15.54 ml; 26.4 mmol; 50% in EtOAc) were added in this order keeping the temperature stable with cooling bath. The reaction mixture was stirred at rt overnight. The mixture was diluted with DCM and washed three times with water. The organic layer was dried with a phase separator funnel and evaporated to dryness to give 4.68 g of (S)-2-amino-4-chloro-N-(1-(4-chlorophenyl)ethyl)-5-fluorobenzamide. ¹H-NMR (400 MHz, CDCl₃): δ 1.89 (d, 3H), 3.51 (s, 3H), 6.36 (q, 1H), 7.24 (d, 1H), 7.25-7.30 (m, 2H), 7.35-7.41 (m, 2H), 7.48-7.53 (m, 2H), 7.94 (d, 1H), 11.68 (br s, 1H).

### (S)-7-Chloro-3-(1-(4-chlorophenyl)ethyl)-6-fluoroquinazoline-2,4(1H,3H)-dione

(S)-2-Amino-4-chloro-N-(1-(4-chlorophenyl)ethyl)-5-fluorobenzamide (4.3 g, 13.14 mmol) was dissolved in dry pyridine (25 ml) under nitrogen and cooled to 0° C. Ethyl chloroformate (3.75 ml; 39.4 mmol) was added slowly and the mixture was stirred for 90 min at rt to complete carbamate formation. The solution was cooled to 0° C. and 2 M NaOH (19.71 ml; 39.4 mmol) was added slowly. The solution was stirred for 3.5 h at 50° C. to complete the reaction and then over the weekend at rt. The reaction mixture was evaporated close to dryness. 50 ml of DCM was added and pH was adjusted to acidic with 1 M HCl. The phases were separated. Organic phase was washed with water, dried with a phase separator funnel, and evaporated to dryness. The product was dried at 50° C. to give 4.56 g of (S)-7-chloro-3-(1-(4-chlorophenyl)ethyl)-6-fluoroquinazoline-2,4(1H,3H)-dione. ¹H-NMR (400 MHz, d₆-DMSO): δ 1.79 (d, 3H), 6.13 (q, 1H), 7.31 (d, 1H), 7.33-7.36 (m, 4H), 7.36-7.44 (m, 1H), 11.54 (br s, 1H).

### (S)-7-Chloro-3-(1-(4-chlorophenyl)ethyl)-6-fluoro-1-methylquinazoline-2,4(1H,3H)-dione

(S)-7-Chloro-3-(1-(4-chlorophenyl)ethyl)-6-fluoroquinazoline-2,4(1H,3H)-dione (150 mg; 0.43 mmol), K₂CO₃ (117 mg; 0.85 mmol), and 4 ml of ACN were stirred for 15 min under nitrogen. Iodomethane (0.106 ml; 1.70 mmol) was added and the reaction mixture was stirred overnight at rt. DCM (25 ml) was added. Organic phase was washed three times with 25 ml of water, dried by filtration through a phase separator, and evaporated to dryness. The crude material was purified with column chromatography (normal phase; EtOAc:heptane gradient) to give 108 mg of (S)-7-chloro-3-(1-(4-chlorophenyl)ethyl)-6-fluoro-1-methylquinazoline-2,4(1H,3H)-dione. ¹H-NMR (400 MHz, CDCl₃): δ 1.89 (d, 3H), 3.51 (s, 3H), 6.36 (q, 1H), 7.23 (d, 1H), 7.24-7.30 (m, 2H), 7.35-7.41 (m, 2H), 7.94 (d, 1H).

### Example 99: 7-Chloro-3-(4-chlorobenzyl)-8-fluoro-1-methylquinazoline-2,4(1H,3H)-dione

The preparation of 7-chloro-3-(4-chlorobenzyl)-8-fluoroquinazoline-2,4(1H,3H)-dione is described in Example 67. 7-Chloro-3-(4-chlorobenzyl)-8-fluoroquinazoline-2,4(1H,3H)-dione (1.0 g; 2.95 mmol), K₂CO₃ (0.815 g; 5.90 mmol), and 10 ml of dry DMF were charged in a flask under nitrogen. Iodomethane (0.551 ml; 8.85 mmol) was added slowly and the reaction mixture was stirred at rt overnight. Water was added to the reaction mixture and the precipitation formed was filtered, washed with water, and dried in a vacuum oven to yield 0.993 mg of 7-chloro-3-(4-chlorobenzyl)-8-fluoro-1-methylquinazoline-2,4(1H,3H)-dione. ¹H-NMR (400 MHz, d₆-DMSO): δ 3.68 (d, 3H), 5.10 (s, 2H), 7.33-7.40 (m, 4H), 7.49 (dd, 1H), 7.90 (dd, 1H).

### Example 100: 3-(4-Bromobenzyl)-7-chloro-6-fluoro-1-(oxetan-3-yl)quinazoline-2,4(1H,3H)-dione

3-(4-Bromobenzyl)-7-chloro-6-fluoroquinazoline-2,4(1H,3H)-dione (200 mg; 0.52 mmol) prepared in Example 48, sodium hydride (42 mg; 1.04 mmol, 60% in oil), and DMF (2 ml) were placed in a microwave reaction vial under nitrogen and the mixture was stirred at rt for 15 min. 3-Iodooxetane (288 mg; 1.56 mmol) in 0.5 ml of DMF was added and the reaction mixture was heated in a microwave reactor at 120° C. for 6 h. After cooling to rt, MeOH was added and the mixture was concentrated. The residue was diluted with DCM. The mixture was washed with saturated NaHCO₃, water, and brine, dried with a phase separator, and evaporated to dryness. The crude product was purified with column chromatography (EtOAc:heptane) and MS-Trigger to give 7 mg of 3-(4-bromobenzyl)-7-chloro-6-fluoro-1-(oxetan-3-yl)quinazoline-2,4(1H,3H)-dione. ¹H-NMR (400 MHz, CDCl₃): δ 4.85 (dd, 2H), 5.01 (dd, 2H), 5.14 (s, 2H), 5.36 (quint, 1H), 6.84 (d, 1H), 7.33-7.39 (m, 2H), 7.41-7.46 (m, 2H), 7.99 (d, 1H).

### Example 101: 7-Chloro-3-(4-chlorobenzyl)-8-fluoro-1-(2-hydroxyethyl)quinazoline-2,4(1H,3H)-dione

The preparation of 7-chloro-3-(4-chlorobenzyl)-8-fluoroquinazoline-2,4(1H,3H)-dione is described in Example 67. 7-Chloro-3-(4-chlorobenzyl)-8-fluoroquinazoline-2,4(1H,3H)-dione (100 mg; 0.30 mmol), yttrium(III) nitrate hexahydrate (11 mg; 0.030 mmol), 1 ml of dry DMF, and ethylene oxide (0.18 ml; 0.44 mmol; 2.5 M solution in THF) were charged in a microwave tube and heated at 160° C. for 1 h. After cooling to rt, the mixture was diluted with DCM and washed three times with water. The organic phase was dried with a phase separator and concentrated under reduced pressure. The residue was purified with CombiFlash (normal phase silica) to yield 15 mg of 7-chloro-3-(4-chlorobenzyl)-8-fluoro-1-(2-hydroxyethyDquinazoline-2,4(1H,3H)-dione. ¹H-NMR (400 MHz, CDCl₃): δ 2.21 (t, 1H), 3.96-4.06 (m, 2H), 4.45-4.55 (m, 2H), 5.20 (s, 2H), 7.25-7.33 (m, 3H), 7.41-7.47 (m, 2H), 8.01 (dd, 1H).

### Example 102: 10-Fluoro-6-(4-methoxybenzyl)-2,2-dimethyl-2H-[1,4]oxazino[2,3,4-ij]quinazoline-5,7(3H,6H)-dione

### 2-Amino-3,4-difluoro-N-(4-methoxybenzyl)benzamide

2-Amino-3,4-difluorobenzoic acid (1.0 g; 5.8 mmol), 10 ml of dry DCM, and TEA (3.2 ml; 23 mmol) were placed in a reaction flask under nitrogen. 4-Methoxybenzylamine (0.91 ml; 6.9 mmol) was added slowly and then T3P (6.8 ml; 12 mmol; 50% in EtOAc) was added keeping the temperature below 30° C. The mixture was stirred at rt overnight. DCM was added and the mixture was washed twice with water. The organic phase was dried with a phase separator and concentrated under reduced pressure to yield 1.6 g of 2-amino-3,4-difluoro-N-(4-methoxybenzyl)benzamide. ¹H-NMR (400 MHz, d₆-DMSO): δ 3.72 (s, 3H), 4.35 (d, 2H), 6.56 (ddd, 1H), 6.72 (s, 2H), 6.85-6.92 (m, 2H), 7.21-7.27 (m, 2H), 7.45 (ddd, 1H), 8.87 (t, 1H).

### 7,8-Difluoro-3-(4-methoxybenzyl)quinazoline-2,4(1H,3H)-dione

2-Amino-3,4-difluoro-N-(4-methoxybenzyl)benzamide (1.6 g; 5.3 mmol) and 7 nil of dry pyridine were placed in a reaction flask under nitrogen. Ethyl chloroformate (1.5 ml; 16 mmol) was added dropwise at 0° C. The mixture was stirred at rt overnight to complete carbamate formation. 2 M NaOH (8.0 ml; 16 mmol) was added dropwise and the mixture was heated at 50° C. for 3 h and stirred at rt overnight. The mixture was partially concentrated and the residue was diluted with DCM. Water and 1 M HCl were added till pH<4 and the precipitation formed was filtered, washed with water, and dried under reduced pressure at 50° C. to yield 0.96 g of 7,8-difluoro-3-(4-methoxybenzyl)quinazoline-2,4(1H,3H)-dione. ¹H-NMR (400 MHz, d₆-DMSO): δ 3.71 (s, 3H), 5.01 (s, 2H), 6.84-6.89 (m, 2H), 7.21-7.32 (m, 3H), 7.81 (ddd, 1H), 11.95 (s, 1H).

### 7,8-Difluoro-1-(2-hydroxy-2-methylpropyl)-3-(4-methoxybenzyl)quinazoline-2,4(1H,3H)-dione

7,8-Difluoro-3-(4-methoxybenzyl)quinazoline-2,4(1H,3H)-dione (200 mg; 0.63 mmol), yttrium(III) nitrate hexahydrate (24 mg; 0.063 mmol), 2 ml of dry DMF, and isobutylene oxide (1.67 ml; 18.9 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with CombiFlash (normal phase silica) to yield 160 mg of 7,8-difluoro-1-(2-hydroxy-2-methylpropyl)-3-(4-methoxybenzyl)quinazoline-2,4(1H,3H)-dione. ¹H-NMR (400 MHz, CDCl₃): δ 1.26 (s, 6H), 2.88 (s, 1H), 3.77 (s, 3H), 4.48 (s, 2H), 5.20 (s, 2H), 6.80-6.85 (m, 2H), 7.06 (td, 1H), 7.42-7.48 (m, 2H), 8.07 (ddd, 1H).

### 10-Fluoro-6-(4-methoxybenzyl)-2,2-dimethyl-2H-[1,4]oxazino[2,3,4-ij]quinazoline-5,7(3H,6H)-dione

7,8-Difluoro-1-(2-hydroxy-2-methylpropyl)-3-(4-methoxybenzyl)quinazoline-2,4(1H,3H)-dione (140 mg; 0.36 mmol), sodium hydride (29 mg; 0.73 mmol; 60% in oil), and 2 ml of dry THF were stirred under nitrogen at rt for 2 h after which the reaction was quenched with dropwise addition of water. The mixture was diluted with DCM and washed twice with water. The organic phase was dried with a phase separator and concentrated under reduced pressure. The residue was purified with CombiFlash (normal phase silica) to yield 86 mg of 10-fluoro-6-(4-methoxybenzyl)-2,2-dimethyl-2H-[1,4]oxazino[2,3,4-ij]quinazoline-5,7(3H,6H)-dione. ¹H-NMR (400 MHz, CDCl₃): δ 1.44 (s, 6H), 3.77 (s, 3H), 3.89 (s, 2H), 5.17 (s, 2H), 6.81-6.86 (m, 2H), 6.96 (dd, 1H), 7.46-7.52 (m, 2H), 7.73 (dd, 1H).

### Example 103: 3-(4-Bromobenzyl)-7-chloro-1-(1-cyclopropyl-1-oxopropan-2-yl)-6-fluoroquinazoline-2,4(1H,3H)-dione

### 2-(3-(4-Bromobenzyl)-7-chloro-6-fluoro-2,4-dioxo-3,4-dihydroquinazolin-1(2H)-yl)-N-methoxy-N-methylpropanamide

3-(4-Bromobenzyl)-7-chloro-6-fluoroquinazoline-2,4(1H,3H)-dione (300 mg; 0.78 mmol) prepared in Example 48, sodium hydride (63 mg; 1.6 mmol; 60% in oil), and 3 ml of dry DMF were charged in a microwave tube and stirred under nitrogen at rt for 15 min. 2-Bromo-N-methoxy-N-methylpropanamide (550 mg; 2.4 mmol; approximately 85% purity) dissolved in DMF was added and the mixture was heated at 120° C. for 6 h. The mixture was cooled to rt after which the reaction was quenched with dropwise addition of MeOH. The mixture was concentrated under reduced pressure. The residue was dissolved in DCM and washed once with saturated NaHCO₃, three times with water, and once with saturated aqueous NaCl. The organic phase was dried with a phase separator and concentrated under reduced pressure to yield 290 mg of 2-(3-(4-bromobenzyl)-7-chloro-6-fluoro-2,4-dioxo-3,4-dihydroquinazolin-1(2H)-yl)-N-methoxy-N-methylpropanamide. ¹H-NMR (400 MHz, CDCl₃): δ 1.64 (d, 3H), 3.11 (s, 3H), 3.25 (s, 3H), 5.10-5.32 (m, 2H), 5.93-6.03 (m, 1H), 7.36-7.40 (m, 2H), 7.41-7.45 (m, 2H), 7.57 (d, 1H), 7.98 (d, 1H).

### 3-(4-Bromobenzyl)-7-chloro-1-(1-cyclopropyl-1-oxopropan-2-yl)-6-fluoroquinazoline-2,4(1H,3H)-dione

2-(3-(4-Bromobenzyl)-7-chloro-6-fluoro-2,4-dioxo-3,4-dihydroquinazolin-1(2H)-yl)-N-methoxy-N-methylpropanamide (50 mg; 0.10 mmol), a small piece of iodine (approximately 1 mg), and dry THF were placed in a reaction flask under nitrogen and cooled to 0° C. Cyclopropylmagnesium bromide (0.38 ml; 0.20 mmol; 0.5 M in THF) was added dropwise. The mixture was stirred at 0° C. for 0.5 h and at rt for 2 h. The reaction was quenched with dropwise addition of water and the mixture was diluted with 1 M HCl. The mixture was extracted twice with DCM and the organic phase was washed with water and saturated aqueous NaCl. The organic phase was dried with a phase separator, concentrated under reduced pressure, and purified with MS-Trigger to yield 2 mg of 3-(4-bromobenzyl)-7-chloro-1-(1-cyclopropyl-1-oxopropan-2-yl)-6-fluoroquinazoline-2,4(1H,3H)-dione. ¹H-NMR (400 MHz, CDCl₃): δ 0.65-0.75 (m, 1H), 0.84-0.92 (m, 1H), 0.95-1.02 (m, 1H), 1.13-1.21 (m, 1H), 1.64 (d, 3H), 1.77-1.85 (m, 1H), 5.19 (q, 2H), 5.30-5.45 (br s, 1H), 7.10 (d, 1H), 7.39-7.46 (m, 4H), 8.01 (d, 1H).

### Example 104: 3-(4-Bromobenzyl)-7-chloro-8-fluoro-1-(2-hydroxy-2,3-dimethylbutyl)quinazoline-2,4(1H,3H)-dione

### 6-(4-Bromobenzyl)-10-chloro-11-fluoro-3-isopropyl-3-methyl-2,3-dihydrobenzo[e][1,3,7]oxadiazonine-5,7(1H,6H)-dione

3-(4-Bromobenzyl)-7-chloro-8-fluoroquinazoline-2,4(1H,3H)-dione (280 mg; 0.72 mmol) prepared in Example 91, yttrium(III) nitrate hexahydrate (28 mg; 0.072 mmol), 2 ml of dry DMF, and 2-isopropyl-2-methyloxirane (0.2 ml; 1.7 mmol) were charged in a microwave tube and heated at 160° C. for 3 h. After cooling to rt, the mixture was diluted with DCM and washed with saturated aqueous NaHCO₃, water, and saturated aqueous NaCl. The organic phase was dried with a phase separator and concentrated under reduced pressure. The residue was purified with CombiFlash (normal phase silica) to yield 23 mg of 6-(4-bromobenzyl)-10-chloro-11-fluoro-3-isopropyl-3-methyl-2,3-dihydrobenzo[e][1,3,7]oxadiazonine-5,7(1H,6H)-dione. LC-MS (ES) [M+H]⁺: 485.0.

### 3-(4-Bromobenzyl)-7-chloro-8-fluoro-1-(2-hydroxy-2,3-dimethylbutyl)quinazoline-2,4(1H,3H)-dione

6-(4-Bromobenzyl)-10-chloro-11-fluoro-3-isopropyl-3-methyl-2,3-dihydrobenzo[e][1,3,7]oxadiazonine-5,7(1H,6H)-dione (23 mg; 0.048 mmol), LiOH (10 mg; 0.43 mmol), and 1 ml of THF were charged in a microwave tube and heated at 100° C. for 2 h. After cooling to rt, the mixture was diluted with water and extracted twice with DCM. The combined organic phases were washed with saturated aqueous NaCl, dried with a phase separator, and concentrated under reduced pressure. The residue was purified with CombiFlash (normal phase silica) to yield 9 mg of 3-(4-bromobenzyl)-7-chloro-8-fluoro-1-(2-hydroxy-2,3-dimethylbutyl)quinazoline-2,4(1H,3H)-dione. ¹H-NMR (400 MHz, CDCl₃): δ 1.00 (d, 3H), 1.02 (dd, 6H), 1.79 (m, 1H), 2.25-2.60 (br s, 1H), 4.56 (dd, 2H), 5.20 (dd, 2H), 7.25-7.30 (m, 1H), 7.34-7.38 (m, 2H), 7.40-7.45 (m, 2H), 8.01 (dd, 1H).

### Example 105: 7-Chloro-8-fluoro-1-(2-hydroxy-2,3-dimethylbutyl)-3-(4-methoxybenzyl)quinazoline-2,4(1H,3H)-dione

### 2-Amino-4-chloro-3-fluoro-N-(4-methoxybenzyl)benzamide

2-Amino-4-chloro-3-fluorobenzoic acid (0.50 g; 2.6 mmol), 10 ml of dry DCM, and TEA (1.5 ml; 11 mmol) were placed in a reaction flask under nitrogen. 4-Methoxybenzylamine (0.41 ml; 3.2 mmol) was added slowly and then T3P (3.1 ml; 5.3 mmol; 50% in EtOAc) was added keeping the temperature below 30° C. The mixture was stirred at rt for 1 h. DCM was added and the mixture was washed twice with water. The organic phase was dried with a phase separator and concentrated under reduced pressure to yield 0.72 g of 2-amino-4-chloro-3-fluoro-N-(4-methoxybenzyl)benzamide. ¹H-NMR (400 MHz, d₆-DMSO): δ 3.72 (s, 3H), 4.35 (d, 2H), 6.65-6.72 (m, 3H), 6.86-6.91 (m, 2H), 7.21-7.27 (m, 2H), 7.43 (dd, 1H), 8.93 (t, 1H).

### 7-Chloro-8-fluoro-3-(4-methoxybenzyl)quinazoline-2,4(1H,3H)-dione

2-Amino-4-chloro-3-fluoro-N-(4-methoxybenzyl)benzamide (0.72 g; 2.3 mmol) and 4 ml of dry pyridine were placed in a reaction flask under nitrogen. Ethyl chloroformate (0.67 ml; 7.0 mmol) was added dropwise at 0° C. The mixture was stirred at rt for 2 h to complete carbamate formation. 2 M NaOH (3.5 ml; 7.0 mmol) was added dropwise and the mixture was stirred at rt overnight. The mixture was partially concentrated and the residue was diluted with DCM. Water and 1 M HCl were added till pH<4 and the precipitation formed was filtered, washed with water, and dried under reduced pressure at 50° C. to yield 0.25 g of 7-chloro-8-fluoro-3-(4-methoxybenzyl)quinazoline-2,4(1H,3H)-dione. ¹H-NMR (400 MHz, d₆-DMSO): δ 3.71 (s, 3H), 5.01 (s, 2H), 6.84-6.89 (m, 2H), 7.26-7.32 (m, 2H), 7.33-7.39 (dd, 1H), 7.77 (dd, 1H), 11.89 (br s, 1H).

### 7-Chloro-8-fluoro-1-(2-hydroxy-2,3-dimethylbutyl)-3-(4-methoxybenzyl)quinazoline-2,4(1H,3H)-dione

7-Chloro-8-fluoro-3-(4-methoxybenzyl)quinazoline-2,4(1H,3H)-dione (90 mg; 0.26 mmol), yttrium(III) nitrate hexahydrate (10 mg; 0.03 mmol), 1 ml of dry DMF, and 2-isopropyl-2-methyloxirane (0.39 ml; 3.40 mmol) were charged in a microwave tube and heated at 160° C. for 2.5 h. After cooling to rt, the mixture was diluted with saturated NaHCO₃. The mixture was extracted twice with DCM and washed twice with water and once with saturated aqueous NaCl. The organic phase was dried with a phase separator and concentrated under reduced pressure. The residue was purified with CombiFlash (normal phase silica) to yield 25 mg of ¹H-NMR (400 MHz, CDCl₃): δ 0.99-1.05 (m, 9H), 1.73-1.87 (m, 1H), 2.45-2.85 (br s, 1H), 3.76 (s, 3H), 4.55 (dd, 2H), 5.19 (dd, 2H), 6.79-6.85 (m, 2H), 7.24-7.28 (dd, 1H), 7.41-7-0.48 (m, 2H), 8.00 (dd, 1H).

### Example 106: 7-Chloro-3-((2,3-dihydrobenzofuran-5-yl)methyl)-1-(2-hydroxy-2-methylpropyl)quinazoline-2,4(1H,3H)-dione

### 2-Amino-4-chloro-N-((2,3-dihydrobenzofuran-5-yl)methyl)benzamide

2-Amino-4-chlorobenzoic acid (0.5 g; 2.91 mmol), 15 ml of dry DCM, and TEA (1.625 ml; 11.66 mmol) were placed in a reaction flask under nitrogen. (2,3-Dihydrobenzofuran-5-yl)methanamine (0.567 ml; 4.37 mmol) was added slowly and then T3P (3.43 ml; 5.83 mmol; 50% in EtOAc) was added keeping the temperature at rt. The mixture was stirred at rt overnight. The reaction mixture was diluted with DCM and washed three times with water. The organic phase was dried with a phase separator and evaporated to dryness to yield 0.546 g of 2-amino-4-chloro-N-((2,3-dihydrobenzofuran-5-yl)methyl)benzamide. LC-MS (ES) [M+1]: 303.1.

7-Chloro-3-((2,3-dihydrobenzofuran-5-yl)methyl)quinazoline-2,4(1H,3H)-dione

2-Amino-4-chloro-N-((2,3-dihydrobenzofuran-5-yl)methyl)benzamide (0.546 g; 1.803 mmol) and 2.5 ml of dry pyridine were placed in a reaction flask under nitrogen. Ethyl chloroformate (0.515 ml; 5.41 mmol) was added dropwise at 0° C. The mixture was stirred at rt overnight to complete carbamate formation. 2 M NaOH (2.71 ml; 5.41 mmol) was added dropwise and the mixture was stirred at 50° C. for 2.5 h. 2M NaOH (2.71 ml; 5.41 mmol) was added again and the mixture was stirred at rt overnight. The mixture was evaporated to dryness and the residue was diluted with DCM. Water and 1 M HCl were added till pH was acidic and the precipitation formed was filtered, washed with water, and dried in a vacuum oven to yield 0.277 g of 7-chloro-3-((2,3-dihydrobenzofuran-5-yl)methyl)quinazoline-2,4(1H,3H)-dione. ¹H-NMR (400 MHz, d₆-DMSO): δ 3.12 (t, 2H), 4.47 (t, 2H), 4.97 (s, 2H), 6.67 (d, 1H), 7.09 (m, 1H), 7.19-7.23 (m, 2H), 7.25 (dd, 1H), 7.94 (d, 1H), 11.60 (s, 1H).

### 7-Chloro-3-((2,3-dihydrobenzofuran-5-yl)methyl)-1-(2-hydroxy-2-methylpropyl)quinazoline-2,4(1H,3H)-dione

7-Chloro-3-((2,3-dihydrobenzofuran-5-yl)methyl)quinazoline-2,4(1H,3H)-dione (150 mg; 0.456 mmol), yttrium(III) nitrate hexahydrate (17.48 mg; 0.046 mmol), 1 ml of dry DMF, and isobutylene oxide (1.216 ml; 13.69 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 94 mg of 7-chloro-3-((2,3-dihydrobenzofuran-5-yl)methyl)-1-(2-hydroxy-2-methylpropyl)quinazoline-2,4(1H,3H)-dione. ¹H-NMR (400 MHz, CDCl₃): δ 1.33 (s, 6H), 2.61 (s, 1H), 3.16 (t, 2H), 4.18 (s, 2H), 4.53 (t, 2H), 5.18 (s, 2H), 6.70 (d, 1H), 7.21 (dd, 1H), 7.29 (m, 1H), 7.38 (m, 1H), 7.47 (d, 1H), 8.16 (d, 1H).

### Example 107: (S)-7-Chloro-6-fluoro-1-(2-hydroxypropyl)-3-(4-methoxybenzyl)quinazoline-2,4(1H,3H)-dione

7-Chloro-6-fluoro-3-(4-methoxybenzyl)quinazoline-2,4(1H,3H)-dione (100 mg; 0.30 mmol) prepared in Example 54, yttrium(III) nitrate hexahydrate (11 mg; 0.03 mmol), 2 ml of dry DMF, and (S)-2-methyloxirane (0.21 ml; 3.0 mmol) were charged in a microwave tube and heated at 160° C. for 1 h. After cooling to rt, the mixture was diluted with saturated NaHCO₃. The mixture was extracted twice with DCM and washed twice with water and once with saturated aqueous NaCl. The organic phase was dried with a phase separator and concentrated under reduced pressure. The residue was purified with CombiFlash (normal phase silica) to yield 64 mg of (S)-7-chloro-6-fluoro-1-(2-hydroxypropyl)-3-(4-methoxybenzyl)quinazoline-2,4(1H,3H)-dione. ¹H-NMR (400 MHz, CDCl₃): δ 1.34 (d, 3H), 2.56 (d, 1H), 3.73 (s, 3H), 3.99-4.11 (m, 2H), 4.18-4.32 (m, 1H), 5.11 (s, 2H), 6.76-6.83 (m, 2H), 7.38-7.47 (m, 3H), 7.85-7.90 (d, 1H).

### Example 108: (S)-7-Chloro-3-(1-(4-chlorophenyl)ethyl)-6-fluoro-1-((3-methyloxetan-3-yl)methyl)quinazoline-2,4(1H,3H)-dione

The preparation of (S)-7-chloro-3-(1-(4-chlorophenyl)ethyl)-6-fluoroquinazoline-2,4(1H,3H)-dione is described in Example 98. (S)-7-Chloro-3-(1-(4-chlorophenyl)ethyl)-6-fluoroquinazoline-2,4(1H,3H)-dione (200 mg; 0.43 mmol), sodium hydride (45 mg; 1.13 mmol; 60% in oil) and 1.5 ml of ACN were placed in a microwave reaction vessel and bubbled with nitrogen. The mixture was stirred at rt for 15 min. 3-(Chloromethyl)-3-methyloxetane (0.25 ml; 2.26 mmol) was added and the microwave reaction was continued for 2 h at 160° C. (absorption high) to complete the reaction. MeOH was added and the reaction mixture was evaporated to dryness. The residue was dissolved in DCM and washed with saturated NaHCO₃ and then twice with water. Organic phase was dried by filtration through a phase separator and evaporated to dryness. The crude material was purified with column chromatography twice (first C18; ACN:water gradient and then normal phase silica; EtOAc:heptane gradient) to give 65 mg of (S)-7-chloro-3-(1-(4-chlorophenyl)ethyl)-6-fluoro-1-((3-methyloxetan-3-yl)methyl)quinazoline-2,4(1H,3H)-dione. ¹H-NMR (400 MHz, CDCl₃): δ 1.43 (s, 3H), 1.89 (d, 3H), 4.07 (q, 2H), 4.18-4.24 (m, 2H), 4.58 (dd, 2H), 6.36 (q, 1H), 7.05 (d, 1H), 7.25-7.31 (m, 2H), 7.33-7.39 (m, 2H), 7.97 (d, 1H).

### Example 109: 10-Chloro-6-(4-methoxybenzyl)-2,2-dimethyl-2H-[1,4]oxazino[2,3,4-ij]quinazoline-5,7(3H,6H)-dione

### 7-Chloro-8-fluoro-1-(2-hydroxy-2-methylpropyl)-3-(4-methoxybenzyl)quinazoline-2,4(1H,3H)-dione

The preparation of 7-chloro-8-fluoro-3-(4-methoxybenzyl)quinazoline-2,4(1H,3H)-dione is described in Example 105. 7-Chloro-8-fluoro-3-(4-methoxybenzyl)quinazoline-2,4(1H,3H)-dione (150 mg; 0.448 mmol), yttrium(III) nitrate hexahydrate (17.16 mg; 0.045 mmol), 1 ml of dry DMF, and isobutylene oxide (1.194 ml; 13.44 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 68 mg of 7-chloro-8-fluoro-1-(2-hydroxy-2-methylpropyl)-3-(4-methoxybenzyl)quinazoline-2,4(1H,3H)-dione. ¹H-NMR (400 MHz, CDCl₃): δ 1.25 (s, 6H), 2.88 (s, 1H), 3.77 (s, 3H), 4.49 (s, 2H), 5.20 (s, 2H), 6.78-6.86 (m, 2H), 7.24-7.30 (m, 1H), 7.40-7.50 (m, 2H), 8.02 (dd, 1H).

### 10-Chloro-6-(4-methoxybenzyl)-2,2-dimethyl-2H-[1,4]oxazino[2,3,4-ij]quinazoline-5,7(3H,6H)-dione

7-Chloro-8-fluoro-1-(2-hydroxy-2-methylpropyl)-3-(4-methoxybenzyl)quinazoline-2,4(1H,3H)-dione (50 mg; 0.123 mmol), sodium hydride (9.83 mg; 0.246 mmol; 60% in oil), and 2 ml of dry THF were placed in a reaction flask under nitrogen. The reaction mixture was stirred at rt for 1 h. The mixture was diluted with DCM and washed twice with water. The organic phase was dried with a phase separator and evaporated to dryness to yield 41 mg of 10-chloro-6-(4-methoxybenzyl)-2,2-dimethyl-2H-[1,4]oxazino[2,3,4-ij]quinazoline-5,7(3H,6H)-dione. ¹H-NMR (400 MHz, CDCl₃): δ 1.44 (s, 6H), 3.77 (s, 3H), 3.90 (s, 2H), 5.18 (s, 2H), 6.79-6.87 (m, 2H), 7.20 (d, 1H), 7.46-7.52 (m, 2H), 7.69 (d, 1H).

### Example 110: 6,8-Difluoro-1-(2-hydroxy-2-methylpropyl)-3-(4-methoxybenzyl)quinazoline-2,4(1H,3H)-dione

### 2-Amino-3,5-difluoro-N-(4-methoxybenzyl)benzamide

2-Amino-3,5-difluorobenzoic acid (1.0 g; 5.78 mmol), 10 ml of dry DCM, and TEA (2.42 ml; 17.3 mmol) were placed in a reaction flask under nitrogen. The reaction mixture was cooled down, 4-methoxybenzylamine (0.755 ml; 5.78 mmol) was added slowly and then T3P (4.1 ml; 6.93 mmol; 50% in EtOAc) was added keeping the temperature at rt. The mixture was stirred at rt over three nights. Water was added and the precipitation was filtered and dried in a vacuum oven to yield 1.0 g of 2-amino-3,5-difluoro-N-(4-methoxybenzyl)benzamide. ¹H-NMR (400 MHz, d₆-DMSO): δ 3.73 (s, 3H), 4.35 (d, 2H), 6.22 (br s, 2H), 6.90 (m, 2H), 7.20-7.37 (m, 4H), 8.91 (t, 1H).

### 6,8-Difluoro-3-(4-methoxybenzyl)quinazoline-2,4(1H,3H)-dione

2-Amino-3,5-difluoro-N-(4-methoxybenzyl)benzamide (1.68 g; 5.78 mmol), 10 ml of dry THF, and 3 ml of dry pyridine were placed in a reaction flask under nitrogen. Ethyl chloroformate (1.65 ml; 17.33 mmol) was added dropwise at 0° C. The mixture was stirred at rt for 2 h to complete carbamate formation. 2 M NaOH (14.4 ml; 28.9 mmol) was added dropwise and the mixture was stirred at rt overnight. The mixture was evaporated to dryness and the residue was diluted with DCM. Water and 1 M HCl were added till pH was acidic and the precipitation formed was filtered, washed with water, and dried in a vacuum oven to yield 0.5 g of 6,8-difluoro-3-(4-methoxybenzyl)quinazoline-2,4(1H,3H)-dione. ¹H-NMR (400 MHz, d₆-DMSO): δ 3.71 (s, 3H), 5.02 (s, 2H), 6.82-6.91 (m, 2H), 7.25-7.33 (m, 2H), 7.50-7.57 (m, 1H), 7.69-7.78 (m, 1H), 11.73 (br s, 1H).

### 6,8-Difluoro-1-(2-hydroxy-2-methylpropyl)-3-(4-methoxybenzyl)quinazoline-2,4(1H,3H)-dione

6,8-Difluoro-3-(4-methoxybenzyl)quinazoline-2,4(1H,3H)-dione (150 mg; 0.47 mmol), yttrium(III) nitrate hexahydrate (18.0 mg; 0.047 mmol), 1 ml of dry DMF, and isobutylene oxide (1.256 ml; 14.14 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 68 mg of 6,8-difluoro-1-(2-hydroxy-2-methylpropyl)-3-(4-methoxybenzyl)quinazoline-2,4(1H,3H)-dione. ¹H-NMR (400 MHz, CDCl₃): δ 1.23 (s, 6H), 2.99 (s, 1H), 3.76 (s, 3H), 4.45 (s, 2H), 5.19 (s, 2H), 6.77-6.85 (m, 2H), 7.11-7.19 (m, 1H), 7.41-7.48 (m, 2H), 7.75-7-80 (m, 1H).

### Example 111: 7-Chloro-3-(4-(difluoromethoxy)benzyl)-1-(2-hydroxy-2-methylpropyl)quinazoline-2,4(1H,3H)-dione

### 2-Amino-4-chloro-N-(4-(difluoromethoxy)benzyl)benzamide

2-Amino-4-chlorobenzoic acid (500 mg; 2.91 mmol), 15 ml of dry DCM, and TEA (1.625 ml; 11.66 mmol) were placed in a reaction flask under nitrogen. 4-(Difluoromethoxy)benzylamine (0.548 ml; 3.79 mmol) was added slowly and then T3P (3.43 ml; 5.83 mmol; 50% in EtOAc) was added keeping the temperature at rt. The mixture was stirred at rt overnight. The reaction mixture was diluted with DCM and washed three times with water. The organic phase was dried with a phase separator and evaporated to dryness to yield 824 mg of 2-amino-4-chloro-N-(4-(difluoromethoxy)benzyl)benzamide. LC-MS (ES) [M+1]: 327.1.

### 7-Chloro-3-(4-(difluoromethoxy)benzyl)quinazoline-2,4(1H,3H)-dione

2-Amino-4-chloro-N-(4-(difluoromethoxy)benzyl)benzamide (824 mg; 2.52 mmol) and 4 ml of dry pyridine were placed in a reaction flask under nitrogen. Ethyl chloroformate (0.720 ml; 7.57 mmol) was added dropwise at 0° C. The mixture was stirred at rt for 3 h to complete carbamate formation. 2 M NaOH (2.71 ml; 5.41 mmol) was added dropwise and the mixture was stirred at 50° C. for few hours. After cooling, the mixture was evaporated to dryness and the residue was diluted with DCM. Water and 1 M HCl were added till pH was acidic and the precipitation formed was filtered, washed with water, and dried in a vacuum oven to yield 634 mg of 7-chloro-3-(4-(difluoromethoxy)benzyl)quinazoline-2,4(1H,3H)-dione. ¹H-NMR (400 MHz, d₆-DMSO): δ 5.05 (s, 2H), 6.97-7.37 (t, 1H), 7.08-7.15 (m, 2H), 7.21 (dd, 1H), 7.26 (dd, 1H), 7.38-7.41 (m, 2H), 7.94 (d, 1H), 11.64 (br s, 1H).

### 7-Chloro-3-(4-(difluoromethoxy)benzyl)-1-(2-hydroxy-2-methylpropyl)quinazoline-2,4(1H,3H)-dione

7-Chloro-3-(4-(difluoromethoxy)benzyl)quinazoline-2,4(1H,3H)-dione (150 mg; 0.425 mmol), yttrium(III) nitrate hexahydrate (16.29 mg; 0.043 mmol), 1 ml of dry DMF, and isobutylene oxide (1.133 ml; 12.76 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 126 mg of 7-chloro-3-(4-(difluoromethoxy)benzyl)-1-(2-hydroxy-2-methylpropyl)quinazoline-2,4(1H,3H)-dione. ¹H-NMR (400 MHz, CDCl₃): δ 1.33 (s, 6H), 2.47 (s, 1H), 4.18 (s, 2H), 5.24 (s, 2H), 6.19-6.69 (t, 1H), 7.02-7.08 (m, 2H), 7.22 (dd, 1H), 7.42-7.57 (m, 3H), 8.16 (d, 1H).

### Example 112: (R)-7-Chloro-3-(1-(4-chlorophenyl)ethyl)-1-methylquinazoline-2,4(1H,3H)-dione

### (R)-2-Amino-4-chloro-N-(1-(4-chlorophenyl)ethyl)benzamide

2-Amino-4-chlorobenzoic acid (2.5 g; 14.57 mmol), DCM (25 ml), and TEA (8.12 ml; 58.3 mmol) were placed in a reaction vessel under nitrogen. (R)-1-(4-Chlorophenyl)ethylamine (2.45 ml; 17.48 mmol) and T3P (17.17 ml; 29.1 mmol; 50% in EtOAc) were added in this order keeping the temperature stable with cooling bath. The reaction mixture was stirred at rt overnight. The mixture was diluted with DCM and washed three times with water. The organic layer was dried with a phase separator funnel and evaporated to dryness to give 4.23 g of (R)-2-amino-4-chloro-N-(1-(4-chlorophenyl)ethyl)benzamide. ¹H-NMR (400 MHz, d₆-DMSO): δ 1.89 (d, 3H), 3.50 (s, 3H), 6.38 (q, 1H), 7.16 (d, 1H), 7.20 (dd, 1H), 7.23-7.29 (m, 2H), 7.34-7.41 (m, 2H), 8.12 (dd, 1H).

### (R)-7-Chloro-3-(1-(4-chlorophenyl)ethyl)quinazoline-2,4(1H,3H)-dione

(R)-2-Amino-4-chloro-N-(1-(4-chlorophenyl)ethyl)benzamide (4.2 g; 13.58 mmol) was dissolved in dry pyridine (25 ml) under nitrogen and cooled to 0° C. Ethyl chloroformate (3.88 ml; 40.8 mmol) was added slowly and the mixture was stirred for 90 min at rt to complete carbamate formation. The solution was cooled to 0° C. and 2 M NaOH (20.38 ml; 40.8 mmol) was added slowly. The solution was stirred for 3.5 h at 50° C. and then over the weekend at rt to complete the reaction. The reaction mixture was evaporated close to dryness. 50 ml of DCM was added and pH was adjusted to acidic with 1 M HCl. The phases were separated and organic phase was washed with water, dried with a phase separator funnel, evaporated to dryness, and dried at 50° C. to give 4.16 g of (R)-7-chloro-3-(1-(4-chlorophenypethyDquinazoline-2,4(1H,3H)-dione. ¹H-NMR (400 MHz, d₆-DMSO): δ 1.80 (d, 3H), 6.13 (q, 1H), 7.19 (d, 1H), 7.24 (dd, 1H), 7.31-7.40 (m, 4H), 7.89 (d, 1H), 11.52 (br s, 1H).

### (R)-7-Chloro-3-(1-(4-chlorophenyl)ethyl)-1-methylquinazoline-2,4(1H,3H)-dione

(R)-7-Chloro-3-(1-(4-chlorophenyl)ethyl)quinazoline-2,4(1H,3H)-dione (0.15 g; 0.45 mmol), K₂CO₃ (124 mg; 0.90 mmol), and 4 ml of ACN were stirred under nitrogen for 15 min. Iodomethane (0.11 ml; 1.79 mmol) was added and the reaction mixture was stirred overnight at rt. DCM (25 ml) was added. Organic phase was washed three times with 25 ml of water, dried by filtration through a phase separator, and evaporated to dryness. The crude material was purified with column chromatography (normal phase silica; EtOAc:heptane gradient) to give 127 mg of (R)-7-chloro-3-(1-(4-chlorophenyl)ethyl)-1-methylquinazoline-2,4(1H,3H)-dione. ¹H-NMR (400 MHz, CDCl₃): δ 1.89 (d, 3H), 3.50 (s, 3H), 6.37 (q, 1H), 7.16 (d, 1H), 7.20 (dd, 1H), 7.23-7.29 (m, 2H), 7.34-7.41 (m, 2H), 8.12 (d, 1H).

### Example 113: 7-Chloro-3-(3-chloro-4-methoxybenzyl)-1-(2-hydroxy-2-methylpropyl)quinazoline-2,4(1H,3H)-dione

### 2-Amino-4-chloro-N-(3-chloro-4-methoxybenzyl)benzamide

2-Amino-4-chlorobenzoic acid (1.0 g; 5.83 mmol), 15 ml of dry DCM, and TEA (4.87 ml; 35.0 mmol) were placed in a reaction flask under nitrogen. 3-Chloro-4-methoxybenzylamine hydrochloride (1.213 g; 5.83 mmol) was added slowly and then T3P (6.87 ml; 11.66 mmol; 50% in EtOAc) was added keeping the temperature at rt. The mixture was stirred at rt overnight. The reaction mixture was diluted with DCM and washed three times with water. The organic phase was dried with a phase separator and evaporated to dryness to yield 1.58 g of 2-amino-4-chloro-N-(3-chloro-4-methoxybenzyl)benzamide. LC-MS (ES) [M+1]: 327.1.

### 7-Chloro-3-(3-chloro-4-methoxybenzyl)quinazoline-2,4(1H,3H)-dione

2-Amino-4-chloro-N-(3-chloro-4-methoxybenzyl)benzamide (1.582 g; 4.86 mmol) and 7.5 ml of dry pyridine were placed in a reaction flask under nitrogen. Ethyl chloroformate (1.389 ml; 14.59 mmol) was added dropwise at 0° C. The mixture was stirred at rt overnight to complete carbamate formation. 2 M NaOH (7.30 ml; 14.59 mmol) was added dropwise and the mixture was stirred at 50° C. for 2.5 h. After cooling, the mixture was evaporated to dryness and the residue was diluted with DCM. Water and 1 M HCl were added till pH was acidic and the precipitation formed was filtered, washed with water, and dried in a vacuum oven to yield 1.148 g of 7-chloro-3-(3-chloro-4-methoxybenzyl)quinazoline-2,4(1H,3H)-dione. LC-MS (ES) [M+1]: 353.0.

### 7-Chloro-3-(3-chloro-4-methoxybenzyl)-1-(2-hydroxy-2-methylpropyl)quinazoline-2,4(1H,3H)-dione

7-Chloro-3-(3-chloro-4-methoxybenzyl)quinazoline-2,4(1H,3H)-dione (150 mg; 0.427 mmol), yttrium(III) nitrate hexahydrate (16.36 mg; 0.043 mmol), 1 ml of dry DMF, and isobutylene oxide (1.138 ml; 12.81 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 142 mg of 7-chloro-3-(3-chloro-4-methoxybenzyl)-1-(2-hydroxy-2-methylpropyl)quinazoline-2,4(1H,3H)-dione. ¹H-NMR (400 MHz, CDCl₃): δ 1.34 (s, 6H), 2.48 (s, 1H), 3.86 (s, 3H), 4.19 (s, 2H), 5.17 (s, 2H), 6.85 (d, 1H), 7.22 (dd, 1H), 7.40 (dd, 1H), 7.52 (dd, 2H), 8.16 (d, 1H).

### Example 114: 9,10-Difluoro-6-(4-methoxybenzyl)-2,2-dimethyl-2H-[1,4]oxazino[2,3,4-ij]quinazoline-5,7(3H,6H)-dione

The preparation of 6,7,8-trifluoro-1-(2-hydroxy-2-methylpropyl)-3-(4-methoxybenzyl)quinazoline-2,4(1H,3H)-dione is described in Example 65. 6,7,8-Trifluoro-1-(2-hydroxy-2-methylpropyl)-3-(4-methoxybenzyl)quinazoline-2,4(1H,3H)-dione (120 mg; 0.29 mmol), sodium hydride (23 mg; 0.58 mmol; 60% in oil), and 4 ml of dry THF were stirred under nitrogen at rt for 4 h after which the reaction was quenched with dropwise addition of MeOH and water. The mixture was diluted with DCM and the phases were separated. The aqueous phase was extracted with DCM and the combined organic phases were washed with saturated aqueous NaCl. The organic phase was dried with a phase separator and concentrated under reduced pressure. The residue was purified with CombiFlash (normal phase silica) to yield 81 mg of 9,10-difluoro-6-(4-methoxybenzyl)-2,2-dimethyl-2H-[1,4]oxazino[2,3,4-ij]quinazoline-5,7(3H,6H)-dione. ¹H-NMR (400 MHz, CDCl₃): δ 1.45 (s, 6H), 3.77 (s, 3H), 3.88 (s, 2H), 5.17 (s, 2H), 6.81-6.87 (m, 2H), 7.46-7.51 (m, 2H), 7.55 (dd, 1H).

### Example 115: 7-Chloro-3-(4-chloro-3-fluorobenzyl)-1-(2-hydroxy-2-methylpropyl)quinazoline-2,4(1H,3H)-dione

The preparation of 7-chloro-3-(4-chloro-3-fluorobenzyl)quinazoline-2,4(1H,3H)-dione is described in Example 88. 7-Chloro-3-(4-chloro-3-fluorobenzyl)quinazoline-2,4(1H,3H)-dione (150 mg; 0.44 mmol), yttrium(III) nitrate hexahydrate (16.9 mg; 0.044 mmol), 1 ml of DMF, and isobutylene oxide (1.18 ml; 13.27 mmol) were placed in a microwave vial and the mixture was heated (absorption high) for 1 h at 160° C. 15 ml of DCM was added to the cooled mixture. The mixture was washed three times with 25 ml of water. Organic phase was dried and evaporated to dryness to obtain 251 mg of crude material, which was purified with column chromatography (normal phase silica; EtOAc:heptane gradient) to obtain 144 mg of 7-chloro-3-(4-chloro-3-fluorobenzyl)-1-(2-hydroxy-2-methylpropyl)quinazoline-2,4(1H,3H)-dione. ¹H-NMR (400 MHz, CDCl₃): δ 1.33 (s, 6H), 2.38 (s, 1H), 4.18 (s, 2H), 5.21 (s, 2H), 7.20-7.25 (m, 2H), 7.27-7.35 (m, 2H), 7.53 (d, 1H), 8.15 (d, 1H).

### Example 116: 7-Chloro-3-(4-(difluoromethoxy)benzyl)-1-((3-methyloxetan-3-yl)methyl)quinazoline-2,4(1H,3H)-dione

The preparation of 7-chloro-3-(4-(difluoromethoxy)benzyl)quinazoline-2,4(1H,3H)-dione is described in Example 111. 7-Chloro-3-(4-(difluoromethoxy)benzyl)quinazoline-2,4(1H,3H)-dione (150 mg; 0.425 mmol), sodium hydride (34 mg; 0.851 mmol; 60% in oil), 2 ml of dry ACN, and 3-(chloromethyl)-3-methyloxetane (205 mg; 1.701 mmol) were charged in a microwave tube. The mixture was flushed with nitrogen and heated at 160° C. for 2 h. After cooling to rt, an additional batch of 3-(chloromethyl)-3-methyloxetane (102 mg; 0.85 mmol) was added and the mixture was heated at 160° C. for 1 h. The mixture was allowed to cool to rt and diluted with DCM. The solution was washed with saturated NaHCO₃ solution and twice with water. The organic phase was dried with a phase separator and concentrated under reduced pressure. The residue was purified with MS-Trigger to yield 14 mg of 7-chloro-3-(4-(dffluoromethoxy)benzyl)-1-((3-methyloxetan-3-yl)methyl)quinazoline-2,4(1H,3H)-dione. ¹H-NMR (400 MHz, CDCl₃): δ 1.48 (s, 3H), 4.14 (s, 2H), 4.26 (d, 2H), 4.67 (d, 2H), 5.23 (s, 2H), 6.26-6.67 (t, 1H), 7.00 (d, 1H), 7.03-7.08 (m, 2H), 7.24 (dd, 1H), 7.47-7.57 (m, 2H), 8.19 (d, 1H).

### Example 117: (R)-7-Chloro-3-(1-(4-chlorophenyl)ethyl)-1-(2-hydroxy-2-methylpropyl)quinazoline-2,4(1H,3H)-dione

The preparation of (R)-7-chloro-3-(1-(4-chlorophenyl)ethyl)quinazoline-2,4(1H,3H)-dione is described in Example 112. (R)-7-Chloro-3-(1-(4-chlorophenyl)ethyl)quinazoline-2,4(1H,3H)-dione (200 mg; 0.60 mmol), yttrium(III) nitrate hexahydrate (23 mg; 0.06 mmol), 1 ml of DMF, and isobutylene oxide (1.59 ml; 17.9 mmol) were placed in a microwave vial and the mixture was heated (absorption high) for 1 h at 160° C. DCM (15 ml) was added to the cooled mixture and the mixture was washed three times with 25 ml of water. Organic phase was dried and evaporated to dryness to obtain 296 mg of crude material, which was purified with column chromatography (normal phase silica; EtOAc:heptane gradient) to obtain 41 mg of (R)-7-chloro-3-(1-(4-chlorophenyl)ethyl)-1-(2-hydroxy-2-methylpropyl)quinazoline-2,4(1H,3H)-dione. ¹H-NMR (400 MHz, CDCl₃): δ 1.25 (s, 3H), 1.31 (s, 3H), 1.89 (d, 3H), 2.33 (s, 1H), 4.12 (dd, 2H), 6.38 (q, 1H), 7.20 (dd, 1H), 7.25-7.30 (m, 2H), 7.33-7.39 (m, 2H), 7.49 (d, 1H), 8.13 (d, 1H).

### Example 118: (R)-7-Chloro-6-fluoro-1-(2-hydroxypropyl)-3-(4-methoxybenzyl)quinazoline-2,4(1H,3H)-dione

(R)-7-Chloro-6-fluoro-1-(2-hydroxypropyl)-3-(4-methoxybenzyl)quinazoline-2,4(1H,3H)-dione was prepared similarly to (S)-7-chloro-6-fluoro-1-(2-hydroxypropyl)-3-(4-methoxybenzyl)quinazoline-2,4(1H,3H)-dione in Example 107. The reaction of 7-chloro-6-fluoro-3-(4-methoxybenzyl)quinazoline-2,4(1H,3H)-dione (100 mg; 0.30 mmol) with (R)-2-methyloxirane (0.21 ml; 3.0 mmol) yielded 56 mg of (R)-7-chloro-6-fluoro-1-(2-hydroxypropyl)-3-(4-methoxybenzyl)quinazoline-2,4(1H,3H)-di one. ¹H-NMR (400 MHz, CDCl₃): identical to that of (S)-7-chloro-6-fluoro-1-(2-hydroxypropyl)-3-(4-methoxybenzyl)quinazoline-2,4(1H,3H)-dione.

### Example 119: 7-Chloro-8-fluoro-1-(2-hydroxy-2-methylpropyl)-3-(4-(trifluoromethoxy)benzyl)quinazoline-2,4(1H,3H)-dione

### 2-Amino-4-chloro-3-fluoro-N-(4-(trifluoromethoxy)benzyl)benzamide

2-Amino-4-chloro-3-fluorobenzoic acid (500 mg; 2.64 mmol), 10 ml of dry DCM, and TEA (1.47 ml; 10.55 mmol) were placed in a reaction flask under nitrogen. 4-(Trifluoromethoxy)benzylamine (0.423 ml; 2.77 mmol) was added slowly and then T3P (3.11 ml; 5.28 mmol; 50% in EtOAc) was added keeping the temperature at rt. The mixture was stirred at rt overnight. The reaction mixture was diluted with DCM and washed three times with water. The organic phase was dried with a phase separator and evaporated to dryness to yield 1.09 g of 2-amino-4-chloro-3-fluoro-N-(4-(trifluoromethoxy)benzyl)benzamide. LC-MS (ES) [M+1] 363.0.

### 7-Chloro-8-fluoro-3-(4-(trifluoromethoxy)benzyl)quinazoline-2,4(1H,3H)-dione

2-Amino-4-chloro-3-fluoro-N-(4-(trifluoromethoxy)benzyl)benzamide (957 mg; 2.64 mmol), 15 ml of dry THF, and 5 ml of dry pyridine were placed in a reaction flask under nitrogen. Ethyl chloroformate (0.754 ml; 7.92 mmol) was added dropwise at 0° C. The mixture was stirred at rt for 2 h to complete carbamate formation. 2 M NaOH (3.96 ml; 7.92 mmol) was added dropwise and the mixture was stirred at rt overnight. The mixture was evaporated almost to dryness and the residue was diluted with DCM. Water and 1 M HCl were added till pH was acidic and the precipitation formed was filtered, washed with water, and dried in a vacuum oven to yield 473 mg of 7-chloro-8-fluoro-3-(4-(trifluoromethoxy)benzyl)quinazoline-2,4(1H,3H)-dione. ¹H-NMR (400 MHz, d₆-DMSO): δ 5.10 (s, 2H), 7.29-7.33 (m, 2H), 7.35-7.41 (m, 1H), 7.43-7.50 (m, 2H), 7.78 (dd, 1H), 11.95 (s, 1H).

### 7-Chloro-8-fluoro-1-(2-hydroxy-2-methylpropyl)-3-(4-(trifluoromethoxy)benzyl)quinazoline-2,4(1H,3H)-dione

7-Chloro-8-fluoro-3-(4-(trifluoromethoxy)benzyl)quinazoline-2,4(1H,3H)-dione (200 mg; 0.515 mmol), yttrium(III) nitrate hexahydrate (19.71 mg; 0.051 mmol), 1 ml of dry DMF, and isobutylene oxide (1.371 ml; 15.44 mmol) were charged in a microwave tube and heated at 160° C. for 1 h. After cooling to rt, the mixture was diluted with DCM and washed four times with water. The organic phase was dried with a phase separator and concentrated under reduced pressure. The residue was purified with flash chromatography to yield 120 mg of 7-chloro-8-fluoro-1-(2-hydroxy-2-methylpropyl)-3-(4-(trifluoromethoxy)benzyl)quinazoline-2,4(1H,3H)-dione. ¹H-NMR (400 MHz, CDCl₃): δ 1.25 (s, 6H), 2.68 (s, 1H), 4.51 (s, 2H), 5.25 (s, 2H), 7.11-7.17 (m, 2H), 7.29 (dd, 1H), 7.49-7.56 (m, 2H), 8.02 (dd, 1H).

### Example 120: (R)-7-Chloro-6-fluoro-1-(2-hydroxy-2-methylpropyl)-3-(1-(4-methoxyphenyl)ethyDquinazoline-2,4(1H,3H)-dione

### (R)-2-Amino-4-chloro-5-fluoro-N-(1-(4-methoxyphenyl)ethyl)benzamide

2-Amino-4-chloro-5-fluorobenzoic acid (2.5 g; 13.19 mmol), DCM (25 ml), and TEA (7.35 ml; 52.8 mmol) were placed in a reaction vessel under nitrogen. (R)-1-(4-Methoxyphenyl)ethylamine (2.34 ml; 15.83 mmol) and T3P (15.54 ml; 26.4 mmol; 50% in EtOAc) were added in this order keeping the temperature stable with cooling bath. The reaction mixture was stirred at rt for 2 h. The mixture was diluted with 25 ml of DCM and washed three times with 50 ml of water. 5 ml of brine was added to the last wash. The organic layer was dried by filtration through a phase separator funnel and evaporated to dryness to give 4.1 g of (R)-2-amino-4-chloro-5-fluoro-N-(1-(4-methoxyphenyl)ethyl)benzamide. ¹H-NMR (400 MHz, d₆-DMSO): δ 1.43 (d, 3H), 3.72 (s, 3H), 5.05 (quint, 1H), 6.46 (br s, 2H), 6.81-6.92 (m, 3H), 7.25-7.32 (m, 2H), 7.66 (d, 1H), 8.58 (d, 1H).

### (R)-7-Chloro-6-fluoro-3-(1-(4-methoxyphenyl)ethyl)quinazoline-2,4(1H,3H)-dione

(R)-2-Amino-4-chloro-5-fluoro-N-(1-(4-methoxyphenyl)ethyl)benzamide (4.1 g; 12.70 mmol) was dissolved in 20 ml of dry pyridine under nitrogen and cooled to 0° C. Ethyl chloroformate (3.88 ml; 40.8 mmol) was added slowly keeping the temperature at rt with ice bath and then the mixture was stirred overnight at rt. The solution was cooled to 0° C. and 2 M NaOH (19.0 ml; 38.1 mmol) was added slowly. The solution was stirred for 2 h at 50° C. 10 ml of 2 M NaOH was added and the solution was stirred 5 h at 50° C. and then overnight at rt to complete ring closure. The reaction mixture was evaporated close to dryness. 50 ml of DCM was added and pH was adjusted to acidic with 1 M HCl. The phases were separated. Organic phase was washed with water, dried, and evaporated to dryness to obtain 4.121 g of (R)-7-chloro-6-fluoro-3-(1-(4-methoxyphenyl)ethyl)quinazoline-2,4(1H,3H)-dione. ¹H-NMR (400 MHz, d₆-DMSO): δ 1.79 (d, 3H), 3.72 (s, 3H), 6.11 (q, 1H), 6.81-6.89 (m, 2H), 7.23-7.29 (m, 2H), 7.80 (d, 1H), 11.49 (br s, 1H).

### (R)-7-Chloro-6-fluoro-1-(2-hydroxy-2-methylpropyl)-3-(1-(4-methoxyphenyl)ethyl)quinazoline-2,4(1H,3H)-dione

(R)-7-Chloro-6-fluoro-3-(1-(4-methoxyphenyl)ethyl)quinazoline-2,4(1H,3H)-dione (200 mg; 0.57 mmol), 1 ml of dry DMF, yttrium(III) nitrate hexahydrate (22.0 mg; 0.057 mmol), and isobutylene oxide (1.53 ml; 17.20 mmol) were placed in a microwave vial under nitrogen and the mixture was heated (absorption high) for 1 h at 160° C. 15 ml of DCM was added and the mixture was washed three times with 25 ml of water. Some DCM and brine were added to the last wash. Organic phases were combined, dried by filtration through a phase separation funnel, and evaporated to dryness to obtain 346 mg of crude product. Column chromatography purification (normal phase silica; EtOAc:heptane gradient) gave 169 mg of (R)-7-chloro-6-fluoro-1-(2-hydroxy-2-methylpropyl)-3-(1-(4-methoxyphenyl)ethyl)quinazoline-2,4(1H,3H)-dione. ¹H-NMR (400 MHz, CDCl₃): δ 1.25 (s, 3H), 1.32 (s, 3H), 1.89 (d, 3H), 2.22 (br s, 1H), 3.77 (s, 3H), 4.11 (dd, 2H), 6.37 (q, 1H), 6.80-6.87 (m, 2H), 7.34-7.41 (m, 2H), 7.60 (d, 1H), 7.93 (d, 1H).

### Example 121: 10-Chloro-2-isopropyl-6-(4-methoxybenzyl)-2-methyl-2H-[1,4]oxazino[2,3,4-ij]quinazoline-5,7(3H,6H)-dione

### 10-Chloro-11-fluoro-3-isopropyl-6-(4-methoxybenzyl)-3-methyl-2,3-dihydrobenzo[e][1,3,7]oxadiazonine-5,7(1H,6H)-dione

10-Chloro-11-fluoro-3-isopropyl-6-(4-methoxybenzyl)-3-methyl-2,3-dihydrobenzo[e][1,3,7]oxadiazonine-5,7(1H,6H)-dion was isolated from the reaction of 7-chloro-8-fluoro-3-(4-methoxybenzyl)quinazoline-2,4(1H,3H)-dione (90 mg; 0.26 mmol) with 2-isopropyl-2-methyloxirane (0.39 ml; 3.40 mmol) described in Example 105. Purification with CombiFlash (normal phase silica) yielded 65 mg of 10-chloro-11-fluoro-3-isopropyl-6-(4-methoxybenzyl)-3-methyl-2,3-dihydrobenzo[e][1,3,7]oxadiazonine-5,7(1H,6H)-dione. ¹H-NMR (400 MHz, CDCl₃): δ 0.90-1.06 (br s, 3H), 1.02 (d, 3H), 1.30-1.55 (br s, 3H), 1.80-2.15 (br s, 1H), 3.46-3.56 (m, 1H), 3.70-3.90 (m, 1H), 3.79 (s, 3H), 4.46-4.52 (m, 2H), 6.56-6.75 (br s, 1H), 6.85-6.90 (m, 2H), 7.25-7.31 (m, 3H), 7.44 (dd, 1H).

### 10-Chloro-2-isopropyl-6-(4-methoxybenzyl)-2-methyl-2H-[1,4]oxazino[2,3,4-ij]quinazoline-5,7(3H,6H)-dione

10-Chloro-11-fluoro-3-isopropyl-6-(4-methoxybenzyl)-3-methyl-2,3-dihydrobenzo[e][1,3,7]oxadiazonine-5,7(1H,6H)-dione (65 mg, 0.15 mmol), LiOH (22 mg; 0.92 mmol), and 1 ml of dry THF were charged in a microwave tube and heated at 120° C. for 1 h. After cooling to rt, the mixture was diluted with water, extracted twice with DCM, and washed with saturated aqueous NaCl. The organic phase was dried with a phase separator and concentrated under reduced pressure. The residue was purified with CombiFlash (normal phase silica) to yield 15 mg of 10-chloro-2-isopropyl-6-(4-methoxybenzyl)-2-methyl-2H-[1,4]oxazino[2,3,4-ij]quinazoline-5,7(3H,6H)-dione. ¹H-NMR (400 MHz, CDCl₃): δ 1.01 (d, 3H), 1.08 (d, 3H), 1.25 (s, 3H), 1.90-2.02 (m, 1H), 3.77 (s, 3H), 3.95 (q, 2H), 5.17 (q, 2H), 6.80-6.86 (m, 2H), 7.17-7.21 (d, 1H), 7.47-7.52 (m, 2H), 7.67 (d, 1H).

### Example 122: (S)-3-(4-Bromobenzyl)-7-chloro-1-(2-hydroxypropyl)quinazoline-2,4(1H,3H)-dione

(S)-3-(4-bromobenzyl)-7-chloro-1-(2-hydroxypropyl)quinazoline-2,4(1H,3H)-dione was prepared similarly to (R)-3-(4-bromobenzyl)-7-chloro-1-(2-hydroxypropyl)quinazoline-2,4(1H,3H)-dione in Example 68. The reaction of 3-(4-broniobenzyl)-7-chloroquinazoline-2,4(1H,3H)-dione (300 mg; 034 mmol) with (S)-2-methyloxirane (0.52 ml; 7.4 mmol) yielded 220 mg of (S)-3-(4-bromobenzyl)-7-chloro-1-(2-hydroxypropyl)quinazoline-2,4(1H,3H)-dione. ¹H-NMR (400 MHz, CDCl₃): identical to that of (R)-3-(4-bromobenzyl)-7-chloro-1-(2-hydroxypropyl)quinazoline-2,4(1H,3H)-dione.

### Example 123: 3-(4-Bromobenzyl)-7-chloro-8-fluoro-1-(2-hydroxy-2-methylpropyl)quinazoline-2,4(1H,3H)-dione

The preparation of 3-(4-bromobenzyl)-7-chloro-8-fluoroquinazoline-2,4(1H,3H)-dione is described in Example 91. 3-(4-Bromobenzyl)-7-chloro-8-fluoroquinazoline-2,4(1H,3H)-dione (150 mg; 0.39 mmol), yttrium(III) nitrate hexahydrate (14.98 mg; 0.039 mmol), 1 ml of dry DMF, and isobutylene oxide (1.042 ml; 11.73 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 85 mg of 3-(4-bromobenzyl)-7-chloro-8-fluoro-1-(2-hydroxy-2-methylpropyl)quinazoline-2,4(1H,3H)-dione. ¹H-NMR (400 MHz, CDCl₃): δ 1.24 (s, 6H), 2.79 (s, 1H), 4.49 (s, 2H), 5.18 (s, 2H), 7.21-7.31 (m, 1H), 7.32-7.45 (m, 4H), 7.89-8.05 (m, 1H).

### Example 124: (R)-7-Chloro-3-(4-chlorobenzyl)-1-(2-hydroxypropyl)quinazoline-2,4(1H,3H)-dione

### 2-Amino-4-chloro-N-(4-chlorobenzyl)benzamide

2-Amino-4-chlorobenzoic acid (0.6 g; 3.5 mmol), 12 ml of dry DCM, and TEA (1.5 ml; 11 mmol) were placed in a reaction flask under nitrogen. 4-Chlorobenzylamine (0.47 ml; 3.9 mmol) was added slowly and then T3P (2.7 ml; 4.6 mmol; 50% in DMF) was added keeping the temperature below 30° C. The mixture was stirred at rt overnight. DCM was added and the mixture was washed four times with water and once with saturated aqueous NaCl. The organic phase was dried with a phase separator and concentrated under reduced pressure to yield 0.77 g of 2-amino-4-chloro-N-(4-chlorobenzyl)benzamide. ¹H-NMR (400 MHz, CDCl₃): δ 4.54 (d, 2H), 5.55-5.85 (br s, 2H), 6.20-6.40 (br s, 1H), 6.58 (dd, 1H), 6.68 (d, 1H), 7.20-7.29 (m, 3H), 7.29-7.34 (m, 2H).

### 7-Chloro-3-(4-chlorobenzyl)quinazoline-2,4(1H,3H)-dione

2-Amino-4-chloro-N-(4-chlorobenzyl)benzamide (0.76 g; 2.6 mmol), 4 ml of dry THF, and 1 ml of dry pyridine were placed in a reaction flask under nitrogen. Ethyl chloroformate (0.74 ml; 7.7 mmol) was added dropwise at 0° C. The mixture was stirred at rt for 2 h to complete carbamate formation. 2 M NaOH (6.4 ml; 12.9 mmol) was added dropwise and the mixture was stirred at rt overnight. The mixture was partially concentrated and the residue was diluted with DCM. Water and 1 M HCl were added till pH<4 and the precipitation formed was filtered, washed with water, and dried under reduced pressure at 40° C. to yield 0.93 g of crude product. ¹H-NMR (400 MHz, d₆-DMSO): δ 5.04 (s, 2H), 6.95-7.00 (m, 1H), 7.08 (d, 1H), 7.29-7.36 (m, 4H), 7.80 (d, 1H).

### (R)-7-Chloro-3-(4-chlorobenzyl)-1-(2-hydroxypropyl)quinazoline-2,4(1H,3H)-dione

7-Chloro-3-(4-chlorobenzyl)quinazoline-2,4(1H,3H)-dione (300 mg; 0.84 mmol), yttrium(III) nitrate hexahydrate (32 mg; 0.084 mmol), 3 ml of dry DMF, and (R)-2-methyloxirane (0.59 ml; 8.4 mmol) were charged in a microwave tube and heated at 160° C. for 1 h. After cooling to rt, the mixture was diluted with DCM and washed with saturated NaHCO₃, water, and saturated aqueous NaCl. The organic phase was dried with a phase separator and concentrated under reduced pressure to yield 230 mg of (R)-7-chloro-3-(4-¹H-NMR (400 MHz, CDCl₃): δ 1.35 (d, 3H), 2.15-2.40 (br s, 1H), 4.05 (dd, 1H), 4.14 (dd, 1H), 4.19-4.29 (m, 1H), 5.19 (d, 2H), 7.21 (dd, 1H), 7.24-7.28 (m, 2H), 7.34 (d, 1H), 7.40-7.46 (m, 2H), 8.14 (d, 1H).

### Example 125: (S)-7-Chloro-3-(1-(4-chlorophenyl)ethyl)-6-fluoro-1-(2-hydroxy-2-methylpropyl)quinazoline-2,4(1H,3H)-dione

The preparation of (S)-7-chloro-3-(1-(4-chlorophenyl)ethyl)-6-fluoroquinazoline-2,4(1H,3H)-dione is described in Example 98. (S)-7-Chloro-3-(1-(4-chlorophenyl)ethyl)-6-fluoroquinazoline-2,4(1H,3H)-dione (200 mg; 0.57 mmol), 1 ml of dry DMF, yttrium(HI) nitrate hexahydrate (21.7 mg; 0.057 mmol), and isobutylene oxide (1.51 ml; 16.99 mmol) were placed in a microwave vial under nitrogen and the mixture was heated (absorption high) for 1 h at 160° C. 15 ml of DCM was added and the mixture was washed three times with 25 ml of water. Organic phase was dried and evaporated to dryness to obtain 243 mg of crude product. Column chromatography purification (normal phase silica; EtOAc:heptane gradient) gave 128 mg of (S)-7-chloro-3-(1-(4-chlorophenyl)ethyl)-6-fluoro-1-(2-hydroxy-2-methylpropyl)quinazoline-2,4(1H,3H)-dione. ¹H-NMR (400 MHz, CDCl₃): δ 1.25 (s, 3H), 1.32 (s, 3H), 1.89 (d, 3H), 2.10 (br s, 1H), 4.10 (dd, 2H), 6.36 (q, 1H), 7.24-7.30 (m, 2H), 7.32-7.38 (m, 2H), 7.63 (d, 1H), 7.93 (d, 1H).

### Example 126: (S)-7-Chloro-3-(4-chlorobenzyl)-1-(2-hydroxypropyl)quinazoline-2,4(1H,3H)-dione

(S)-7-Chloro-3-(4-chlorobenzyl)-1-(2-hydroxypropyl)quinazoline-2,4(1H,3H)-dione was prepared similarly to (R)-7-chloro-3-(4-chlorobenzyl)-1-(2-hydroxypropyl)quinazoline-2,4(1H,3H)-dione in Example 124. The reaction of 7-chloro-3-(4-chlorobenzyl)quinazoline-2,4(1H,3H)-dione (300 mg; 0.84 mmol) with (S)-2-methyloxirane (0.59 ml; 8.4 mmol) yielded 230 mg of (S)-7-chloro-3-(4-chlorobenzyl)-1-(2-hydroxypropyl)quinazoline-2,4(1H,3H)-dione. ¹H-NMR (400 MHz, CDCl₃): identical to that of (R)-7-chloro-3-(4-chlorobenzyl)-1-(2-hydroxypropyl)quinazoline-2,4(1H,3H)-dione.

### Example 127: 6-(4-Bromobenzyl)-10-chloro-2,2-dimethyl-2H-[1,4]oxazino[2,3,4-ij]quinazoline-5,7(3H,6H)-dione

The preparation of 3-(4-bromobenzyl)-7-chloro-8-fluoro-1-(2-hydroxy-2-methylpropyl)quinazoline-2,4(1H,3H)-dione is described in Example 123. 3-(4-Bromobenzyl)-7-chloro-8-fluoro-1-(2-hydroxy-2-methylpropyl)quinazoline-2,4(1H,3H)-dione (50 mg; 0.110 mmol), sodium hydride (8.78 mg; 0.219 mmol; 60% in oil), and 2 ml of dry THF were placed in a reaction flask under nitrogen. The reaction mixture was stirred at rt for 1 h. DCM was added and the mixture was washed twice with water. The organic phase was dried with a phase separator and evaporated to dryness. The residue was purified with flash chromatography to yield 35 mg of 6-(4-bromobenzyl)-10-chloro-2,2-dimethyl-2H-[1,4]oxazino[2,3,4-ij]quinazoline-5,7(3H,6H)-dione. ¹H-NMR (400 MHz, CDCl₃): δ 1.44 (s, 6H), 3.89 (s, 2H), 5.18 (s, 2H), 7.21 (d, 1H), 7.41 (m, 4H), 7.65-7.71 (d, 1H).

### Example 128: 7-Chloro-6-fluoro-1-(2-hydroxy-2,3-dimethylbutyl)-3-(4-methoxybenzyl)quinazoline-2,4(1H,3H)-dione

7-Chloro-6-fluoro-3-(4-methoxybenzyl)quinazoline-2,4(1H,3H)-dione (50 mg; 0.15 mmol) prepared in Example 54, yttrium(III) nitrate hexahydrate (6 mg; 0.02 mmol), 0.6 ml of dry DMF, and 2-isopropyl-2-methyloxirane (0.17 ml; 1.5 mmol) were charged in a microwave tube and heated at 160° C. for 2.5 h. After cooling to rt, the mixture was diluted with saturated NaHCO₃. The mixture was extracted twice with DCM and washed twice with water and once with saturated aqueous NaCl. The organic phase was dried with a phase separator and concentrated under reduced pressure. The residue was purified with CombiFlash (normal phase silica) to yield 44 mg of 7-chloro-6-fluoro-1-(2-hydroxy-2,3-dimethylbutyl)-3-(4-methoxybenzyl)quinazoline-2,4(1H,3H)-dione. ¹H-NMR (400 MHz, CDCl₃): δ 1.06-1.11 (m, 9H), 1.81-1.93 (m, 1H), 2.15-2.40 (br s, 1H), 3.77 (s, 3H), 4.19 (dd, 2H), 5.19 (s, 2H), 6.80-6.85 (m, 2H), 7.43-7.48 (m, 2H), 7.61 (d, 1H), 7.95 (d, 1H).

### Example 129: 9-Fluoro-10-methoxy-6-(4-methoxybenzyl)-2,2-dimethyl-2H-[1,4]oxazino[2,3,4-ij]quinazoline-5,7(3H,6H)-dione

The preparation of 9,10-difluoro-6-(4-methoxybenzyl)-2,2-dimethyl-2H-[1,4]oxazino[2,3,4-ij]quinazoline-5,7(3H,6H)-dione is described in Example 114. 9,10-Difluoro-6-(4-methoxybenzyl)-2,2-dimethyl-2H-[1,4]oxazino[2,3,4-ij]quinazoline-5,7(3H,6H)-dione (55 mg; 0.14 mmol), sodium hydride (10 mg; 0.43 mmol; 60% in oil), and 0.5 ml of dry DMF were placed in a reaction flask under nitrogen. The mixture was cooled to 0° C., MeOH (0.1 ml; 2.5 mmol) was added, and the mixture was stirred at rt for 3 h. The mixture was diluted with DCM and washed twice with water and once with saturated aqueous NaCl. The organic phase was dried with a phase separator and concentrated under reduced pressure to yield 54 mg of 9-fluoro-10-methoxy-6-(4-methoxybenzyl)-2,2-dimethyl-2H-[1,4]oxazino[2,3,4-ij]quinazoline-5,7(3H,6H)-dione. ¹H-NMR (400 MHz, CDCl₃): δ 1.44 (s, 6H), 3.77 (s, 3H), 3.86 (s, 2H), 4.03 (d, 3H), 5.16 (s, 2H), 6.80-6.87 (m, 2H), 7.45-7.52 (m, 3H).

### Example 130: (Z)-7-Chloro-3-(4-chlorobenzyl)-1-(prop-1-en-1-yl)quinazoline-2,4(1H,3H)-dione

The preparation of (R)-7-chloro-3-(4-chlorobenzyl)-1-(2-hydroxypropyl)quinazoline-2,4(1H,3H)-dione is described in Example 124. (R)-7-Chloro-3-(4-chlorobenzyl)-1-(2-hydroxypropyl)quinazoline-2,4(1H,3H)-dione (200 mg; 0.53 mmol) was placed in a reaction flask and 5 ml of dry toluene was added. Thionyl chloride (0.12 ml; 1.6 mmol) was added dropwise and the mixture was stirred at rt overnight. The mixture was concentrated under reduced pressure and the residue was transferred to a microwave tube. 5 ml of dry MeOH and sodium methoxide (140 mg; 2.6 mmol) were added and the mixture was heated at 120° C. for 10 min. After cooling to rt, the mixture was diluted with water and extracted with DCM. The organic phase was washed with saturated aqueous NaCl, dried with a phase separator, and concentrated under reduced pressure. The residue was purified with CombiFlash (normal phase silica) to yield 37 mg of (Z)-7-chloro-3-(4-chlorobenzyl)-1-(prop-1-en-1-yl)quinazoline-2,4(1H,3H)-dione. ¹H-NMR (400 MHz, CDCl₃): δ 1.98 (dd, 3H), 5.19 (s, 2H), 5.98-6.08 (m, 1H), 6.12-6.18 (m, 1H), 7.18-7.22 (m, 1H), 7.23-7.30 (m, 3H), 7.47 (d, 2H), 8.13 (dd, 1H).

### Example 131: 6-Chloro-3-(4-chlorobenzyl)-8-fluoro-1-(2-hydroxy-2-methylpropyl)quinazoline-2,4(1H,3H)-dione

### 2-Amino-5-chloro-N-(4-chlorobenzyl)-3-fluorobenzamide

2-Amino-5-chloro-3-fluorobenzoic acid (2.0 g; 10.55 mmol), 25 ml of dry DCM, and TEA (5.88 ml; 42.2 mmol) were placed in a reaction flask under nitrogen. 4-Chlorobenzylamine (1.668 ml; 13.72 mmol) was added slowly and then T3P (12.43 ml; 21.10 mmol; 50% in EtOAc) was added keeping the temperature at rt. The mixture was stirred at rt overnight. The reaction mixture was diluted with DCM and washed three times with water. The organic phase was dried with a phase separator and evaporated to dryness to yield 3.38 g of 2-amino-5-chloro-N-(4-chlorobenzyl)-3-fluorobenzamide. ¹H-NMR (400 MHz, d₆-DMSO): δ 4.40 (d, 2H), 6.51 (br s, 2H), 7.30-7.43 (m, 5H), 7.52-7.56 (m, 1H), 9.05 (t, 1H).

### 6-Chloro-3-(4-chlorobenzyl)-8-fluoroquinazoline-2,4(1H,3H)-dione

2-Amino-5-chloro-N-(4-chlorobenzyl)-3-fluorobenzamide (3.38 g; 10.79 mmol), 25 ml of dry THF, and 15 ml of dry pyridine were placed in a reaction flask under nitrogen. Ethyl chloroformate (3.08 ml; 32.4 mmol) was added dropwise at 0° C. The mixture was stirred at rt for 2 h to complete carbamate formation. 2 M NaOH (16.19 ml; 32.4 mmol) was added dropwise and the mixture was stirred at rt overnight. The mixture was evaporated almost to dryness and the residue was diluted with DCM. Water and 1 M HCl were added till pH was acidic and the precipitation formed was filtered, washed with water, and dried in a vacuum oven to yield 1.7 g of 6-chloro-3-(4-chlorobenzyl)-8-fluoroquinazoline-2,4(1H,3H)-dione. ¹H-NMR (400 MHz, d₆-DMSO): δ 5.06 (s, 2H), 7.30-7.41 (m, 4H), 7.73 (dd, 1H), 7.84 (dd, 1H), 11.90 (br s, 1H).

### 6-Chloro-3-(4-chlorobenzyl)-8-fluoro-1-(2-hydroxy-2-methylpropyl)quinazoline-2,4(1H,3H)-dione

6-Chloro-3-(4-chlorobenzyl)-8-fluoroquinazoline-2,4(1H,3H)-dione (200 mg; 0.590 mmol), yttrium(III) nitrate hexahydrate (22.59 mg; 0.059 mmol), 1 ml of dry DMF, and isobutylene oxide (1.571 ml; 17.69 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 96 mg of 6-chloro-3-(4-chlorobenzyl)-8-fluoro-1-(2-hydroxy-2-methylpropyl)quinazoline-2,4(1H,3H)-dione. ¹H-NMR (400 MHz, CDCl₃): δ 1.23 (s, 6H), 2.65 (s, 1H), 4.47 (s, 2H), 5.22 (s, 2H), 7.24-7.29 (m, 2H), 7.36-7.46 (m, 3H), 8.06 (dd, 1H).

### Example 132: 7-Chloro-3-(4-chlorobenzyl)-1-(2-hydroxy-2-methylpropyl)quinazoline-2,4(1H,3H)-dione

The preparation of 7-chloro-3-(4-chlorobenzyl)quinazoline-2,4(1H,3H)-dione is described in Example 124. 7-Chloro-3-(4-chlorobenzyl)quinazoline-2,4(1H,3H)-dione (150 mg; 0.47 mmol), yttrium(III) nitrate hexahydrate (17.9 mg; 0.047 mmol), 1 ml of dry DMF, and isobutylene oxide (1.24 ml; 14.01 mmol) were placed in a microwave reaction vial under nitrogen and heated (absorption high) for 1 h at 160° C. DCM (15 ml) was added and the mixture was washed three times with 25 ml of water. Organic phase was dried and evaporated to dryness to obtain 202 mg of crude product, which was purified with column chromatography (normal phase silica; EtOAc:heptane gradient) to give 142 mg of 7-chloro-3-(4-chlorobenzyl)-1-(2-hydroxy-2-methylpropyl)quinazoline-2,4(1H,3H)-dione. ¹H-NMR (400 MHz, CDCl₃): δ 1.33 (s, 6H), 2.46 (s, 1H), 4.18 (s, 2H), 5.22 (s, 2H), 7.22 (dd, 1H), 7.24-7.29 (m, 2H), 7.41-7.47 (m, 2H), 7.51 (dd, 1H), 8.16 (d, 1H).

### Example 133: 3-(4-Bromobenzyl)-6,8-difluoro-1-(2-hydroxy-2-methylpropyl)quinazoline-2,4(1H,3H)-dione

### 2-Amino-N-(4-bromobenzyl)-3,5-difluorobenzamide

2-Amino-3,5-difluorobenzoic acid (1.0 g; 5.78 mmol), 10 ml of dry DCM, and TEA (2.415 ml; 17.33 mmol) were placed in a reaction flask under nitrogen. 4-Bromobenzylamine (0.803 ml; 6.35 mmol) was added slowly and then T3P (4.12 ml; 6.93 mmol; 50% in EtOAc) was added keeping the temperature at rt. The mixture was stirred at rt over three nights. Water was added to the mixture and the precipitation formed was filtered, washed with water, and dried in a vacuum oven to yield 1.03 g of 2-amino-N-(4-bromobenzyl)-3,5-difluorobenzamide. LC-MS (ES) [M+1]: 343.0.

### 3-(4-Bromobenzyl)-6,8-difluoroquinazoline-2,4(1H,3H)-dione

2-Amino-N-(4-bromobenzyl)-3,5-difluorobenzamide (1.03 g; 3.03 mmol), 7.5 ml of dry THF, and 2.5 ml of dry pyridine were placed in a reaction flask under nitrogen. Ethyl chloroformate (0.869 nil; 9.09 mmol) was added dropwise at 0° C. The mixture was stirred at rt for 2 h to complete carbamate formation. 2 M NaOH (7.58 ml; 15.15 mmol) was added dropwise and the mixture was stirred at rt overnight. The mixture was evaporated to dryness and the residue was diluted with DCM. Water and 1 M HCl were added till pH was acidic and the precipitation formed was filtered, washed with water, and dried in a vacuum oven to yield 0.374 g of 3-(4-bromobenzyl)-6,8-difluoroquinazoline-2,4(1H,3H)-dione. ¹H-NMR (400 MHz, d₆-DMSO): δ 5.05 (s, 2H), 7.26-7.33 (m, 2H), 7.47-7.53 (m, 2H), 7.54-7.58 (m, 1H), 7.73-7.82 (m, 1H), 11.80 (br s, 1H).

### 3-(4-Bromobenzyl)-6,8-difluoro-1-(2-hydroxy-2-methylpropyl)quinazoline-2,4(1H,3H)-dione

3-(4-Bromobenzyl)-6,8-difluoroquinazoline-2,4(1H,3H)-dione (225 mg; 0.613 mmol), yttrium(III) nitrate hexahydrate (23.47 mg; 0.061 mmol), 2 ml of dry DMF, and isobutylene oxide (1.633 ml; 18.39 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 55 mg of 3-(4-bromobenzyl)-6,8-difluoro-1-(2-hydroxy-2-methylpropyl)quinazoline-2,4(1H,3H)-dione. ¹H-NMR (400 MHz, CDCl₃): δ 1.24 (s, 6H), 2.73 (s, 1H), 4.47 (s, 2H), 5.21 (s, 2H), 7.18 (ddd, 1H), 7.33-7.46 (m, 4H), 7.79 (ddd, 1H).

### Example 134: 7-Chloro-8-fluoro-1-(2-hydroxy-2-methylpropyl)-3-(4-methoxybenzyl)quinazoline-2,4(1H,3H)-dione

The preparation of 7-chloro-8-fluoro-3-(4-methoxybenzyl)quinazoline-2,4(1H,3H)-dione is described in Example 105. 7-Chloro-8-fluoro-3-(4-methoxybenzyl)quinazoline-2,4(1H,3H)-dione (150 mg; 0.448 mmol), yttrium(III) nitrate hexahydrate (17.16 mg; 0.045 mmol), 1 ml of dry DMF, and isobutylene oxide (1.194 ml; 13.44 mmol) were charged in a microwave tube and heated at 160° C. for 1 h. After cooling to rt, the mixture was diluted with DCM and washed three times with water. The organic phase was dried with a phase separator and concentrated under reduced pressure. The residue was purified with flash chromatography to yield 68 mg of 7-chloro-8-fluoro-1-(2-hydroxy-2-methylpropyl)-3-(4-methoxybenzyl)quinazoline-2,4(1H,3H)-dione. ¹H-NMR (400 MHz, CDCl₃): δ 1.08-1.37 (m, 6H), 2.88 (s, 1H), 3.77 (s, 3H), 4.49 (s, 2H), 5.20 (s, 2H), 6.76-6.88 (m, 2H), 7.23-7.30 (m, 1H), 7.41-7.50 (m, 2H) 8.02 (dd, 1H).

### Example 135: 3-(Benzo[d][1,3]dioxol-5-ylmethyl)-7-chloro-1-(2-hydroxy-2-methylpropyl)quinazoline-2,4(1H,3H)-dione

### 2-Amino-N-(benzo[d][1,3]dioxol-5-ylmethyl)-4-chlorobenzamide

2-Amino-4-chlorobenzoic acid (500 mg; 2.91 mmol), 15 ml of DCM, TEA (1.63 ml; 11.66 mmol), and benzo[d][1,3]dioxol-5-ylmethanamine (0.54 ml; 4.37 mmol) were charged in a reaction flask under nitrogen and cooled to 0° C. T3P (3.43 ml; 5.83 mmol; 50% in EtOAc) was added slowly and the reaction was stirred at rt for 3 days. DCM was added and the solution was washed three times with water, dried by filtration through a phase separator funnel, and evaporated to dryness to give 941 mg of 2-amino-N-(benzo[d][1,3]dioxol-5-yl-methyl)-4-chlorobenzamide. ¹H-NMR (400 MHz, d₆-DMSO): δ 4.31 (d, 2H), 5.97 (s, 2H), 6.53 (dd, 1H), 6.69 (br s, 2H), 6.74-6.80 (m, 2H), 6.85 (d, 1H), 6.86 (d, 1H), 7.53 (d, 1H), 8.77 (t, 1H).

### 3-(Benzo[d][1,3]dioxol-5-ylmethyl)-7-chloroquinazoline-2,4(1H,3H)-dione

2-Amino-N-(benzo[d][1,3]dioxol-5-ylmethyl)-4-chlorobenzamide (0.888 g; 2.91 mmol) was dissolved in 4 ml of dry pyridine under nitrogen and cooled to 0° C. Ethyl chloroformate (0.83 ml, 8.74 mmol) was added slowly and the reaction mixture was stirred at rt for 3 days. 2 M NaOH (4.37 ml; 8.74 mmol) was added slowly at 0° C. and the mixture was stirred 2 h at 50° C. to complete ring closure. The reaction mixture was evaporated to dryness. 15 ml of DCM and 20 ml of water were added and pH was adjusted to acidic with 10 ml of 1 M HCl. The precipitation was filtered, washed with water, and dried to obtain 938 mg of 3-(benzo[d][1,3]dioxol-5-ylmethyl)-7-chloroquinazoline-2,4(1H,3H)-dione. ¹H-NMR (400 MHz, d₆-DMSO): δ 4.97 (s, 2H), 5.96 (s, 2H), 6.80-6.85 (m, 1H), 6.89-5.92 (m, 1H), 7.17-7.12 (m, 1H), 7.22 (dd, 1H), 7.92 (dd, 1H), 11.62 (br s, 1H).

### 3-(Benzo[d][1,3]dioxol-5-ylmethyl)-7-chloro-1-(2-hydroxy-2-methylpropyl)quinazoline-2,4(1H,3H)-dione

3-(Benzo[d][1,3]dioxol-5-ylmethyl)-7-chloroquinazoline-2,4(1H,3H)-dione (150 mg; 0.45 mmol), 1 ml of dry DMF, yttrium(III) nitrate hexahydrate (17.4 mg; 0.045 mmol), and isobutylene oxide (1.21 ml; 13.61 mmol) were placed in a microwave vial under nitrogen and the mixture was heated (absorption high) for 1 h at 160° C. 15 ml of DCM was added and the mixture was washed three times with 25 ml of water. Organic phase was dried and evaporated to dryness. Column chromatography purification (normal phase silica; EtOAc:heptane gradient) gave 56 mg of 3-(benzo[d][1,3]dioxol-5-ylmethyl)-7-chloro-1-(2-hydroxy-2-methylpropyl)quinazoline-2,4(1H,3H)-dione. ¹H-NMR (400 MHz, CDCl₃): δ 1.33 (s, 6H), 2.54 (s, 1H), 4.18 (s, 2H), 5.17 (s, 2H), 5.91 (s, 2H), 6.73 (d, 1H), 6.98-7.05 (m, 2H), 7.21 (dd, 1H), 7.49 (d, 1H), 8.16 (d, 1H).

### Example 136: 3-(4-Bromobenzyl)-6,7,8-trifluoro-1-methylquinazoline-2,4(1H,3H)-dione

3-(4-Bromobenzyl)-6,7,8-trifluoroquinazoline-2,4(1H,3H)-dione (100 mg, 0.26 mmol) prepared in Example 60, K₂CO₃ (72 mg, 0.52 mmol), and 2 ml of dry DMF were charged in a microwave tube. The mixture was flushed with nitrogen, iodomethane (150 mg, 1.0 mmol) was added, and the mixture was heated at 120° C. for 1 h. After cooling to rt, additional batches of K₂CO₃ (72 mg; 0.52 mmol) and iodomethane (150 mg; 1.0 mmol) were added and the mixture was heated at 160° C. for 1 h. The mixture was allowed to cool to rt. Water and DCM were added and the phases were separated. The organic phase was washed once with water and once with saturated aqueous NaCl, dried with a phase separator, and concentrated under reduced pressure. The residue was purified with CombiFlash (normal phase silica) to yield 3 mg of 3-(4-bromobenzyl)-6,7,8-trifluoro-1-methylquinazoline-2,4(1H,3H)-dione. ¹H-NMR (400 MHz, CDCl₃): δ 3.78 (d, 3H), 5.18 (s, 2H), 7.36-7.40 (m, 2H), 7.40-7.45 (m, 2H), 7.89 (ddd, 1H).

### Example 137: (R)-7-Chloro-3-(1-(4-chlorophenyl)ethyl)-1-((3-methyloxetan-3-yl)methyl)quinazoline-2,4(1H,3H)-dione

The preparation of (R)-7-chloro-3-(1-(4-chlorophenyl)ethyl)quinazoline-2,4(1H,3H)-dione is described in Example 112. (R)-7-Chloro-3-(1-(4-chlorophenyl)ethyl)quinazoline-2,4(1H,3H)-dione (200 mg; 0.60 mmol), sodium hydride (288 mg; 1.19 mmol; 60% in oil), and 2 ml of ACN were placed in a microwave vial, bubbled with nitrogen, and stirred for 15 min at rt. 3-(Chloromethyl)-3-methyloxetane (0.26 ml; 2.39 mmol) was added and the mixture was heated (absorption high) for 2 h at 160° C. MeOH was added to the reaction mixture and the mixture was evaporated to dryness. The evaporation residue was dissolved in DCM, washed with saturated NaHCO₃ and then twice with water. Organic phase was dried by filtration through a phase separator and evaporated to dryness. The crude material was purified with column chromatography twice (first C18; ACN:water gradient and then normal phase silica; EtOAc:heptane gradient) to give 65 mg of (R)-7-chloro-3-(1-(4-chlorophenyl)ethyl)-1-((3-methyloxetan-3-yl)methyl)quinazoline-2,4(1H,3H)-dione. ¹H-NMR (400 MHz, CDCl₃): δ 1.44 (s, 3H), 1.90 (d, 3H), 4.07 (dd, 2H), 4.16-4.26 (m, 2H), 4.59 (dd, 2H), 6.37 (q, 1H), 6.98 (d, 1H), 7.22 (dd, 1H), 7.25-7.30 (m, 2H), 7.34-7.39 (m, 2H), 8.17 (d, 1H).

### Example 138: 3-(Benzo[d][1,3]dioxol-5-ylmethyl)-7-chloro-1-((3-methyloxetan-3-yl)methyl)quinazoline-2,4(1H,3H)-dione

The preparation of 3-(benzo[d][1,3]dioxol-5-ylmethyl)-7-chloroquinazoline-2,4(1H,3H)-dione is described in Example 135. 3-(Benzo[d][1,3]dioxol-5-ylmethyl)-7-chloroquinazoline-2,4(1H,3H)-dione (150 mg; 0.454 mmol), sodium hydride (36.3 mg; 0.907 mmol; 60% in oil), 2 ml of dry ACN, and 3-(chloromethyl)-3-methyloxetane (219 mg; 1.814 mmol) were charged in a microwave tube. The mixture was flushed with nitrogen and heated at 160° C. for 3 h. After cooling to rt, additional batches of 3-(chloromethyl)-3-methyloxetane (219 mg) and sodium hydride (36.3 mg) were added and the mixture was heated at 160° C. for 4 h. The mixture was allowed to cool to rt and diluted with DCM. The solution was washed with saturated NaHCO₃ solution and twice with water. The organic phase was dried with a phase separator and concentrated under reduced pressure. The residue was purified with MS-Trigger to yield 113 mg of 3-(benzo[d][1,3]dioxol-5-ylmethyl)-7-chloro-1-((3-methyloxetan-3-yl)methyl)quinazoline-2,4(1H,3H)-dione. ¹H-NMR (400 MHz, CDCl₃): δ 1.47 (s, 3H), 4.14 (s, 2H), 4.25 (d, 2H), 4.67 (d, 2H), 5.15 (s, 2H), 5.90 (s, 2H), 6.72 (d, 1H), 6.96-7.05 (m, 3H), 7.22 (dd, 1H), 8.18 (d, 1H).

### Example 139: (R)-3-(4-Bromobenzyl)-7-chloro-8-fluoro-1-(2-hydroxypropyl)quinazoline-2,4(1H,3H)-dione

(R)-3-(4-bromobenzyl)-7-chloro-8-fluoro-1-(2-hydroxypropyl)quinazoline-2,4(1H,3H)-dione was prepared similarly to (S)-3-(4-bromobenzyl)-7-chloro-8-fluoro-1-(2-hydroxypropyl)quinazoline-2,4(1H,3H)-dione in Example 91. The reaction of 3-(4-bromobenzyl)-7-chloro-8-fluoroquinazoline-2,4(1H,3H)-dione (150 mg; 0.39 mmol) with (R)-2-methyloxirane (0.27 ml; 3.9 mmol) yielded 100 mg of (R)-3-(4-bromobenzyl)-7-chloro-8-fluoro-1-(2-hydroxypropyl)quinazoline-2,4(1H,3H)-dione. ¹H-NMR (400 MHz, CDCl₃): identical to that of (S)-3-(4-bromobenzyl)-7-chloro-8-fluoro-1-(2-hydroxypropyl)quinazoline-2,4(1H,3H)-dione.

### Example 140: 6-(4-Bromobenzyl)-9-fluoro-2,2-dimethyl-2H-[1,4]oxazino[2,3,4-ij]quinazoline-5,7(3H,6H)-dione

The preparation of 3-(4-bromobenzyl)-6,8-difluoro-1-(2-hydroxy-2-methylpropyl)quinazoline-2,4(1H,3H)-dione is described in Example 133. 3-(4-Bromobenzyl)-6,8-difluoro-1-(2-hydroxy-2-methylpropyl)quinazoline-2,4(1H,3H)-dione (50 mg; 0.114 mmol), sodium hydride (9.11 mg; 0.228 mmol; 60% in oil), and 3 ml of dry THF were placed in a reaction flask under nitrogen. The reaction mixture was stirred at rt for 1.5 h. The reaction mixture was diluted with DCM and washed twice with water. The organic phase was dried with a phase separator and evaporated to dryness. The residue was purified with chromatography to yield 36 mg of 6-(4-bromobenzyl)-9-fluoro-2,2-dimethyl-2H-[1,4]oxazino[2,3,4-ij]quinazoline-5,7(3H,6H)-dione. ¹H-NMR (400 MHz, CDCl₃): 1.40 (s, 6H), 3.86 (s, 2H), 5.19 (s, 2H), 6.92 (dd, 1H), 7.35-7.46 (m, 5H).

### Example 141: 7-Chloro-3-(3-chloro-4-methoxybenzyl)-1-((3-methyloxetan-3-yl)methyl)quinazoline-2,4(1H,3H)-dione

The preparation of 7-chloro-3-(3-chloro-4-methoxybenzyl)quinazoline-2,4(1H,3H)-dione is described in Example 113. 7-Chloro-3-(3-chloro-4-methoxybenzyl)quinazoline-2,4(1H,3H)-dione (150 mg; 0.427 mmol), sodium hydride (34.2 mg; 0.854 mmol; 60% in oil), 2 ml of dry ACN, and 3-(chloromethyl)-3-methyloxetane (206 mg; 1.709 mmol) were charged in a microwave tube. The mixture was flushed with nitrogen and heated at 160° C. for 11 h. The mixture was allowed to cool to rt and diluted with DCM. The solution was washed with saturated NaHCO₃ solution and twice with water. The organic phase was dried with a phase separator and concentrated under reduced pressure. The residue was purified with MS-Trigger to yield 84 mg of 7-chloro-3-(3-chloro-4-methoxybenzyl)-1-((3-methyloxetan-3-yl)methyl)quinazoline-2,4(1H,3H)-dione. ¹H-NMR (400 MHz, CDCl₃): δ 1.47 (s, 3H), 3.85 (s, 3H), 4.15 (s, 2H), 4.25 (d, 2H), 4.67 (d, 2H), 5.15 (s, 2H), 6.84 (d, 1H), 7.00 (d, 1H), 7.22 (dd, 1H), 7.40 (dd, 1H), 7.54 (d, 1H), 8.18 (d, 1H).

### Example 142: 6-(4-Bromobenzyl)-9-chloro-2,2-dimethyl-2H-[1,4]oxazino[2,3,4-ij]quinazoline-5,7(3H,6H)-dione

The preparation of 3-(4-bromobenzyl)-6-chloro-8-fluoro-1-(2-hydroxy-2-methylpropyl)quinazoline-2,4(1H,3H)-dione is described in Example 80. 3-(4-Bromobenzyl)-6-chloro-8-fluoro-1-(2-hydroxy-2-methylpropyl)quinazoline-2,4(1H,3H)-dione (50 mg; 0.110 mmol), sodium hydride (8.78 mg; 0.219 mmol; 60% in oil), and 2 ml of dry THF were placed in a reaction flask under nitrogen. The reaction mixture was stirred at rt for 2 h. DCM was added and the mixture was washed twice with water. The organic phase was dried with a phase separator and evaporated to dryness. The residue was purified with flash chromatography to yield 38 mg of 6-(4-bromobenzyl)-9-chloro-2,2-dimethyl-2H-[1,4]oxazino[2,3,4-ij]quinazoline-5,7(3H,6H)-dione. ¹H-NMR (400 MHz, CDCl₃): δ 1.40 (s, 6H), 3.85 (s, 2H), 5.18 (s, 2H), 7.14 (d, 1H), 7.36-7.47 (m, 4H), 7.71 (d, 1H).

### Example 143: (R)-3-(1-(4-Chlorophenyl)ethyl)-1-methyl-7-nitroquinazoline-2,4(1H,3H)-dione

### (R)-2-Amino-N-(1-(4-chlorophenyl)ethyl)-4-nitrobenzamide

4-Nitroanthranilic acid (10 g; 54.90 mmol), DCM (25 ml), and TEA (22.96 ml; 165 mmol) were placed in a reaction vessel under nitrogen. (R)-1-(4-Chlorophenyl)ethylamine (7.70 ml; 54.90 mmol) and T3P (38.8 ml; 65.9 mmol; 50% in EtOAc) were added in this order slowly and stirred overnight at rt. 150 ml of DCM was added and the mixture was washed with 100 ml of water and then twice with 150 ml of water. In the last wash, additionally 100 ml of DCM was used to dissolve precipitation. The organic layer was dried by filtration through a phase separator funnel, evaporated to dryness, and dried under vacuum at 40° C. to give 16.7 g of crude (R)-2-amino-N-(1-(4-chlorophenyl)ethyl)-4-nitrobenzamide. ¹H-NMR (400 MHz, CDCl₃): δ 1.59 (d, 3H), 5.24 (quint, 1H), 5.80 (br s, 2H), 6.23 (d, 1H), 7.29-7.37 (m, 4H), 7.42-7.44 (m, 1H), 7.50 (dd, 1H).

### (R)-3-(1-(4-Chlorophenyl)ethyl)-7-nitroquinazoline-2,4(1H,3H)-dione

(R)-2-Amino-N-(1-(4-chlorophenyl)ethyl)-4-nitrobenzamide (16.7 g; 52.20 mmol) was dissolved in 75 ml of dry pyridine under nitrogen and cooled to 0° C. Ethyl chloroformate (14.97 ml; 157 mmol) was added slowly and the mixture was stirred overnight at rt. The solution was cooled to 0° C. and 2 M NaOH (120 ml; 240 mmol) was added slowly. The solution was stirred for 2 h at 50° C., cooled to rt, and evaporated to dryness. 250 ml of DCM was added and pH was adjusted to 5 with 2 M HCl (125 ml). 150 ml of DCM was added and organic phase was washed with 275 ml of water. The glassware was flushed with 100 ml of DCM and this organic phase was washed with 150 ml of water. All organic phases were separated, dried by filtration through a phase separator funnel, evaporated to dryness, and dried under vacuum at 40° C. to obtain 46.8 g of crude material. The evaporation residue was dissolved in 150 ml of EtOAc. The solution was washed with a mixture of 1 M HCl (35 ml) and 130 ml of water. Organic layer was separated and evaporated to dryness to obtain 18.4 g of crude (R)-3-(1-(4-Chlorophenyl)ethyl)-7-nitroquinazoline-2,4(1H,3H)-dione. ¹H-NMR (400 MHz, d₆-DMSO): δ 1.81 (d, 3H), 6.15 (q, 1H), 7.31-7.41 (m, 4H), 7.92-7.98 (m, 2H), 8.14 (d, 1H), 11.79 (br s, 1H).

### (R)-3-(1-(4-Chlorophenyl)ethyl)-1-methyl-7-nitroquinazoline-2,4(1H,3H)-dione

(R)-3-(1-(4-Chlorophenyl)ethyl)-7-nitroquinazoline-2,4(1H,3H)-dione (5 g; 14.46 mmol), KOH pellets (0.87 g; 15.51 mmol), and 25 ml of dry DMF were mixed under nitrogen and stirred for 15 min at rt. Iodomethane (1.35 ml; 21.69 mmol) was added and the reaction mixture was stirred overnight at rt Water (50 ml) was added to give tar, which was desiccated and dissolved in 100 ml of DCM. Water phase was washed three times with 50 ml of DCM. All organic phases were combined, washed three times with 250 ml of water, and dried by filtration through a phase separation funnel. Organic phase was evaporated to dryness to obtain 5.21 g of tar like crude product. 2.6 g of crude material was purified twice with column chromatography (normal phase silica; EtOAc:heptane gradient; the second time with slower EtOAc increase) to give 1.90 g of (R)-3-(1-(4-Chlorophenyl)ethyl)-1-methyl-7-nitroquinazoline-2,4(1H,3H)-dione. ¹H-NMR (400 MHz, d₆-DMSO): δ 1.81 (d, 3H), 3.56 (s, 3H), 6.21 (q, 1H), 7.32-7.41 (m, 4H), 8.04 (dd, 1H), 8.14 (d, 1H), 8.24 (d, 1H).

### Example 144: 7-Chloro-3-(4-chlorobenzyl)-1-(2,3-dihydroxy-2-methylbutyl)-8-fluoroquinazoline-2,4(1H,3H)-dione

### 7-Chloro-3-(4-chlorobenzyl)-8-fluoro-1-(2-hydroxy-2-methyl-3-oxobutyl)quinazoline-2,4(1H,3H)-dione

7-Chloro-3-(4-chlorobenzyl)-8-fluoroquinazoline-2,4(1H,3H)-dione (0.25 g, 0.74 mmol) prepared in Example 67, yttrium(III) nitrate hexahydrate (28 mg; 0.07 mmol), 1 ml of dry DMF, and 1-(2-methyloxiran-2-yl)ethanone (0.69 ml; 7.4 mmol) were charged in a microwave tube and heated at 160° C. for 1 h. After cooling to rt, the mixture was diluted with DCM and washed twice with water. The organic phase was dried with a phase separator and concentrated under reduced pressure. The residue was purified with MS-Trigger to yield 60 mg of 7-chloro-3-(4-chlorobenzyl)-8-fluoro-1-(2-hydroxy-2-methyl-3-oxobutyl)quinazoline-2,4(1H,3H)-dione. ¹H-NMR (400 MHz, CDCl₃): δ 1.48 (s, 3H), 2.40 (s, 3H), 4.07 (d, 1H), 4.71 (ddd, 2H), 5.12 (ddd, 2H), 7.24-7.30 (m, 3H), 7.33-7.37 (m, 2H), 7.96 (dd, 1H).

### 7-Chloro-3-(4-chlorobenzyl)-1-(2,3-dihydroxy-2-methylbutyl)-8-fluoroquinazoline-2,4(1H,3H)-dione

7-Chloro-3-(4-chlorobenzyl)-8-fluoro-1-(2-hydroxy-2-methyl-3-oxobutyl)quinazoline-2,4(1H,3H)-dione (60 mg; 0.13 mmol) and 2 ml of dry EtOH were placed in a reaction flask under nitrogen and cooled to 0° C. Sodium borohydride (10 mg; 0.26 mmol; suspended in approximately 5 ml of dry EtOH) was added and the mixture was stirred at rt overnight. The reaction was quenched with the addition of water and the mixture was neutralized with 1 M HCl. The mixture was diluted with DCM. The organic phase was washed with water and saturated aqueous NaCl, dried with a phase separator, and concentrated under reduced pressure. The residue was purified with MS-Trigger to yield 5 mg of 7-chloro-3-(4-chlorobenzyl)-1-(2,3-dihydroxy-2-methylbutyl)-8-fluoroquinazoline-2,4(1H,3H)-dione. ¹H-NMR (400 MHz, CDCl₃): δ 1.03-1.18 (m, 3H), 1.20-1.26 (m, 3H), 2.20-2.89 (br d, 1H), 2.90-3.45 (br d, 1H), 3.53-3.72 (m, 1H), 4.45-4.75 (m, 2H), 5.16-5.29 (m, 2H), 7.26-7.32 (m, 3H), 7.41-7.46 (m, 2H), 8.02 (dd, 1H).

### Example 145: 9-Fluoro-6-(4-methoxybenzyl)-2,2-dimethyl-2H-[1,4]oxazino[2,3,4-ij]quinazoline-5,7(3H,6H)-dione

The preparation of 6,8-difluoro-1-(2-hydroxy-2-methylpropyl)-3-(4-methoxybenzyl)quinazoline-2,4(1H,3H)-dione is described in Example 110. 6,8-Difluoro-1-(2-hydroxy-2-methylpropyl)-3-(4-methoxybenzyl)quinazoline-2,4(1H,3H)-dione (210 mg; 0.538 mmol), sodium hydride (43.0 mg; 1.076 mmol; 60% in oil), and 2 ml of dry THF were placed in a reaction flask under nitrogen. The reaction mixture was stirred at rt for 2 h. DCM was added and the mixture was washed twice with water. The organic phase was dried with a phase separator and evaporated to dryness. The residue was purified with flash chromatography to yield 142 mg of 9-fluoro-6-(4-methoxybenzyl)-2,2-dimethyl-2H-[1,4]oxazino[2,3,4-ij]quinazoline-5,7(3H,6H)-dione. ¹H-NMR (400 MHz, CDCl₃): δ 1.40 (s, 6H), 3.77 (s, 3H), 3.86 (s, 2H), 5.18 (s, 2H), 6.81-6.87 (m, 2H), 6.88-6.92 (m, 1H), 7.41-7.45 (m, 1H), 7.47-7.52 (m, 2H).

### Example 146: 3-(4-Bromo-2-fluorobenzyl)-7-chloro-1-((3-methyloxetan-3-yl)methyl)quinazoline-2,4(1H,3H)-dione

The preparation of 3-(4-bromo-2-fluorobenzyl)-7-chloroquinazoline-2,4(1H,3H)-dione is described in Example 92. 3-(4-Bromo-2-fluorobenzyl)-7-chloroquinazoline-2,4(1H,3H)-dione (200 mg; 0.52 mmol), sodium hydride (41.7 mg; 1.04 mmol; 60% in oil), and 2 ml of dry ACN were placed in a microwave vial, bubbled with nitrogen, and stirred for 15 min at rt. 3-(Chloromethyl)-3-methyloxetane (0.23 ml; 2.09 mmol) was added and the mixture was heated (absorption high) for 2 h at 160° C. Additional 3-(chloromethyl)-3-methyloxetane (0.11 ml; 1.04 mmol) was added and the microwave reaction continued for 1 h at 160° C. (absorption high). 2 ml of MeOH was added and the reaction mixture was evaporated to dryness. The evaporation residue was dissolved in 15 ml of DCM, washed with 20 ml of brine and then twice with 20 ml of water. Organic phase was dried by filtration through a phase separator and evaporated to dryness to give 323 mg of crude product. Preparative LC-MS purification gave 96 mg of 3-(4-bromo-2-fluorobenzyl)-7-chloro-1:((3-methyloxetan-3-yl)methyl)quinazoline-2,4(1H,3H)-dione. ¹H-NMR (400 MHz, CDCl₃): δ 1.47 (s, 3H), 4.16 (s, 2H), 4.26 (d, 2H), 4.67 (d, 2H), 5.29 (s, 2H), 7.02 (d, 1H), 7.15-7.26 (m, 4H), 8.20 (d, 1H).

### Example 147: 2-Acetyl-10-chloro-6-(4-chlorobenzyl)-2-methyl-2H-[1,4]oxazino[2,3,4-ij]quinazoline-5,7(3H,6H)-dione

The preparation of 7-chloro-3-(4-chlorobenzyl)-8-fluoroquinazoline-2,4(1H,3H)-dione is described in Example 67. 7-Chloro-3-(4-chlorobenzyl)-8-fluoroquinazoline-2,4(1H,3H)-dione (250 mg; 0.737 mmol), yttrium(III) nitrate hexahydrate (28.2 mg; 0.074 mmol), 1 ml of dry DMF, and 1-(2-methyloxiran-2-yl)ethanone (0.69 ml; 7.37 mmol) were charged in a microwave tube and heated at 160° C. for 1 h. After cooling to rt, the mixture was diluted with DCM and washed four times with water. The organic phase was dried with a phase separator and concentrated under reduced pressure. The residue was purified with flash chromatography to yield 126 mg of 2-acetyl-10-chloro-6-(4-chlorobenzyl)-2-methyl-2H-[1,4]oxazino[2,3,4-ij]quinazoline-5,7(3H,6H)-dione. ¹H-NMR (400 MHz, CDCl₃): δ 1.63 (s, 3H), 2.20 (s, 3H), 3.60 (d, 1H), 4.80 (d, 1H), 5.17 (q, 2H), 7.23-7.32 (m, 3H), 7.42-7.49 (m, 2H), 7.73 (d, 1H).

### Example 148: Diastereomer 1 and diastereomer 2 of 10-chloro-6-(4-chlorobenzyl)-2-(1-hydroxyethyl)-2-methyl-2H-[1,4]oxazino[2,3,4-ij]quinazoline-5,7(3H,6H)-dione

The preparation of 2-acetyl-10-chloro-6-(4-chlorobenzyl)-2-methyl-2H-[1,4]oxazino[2,3,4-ij]quinazoline-5,7(3H,6H)-dione is described in Example 147. 2-Acetyl-10-chloro-6-(4-chlorobenzyl)-2-methyl-2H-[1,4]oxazino[2,3,4-ij]quinazoline-5,7(3H,6H)-dione (40 mg; 0.095 mmol), sodium borohydride (10.83 mg: 0.286 mmol), and 4 ml of dry EtOH was placed in a flask under nitrogen. The reaction mixture was stirred at rt for 2 h. Few drops of water and 1 M HCl were added and the mixture was diluted with DCM and washed with water and brine. The organic phase was dried with a phase separator and concentrated under reduced pressure. The residue was purified with MS-Trigger to yield 6 mg of diastereomer 1 and 24 mg of diastereomer 2 of 10-chloro-6-(4-chlorobenzyl)-2-(1-hydroxyethyl)-2-methyl-2H-[1,4]oxazino[2,3,4-ij]quinazoline-5,7(3H,6H)-dione. Diastereomer 1 ¹H-NMR (400 MHz, CDCl₃): δ 1.28 (s, 3H), 1.35 (d, 3H), 2.24 (br s, 1H), 3.80 (d, 1H), 3.94 (q, 1H), 4.18 (d, 1H), 5.20 (s, 2H), 7.23 (d, 1H), 7.26-7.30 (m, 2H), 7.43-7.50 (m, 2H), 7.71 (d, 1H). Diastereomer 2 ¹H-NMR (400 MHz, CDCl₃): δ 1.27 (s, 3H), 1.34 (d, 3H), 2.25 (br s, 1H), 3.94 (d, 1H), 4.06 (q, 1H), 4.22 (d, 1H), 5.20 (s, 2H), 7.22 (d, 1H), 7.26-7.30 (m, 2H), 7.44-7.51 (m, 2H), 7.71 (d, 1H).

As already mentioned hereinbefore, the compounds of formula I show interesting pharmacological properties, namely they exhibit an enhanced positive allosteric GABA_{B} modulator effect and possess decreased agonism. Said properties are demonstrated with the pharmacological test presented below.

### Determination of Agonism and Positive Allosteric Modulator Pharmacology In Vitro

CHO cells expressing the human GABA_{B1} and GABA_{B2} receptor subunits co-expressing promiscuous Galpha16 proteins to force couple the receptor to the intracellular calcium signalling pathway were used to study GABA_{B} pharmacology in vitro. The cells were maintained at 37° C. in a 5% CO₂ 95% air atmosphere in HAM F-12 medium supplemented with 10% foetal bovine serum and selection antibiotics.

The day before the experiment the cells were detached and plated into black-walled clear bottom 384-well plates at a cell density of 20 000 cells per well. The growth medium was removed and the cells were incubated with FLIPR Calcium 5 Assay reagent (Molecular Devices, CA, USA) diluted 1:1 in Probenecid-Ringer for 1 h at 37° C. in dark. Probenecid-Ringer buffer consisted of 150 mM NaCl, 3 mM KCl, 1.2 mM MgCl₂, 1 mM CaCl₂, 5 mM glucose, 20 mM 2-(4-(2-hydroxyethyl)piperazin-1-yl)ethanesulfonic acid (HEPES), and 2.5 mM Probenecid (pH 7.4 adjusted with 1.0 M NaOH and osmolarity adjusted to 322 Osm). Compounds were dissolved in Probenecid-Ringer. Changes in intracellular calcium were monitored using FLIPRtetra (Molecular Devices, CA, USA) and displayed using Screen Works software. The samples were excited at 470-495 nm and emission was detected at 515-575 nm. All experiments were performed at 37° C.

A two-addition protocol was used to determine agonism and positive allosteric modulator pharmacology in the same assay using a kinetic read out. Agonism pharmacology of compounds was studied in the first addition in the concentration range 0.123-30 μM in quadruplicate at each concentration. In the second addition, a low (EC_{10.5}) concentration of GABA was applied to detect the positive allosteric modulator pharmacology of the compounds to be studied. The EC_{10.5} concentration was determined from the GABA EC₅₀ value using the formula EC_{F}≈(F/(100-F))^{1/H}·EC₅₀, wherein F is the fraction of full response and H is Hill slope.

Baseline fluorescence minimum was subtracted from maximum for the calculation of agonism and positive allosteric modulator pharmacology. Both in the agonism mode and in the allosteric mode, the efficacy was related to the intrinsic activity (IA) of the endogenous agonist GABA at 30 μM (positive allosteric modulator efficacy at 10 μM/GABA efficacy or agonism efficacy at 30 μM/GABA efficacy). To determine potentiation of GABA dose-response, the compounds, at a concentration of 10 μM diluted in Probenecid-Ringer, were added by FLIPR tetra followed by addition of GABA (diluted in water in the concentration range 0.123-10 μM). The EC₅₀ values were determined using a four-parameter logistic fit (model 205 in ActivityBase XE). The EC₅₀ value of GABA was divided with the EC₅₀ value of GABA in the presence of positive allosteric modulator to determine potentiation of GABA dose-response EC₅₀ value.

The results of the determination of agonism and positive allosteric modulator pharmacology in vitro are shown in Table 1. The results show that the compounds of formula I exhibit an enhanced positive allosteric GABA_{B} modulator effect and possess decreased agonism.

TABLE 1
Agonism and positive allosteric modulator pharmacology in vitro.

| | IA (GABA_{B} | | |
|---|---|---|---|
| | positive | | Potentiation |
| | allosteric | IA (GABA_{B} | of GABA |
| Compound | modulation) | agonism) | (fold) |
| Compound of Example 1 | 0.64 | 0.22 | 6.4 |
| Compound of Example 2 | 0.65 | 0.14 | 11 |
| Compound of Example 4 | 0.58 | 0.19 | 10.3 |
| Compound of Example 12 | 0.67 | 0.20 | 25.8 |
| Compound of Example 18 | 0.43 | 0.06 | 1.6 |
| Compound of Example 26 | 0.48 | 0.08 | 1.7 |
| Compound of Example 33 | 0.45 | 0.23 | 4.8 |
| Compound of Example 37 | 0.39 | 0.21 | 1.9 |
| Compound of Example 38 | 0.21 | 0.13 | 3.5 |
| Compound of Example 39 | 0.85 | 0.30 | 23.1 |
| Compound of Example 40 | 0.26 | 0.04 | 1.5 |
| Compound of Example 41 | 0.47 | 0.10 | 2.8 |
| Compound of Example 42 | 0.37 | 0.18 | 7.3 |
| Compound of Example 43 | 0.51 | 0.17 | 3.8 |
| Compound of Example 49 | 0.61 | 0.28 | 31 |
| Compound of Example 50 | 0.73 | 0.04 | 22 |
| Compound of Example 60 | 0.85 | 0.20 | 89 |
| Compound of Example 66 | 0.77 | 0.04 | 12.7 |
| Compound of Example 67 | 0.71 | 0.02 | 9.5 |
| Compound of Example 82 | 0.71 | 0.18 | 24.5 |
| Compound of Example 84 | 0.86 | 0.10 | 33.4 |
| Compound of Example 87 | 0.44 | 0.11 | 3.3 |
| Compound of Example 117 | 0.51 | 0.09 | 19.8 |
| Compound of Example 123 | 0.71 | 0.13 | 60.4 |
| Compound of Example 127 | 0.53 | 0.02 | 6.7 |
| Compound of Example 131 | 0.89 | 0.19 | 42.6 |
| Compound of Example 137 | 0.64 | 0.05 | 19.7 |

Effects of the compounds of formula I in vivo can be studied, for example, according to the method described in Martin, F. C. et al. Movement Disorders, 20 (2005) 298.

The compounds of formula I can be administered, for example, enterally, topically, or parenterally by means of any pharmaceutical formulation useful for said administration and containing at least one active compound of formula I in pharmaceutically acceptable and effective amounts together with pharmaceutically acceptable excipients known in the art.

The therapeutic dose to be given to a subject in need of the treatment will vary depending on the compound being administered, the age and the sex of the subject being treated, the particular condition being treated, as well as the route and method of administration, and may be determined by a person skilled in the art. A typical dosage for oral administration is from 10 μg/kg to 900 mg/kg per day and for parenteral administration from 1 μg/kg to 100 mg/kg for an adult mammal.

The compounds of formula I exhibit a positive allosteric GABA_{B} modulator effect. The present disclosure thus provides compounds for use as a medicament. Compounds for use in the treatment of a disease where a positive allosteric modulator of the GABA_{B} receptor is indicated to be useful are also provided. Furthermore, a method for the treatment of a disease where a positive allosteric modulator of the GABA_{B} receptor is indicated to be useful is provided. In said method a therapeutically effective amount of at least one compound of formula I is administered to a mammal, such as a human, in need of such treatment. Use of the compounds of formula I for the manufacture of a medicament for the treatment of a disease where a positive allosteric modulator of the GABA_{B} receptor is indicated to be useful is also provided.

In one embodiment the aforementioned disease where a positive allosteric modulator of the GABA_{B} receptor is indicated to be useful is essential tremor, Parkinsonian tremor, levodopa-induced dyskinesia, Parkinsonian motor symptoms, Parkinsonian non-motor symptoms, spasticity related to multiple sclerosis, spasticity related to amyotrophic lateral sclerosis, spasticity related to spinal cord injury, spasticity related to cerebral injury, dystonia, chronic pain, addiction, anxiety, epilepsy, autism, fragile X syndrome, amyotrophic lateral sclerosis, post-traumatic stress disorder, depression, insomnia, narcolepsy, Alzheimer's disease, dementia, Charcot Marie Tooth 1A neuropathy, overactive bladder, gastro-esophageal reflux disease, inflammatory bowel disease, or chronic tinnitus.

In one embodiment the aforementioned disease where a positive allosteric modulator of the GABA_{B} receptor is indicated to be useful is essential tremor, Parkinsonian tremor, levodopa-induced dyskinesia, dystonia, or chronic pain.

In one embodiment the aforementioned disease where a positive allosteric modulator of the GABA_{B} receptor is indicated to be useful is essential tremor. In one embodiment potentiation of therapy with propranolol or primidone is provided. In said method a therapeutically effective amount of at least one compound of formula I is administered together with propranolol or primidone, each in its own composition or combined in a single composition. Also combination with alprazolam, atenolol, gabapentin, sotalol, topiramate, olanzapine, pregabalin, zonisamide, or clozapine may be useful.

The daily dose of propranolol is typically from 40 mg to 320 mg divided into 1 to 4 individual doses, e.g. 2 to 3 individual doses. The daily dose of primidone is typically from 50 mg to 750 mg divided into several individual doses, e.g. 2 individual doses. In combination with a positive allosteric modulator of the GABA_{B} receptor lower doses of propranolol or primidone may be effective.

Alprazolam, atenolol, gabapentin, sotalol, topiramate, olanzapine, pregabalin, zonisamide, or clozapine may be useful at recommended doses. In combination with a positive allosteric modulator of the GABA_{B} receptor lower doses may be effective.

In one embodiment the aforementioned disease where a positive allosteric modulator of the GABA_{B} receptor is indicated to be useful is Parkinsonian tremor. In one embodiment potentiation of therapy with levodopa, a dopamine agonist, an anticholinergic, or a monoamine oxidase B (MAO-B) inhibitor is provided. In said method a therapeutically effective amount of at least one compound of formula I is administered together with levodopa, a dopamine agonist, an anticholinergic, or a MAO-B inhibitor, each in its own composition or combined in a single composition. Also combination with clozapine, amantadine, clonazepam, propranolol, or gabapentin may be useful.

Levodopa may be administered with a dopa decarboxylase (DDC) inhibitor, such as benserazide or carbidopa, and a catechol O-methyltransferase COMT inhibitor, such as entacapone or tolcapone. The amount of levodopa can be from 50 mg to 400 mg. The amount of carbidopa can be from 5 mg to 200 mg. The carbidopa:levodopa ratio can be from 1:1 to 1:40. The amount of entacapone can be 200 mg taken 1 to 10 times per day.

Dopamine agonists include, but are not limited to, bromocriptine, pramipexole, ropinirole, transdermal rotigotine, piribedil, and apomorphine. The daily dose of bromocriptine is typically from 1 mg to 30 mg divided into several individual doses, e.g. 3 individual doses. The daily dose of pramipexole is typically from 0.26 mg to 3.3 mg divided into several individual doses, e.g. 3 individual doses. Pramipexole is available also as a once per day preparation. The daily dose of ropinirole is typically from 0.75 mg to 24 mg divided into several individual doses, e.g. 3 individual doses. Ropinirole is available also as a once per day preparation. The daily dose of transdermal rotigotine is typically from 1 mg to 16 mg, e.g. from 2 mg to 8 mg, and is usually applied every 24 h. The daily dose of piribedil is typically from 40 mg to 250 mg divided into 1 to 10 individual doses. The daily dose of apomorphine is typically from 1 mg to 100 mg divided into 1 to 12 individual doses, e.g. 1 to 10 individual doses. Sometimes apomorphine is administered as a continuous subcutaneous infusion. In combination with a positive allosteric modulator of the GABA_{B} receptor lower doses of the dopamine agonist may be effective.

Anticholinergics include, but are not limited to, trihexyphenidyl, benztropine, orphenadrine, procyclidine, and biperiden. The daily dose of trihexyphenidyl is typically from 2 mg to 20 mg divided into several individual doses, e.g. 3 to 4 individual doses. The daily dose of oral benztropine is typically from 0.5 mg to 6 mg divided into several individual doses, e.g. 2 to 4 individual doses. The daily dose of orphenadrine is typically from 100 mg to 400 mg divided into several individual doses, e.g. from 150 mg to 300 mg divided into 2 individual doses. The daily dose of procyclidine is typically from 7.5 mg to 60 mg divided into several individual doses, e.g. from 7.5 mg to 30 mg divided into 3 individual doses. The daily dose of biperiden is typically from 1 mg to 16 mg divided into several individual doses, e.g. 2 individual doses. In combination with a positive allosteric modulator of the GABA_{B} receptor lower doses of the anticholinergic may be effective.

MAO-B inhibitors include, but are not limited to, selegiline, lazabemide, rasagiline, and safinamide. The daily dose of selegiline is typically from 1 mg to 20 mg, e.g. from 5 mg to 10 mg, divided into 1 to 10 individual doses, e.g. 1 to 2 individual doses. The daily dose of lazabemide is typically from 100 mg to 800 mg, e.g. from 100 mg to 200 mg, divided into 1 to 10 individual doses, e.g. 1 to 2 individual doses. The daily dose of rasagiline is typically from 0.1 mg to 5 mg divided into 1 to 10 individual doses, e.g. 1 to 2 individual doses. The daily dose of safinamide is typically from 10 mg to 600 mg, e.g. from 50 mg to 150 mg, divided into 1 to 10 individual doses, e.g. 1 to 2 individual doses. In combination with a positive allosteric modulator of the GABA_{B} receptor lower doses of the MAO-B inhibitor may be effective.

The daily dose of clozapine is typically from 5 mg to 50 mg divided into 1 to 10 individual doses. The daily dose of amantadine is typically from 10 mg to 1,000 mg, e.g. from 100 mg to 400 mg, divided into 1 to 10 individual doses. The daily dose of clonazepam is typically from 1 mg to 20 mg divided into 1 to 10 individual doses. The daily dose of propranolol is typically from 40 mg to 320 mg divided into 1 to 10 individual doses. The daily dose of gabapentin is typically from 900 mg to 4,800 mg divided into 1 to 10 individual doses. In combination with a positive allosteric modulator of the GABA_{B} receptor lower doses of clozapine, amantadine, clonazepam, propranolol, or gabapentin may be effective.

In one embodiment the aforementioned disease where a positive allosteric modulator of the GABA_{B} receptor is indicated to be useful is levodopa-induced dyskinesia. In one embodiment potentiation of therapy with amantadine is provided. In said method a therapeutically effective amount of at least one compound of formula I is administered together with amantadine, each in its own composition or combined in a single composition. Also combination with enteral levodopa gel, sarizotan, levetiracetam, clozapine, aripiprazole, or subcutaneous infusion of apomorphine may be useful.

The daily dose of amantadine is typically from 10 mg to 1,000 mg, e.g. from 100 mg to 400 mg, divided into 1 to 10 individual doses, e.g. 1 to 2 individual doses. The daily dose of sarizotan is typically from 1 mg to 10 mg divided into 1 to 10 individual doses. The daily dose of levetiracetam is typically from 500 mg to 3,000 mg divided into 1 to 10 individual doses. The daily dose of clozapine is typically from 5 mg to 50 mg divided into 1 to 10 individual doses. The daily dose of aripiprazole is typically from 10 mg to 30 mg divided into 1 to 10 individual doses. In combination with a positive allosteric modulator of the GABA_{B} receptor lower doses of amantadine, sarizotan, levetiracetam, clozapine, or aripiprazole may be effective.

In one embodiment the aforementioned disease where a positive allosteric modulator of the GABA_{B} receptor is indicated to be useful is dystonia. In one embodiment potentiation of therapy with an anticholinergic, a benzodiazepine, baclofen, a dopamine agonist, a dopamine depleter, or tizanidine is provided. In said method a therapeutically effective amount of at least one compound of formula I is administered together with an anticholinergic, a benzodiazepine, baclofen, a dopamine agonist, a dopamine depleter, or tizanidine, each in its own composition or combined in a single composition. In one embodiment potentiation of therapy with injections of botulinum toxin is provided.

Anticholinergics include, but are not limited to, trihexyphenidyl, benztropine, orphenadrine, procyclidine, and biperiden. Administration of trihexyphenidyl, benztropine, orphenadrine, procyclidine, and biperiden has been described above.

Benzodiazepines include, but are not limited to, diazepam, clonazepam, and lorazepam. The daily dose of diazepam is typically from 1 mg to 60 mg divided into several individual doses, e.g. 1 to 10 individual doses, such as 2 to 3 individual doses. The daily dose of clonazepam is typically from 1 mg to 20 mg divided into several individual doses, e.g. 1 to 10 individual doses, such as 3 to 4 individual doses. The daily dose of lorazepam is typically from 1 mg to 10 mg divided into several individual doses, e.g. 1 to 10 individual doses, such as from 1 mg to 4 mg divided into 2 to 3 individual doses. In combination with a positive allosteric modulator of the GABA_{B} receptor lower doses of the benzodiazepine may be effective.

The daily dose of baclofen is typically from 15 mg to 60 mg divided into several individual doses, e.g. 1 to 10 individual doses, such as 3 individual doses. In combination with a positive allosteric modulator of the GABA_{B} receptor lower doses of baclofen may be effective.

Dopamine agonists include, but are not limited to, levodopa and bromocriptine. Administration of levodopa and bromocriptine has been described above.

Dopamine depleters include, but are not limited to, reserpine and tetrabenazine. The daily dose of reserpine is typically from 0.1 mg to 1 mg divided into several individual doses, e.g. 1 to 10 individual doses. The daily dose of tetrabenazine is typically from 12.5 mg to 200 mg divided into several individual doses, e.g. 1 to 10 individual doses, such as 3 individual doses. In combination with a positive allosteric modulator of the GABA_{B} receptor lower doses of the dopamine depleter may be effective.

The daily dose of tizanidine is typically from 2 mg to 36 mg divided into several individual doses, e.g. 1 to 10 individual doses, such as 3 to 4 individual doses. In combination with a positive allosteric modulator of the GABA_{B} receptor lower doses of tizanidine may be effective.

Botulinum toxin is injected into the affected dystonic muscles with or without electromyography (EMG) guidance. The amount given depends on the product but may be titrated from 1 and 1000 units per muscle group, e.g. 25 to 200 units per muscle group, reinjected every 3-4 months.

In one embodiment the aforementioned disease where a positive allosteric modulator of the GABA_{B} receptor is indicated to be useful is chronic pain. In one embodiment potentiation of therapy with paracetamol, an anticonvulsant, an antidepressant, a non-steroidal anti-inflammatory (NSAID), baclofen, or an opiate is provided. In said method a therapeutically effective amount of at least one compound of formula I is administered together with paracetamol, an anticonvulsant, an antidepressant, a NSAID, baclofen, or an opiate, each in its own composition or combined in a single composition. In one embodiment potentiation of therapy with injections of botulinum toxin is provided. Also local applications may be useful. Local applications include, but are not limited to, capsaicin, lidocaine, and electrical stimulation.

The daily dose of paracetamol is typically from 500 mg to 4,000 mg divided into several individual doses, e.g. 1 to 10 individual doses, such as 4 individual doses. In combination with a positive allosteric modulator of the GABA_{B} receptor lower doses of paracetamol may be effective.

Anticonvulsants include, but are not limited to, gabapentin, pregabalin, and carbamazepine. The daily dose of gabapentin is typically from 300 mg to 4,800 mg divided into several individual doses, e.g. 1 to 10 individual doses, such as 3 individual doses. The daily dose of pregabalin is typically from 150 mg to 600 mg divided into several individual doses, e.g. 1 to 10 individual doses, such as 2 to 3 individual doses. The daily dose of carbamazepine is typically from 200 mg to 1,600 mg divided into several individual doses, e.g. 1 to 10 individual doses, such as 3 to 4 individual doses. In combination with a positive allosteric modulator of the GABA_{B} receptor lower doses of the anticonvulsant may be effective.

Antidepressants include, but are not limited to, amitriptyline and duloxetine. The daily dose of amitriptyline is typically from 50 mg to 200 mg divided into several individual doses, e.g. 1 to 10 individual doses, such as 2 to 3 individual doses. The daily dose of duloxetine is typically from 60 mg to 120 mg divided into several individual doses, e.g. 1 to 10 individual doses, such as 1 to 2 individual doses. In combination with a positive allosteric modulator of the GABA_{B} receptor lower doses of the antidepressant may be effective.

NSAIDs include, but are not limited to, aspirin, ibuprofen, and naproxen. The daily dose of aspirin is typically from 300 mg to 3,600 mg divided into several individual doses, e.g. 1 to 10 individual doses, such as 4 to 6 individual doses. The daily dose of ibuprofen is typically from 100 mg to 2,400 mg divided into several individual doses, e.g. 1 to 10 individual doses, such as 3 to 6 individual doses. The daily dose of naproxen is typically from 250 mg to 1,000 mg divided into several individual doses, e.g. 1 to 10 individual doses, such as 1 to 2 individual doses. In combination with a positive allosteric modulator of the GABA_{B} receptor lower doses of the NSAID may be effective.

Administration of baclofen has been described above.

Opiates include, but are not limited to, hydrocodone, codeine, tramadol, and morphine. The daily dose of hydrocodone is typically from 5 mg to 60 mg divided into several individual doses, e.g. 1 to 10 individual doses, such as 4 to 6 individual doses. Hydrocodone is often administered together with paracetamol in amounts of from 5 mg to 10 mg hydrocodone to 300 mg paracetamol. The daily dose of codeine is typically from 30 mg to 240 mg divided into several individual doses, e.g. 1 to 10 individual doses, such as 4 to 6 individual doses. Codeine is often administered together with other analgesics, such as paracetamol. The daily dose of tramadol is typically from 50 mg to 400 mg divided into several individual doses, e.g. 1 to 10 individual doses, such as 4 to 6 individual doses. The daily dose of oral morphine is typically from 10 mg to 120 mg divided into several individual doses, e.g. 1 to 10 individual doses, such as 4 to 6 individual doses. In combination with a positive allosteric modulator of the GABA_{B} receptor lower doses of the opiate may be effective.

The compounds according to the present disclosure are given to a subject as such or in combination with one or more other active ingredients, each in its own composition or combined in a single composition, and/or suitable pharmaceutical excipients. The latter group comprises conventionally used excipients and formulation aids, such as fillers, binders, disintegrating agents, lubricants, solvents, gel forming agents, emulsifiers, stabilizers, colorants, and/or preservatives.

The compounds of formula I are formulated into dosage forms using commonly known pharmaceutical manufacturing methods. The dosage forms can be, for example, tablets, capsules, granules, suppositories, emulsions, suspensions, or solutions. Depending on the route of administration and the galenic form, the amount of the active ingredient in the formulation can typically vary between 0.01% and 100% (w/w).

The present disclosure provides compounds for use as a GABA_{B} receptor autoradiography ligand. Compounds for use as a GABA_{B} receptor PET tracer in a mammal, such as a human, are also provided.

A person skilled in the art will appreciate that the embodiments described in the present disclosure can be modified without departing from the inventive concept. A person skilled in the art also understands that the present disclosure is not limited to the particular embodiments disclosed but is intended to also cover modifications of the embodiments that are within the spirit and scope of the present disclosure.

1. A compound of formula I, 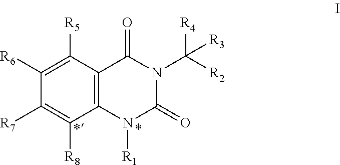
wherein
R₁ is (C₁-C₅)alkyl, (C₂-C₅)alkenyl, (C₂-C₅)alkynyl, (C₄-C₇)cycloalkyl, oxetan-2-yl, oxetan-3-yl, carboxy(C₂-C₅)alkyl, cyano(C₂-C₅)alkyl, aryl(C₂-C₅)alkyl, halohydroxy(C₁-C₅)alkyl, hydroxy(C₁-C₉)alkyl, (C₁-C₃)alkoxy(C₁-C₅)alkyl, methylthio(C₁-C₅)alkyl, methylsulfinyl(C₁-C₅)alkyl, methylsulfonyl(C₁-C₅)alkyl, amino(C₁-C₅)alkyl, ((C₁-C₃)alkylamino)(C₁-C₅)alkyl, (di(C₁-C₃)alkylamino)(C₁-C₅)alkyl, heterocyclyl(C₁-C₅)alkyl, heteroaryl(C₁-C₅)alkyl, (C₁-C₅)alkylcarbonyl(C₁-C₅)alkyl, (C₃-C₆)cycloalkylcarbonyl(C₁-C₅)alkyl, arylcarbonyl(C₁-C₅)alkyl, (C₁-C₃)alkoxycarbonyl(C₂-C₅)alkyl, aminocarbonyl(C₂-C₅)alkyl, ((C₁-C₃)alkylamino)carbonyl(C₂-C₅)alkyl, (di(C₁-C₃)alkylamino)carbonyl(C₂-C₅)alkyl, (N—(C₁-C₃)alkyl-N-methoxyamino)carbonyl(C₂-C₅)alkyl, heterocyclylcarbonyl(C₂-C₅)alkyl, halo(C₁-C₃)alkoxy(C₁-C₅)alkyl, hydroxy(C₁-C₃)alkoxy(C₁-C₅)alkyl, methoxy(C₁-C₃)alkoxy(C₁-C₅)alkyl, or (C₁-C₅)alkylcarbonylhydroxy(C₁-C₅)alkyl, wherein the (C₄-C₇)cycloalkyl, aryl, heterocyclyl, or heteroaryl as such or as part of another group is unsubstituted or substituted with 1 substituent being methyl or hydroxy;
R₂ is phenyl, phenylmethyl, or 2-phenylethyl, wherein the phenyl as such or as part of another group is substituted with 1, 2, or 3 substituent(s) R₉;
R₃ is H, (C₁-C₃)alkyl, phenyl, phenylmethyl, or methoxy(C₁-C₃)alkyl, wherein the phenyl as such or as part of another group is unsubstituted;
or R₂ and R₃ form, together with the carbon atom to which they are attached, cyclopentyl or cyclohexyl, wherein the cyclopentyl or cyclohexyl is substituted with 2 substituents R₁₀;
R₄ is H;
or R₃ and R₄ form, together with the carbon atom to which they are attached, (C₃-C₆)cycloalkyl, wherein the (C₃-C₆)cycloalkyl is unsubstituted;
R₅ is H, halogen, or (C₁-C₅)alkoxy;
R₆ is H, methyl, halogen, hydroxy, (C₁-C₃)alkoxy, halo(C₁-C₃)alkyl, methoxy(C₁-C₃)alkyl, or halo(C₁-C₃)alkoxy;
R₇ is H, (C₁-C₅)alkyl, (C₄-C₇)cycloalkyl, halogen, (C₁-C₃)alkoxy, heterocyclyl, nitro, halo(C₁-C₃)alkyl, methoxy(C₁-C₃)akyl, halo(C₁-C₃)alkoxy, or dimethylamino, wherein the (C₄-C₇)cycloalkyl or heterocyclyl is unsubstituted;
or R₆ and R₇ form, together with the carbon ring atoms to which they are attached, a 5- or 6-membered non-aromatic heterocyclic ring containing 2 ring heteroatoms being O, wherein the heterocyclic ring is unsubstituted;
R₈ is H, halogen, (C₁-C₃)alkoxy, or methoxy(C₁-C₃)alkoxy;
or R₁ and R₈ form together *—CHR₁₁—C(R₁₂)₂—O—*′, wherein * and *′ indicate respective points of attachment;
R₉ is, independently at each occurrence, methyl, cyano, halogen, methoxy, phenyloxy, nitro, phenylmethyl, halomethyl, halomethoxy, or dimethylamino, wherein the phenyl as part of another group is unsubstituted;
or R₉ and R₉ attached to adjacent carbon ring atoms form, together with the carbon ring atoms to which they are attached, a 5- or 6-membered non-aromatic heterocyclic ring containing 1 or 2 ring heteroatom(s) being 0 or a 6-membered aromatic carbocyclic ring, wherein the heterocyclic ring or carbocyclic ring is unsubstituted;
R₁₀ and R₁₀ attached to adjacent carbon ring atoms form, together with the carbon ring atoms to which they are attached, phenyl, wherein the phenyl is unsubstituted or substituted with 1, 2, 3, or 4 substituent(s) being, independently at each occurrence, halogen or methoxy;
R₁₁ is H, (C₁-C₅)alkyl, carboxy, hydroxy(C₁-C₅)alkyl, or (C₁-C₅)alkylcarbonyl; and
R₁₂ is, independently at each occurrence, (C₁-C₅)alkyl, carboxy, hydroxy(C₁-C₅)alkyl, or (C₁-C₅)alkylcarbonyl;
or a pharmaceutically acceptable ester or salt thereof;
with the provisos that
a) when R₁ is (C₂-C₅)alkenyl, R₅, R₆, R₇, and R₈ are not simultaneously H;
b) when R₁ is (di(C₁-C₃)alkylamino)(C₁-C₅)alkyl, R₉ is not methoxy;
c) when R₂ is phenylmethyl, R₉ is not methoxy;
d) the compound is not 3-(4-methoxybenzyl)-1-methylquinazoline-2,4(1H,3H)-dione, 1-methyl-3-(3-methyl-4-nitrobenzyl)-6-(perfluoropropan-2-yl)quinazoline-2,4(1H,3H)-dione, 1-methyl-3-(4-methylbenzyl)quinazoline-2,4(1H,3H)-dione, 3-(4-fluorophenethyl)-1-methyl-7-nitroquinazoline-2,4(1H,3H)-dione, 3-(4-methoxybenzyl)-1-(2-(4-methylpiperazin-1-yl)ethyl)quinazoline-2,4(1H,3H)-dione, 3-(4-methoxybenzyl)-1-(3-(4-methylpiperazin-1-yl)propyl)quinazoline-2,4(1H,3H)-dione, 3-(benzo[d][1,3]dioxol-5-ylmethyl)-1-(2-oxo-2-phenylethyl)quinazoline-2,4(1H,3H)-dione, 1-(3,3-dimethyl-2-oxobutyl)-3-(3-methylphenethyl)quinazoline-2,4(1H,3H)-dione, 3-(4-chlorophenethyl)-1-(furan-2-ylmethyl)quinazoline-2,4(1H,3H)-dione, 3-(4-chlorophenethyl)-1-methylquinazoline-2,4(1H,3H)-dione, 3-(4-fluorobenzyl)-6-iodo-1-methylquinazoline-2,4(1H,3H)-dione, 3-(4-chlorobenzyl)-6-iodo-1-methylquinazoline-2,4(1H,3H)-dione, 3-(3-fluorobenzyl)-6-iodo-1-methylquinazoline-2,4(1H,3H)-dione, 3-(3-chlorobenzyl)-6-iodo-1-methylquinazoline-2,4(1H,3H)-dione, 3-(4-bromobenzyl)-6-iodo-1-methylquinazoline-2,4(1H,3H)-dione, 3-(3,4-difluorobenzyl)-6-iodo-1-methylquinazoline-2,4(1H,3H)-dione, 2-(3-(3,4-dichlorobenzyl)-2,4-dioxo-3,4-dihydroquinazolin-1(2H)-yl)propanoic acid, ethyl 2-(3-(3,4-dichlorobenzyl)-2,4-dioxo-3,4-dihydroquinazolin-1(2H)-yl)propanoate, or 4-((7-nitro-2,4-dioxo-1-propyl-1,2-dihydroquinazolin-3(4H)-yl)methyl)benzonitrile.

2. The compound according to claim 1, wherein R₁₁ is H or (C₁-C₅)alkyl.

3. The compound according to claim 2, wherein R₁₁ is H.

4. The compound according to claim 1, wherein
R₂ is phenyl, wherein the phenyl is substituted with 1 or 2 substituent(s) R₉;
or R₂ and R₃ form, together with the carbon atom to which they are attached, cyclopentyl or cyclohexyl, wherein the cyclopentyl or cyclohexyl is substituted with 2 substituents R₁₀.

5. The compound according to claim 1, wherein R₄ is H.

6. The compound according to claim 1, wherein
R₉ is, independently at each occurrence, cyano, halogen, methoxy, phenyloxy, nitro, halomethyl, halomethoxy, or di ethylamino, wherein the phenyl as part of another group is unsubstituted;
or R₉ and R₉ attached to adjacent carbon ring atoms form, together with the carbon ring atoms to which they are attached, a 5-membered non-aromatic heterocyclic ring containing 1 ring heteroatom being O, wherein the heterocyclic ring is unsubstituted.

7. The compound according to claim 6, wherein R₉ is, independently at each occurrence, halogen, methoxy, or halomethoxy.

8. The compound according to claim 1, wherein
R₇ is H, halogen, (C₁-C₃)alkoxy, or halo(C₁-C₃)alkyl;
or R₆ and R₇ form, together with the carbon ring atoms to which they are attached, a 5- or 6-membered non-aromatic heterocyclic ring containing 2 ring heteroatoms being O, wherein the heterocyclic ring is unsubstituted.

9. The compound according to claim 8, wherein R₇ is halogen or halo(C₁-C₃)alkyl.

10. The compound according to claim 1, wherein
R₁ is (C₁-C₅)alkyl, (C₂-C₅)alkenyl, (C₂-C₅)alkynyl, oxetan-3-yl, carboxy(C₂-C₅)alkyl, cyano(C₂-C₅)alkyl, hydroxy(C₁-C₉)alkyl, (C₁-C₃)alkoxy(C₁-C₅)alkyl, heterocyclyl(C₁-C₅)alkyl, (C₁-C₅)alkylcarbonyl(C₁-C₅)alkyl, (C₁-C₃)alkoxycarbonyl(C₂-C₅)alkyl, am inocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl, wherein the heterocyclyl as part of another group is unsubstituted or substituted with 1 substituent being methyl or hydroxy;
or R₁ and R₈ form together *—CHR₁₁—C(R₁₂)₂—O—*′, wherein * and *′ indicate respective points of attachment.

11. The compound according to claim 10, wherein R₁ is (C₁-C₅)alkyl, oxetan-3-yl, carboxy(C₂-C₅)alkyl, cyano(C₂-C₅)alkyl, hydroxy(C₁-C₉)alkyl, heterocyclyl(C₁-C₅)alkyl, or (C₁-C₅)alkylcarbonyl(C₁-C₅)alkyl, wherein the heterocyclyl as part of another group is unsubstituted or substituted with 1 substituent being methyl or hydroxy;
or R₁ and R₈ form together *—CHR₁₁—C(R₁₂)₂—O—*′, wherein * and *′ indicate respective points of attachment.

12. The compound according to claim 1, wherein
R₃ is H or (C₁-C₃)alkyl;
or R₂ and R₃ form, together with the carbon atom to which they are attached, cyclopentyl or cyclohexyl, wherein the cyclopentyl or cyclohexyl is substituted with 2 substituents R₁₀.

13. The compound according to claim 12, wherein R₃ is H or C₁-C₃)alkyl.

14. The compound according to claim 1, wherein R₆ is H, halogen, (C₁-C₃)alkoxy, halo(C₁-C₃)alkyl, or halo(C₁-C₃)alkoxy.

15. The compound according to claim 14, wherein R₆ is H, halogen, or (C₁-C₃)alkoxy.

16. The compound according to claim 1, wherein R₅ is H or halogen.

17. The compound according to claim 1, wherein
R₈ is H, halogen, or (C₁-C₃)alkoxy;
or R₁ and R₈ form together *—CHR₁₁—C(R₁₂)₂—O—*′, wherein * and *″ indicate respective points of attachment.

18. The compound according to claim 1, wherein R₁ is (C₁-C₅)alkyl, (C₂-C₅)alkenyl, (C₂-C₅)alkynyl, oxetan-3-yl, carboxy(C₂-C₅)alkyl, cyano(C₂-C₅)alkyl, hydroxy(C₁-C₉)alkyl, (C₁-C₃)alkoxy(C₁-C₅)alkyl, heterocyclyl(C₁-C₅)alkyl, (C₁-C₅)akylcarbonyl(C₁-C₅)alkyl, (C₁-C₃)alkoxycarbonyl(C₂-C₅)alkyl, am inocarbonyl(C₂-C₅)alkyl, ((C₁-C₃)alkylamino)carbonyl(C₂-C₅)alkyl, or (di(C₁-C₃)alkylamino)carbonyl(C₂-C₅)alkyl, wherein the heterocyclyl as part of another group is unsubstituted or substituted with 1 substituent being methyl or hydroxy;
R₂ is phenyl, wherein the phenyl is substituted with 1 or 2 substituent(s) R₉;
R₃ is H or (C₁-C₃)alkyl;
or R₂ and R₃ form, together with the carbon atom to which they are attached, cyclopentyl or cyclohexyl, wherein the cyclopentyl or cyclohexyl is substituted with 2 substituents R₁₀;
R₄ is H;
R₅ is H, halogen, or (C₁-C₅)alkoxy;
R₆ is H, halogen, (C₁-C₃)alkoxy, halo(C₁-C₃)alkyl, or halo(C₁-C₃)alkoxy;
R₇ is H, halogen, (C₁-C₃)alkoxy, or halo(C₁-C₃)alkyl;
or R₆ and R₇ form, together with the carbon ring atoms to which they are attached, a 5- or 6-membered non-aromatic heterocyclic ring containing 2 ring heteroatoms being O, wherein the heterocyclic ring is unsubstituted;
R₈ is H, halogen, or (C₁-C₃)alkoxy;
or R₁ and R₈ form together *—CHR₁₁—C(R₁₂)₂—O—*′, wherein * and indicate respective points of attachment;
R₉ is, independently at each occurrence, cyano, halogen, methoxy, phenyloxy, nitro, halomethyl, halomethoxy, or dimethylamino, wherein the phenyl as part of another group is unsubstituted;
or R₉ and R₉ attached to adjacent carbon ring atoms form, together with the carbon ring atoms to which they are attached, a 5-membered non-aromatic heterocyclic ring containing 1 ring heteroatom being O, wherein the heterocyclic ring is unsubstituted; and
R₁₁ is H or (C₁-C₅)alkyl.

19. The compound according to claim 18, wherein
R₁ is (C₁-C₅)alkyl, oxetan-3-yl, carboxy(C₂-C₅)alkyl, cyano(C₂-C₅)alkyl, hydroxy(C₁-C₉)alkyl, heterocyclyl(C₁-C₅)alkyl, or (C₁-C₅)alkylcarbonyl(C₁-C₅)alkyl, wherein the heterocyclyl as part of another group is unsubstituted or substituted with 1 substituent being methyl or hydroxy;
R₂ is phenyl, wherein the phenyl is substituted with 1 or 2 substituent(s) R₉;
R₃ is H or (C₁-C₃)alkyl;
R₄ is H;
R₅ is H or halogen;
R₆ is H, halogen, or (C₁-C₃)alkoxy;
R₇ is halogen or halo(C₁-C₃)alkyl;
R₈ is H, halogen, or (C₁-C₃)alkoxy;
or R₁ and R₈ form together *—CHR₁₁—C(R₁₂)₂—O—*′, wherein * and *′ indicate respective points of attachment;
R₉ is, independently at each occurrence, halogen, methoxy, or halomethoxy; and
R₁₁ is H.

20. The compound according to claim 1, wherein the compound is 3-(4-bromobenzyl)-5,7-dimethoxy-1-methylquinazoline-2,4(1H,3H)-dione, 3-(3,4-dichlorobenzyl)-1-(2-hydroxy-2-methylpropyl)-7-(trifluoromethyl)quinazoline-2,4(1H,3H)-dione, 6,7-difluoro-1-(2-hydroxy-2-methylpropyl)-3-(4-methoxybenzyl)quinazoline-2,4(1H,3H)-dione, 7-chloro-6-fluoro-1-(2-hydroxy-2-methylpropyl)-3-(4-methoxybenzyl)quinazoline-2,4(1H,3H)-dione, 3-(4-bromobenzyl)-6-(difluoromethoxy)-7-fluoro-1-methylquinazoline-2,4(1H,3H)-dione, 3-(4-bromobenzyl)-7-fluoro-6-methoxy-1-methylquinazoline-2,4(1H,3H)-dione, 3-(4-bromobenzyl)-7-fluoro-6-hydroxy-1-methylquinazoline-2,4(1H,3H)-dione, 7-(4-bromobenzyl)-5-methyl-[1,3]dioxolo[4,5-g]quinazoline-6,8(5H,7H)-dione, 3-(4-bromobenzyl)-1-isopropyl-6,7-dimethoxyquinazoline-2,4 (1H,3H)-dione, 3-(4-bromobenzyl)-1-(2-hydroxy-2-methylpropyl)-6,7-dimethoxyquinazoline-2,4(1H,3H)-dione, 3-(4-bromobenzyl)-6,7-difluoro-1-methylquinazoline-2,4(1H,3H)-dione, 1-methyl-3-(1,2,3,4-tetrahydronaphthalen-1-yl)-7-(trifluoromethyl)quinazoline-2,4(1H,3H)-dione, 3-(4-bromobenzyl)-1-methyl-6-(trifluoromethyl)quinazoline-2,4(1H,3H)-dione, dichlorobenzyl)-1-methyl-7-(trifluoromethyl)quinazoline-2,4(1H,3H)-dione, 3-(4-bromobenzyl)-1-methyl-7-(trifluoromethyl)quinazoline-2,4(1H,3H)-dione, 7-fluoro-1-methyl-3-(4-(trifluoromethyl)benzyl)quinazoline-2,4(1H,3H)-dione, 7-fluoro-3-(3-fluoro-4-methoxybenzyl)-1-methylquinazoline-2,4(1H,3H)-dione, 3-(4-bromobenzyl)-7-chloro-1-methylquinazoline-2,4(1H,3H)-dione, 3-(1-(4-bromophenyl)ethyl)-7-fluoro-1-methylquinazoline-2,4(1H,3H)-dione, 3-((4-chlorophenyl)(phenyl)methyl)-1,7-dimethylquinazoline-2,4(1H,3H)-dione, 3-(4-bromobenzyl)-5,8-dimethoxy-1-methylquinazoline-2,4(1H,3H)-dione, 3-(3,4-dichlorobenzyl)-7-fluoro-1-methylquinazoline-2,4(1H,3H)-dione, 7-fluoro-3-(4-methoxybenzyl)-1-methylquinazoline-2,4(1H,3H)-dione, (S)-3-(1-(4-chlorophenyl)ethyl)-7-fluoro-1-methylquinazoline-2,4(1H,3H)-dione, (R)-3-(1-(4-chlorophenyl)ethyl)-6,7-dimethoxy-1-methylquinazoline-2,4(1H,3H)-dione, 3-(4-bromobenzyl)-1-(3,3-dimethyl-2-oxobutyl)-7-fluoroquinazoline-2,4(1H,3H)-dione, 3-(4-bromobenzyl)-7-fluoro-1-(3-oxobutan-2-yl)quinazoline-2,4(1H,3H)-dione, 3-(4-bromobenzyl)-7-fluoro-1-(2-methoxyethyl)quinazoline-2,4(1H,3H)-dione, 3-(4-bromobenzyl)-7-fluoro-1-(2-(2-methoxyethoxy)ethyl)quinazoline-2,4(1H,3H)-dione, 2-(3-(4-bromobenzyl)-7-fluoro-2,4-dioxo-3,4-dihydroquinazolin-1(2H)-yl)-propanenitrile, 3-(4-bromobenzyl)-7-fluoro-1-(3-oxobutyl)quinazoline-2,4(1H,3H)-dione, 3-(4-bromobenzyl)-7-fluoro-1-(2-hydroxypropyl)quinazoline-2,4(1H,3H)-dione, 3-(4-bromobenzyl)-7-fluoro-1-methylquinazoline-2,4(1H,3H)-dione, 3-(3-(4-bromobenzyl)-7-fluoro-2,4-dioxo-3,4-dihydroquinazolin-1(2H)-yl)propano c acid, 3-(3-(4-bromobenzyl)-7-fluoro-2,4-dioxo-3,4-dihydroquinazolin-1(2H)-yl)-propanamide, 3-(3-(4-bromobenzyl)-7-fluoro-2,4-dioxo-3,4-dihydroquinazolin-1(2H)-yl)-N,N-dimethylpropanamide, 2-(3-(4-bromobenzyl)-7-fluoro-2,4-dioxo-3,4-dihydroquinazolin-1(2H)-yl)propanamide, 3-(4-bromobenzyl)-7-fluoro-1-isopropylquinazoline-2,4(1H,3H)-dione, 3-(4-bromobenzyl)-7-fluoro-1-(2-hydroxy-2-methylpropyl)quinazoline-2,4(1H,3H)-dione, 7-fluoro-1-methyl-3-(4-nitrobenzyl)quinazoline-2,4(1H,3H)-dione, 3-(4-chloro-3-phenoxybenzyl)-7-fluoro-1-(2-hydroxy-2-methylpropyl)quinazoline-2,4(1H,3H)-dione, (R)-3-(1-(4-chlorophenyl)ethyl)-7-fluoro-1-methylquinazoline-2,4(1H,3H)-dione, 3-(1-(4-chlorophenyl)cyclopropyl)-7-fluoro-1-methylquinazoline-2,4(1H,3H)-dione, 3-(1-(4-chlorophenyl)-3-methoxypropyl)-7-fluoro-1-methylquinazoline-2,4(1H,3H)-dione, 3-(4-bromobenzyl)-7-chloro-1-(2-hydroxy-2-methylpropyl)quinazoline-2,4(1H,3H)-dione, 3-(4-bromobenzyl)-7-chloro-1-((3-methyloxetan-3-yl)methyl)quinazoline-2,4(1H,3H)-dione, 3-(4-bromobenzyl)-7-chloro-6-fluoro-1-methylquinazoline-2,4(1H,3H)-dione, 3-(4-bromobenzyl)-7-chloro-6-fluoro-1-(2-hydroxy-2-methylpropyl)quinazoline-2,4(1H,3H)-dione, 3-(4-bromobenzyl)-7-chloro-6-fluoro-1-((3-methyloxetan-3-yl)methyl)quinazoline-2,4(1H,3H)-dione, methyl 2-(3-(4-bromobenzyl)-7-chloro-6-fluoro-2,4-dioxo-3,4-dihydroquinazolin-1(2H)-yl)propanoate, 3-(4-bromobenzyl)-7-fluoro-1-neopentylquinazoline-2,4(1H,3H)-dione, 3-(4-bromobenzyl)-7-chloro-6-fluoro-1-(2-methoxy-2-methylpropyl)quinazoline-2,4(1H,3H)-dione, 7-chloro-6-fluoro-3-(4-methoxybenzyl)-1-((3-methyloxetan-3-yl)methyl)quinazoline-2,4(1H,3H)-dione, methyl 2-(7-chloro-6-fluoro-3-(4-methoxybenzyl)-2,4-dioxo-3,4-dihydroquinazolin-1(2H)-yl)propanoate, 7-chloro-6-fluoro-1-(3-hydroxy-3-methylbutan-2-yl)-3-(4-methoxybenzyl)qunazoline-2,4(1H,3H)-dione, (R)-7-chloro-3-(1-(4-chlorophenyl)ethyl)-6-fluoro-1-methylquinazoline-2,4(1H,3H)-dione, (R)-7-chloro-3-(1-(4-chlorophenyl)ethyl)-6-fluoro-1-((3-methyloxetan-3-yl)-methyl)quinazoline-2,4(1H,3H)-dione, 3-(4-bromobenzyl)-7,8-difluoro-1-methylquinazoline-2,4(1H,3H)-dione, 3-(4-bromobenzyl)-6,7,8-trifluoro-1-(2-hydroxy-2-methylpropyl)quinazoline-2,4(1H,3H)-dione, 3-(4-bromobenzyl)-6,7-difluoro-1-(2-hydroxy-2-methylpropyl)quinazoline-2,4(1H,3H)-dione, 3-(4-(difluoromethoxy)benzyl)-6,7-difluoro-1-(2-hydroxy-2-methylpropyl)quinazoline-2,4(1H,3H)-dione, 3-((2,3-dihydrobenzofuran-5-yl)methyl)-6,7-difluoro-1-methylquinazoline-2,4(1H,3H)-dione, 3-(4-bromobenzyl)-1-(but-3-yn-2-yl)-7-fluoroquinazoline-2,4(1H,3H)-dione, 6,7,8-trifluoro-1-(2-hydroxy-2-methylpropyl)-3-(4-methoxybenzyl)quinazoline-2,4(1H,3H)-dione, 6-(4-bromobenzyl)-9,10-difluoro-2,2-dimethyl-2H-[1,4]oxazino[2,3,4-ij]quinazoline-5,7(3H,6H)-dione, 10-chloro-6-(4-chlorobenzyl)-2,2-dimethyl-2H-[1,4]oxazino[2,3,4-ij]quinazoline-5,7(3H,6H)-dione, (R)-3-(4-bomobenzyl)-7-chloro-1-(2-hydroxypropyl)quinazoline-2,4(1H,3H)-dione, 7-chloro-1-((3-methyloxetan-3-yl)methyl)-3-(4-(trifluoromethoxy)benzyl)quinazoline-2,4(1H,3H)-done, 2-(3-(4-bromobenzyl)-7-chloro-6-fluoro-2,4-dioxo-3,4-dihydroquinazolin-1(2H)-yl)-N-methylpropanamide, 6,8-difluoro-1-(2-hydroxy-2-methylpropyl)-3-(4-(trifluoromethoxy)benzyl)quinazoline-2,4(1H,3H)-dione, 3-(4-bromobenzyl)-6,7-difluoro-1-(3-oxobutyl)quinazoline-2,4(1H,3H)-dione, 7-chloro-3-(4-(dimethylamino)benzyl)-1-(2-hydroxy-2-methylpropyl)quinazoline-2,4(1H,3H)-dione, 6-chloro-8-fluoro-1-(2-hydroxy-2-methylpropyl)-3-(4-methoxybenzyl)quinazoline-2,4(1H,3H)-dione, 3-((2,3-dihydrobenzofuran-5-yl)methyl)-6,7-difluoro-1-(2-hydroxy-2-methylpropyl)quinazoline-2,4(1H,3H)-dione, (R)-3-(1-(4-chlorophenyl)ethyl)-1-methylquinazoline-2,4(1H,3H)-dione, 6,8-dichloro-1-(2-hydroxy-2-methylpropyl)-3-(4-methoxybenzyl)quinazoline-2,4(1H,3H)-dione, 6-(4-bromobenzyl)-10-fluoro-2,2-dimethyl-2H-[1,4]oxazino[2,3,4-ij]quinazoline-5,7(3H,6H)-dione, 7-chloro-3-(5-chloro-2,3-dihydro-1H-inden-1-yl)-1-(2-hydroxy-2-methylpropyl)quinazoline-2,4(1H,3H)-dione, 3-(4-bromobenzyl)-6-chloro-8-fluoro-1-(2-hydroxy-2-methylpropyl)quinazoline-2,4(1H,3H)-dione, 6-(4-chlorobenzyl)-10-fluoro-2,2-dimethyl-2H-[1,4]oxazino[2,3,4-ij]quinazoline-5,7(3H,6H)-dione, (R)-7-chloro-3-(4-chlorobenzyl)-8-fluoro-1-(2-hydroxypropyl)quinazoline-2,4(1H,3H)-dione, 10-chloro-6-(4-chlorobenzyl)-2-methyl-5,7-dioxo-3,5,6,7-tetrahydro-2H-[1,4]oxazino[2,3,4-ij]quinazoline-2-carboxylic acid, 6-(4-bromobenzyl)-9-fluoro-10-methoxy-2,2-dimethyl-2H-[1,4]oxazino[2,3,4-ij]quinazoline-5,7(3H,6H)-dione, 7-chloro-3-((2,3-dihydrobenzofuran-5-yl)methyl)-6-fluoro-1-(2-hydroxy-2-methylpropyl)quinazoline-2,4(1H,3H)-dione, 7-chloro-3-(4-chlorobenzyl)-8-fluoro-1-(2-hydroxy-2-methyl-3-oxobutyl)quinazoline-2,4(1H,3H)-dione, 3-(4-bromobenzyl)-1-ethyl-7-fluoroquinazoline-2,4(1H,3H)-dione, 7-chloro-3-(4-chloro-3-fluorobenzyl)-1-((3-methyloxetan-3-yl)methyl)quinazoline-2,4(1H,3H)-dione, 5,7-dichloro-3-(4-chlorobenzyl)-1-(2-hydroxy-2-methylpropyl)quinazoline-2,4(1H,3H)-dione, 7-chloro-3-(4-(difluoromethoxy)benzyl)-8-fluoro-1-(2-hydroxy-2-methylpropyl)quinazoline-2,4(1H,3H)-dione, (S)-3-(4-bromobenzyl)-7-chloro-8-fluoro-1-(2-hydroxypropyl)quinazoline-2,4(1H,3H)-dione, 3-(4-bromo-2-fluorobenzyl)-7-chloro-1-(2-hydroxy-2-methylpropyl)quinazoline-2,4(1H,3H)-dione, 3-(2,4-dichlorobenzyl)-6,7-difluoro-1-(2-hydroxy-2-methylpropyl)quinazoline-2,4(1H,3H)-dione, 9-chloro-6-(4-methoxybenzyl)-2,2-dimethyl-2H-[1,4]oxazino[2,3,4-ij]quinazoline-5,7(3H,6H)-dione, 3-(4-(difluoromethoxy)benzyl)-6,8-difluoro-1-(2-hydroxy-2-methylpropyl)quinazoline-2,4(1H,3H)-dione, 3-(4-bromobenzyl)-7-chloro-1-(2-hydroxy-2-methylpropyl)-8-(2-methoxyethoxy)quinazoline-2,4(1H,3H)-dione, 1-(3 bromo-2-(hydroxymethyl)-2-methylpropyl)-3-(4-bromobenzyl)-7-chloro-6-fluoroquinazoline-2,4(1H,3H)-dione, (S)-7-chloro-3-(1-(4-chlorophenyl)ethyl)-6-fluoro-1-methylquinazoline-2,4(1H,3H)-dione, 7-chloro-3-(4-chlorobenzyl)-8-fluoro-1-methylquinazoline-2,4(1H,3H)-dione, 3-(4-bromobenzyl)-7-chloro-6-fluoro-1-(oxetan-3-yl)quinazoline-2,4(1H,3H)-dione, 7-chloro-3-(4-chlorobenzyl)-8-fluoro-1-(2-hydroxyethyl)quinazoline-2,4(1H,3H)-dione, 10-fluoro-6-(4-methoxybenzyl)-2,2-dimethyl-2H-[1,4]oxazino[2,3,4-ij]quinazoline-5,7(3H,6H)-dione, 3-(4-bromobenzyl)-7-chloro-1-(1-cyclopropyl-1-oxopropan-2-yl)-6-fluoroquinazoline-2,4(1H,3H)-dione, 3-(4-bromobenzyl)-7-chloro-8-fluoro-1-(2-hydroxy-2,3-dimethylbutyl)quinazoline-2,4(1H,3H)-dione, 7-chloro-8-fluoro-1-(2-hydroxy-2,3-dimethylbutyl)-3-(4-methoxybenzyl)quinazoline-2,4(1H,3H)-dione, 7-chloro-3-((2,3-dihydrobenzofuran-5-yl)methyl)-1-(2-hydroxy-2-methylpropyl)quinazoline-2,4(1H,3H)-dione, (S)-7-chloro-6-fluoro-1-(2-hydroxypropyl)-3-(4-methoxybenzyl)quinazoline-2,4(1H,3H)-dione, (S)-7-chloro-3-(1-(4-chlorophenyl)ethyl)-6-fluoro-1-((3-methyloxetan-3-yl)methyl)quinazoline-2,4(1H,3H)-dione, 10-chloro-6-(4-methoxybenzyl)-2,2-dimethyl-2H-[1,4]oxazino[2,3,4-ij]quinazoline-5,7(3H,6H)-dione, 6,8-difluoro-1-(2-hydroxy-2-methylpropyl)-3-(4-methoxybenzyl)quinazoline-2,4(1H,3H)-dione, 7-chloro-3-(4-(difluoromethoxy)benzyl)-1-(2-hydroxy-2-methylpropyl)quinazoline-2,4(1H,3H)-dione, (R)-7-chloro-3-(1-(4-chlorophenyl)ethyl)-1-methylquinazoline-2,4(1H,3H)-dione, 7-chloro-3-(3-chloro-4-methoxybenzyl)-1-(2-hydroxy-2-methylpropyl)quinazoline-2,4(1H,3H)-dione, 9,10-difluoro-6-(4-methoxybenzyl)-2,2-dimethyl-2H-[1,4]oxazino[2,3,4-ij]quinazoline-5,7(3H,6H)-dione, 7-chloro-3-(4-chloro-3-fluorobenzyl)-1-(2-hydroxy-2-methylpropyl)quinazoline-2,4(1H,3H)-dione, 7-chloro-3-(4-(difluoromethoxy)benzyl)-1-((3-methyloxetan-3-yl)methyl)quinazoline-2,4(1H,3H)-dione, (R)-7-chloro-3-(1-(4-chlorophenyl)ethyl)-1-(2-hydroxy-2-methylpropyl)quinazoline-2,4(1H,3H)-dione, (R)-7-chloro-6-fluoro-1-(2-hydroxypropyl)-3-(4-methoxybenzyl)quinazoline-2,4(1H,3H)-dione, 7-chloro-8-fluoro-1-(2-hydroxy-2-methylpropyl)-3-(4-(trifluoromethoxy)benzyl)quinazoline-2,4(1H,3H)-dione, (R)-7-chloro-6-fluoro-1-(2-hydroxy-2-methylpropyl)-3-(1-(4-methoxyphenyl)ethyl)quinazoline-2,4(1H,3H)-dione, 10-chloro-2-isopropyl-6-(4-methoxybenzyl)-2-methyl-2H-[1,4]oxazino[2,3,4-ij]quinazoline-5,7(3H,6H)-dione, (S)-3-(4-bromobenzyl)-7-chloro-1-(2-hydroxypropyl)quinazoline-2,4(1H,3H)-dione, 3-(4-bromobenzyl)-7-chloro-8-fluoro-1-(2-hydroxy-2-methylpropyl)quinazoline-2,4(1H,3H)-dione, (R)-7-chloro-3-(4-chlorobenzyl)-1-(2-hydroxypropyl)quinazoline-2,4(1H,3H)-dione, (S)-7-chloro-3-(1-(4-chlorophenyl)ethyl)-6-fluoro-1-(2-hydroxy-2-methylpropyl)quinazoline-2,4(1H,3H)-dione, (S)-7-chloro-3-(4-chlorobenzyl)-1-(2-hydroxypropyl)quinazoline-2,4(1H,3H)-dione, 6-(4-bromobenzyl)-10-chloro-2,2-dimethyl-2H-[1,4]oxazino[2,3,4-ij]quinazoline-5,7(3H,6H)-dione, 7-chloro-6-fluoro-1-(2-hydroxy-2,3-dimethylbutyl)-3-(4-methoxybenzyl)quinazoline-2,4(1H,3H)-dione, 9-fluoro-10-methoxy-6-(4-methoxybenzyl)-2,2-dimethyl-2H-[1,4]oxazino[2,3,4-ij]quinazoline-5,7(3H,6H)-dione, (Z)-7-chloro-3-(4-chlorobenzyl)-1-(prop-1-en-1-yl)quinazoline-2,4(1H,3H)-dione, 6-chloro-3-(4-chlorobenzyl)-8-fluoro-1-(2-hydroxy-2-methylpropyl)quinazoline-2,4(1H,3H)-dione, 7-chloro-3-(4-chlorobenzyl)-1-(2-hydroxy-2-methylpropyl)quinazoline-2,4(1H,3H)-dione, 3-(4-bromobenzyl)-6,8-difluoro-1-(2-hydroxy-2-methylpropyl)quinazoline-2,4(1H,3H)-dione, 7-chloro-8-fluoro-1-(2-hydroxy-2-methylpropyl)-3-(4-methoxybenzyl)quinazoline-2,4(1H,3H)-dione, 3-(benzo[d][1,3]dioxol-5-ylmethyl)-7-chloro-1-(2-hydroxy-2-methylpropyl)quinazoline-2,4(1H,3H)-dione, 3-(4-bromobenzyl)-6,7,8-trifluoro-1-methylquinazoline-2,4(1H,3H)-dione, (R)-7-chloro-3-(1-(4-chlorophenyl)ethyl)-1-((3-methyloxetan-3-yl)methyl)quinazoline-2,4(1H,3H)-dione, 3-(benzo[d][1,3]dioxol-5-ylmethyl)-7-chloro-1-((3-methyloxetan-3-yl)methyl)quinazoline-2,4(1H,3H)-dione, (R)-3-(4-bromobenzyl)-7-chloro-8-fluoro-1-(2-hydroxypropyl)quinazoline-2,4(1H,3H)-dione, 6-(4-bromobenzyl)-9-fluoro-2,2-dimethyl-2H-[1,4]oxazino[2,3,4-ij]quinazoline-5,7(3H,6H)-dione, 7-chloro-3-(3-chloro-4-methoxybenzyl)-1-((3-methyloxetan-3-yl)methyl)quinazoline-2,4(1H,3H)-dione, 6-(4-bromobenzyl)-9-chloro-2,2-dimethyl-2H-[1,4]oxazino[2,3,4-ij]quinazoline-5,7(3H,6H)-dione, (R)-3-(1-(4-chlorophenyl)ethyl)-1-methyl-7-nitroquinazoline-2,4(1H,3H)-dione, 7-chloro-3-(4-chlorobenzyl)-1-(2,3-dihydroxy-2-methylbutyl)-8-fluoroquinazoline-2,4(1H,3H)-dione, 9-fluoro-6-(4-methoxybenzyl)-2,2-dimethyl-2H-[1,4]oxazino[2,3,4-ij]quinazoline-5,7(3H,6H)-dione, 3-(4-bromo-2-fluorobenzyl)-7-chloro-1-((3-methyloxetan-3-yl)methyl)quinazoline-2,4(1H,3H)-dione, 2-acetyl-10-chloro-6-(4-chlorobenzyl)-2-methyl-2H-[1,4]oxazino[2,3,4-ij]quinazoline-5,7(3H,6H)-dione, diastereomer 1 of 10-chloro-6-(4-chlorobenzyl)-2-(1-hydroxyethyl)-2-methyl-2H-[1,4]oxazino[2,3,4-ij]quinazoline-5,7(3H,6H)-dione, or diastereomer 2 of 10-chloro-6(4-chlorobenzyl)-2-(1-hydroxyethyl)-2-methyl-2H-[1,4]oxazino[2,3,4-ij]quinazoline-5,7(3H,6H)-dione.

21. The compound according to claim 1, wherein the compound is in isotopically unlabeled form.

22. The compound according to claim 1, wherein the compound is in isotopically labeled form.

23. The compound according to claim 22, wherein the compound is ³H labeled.

24. The compound according to claim 22, wherein the compound is ¹¹C labeled.

25. The compound according to claim 22, wherein the compound is ¹⁸F labeled.

26-32. (canceled)

33. A method for the treatment of a disease where a positive allosteric modulator of the GABA_{B} receptor is indicated to be useful comprising administering to a mammal in need of such treatment an effective amount of at least one compound according to claim 1.

34. The method according to claim 33, wherein the disease is essential tremor, Parkinsonian tremor, levodopa-induced dyskinesia, Parkinsonian motor symptoms, Parkinsonian non-motor symptoms, spasticity related to multiple sclerosis, spasticity related to amyotrophic lateral sclerosis, spasticity related to spinal cord injury, spasticity related to cerebral injury, dystonia, chronic pain, addiction, anxiety, epilepsy, autism, fragile X syndrome, amyotrophic lateral sclerosis, post-traumatic stress disorder, depression, insomnia, narcolepsy, Alzheimer's disease, dementia, Charcot Marie Tooth 1A neuropathy, overactive bladder, gastro-esophageal reflux disease, inflammatory bowel disease, or chronic tinnitus.

35. A pharmaceutical composition comprising as an active ingredient at least one compound according to claim 1 and a pharmaceutically acceptable excipient.

36. The pharmaceutical composition according to claim 35, wherein the composition further comprises one or more other active ingredients.

37. A method of detecting a GABA_{B} receptor, comprising applying a compound according to claim 23, wherein the compound is an autoradiography ligand for the receptor.

38. A method of detecting a GABA_{B} receptor, comprising applying a compound according to claim 24, wherein the compound is a PET tracer in a mammal for the receptor.

39. A method of detecting a GABA_{B} receptor, comprising applying a compound according to claim 25, wherein the compound is a PET tracer in a mammal for the receptor.
